(19) United States
(12) Patent Application Publication
(10) Pub. No.: US 2024/0280490 A1
(43) Pub. Date: Aug. 22, 2024

(54) SYSTEMS AND METHODS FOR SIMULTANEOUS NEAR-INFRARED LIGHT AND VISIBLE LIGHT IMAGING

(71) Applicant: Blaze Bioscience, Inc., Seattle, WA (US)

(72) Inventors: Jeffrey Perry, Seattle, WA (US); David Kittle, Seattle, WA (US); Julia Novak, Seattle, WA (US); Teri Dee Koller, Seattle, WA (US); Dennis Miller, Seattle, WA (US)

(21) Appl. No.: 18/012,621

(22) PCT Filed: Jun. 25, 2021
(86) PCT No.: PCT/US2021/039177

§ 371 Date: Dec. 22, 2022

Related U.S. Application Data
(60) Provisional application No. 63/044,303, filed on Jun. 25, 2020.

(51) Int. Cl. G01N 21/64; A61B 1/00; A61B 1/04; A61B 1/06; G01J 1/42; G02B 21/00; G02B 21/36; G02B 27/00; G06T 5/50; H04N 7/18; H04N 23/11; H04N 23/50; H04N 23/55; H04N 23/56

(52) U.S. Cl. CPC G01N 21/6456; A61B 1/0005; A61B 1/043; A61B 1/0638; G01J 1/4257; G01N 21/6428; G02B 21/0032; G02B 21/0076; G02B 21/36; G06T 5/50; H04N 23/11; H04N 23/55; H04N 23/555; H04N 23/56; G01N 2021/6439; G01N 2201/0636; G02B 21/0012; G02B 27/0018; G06T 2207/10048; G06T 2207/10064; G06T 2207/20221; G06T 2207/20224; G06T 2207/30096

(57) ABSTRACT

Disclosed herein are imaging systems and methods for simultaneous near-infrared light or infrared and visible light imaging of a sample comprising: a detector to form a fluorescence image of the sample and a visible image of the sample; a light source configured to emit near infrared or infrared light to induce fluorescence from the sample; and a plurality of optics arranged to direct the near infrared or infrared light toward the sample and form the fluorescence image of the sample and the visible light image of the sample on the detector, including methods to reduce ghosting, shadowing and motion artifacts.

### BACKGROUND

Fluorescence, including the use of fluorescent molecules tagged to other structures such as cells, nanoparticles, small molecules and peptides are useful for organ, organ substructure, tissue and potentially cellular identification in medical imaging. For example, fluorescent dyes emit in visible (e.g., blue, green, yellow, red) and/or infrared, ultraviolet, or near infrared wavelengths. Although visible light fluorescence is generally detected by naked eye, detection of infrared (IR) light and near infrared (NIR) light typically requires additional instrumentation for viewing. Infrared and near infrared are a beneficial wavelength range for medical imaging. The benefits of infrared, near infrared and long wavelength visible light are generally related to increased penetration depth, absence of significant intrinsic fluorescence, low absorption by blood (hemoglobin) or water. In medical applications imaging systems capable of imaging both visible and infrared or near infrared images simultaneously are advantageous, so that the surgeons are able to operate in tissues, for example, tagged with infrared fluorophore and do so seamlessly without having to switch between imaging modalities.

Moreover, to image fluorescence from tissue, the imaging system will need to have ability and sensitivity to detect small amount of fluorescence, for example, from a fluorescent dye that adheres to or has been absorbed by the tissue. Traditionally, infrared fluorescence systems have used sensitive sensors to detect infrared light, while using traditional halogen light sources for exciting the dye. Although such prior instrumentation produces images from such infrared light sources, sensitivity is often less than ideal due to inefficient halogen or broadband lighting as well as lower energy light sources surrounding excitation wavelengths, leading to inefficient and non-optimal infrared images. Although lasers have been used to achieve higher absorption and as a result increase fluorescence of the infrared or near infrared dyes, the images generated are often less than ideal in at least some instances.

### SUMMARY

The present disclosure describes systems and methods for fluorescence and visible light imaging which solve at least some of the problems in prior systems. The systems and methods disclosed herein are capable of generating and combining visible and fluorescent images with imperceptible delays, and providing high fluorescence sensitivity, decreasing disruption to the surgical workflow, and improving ease of use with an operating microscope. The systems and methods relate to stand-alone imaging devices or in combination with a surgical instrument, such as an operating microscope, exoscope, or a surgical robot. In some embodiments, excitation light is directed to the sample coaxially with fluorescence light received from the sample, which decreases shadows and helps to ensure that tissue tagged with a fluorescent marker is properly identified. In some embodiments, the viewing axis of the visible light imaging optics is coaxial with the excitation light and fluorescent light axes in order to improve registration of the fluorescence image and the visible image over a range of distances extending between the optics and the imaged tissue. The systems and methods comprise a beam splitter to transmit visible light toward eye pieces and reflect fluorescent light toward a detector, in which a portion of the visible light is reflected toward a detector to generate a visible image with the reflected light. The amount of reflected visible light much less than the transmitted light, in order for the user such as a surgeon to readily view the tissue through the eyepieces while the visible light image is being generated with the detector for combination with the fluorescence image. In some embodiments, the excitation light and the fluorescent light comprise light having wavelengths longer than about 650 nm in order to provide an increased penetration depth into the tissue as compared with light used to generate the visible image.

In some embodiments, the system comprises one or more illumination sources, one or more of which is a narrowband laser/s with or without visible light illumination controlled by the instrumentation, a set of optics to illuminate the target, a set of optics to collect the generated fluorescence, filters to remove the laser illumination light, and one or more sensors to capture the fluorescence and visible light.

In one aspect, disclosed herein is an imaging system for imaging a sample, comprising: a detector to form a fluorescence image of the sample and a visible image of the sample; a light source configured to emit excitation light to induce fluorescence from the sample; and a plurality of optics arranged to direct the excitation light toward the sample and receive fluorescent light and visible light from the sample in order to form the fluorescence image of the sample and the visible light image of the sample on the detector, wherein the excitation light is directed to the sample substantially coaxially with fluorescence light received from the sample in order to decrease shadows. In some embodiments, the excitation light comprises infrared light and optionally wherein the infrared light comprises near infrared light. In some embodiments, the plurality of optics comprises a dichroic shortpass beam splitter to direct infrared light and visible light to the detector. In some embodiments, the detector comprises a plurality of detectors and optionally wherein the visible image comprises a color image. In some embodiments, the plurality of detectors comprises a first detector to generate a color image and a second detector to generate the infrared image. In some embodiments, the imaging system herein further comprises an ASIC or a processor configured with instructions to generate a composite image of the sample, the composite image comprising the fluorescence image overlaid with the visible image from the sample. In some embodiments, the light source comprises: a laser or narrow-band light source; an optical light guide coupled to the laser or narrow-band light source; a collimating lens into which the light guide ends; a laser clean-up filter; a dielectric mirror; a diffuser; a hole; or a combination thereof. In some embodiments, the narrow-band light source generates light with a wavelength in the range of 700 nm to 800 nm, 650 to 900 nm, or 700 nm to 900 nm. In some embodiments, the laser generates light with a wavelength in the range of 650 nm to 4000 nm, or 700 nm to 3000 nm. In some embodiments, the wavelength comprises 750 nm to 950 nm, 760 nm to 825 nm, 775 nm to 795 nm, 780 nm to 795 nm, 785 nm to 795 nm, 780 nm to 790 nm, 785 nm to 792 nm, 790 nm to 795 nm, or 785 nm. In some embodiments, the collimating lens is configured to collimate the transmitted light from the optical light guide, thereby generating collimated light. In some embodiments, the optical light guide is a fiber optic cable, liquid or solid/plastic light guide, liquid light guide, waveguide, or any other light guide that is capable of transmitting infrared or near infrared light. In some embodiments, the laser clean-up filter is configured to reduce bandwidth of the infrared light. In some embodiments, the dielectric mirror is configured to reflect the infrared light so that incident light and reflected light of the dielectric mirror are of an intersection angle of about 90 degrees. In some embodiments, the dielectric mirror is configured to reflect the infrared light so that incident light and reflected light of the dielectric mirror are of an intersection angle of about 60 to about 120 degrees. In some embodiments, the diffuser is configured to diffuse the infrared light at one or more calculated angles. In some embodiments, the one or more calculate angles are within a range from 30 to 150 degrees. In some embodiments, the hole is configured to let pass at least part of the infrared light. The system of any one of the preceding claims, wherein excitation by the infrared light is substantially coaxial to the fluorescence or visible light collected from the sample. In some embodiments, the hole is in a near-infrared mirror. In some embodiments, the hole is shaped and sized to allow evenly distributed illumination of the sample within a field of view of a microscope. In some embodiments, the plurality of optics comprises a dichroic shortpass beam splitter, wherein the dichroic shortpass beam splitter is configured to let pass light with wavelength of no greater than 700 nm with 90% to 95% efficiency at one or more specified angle of incidence. In some embodiments, the shortpass filter 8 only allows a wavelength of about 400 nm to about 800 nm to pass through. In some embodiments, visible light is directed from a microscope, endoscope, exoscope, surgical robot, or operating room lighting external to the imaging system. In some embodiments, the plurality of optics further comprises a secondary dichroic shortpass beam splitter. In some embodiments, the imaging system herein further comprises a dichroic longpass beam splitter. In some embodiments, the infrared light is delivered to the sample along an infrared optical path and the fluorescent light received from the sample is received along a fluorescence optical path and wherein the fluorescence optical path overlaps with the infrared optical path at a beam splitter. In some embodiments, the infrared optical path and the fluorescence optical path are substantially coaxial. In some embodiments, substantially coaxial comprises an intersection angle of two optical paths to be less than 20 degrees, 15 degrees, 10 degrees, 5 degrees, 2 degrees, or 1 degree.

In another aspect, disclosed herein is a method for imaging a sample, comprising: emitting, by a light source, infrared or near infrared light to induce fluorescence from a sample; directing, by a plurality of optics, the infrared or near infrared light to the sample; receiving, by the plurality of optics, the fluorescence from the sample at a detector, wherein the infrared or near infrared light is directed to the sample substantially coaxially with fluorescence light received from the sample in order to decrease shadows; and forming a fluorescence image of the sample and a visible light image of the sample on the detector. In some embodiments, the method herein comprising using the imaging system disclosed herein. In some embodiments, the sample is an organ, organ substructure, tissue or cell. In some embodiments, the method of imaging an organ, organ substructure, tissue or cell, comprises imaging the organ, organ substructure, tissue or cell with an imaging system herein. In some embodiments, the method further comprises detecting a cancer or diseased region, tissue, structure or cell. In some embodiments, the method further comprises performing surgery on the subject. In some embodiments, the method further comprises treating the cancer. In some embodiments, the method further comprises removing the cancer or the diseased region, tissue, structure or cell of the subject. In some embodiments, the method further comprises imaging the cancer or diseased region, tissue, structure, or cell of the subject after surgical removal. In some embodiments, the detecting is performed using fluorescence imaging. In some embodiments, the fluorescence imaging detects a detectable agent, the detectable agent comprising a dye, a fluorophore, a fluorescent biotin compound, a luminescent compound, or a chemiluminescent compound.

In another aspect, as disclosed herein is a method of treating or detecting in a subject in need thereof the method comprising administering a companion diagnostic, therapeutic agent, or imaging agent, wherein the companion diagnostic or imaging agent detected by the systems and methods described herein. In another embodiment, the method of administering a companion diagnostic comprises any one of the various methods of using the systems described herein. In another embodiment, the diagnostic or imaging agent comprises a chemical agent, a radiolabel agent, radiosensitizing agent, fluorophore, an imaging agent, a photosensitizing agent, a diagnostic agent, a protein, a peptide, a nanoparticle or a small molecule. In another embodiment, the system incorporates radiology or fluorescence, including the X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), surgical instrument, operating microscope, confocal microscope, fluorescence scope, exoscope, or a surgical robot. In another embodiment, the systems and methods are used to detect a therapeutic agent or to assess the agent's safety and physiologic effect. In yet another embodiment, the safety and physiologic effect detected by the systems and methods is the agent's bioavailability, uptake, concentration, presence, distribution and clearance, metabolism, pharmacokinetics, localization, blood concentration, tissue concentration, ratio, measurement of concentrations in blood and/or tissues, assessing therapeutic window, range and optimization.

In another embodiment, method of the disclosure is combined with or integrated into surgical microscope, confocal microscope, fluorescence scope, exoscope, endoscope, or a surgical robot. In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica ARveo system, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., Retina Vue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPALI NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

Another aspect provided herein is an imaging system for imaging a sample, comprising: a detector configured to form a fluorescence image of the sample and form a visible image of the sample; a light source configured to emit an excitation light to induce fluorescence off the sample; and a plurality of optics arranged to: direct the excitation light toward the sample; and direct a fluorescent light and a visible light from the sample to the detector; wherein the excitation light and the fluorescence light are directed substantially coaxially.

In some embodiments, the excitation light comprises infrared light. In some embodiments, the infrared light comprises near infrared light. In some embodiments, the plurality of optics comprises a dichroic shortpass beam splitter to direct the infrared light and the visible light to the detector. In some embodiments, the detector comprises a plurality of detectors, and wherein the visible image comprises a color image. In some embodiments, the plurality of detectors comprises a first detector to generate a color image and a second detector to generate the infrared image. In some embodiments, the system further comprises: a laser; an optical light guide coupled to the laser or narrow-band light source; a collimating lens into which the light guide ends; a laser clean-up filter; a dielectric mirror; a diffuser; a hole; or a combination thereof. In some embodiments, the light source emits a wavelength absorbed by a fluorophore. In some embodiments, the light source is a narrow-band light source.

In some embodiments, the narrow-band light source generates light with a wavelength of 700 nm to 800 nm, 650 to 900 nm, 700 nm to 900 nm, 340 nm to 400 nm, 360 to 420 nm, 380 nm to 440 nm, or 400 nm to 450 nm. In some embodiments, the narrow-band light source generates light with a wavelength of about 300 nm to about 900 nm. In some embodiments, the narrow-band light source generates light with a wavelength of about 300 nm to about 350 nm, about 300 nm to about 400 nm, about 300 nm to about 450 nm, about 300 nm to about 500 nm, about 300 nm to about 550 nm, about 300 nm to about 600 nm, about 300 nm to about 650 nm, about 300 nm to about 700 nm, about 300 nm to about 750 nm, about 300 nm to about 800 nm, about 300 nm to about 900 nm, about 350 nm to about 400 nm, about 350 nm to about 450 nm, about 350 nm to about 500 nm, about 350 nm to about 550 nm, about 350 nm to about 600 nm, about 350 nm to about 650 nm, about 350 nm to about 700 nm, about 350 nm to about 750 nm, about 350 nm to about 800 nm, about 350 nm to about 900 nm, about 400 nm to about 450 nm, about 400 nm to about 500 nm, about 400 nm to about 550 nm, about 400 nm to about 600 nm, about 400 nm to about 650 nm, about 400 nm to about 700 nm, about 400 nm to about 750 nm, about 400 nm to about 800 nm, about 400 nm to about 900 nm, about 450 nm to about 500 nm, about 450 nm to about 550 nm, about 450 nm to about 600 nm, about 450 nm to about 650 nm, about 450 nm to about 700 nm, about 450 nm to about 750 nm, about 450 nm to about 800 nm, about 450 nm to about 900 nm, about 500 nm to about 550 nm, about 500 nm to about 600 nm, about 500 nm to about 650 nm, about 500 nm to about 700 nm, about 500 nm to about 750 nm, about 500 nm to about 800 nm, about 500 nm to about 900 nm, about 550 nm to about 600 nm, about 550 nm to about 650 nm, about 550 nm to about 700 nm, about 550 nm to about 750 nm, about 550 nm to about 800 nm, about 550 nm to about 900 nm, about 600 nm to about 650 nm, about 600 nm to about 700 nm, about 600 nm to about 750 nm, about 600 nm to about 800 nm, about 600 nm to about 900 nm, about 650 nm to about 700 nm, about 650 nm to about 750 nm, about 650 nm to about 800 nm, about 650 nm to about 900 nm, about 700 nm to about 750 nm, about 700 nm to about 800 nm, about 700 nm to about 900 nm, about 750 nm to about 800 nm, about 750 nm to about 900 nm, or about 800 nm to about 900 nm. In some embodiments, the narrow-band light source generates light with a wavelength of about 300 nm, about 350 nm, about 400 nm, about 450 nm, about 500 nm, about 550 nm, about 600 nm, about 650 nm, about 700 nm, about 750 nm, about 800 nm, or about 900 nm. In some embodiments, the narrow-band light source generates light with a wavelength of at least about 300 nm, about 350 nm, about 400 nm, about 450 nm, about 500 nm, about 550 nm, about 600 nm, about 650 nm, about 700 nm, about 750 nm, or about 800 nm. In some embodiments, the narrow-band light source generates light with a wavelength of at most about 350 nm, about 400 nm, about 450 nm, about 500 nm, about 550 nm, about 600 nm, about 650 nm, about 700 nm, about 750 nm, about 800 nm, or about 900 nm.

In some embodiments, the narrow-band light source emits light with a frequency visible by an NIR camera, and wherein the system further comprises a lens coupled to the optical light guide.

In some embodiments, the laser generates light with a wavelength of 650 nm to 4000 nm, 700 nm to 3000 nm, or 340 nm to 450 nm. In some embodiments, the laser generates light with a wavelength of 750 nm to 950 nm, 760 nm 825 nm, 775 nm to 795 nm, 780 nm to 795 nm, 785 nm to 795 nm, 780 nm to 790 nm, 785 nm to 792 nm, or 790 nm to 795. In some embodiments, the laser generates light with a wavelength of about 300 nm to about 1,000 nm. In some embodiments, the laser generates light with a wavelength of about 300 nm to about 350 nm, about 300 nm to about 400 nm, about 300 nm to about 450 nm, about 300 nm to about 500 nm, about 300 nm to about 550 nm, about 300 nm to about 600 nm, about 300 nm to about 650 nm, about 300 nm to about 700 nm, about 300 nm to about 800 nm, about 300 nm to about 900 nm, about 300 nm to about 1,000 nm, about 350 nm to about 400 nm, about 350 nm to about 450 nm, about 350 nm to about 500 nm, about 350 nm to about 550 nm, about 350 nm to about 600 nm, about 350 nm to about 650 nm, about 350 nm to about 700 nm, about 350 nm to about 800 nm, about 350 nm to about 900 nm, about 350 nm to about 1,000 nm, about 400 nm to about 450 nm, about 400 nm to about 500 nm, about 400 nm to about 550 nm, about 400 nm to about 600 nm, about 400 nm to about 650 nm, about 400 nm to about 700 nm, about 400 nm to about 800 nm, about 400 nm to about 900 nm, about 400 nm to about 1,000 nm, about 450 nm to about 500 nm, about 450 nm to about 550 nm, about 450 nm to about 600 nm, about 450 nm to about 650 nm, about 450 nm to about 700 nm, about 450 nm to about 800 nm, about 450 nm to about 900 nm, about 450 nm to about 1,000 nm, about 500 nm to about 550 nm, about 500 nm to about 600 nm, about 500 nm to about 650 nm, about 500 nm to about 700 nm, about 500 nm to about 800 nm, about 500 nm to about 900 nm, about 500 nm to about 1,000 nm, about 550 nm to about 600 nm, about 550 nm to about 650 nm, about 550 nm to about 700 nm, about 550 nm to about 800 nm, about 550 nm to about 900 nm, about 550 nm to about 1,000 nm, about 600 nm to about 650 nm, about 600 nm to about 700 nm, about 600 nm to about 800 nm, about 600 nm to about 900 nm, about 600 nm to about 1,000 nm, about 650 nm to about 700 nm, about 650 nm to about 800 nm, about 650 nm to about 900 nm, about 650 nm to about 1,000 nm, about 700 nm to about 800 nm, about 700 nm to about 900 nm, about 700 nm to about 1,000 nm, about 800 nm to about 900 nm, about 800 nm to about 1,000 nm, or about 900 nm to about 1,000 nm. In some embodiments, the laser generates light with a wavelength of about 300 nm, about 350 nm, about 400 nm, about 450 nm, about 500 nm, about 550 nm, about 600 nm, about 650 nm, about 700 nm, about 800 nm, about 900 nm, or about 1,000 nm. In some embodiments, the laser generates light with a wavelength of at least about 300 nm, about 350 nm, about 400 nm, about 450 nm, about 500 nm, about 550 nm, about 600 nm, about 650 nm, about 700 nm, about 800 nm, or about 900 nm. In some embodiments, the laser generates light with a wavelength of at most about 350 nm, about 400 nm, about 450 nm, about 500 nm, about 550 nm, about 600 nm, about 650 nm, about 700 nm, about 800 nm, about 900 nm, or about 1,000 nm.

In some embodiments, the collimating lens is configured to collimate the excitation light, the fluorescent light, and the visible light. In some embodiments, the optical light guide is a fiber optic cable, a solid light guide, a plastic light guide, a liquid light guide, a waveguide, or any combination thereof. In some embodiments, wherein the laser clean-up filter is configured to reduce bandwidth of the excitation light. In some embodiments, the light source comprises: a broadband light source; an optical light guide coupled to the broadband light source; or both. In some embodiments, the broadband light source comprises one or more LEDs, a Xenon bulb, a halogen bulb, one or more or lasers, sunlight, fluorescent lighting or a combination thereof. In some embodiments, the broadband light source emits a visible wavelength, a wavelength absorbed by a fluorophore, or both. In some embodiments, the broadband light source emits light with a frequency visible by an NIR camera, and wherein the system further comprises a lens coupled to the optical light guide. In some embodiments, the system comprises a plurality of light sources, wherein the system further comprises one or more of the following to combine the plurality of light sources into a single coaxial path: an optical attenuator comprising a dichroic filter, a dichroic mirror, a shutter, or any combination thereof; a filter at each light source a clean-up filter for a wavelength range of the excitation light; a shortpass filter for a wavelength range of the excitation light; an optical light guide; or an illumination optic. In some embodiments, the system further comprises: a laser clean-up filter; a shortpass (SP) mirror; a longpass (LP) mirror; a dielectric mirror; a diffuser; a hole; or a combination thereof.

In some embodiments, the dielectric mirror is configured to reflect the excitation light such that excitation light and the reflected excitation light have an intersection angle of about 60 degrees to about 120 degrees. In some embodiments, the dielectric mirror is configured to reflect the excitation light such that excitation light and the reflected excitation light have an intersection angle of about 60 degrees to about 75 degrees, about 60 degrees to about 80 degrees, about 60 degrees to about 85 degrees, about 60 degrees to about 90 degrees, about 60 degrees to about 95 degrees, about 60 degrees to about 100 degrees, about 60 degrees to about 105 degrees, about 60 degrees to about 110 degrees, about 60 degrees to about 115 degrees, about 60 degrees to about 120 degrees, about 75 degrees to about 80 degrees, about 75 degrees to about 85 degrees, about 75 degrees to about 90 degrees, about 75 degrees to about 95 degrees, about 75 degrees to about 100 degrees, about 75 degrees to about 105 degrees, about 75 degrees to about 110 degrees, about 75 degrees to about 115 degrees, about 75 degrees to about 120 degrees, about 80 degrees to about 85 degrees, about 80 degrees to about 90 degrees, about 80 degrees to about 95 degrees, about 80 degrees to about 100 degrees, about 80 degrees to about 105 degrees, about 80 degrees to about 110 degrees, about 80 degrees to about 115 degrees, about 80 degrees to about 120 degrees, about 85 degrees to about 90 degrees, about 85 degrees to about 95 degrees, about 85 degrees to about 100 degrees, about 85 degrees to about 105 degrees, about 85 degrees to about 110 degrees, about 85 degrees to about 115 degrees, about 85 degrees to about 120 degrees, about 90 degrees to about 95 degrees, about 90 degrees to about 100 degrees, about 90 degrees to about 105 degrees, about 90 degrees to about 110 degrees, about 90 degrees to about 115 degrees, about 90 degrees to about 120 degrees, about 95 degrees to about 100 degrees, about 95 degrees to about 105 degrees, about 95 degrees to about 110 degrees, about 95 degrees to about 115 degrees, about 95 degrees to about 120 degrees, about 100 degrees to about 105 degrees, about 100 degrees to about 110 degrees, about 100 degrees to about 115 degrees, about 100 degrees to about 120 degrees, about 105 degrees to about 110 degrees, about 105 degrees to about 115 degrees, about 105 degrees to about 120 degrees, about 110 degrees to about 115 degrees, about 110 degrees to about 120 degrees, or about 115 degrees to about 120 degrees. In some embodiments, the dielectric mirror is configured to reflect the excitation light such that excitation light and the reflected excitation light have an intersection angle of about 60 degrees, about 75 degrees, about 80 degrees, about 85 degrees, about 90 degrees, about 95 degrees, about 100 degrees, about 105 degrees, about 110 degrees, about 115 degrees, or about 120 degrees. In some embodiments, the dielectric mirror is configured to reflect the excitation light such that excitation light and the reflected excitation light have an intersection angle of at least about 60 degrees, about 75 degrees, about 80 degrees, about 85 degrees, about 90 degrees, about 95 degrees, about 100 degrees, about 105 degrees, about 110 degrees, or about 115 degrees. In some embodiments, the dielectric mirror is configured to reflect the excitation light such that excitation light and the reflected excitation light have an intersection angle of at most about 75 degrees, about 80 degrees, about 85 degrees, about 90 degrees, about 95 degrees, about 100 degrees, about 105 degrees, about 110 degrees, about 115 degrees, or about 120 degrees.

In some embodiments, the diffuser is configured to diffuse the excitation light. In some embodiments, the hole is configured to let pass at least part of the excitation light. In some embodiments, the hole is in a near-infrared mirror. In some embodiments, the hole has a shape, and a size, and wherein at least one of the shape of the hole and the size of the hole are configured to allow an even distribution illumination of the sample within a field of view of a microscope. In some embodiments, excitation light comprises blue or ultraviolet light.

In some embodiments, the blue or ultraviolet light comprises a light having a wavelength of 10 nm to about 460 nm, about 10 nm to about 400 nm, or about 400 nm to about 460 nm. In some embodiments, the blue or ultraviolet light comprises a light having a wavelength of about 10 nm to about 500 nm. In some embodiments, the blue or ultraviolet light comprises a light having a wavelength of about 10 nm to about 50 nm, about 10 nm to about 100 nm, about 10 nm to about 150 nm, about 10 nm to about 200 nm, about 10 nm to about 250 nm, about 10 nm to about 300 nm, about 10 nm to about 350 nm, about 10 nm to about 400 nm, about 10 nm to about 450 nm, about 10 nm to about 500 nm, about 50 nm to about 100 nm, about 50 nm to about 150 nm, about 50 nm to about 200 nm, about 50 nm to about 250 nm, about 50 nm to about 300 nm, about 50 nm to about 350 nm, about 50 nm to about 400 nm, about 50 nm to about 450 nm, about 50 nm to about 500 nm, about 100 nm to about 150 nm, about 100 nm to about 200 nm, about 100 nm to about 250 nm, about 100 nm to about 300 nm, about 100 nm to about 350 nm, about 100 nm to about 400 nm, about 100 nm to about 450 nm, about 100 nm to about 500 nm, about 150 nm to about 200 nm, about 150 nm to about 250 nm, about 150 nm to about 300 nm, about 150 nm to about 350 nm, about 150 nm to about 400 nm, about 150 nm to about 450 nm, about 150 nm to about 500 nm, about 200 nm to about 250 nm, about 200 nm to about 300 nm, about 200 nm to about 350 nm, about 200 nm to about 400 nm, about 200 nm to about 450 nm, about 200 nm to about 500 nm, about 250 nm to about 300 nm, about 250 nm to about 350 nm, about 250 nm to about 400 nm, about 250 nm to about 450 nm, about 250 nm to about 500 nm, about 300 nm to about 350 nm, about 300 nm to about 400 nm, about 300 nm to about 450 nm, about 300 nm to about 500 nm, about 350 nm to about 400 nm, about 350 nm to about 450 nm, about 350 nm to about 500 nm, about 400 nm to about 450 nm, about 400 nm to about 500 nm, or about 450 nm to about 500 nm. In some embodiments, the blue or ultraviolet light comprises a light having a wavelength of about 10 nm, about 50 nm, about 100 nm, about 150 nm, about 200 nm, about 250 nm, about 300 nm, about 350 nm, about 400 nm, about 450 nm, or about 500 nm. In some embodiments, the blue or ultraviolet light comprises a light having a wavelength of at least about 10 nm, about 50 nm, about 100 nm, about 150 nm, about 200 nm, about 250 nm, about 300 nm, about 350 nm, about 400 nm, or about 450 nm. In some embodiments, the blue or ultraviolet light comprises a light having a wavelength of at most about 50 nm, about 100 nm, about 150 nm, about 200 nm, about 250 nm, about 300 nm, about 350 nm, about 400 nm, about 450 nm, or about 500 nm.

In some embodiments, the plurality of optics comprises a dichroic shortpass beam splitter, wherein the dichroic shortpass beam splitter is configured to let pass light with a wavelength of at most 700 nm with 90% to 95% efficiency at one or more specified angles of incidence.

In some embodiments, the one or more specific angles is within a range from 30 to 150 degrees. In some embodiments, the one or more specific angles is about 30 degrees to about 150 degrees. In some embodiments, the one or more specific angles is about 30 degrees to about 40 degrees, about 30 degrees to about 50 degrees, about 30 degrees to about 60 degrees, about 30 degrees to about 70 degrees, about 30 degrees to about 80 degrees, about 30 degrees to about 90 degrees, about 30 degrees to about 100 degrees, about 30 degrees to about 110 degrees, about 30 degrees to about 120 degrees, about 30 degrees to about 130 degrees, about 30 degrees to about 150 degrees, about 40 degrees to about 50 degrees, about 40 degrees to about 60 degrees, about 40 degrees to about 70 degrees, about 40 degrees to about 80 degrees, about 40 degrees to about 90 degrees, about 40 degrees to about 100 degrees, about 40 degrees to about 110 degrees, about 40 degrees to about 120 degrees, about 40 degrees to about 130 degrees, about 40 degrees to about 150 degrees, about 50 degrees to about 60 degrees, about 50 degrees to about 70 degrees, about 50 degrees to about 80 degrees, about 50 degrees to about 90 degrees, about 50 degrees to about 100 degrees, about 50 degrees to about 110 degrees, about 50 degrees to about 120 degrees, about 50 degrees to about 130 degrees, about 50 degrees to about 150 degrees, about 60 degrees to about 70 degrees, about 60 degrees to about 80 degrees, about 60 degrees to about 90 degrees, about 60 degrees to about 100 degrees, about 60 degrees to about 110 degrees, about 60 degrees to about 120 degrees, about 60 degrees to about 130 degrees, about 60 degrees to about 150 degrees, about 70 degrees to about 80 degrees, about 70 degrees to about 90 degrees, about 70 degrees to about 100 degrees, about 70 degrees to about 110 degrees, about 70 degrees to about 120 degrees, about 70 degrees to about 130 degrees, about 70 degrees to about 150 degrees, about 80 degrees to about 90 degrees, about 80 degrees to about 100 degrees, about 80 degrees to about 110 degrees, about 80 degrees to about 120 degrees, about 80 degrees to about 130 degrees, about 80 degrees to about 150 degrees, about 90 degrees to about 100 degrees, about 90 degrees to about 110 degrees, about 90 degrees to about 120 degrees, about 90 degrees to about 130 degrees, about 90 degrees to about 150 degrees, about 100 degrees to about 110 degrees, about 100 degrees to about 120 degrees, about 100 degrees to about 130 degrees, about 100 degrees to about 150 degrees, about 110 degrees to about 120 degrees, about 110 degrees to about 130 degrees, about 110 degrees to about 150 degrees, about 120 degrees to about 130 degrees, about 120 degrees to about 150 degrees, or about 130 degrees to about 150 degrees. In some embodiments, the one or more specific angles is about 30 degrees, about 40 degrees, about 50 degrees, about 60 degrees, about 70 degrees, about 80 degrees, about 90 degrees, about 100 degrees, about 110 degrees, about 120 degrees, about 130 degrees, or about 150 degrees. In some embodiments, the one or more specific angles is at least about 30 degrees, about 40 degrees, about 50 degrees, about 60 degrees, about 70 degrees, about 80 degrees, about 90 degrees, about 100 degrees, about 110 degrees, about 120 degrees, or about 130 degrees. In some embodiments, the one or more specific angles is at most about 40 degrees, about 50 degrees, about 60 degrees, about 70 degrees, about 80 degrees, about 90 degrees, about 100 degrees, about 110 degrees, about 120 degrees, about 130 degrees, or about 150 degrees.

In some embodiments, the visible light is directed from a microscope, an endoscope, an exoscope, a surgical robot, or an operating room lighting external to the imaging system. In some embodiments, the system further comprises a locking key configured to securely lock the imaging head onto the microscope. In some embodiments, the plurality of optics further comprises a secondary dichroic shortpass beam splitter. In some embodiments, the system further comprises a dichroic longpass beam splitter. In some embodiments, the excitation light and the fluorescence light substantially overlap at the beam splitter. In some embodiments, substantially coaxial comprises an intersection angle of two optical paths to be less than 20 degrees, 15 degrees, 10 degrees, 5 degrees, 2 degrees, or 1 degree. In some embodiments, the system further comprises a physical attenuator configured to block an ambient light from one, two or more of the detector, the light source, and the plurality of optics. In some embodiments, the physical attenuator comprises a shield, a hood, a sleeve, a light shroud, or a baffle. In some embodiments, the system further comprises an Application Specific Integrated Circuit (ASIC) or a processor, wherein at least one of the ASIC and the processor is configured with instructions to generate a composite image of the sample, the composite image comprising the fluorescence image overlaid with the visible image.

Another aspect provided herein is a method for imaging a sample, comprising: emitting, by a light source, infrared or near infrared light to induce fluorescence from a sample; directing, by a plurality of optics, the infrared or near infrared light to the sample; receiving, by the plurality of optics, the fluorescence from the sample at a detector, wherein the infrared or near infrared light is directed to the sample substantially coaxially with fluorescence light received from the sample in order to decrease shadows; and forming a fluorescence image of the sample and a visible light image of the sample on the detector. In some embodiments, the method is performed using the systems herein. In some embodiments, the sample is an organ, an organ substructure, a tissue, or a cell.

Another aspect provided herein is a method of imaging an organ, organ substructure, tissue or cell, the method comprising: imaging the organ, organ substructure, tissue or cell with the system herein. In some embodiments, the method further comprises detecting a cancer or diseased region, tissue, structure or cell. In some embodiments, the method further comprises performing surgery on the subject. In some embodiments, the surgery comprises removing the cancer or the diseased region, tissue, structure or cell of the subject. In some embodiments, the method further comprises imaging the cancer or diseased region, tissue, structure, or cell of the subject after surgical removal. In some embodiments, the imaging or detecting is performed using fluorescence imaging. In some embodiments, the fluorescence imaging detects a detectable agent, the detectable agent comprising a dye, a fluorophore, a fluorescent biotin compound, a luminescent compound, or a chemiluminescent compound. In some embodiments, the detectable agent absorbs a wavelength between about 200 mm to about 900 mm. In some embodiments, the detectable agent comprises DyLight-680, DyLight-750, VivoTag-750, DyLight-800, IRDye-800, VivoTag-680, Cy5.5, or an indocyanine green (ICG) and any derivative of the foregoing; fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), 7-aminocoumarin, a dialkylaminocoumarin reactive dye, 6,8-difluoro-7-hydroxycoumarin fluorophore, a hydroxycoumarin derivative, an alkoxycoumarin derivatives, a succinimidyl ester, a pyrene succinimidyl ester, a pyridyloxazole derivative, an aminonaphthalene-based dyes, dansyl chlorides, a dapoxyl dye, Dapoxyl sulfonyl chloride, amine-reactive Dapoxyl succinimidyl ester, carboxylic acid-reactive Dapoxyl (2-aminoethyl)sulfonamide), a bimane dye, bimane mercaptoacetic acid, an NBD dye, a QsY 35, or any combination thereof. In some embodiments, the method further comprises treating the cancer.

Another aspect provided herein is a method of treating or diagnostic detecting comprising administering at least one of a companion diagnostic agent, therapeutic agent, or a companion imaging agent, and detecting at least one such agent by the systems herein.

Another aspect provided herein is a method of treating or diagnostic detecting comprising administering at least one of a companion diagnostic agent, a photosensitizing agent, therapeutic agent, or a companion imaging agent, and detecting at least one such agent by the methods herein. In some embodiments, at least one of the agents comprises a chemical agent, a radiolabel agent, radiosensitizing agent, a photosensitizing agent, fluorophore, therapeutic agent, a protein, a peptide, a nanoparticle a small molecule, or any combination thereof. In some embodiments, the system or method further comprises radiology or fluorescence using one or more of: an X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), microscope, confocal microscope, fluorescence scope, exoscope, surgical robot, surgical instrument, or any combination thereof. In some embodiments, the system or method further measures fluorescence using one or more microscope, confocal microscope, fluorescence scope, exoscope, surgical robot, surgical instrument, or any combination thereof. In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica ARveo, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., Retina Vue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPALI NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

In some embodiments, the method is configured to: detect, image or assess a therapeutic agent; detect, image or assess a safety or a physiologic effect of the companion diagnostic agent; detect, image or assess a safety or a physiologic effect of the therapeutic agent; detect, image or assess a safety or a physiologic effect of the companion imaging agent; or any combination thereof. In some embodiments, the agent's safety or physiologic effect is bioavailability, uptake, concentration, presence, distribution and clearance, metabolism, pharmacokinetics, localization, blood concentration, tissue concentration, ratio, measurement of concentrations in blood or tissues, therapeutic window, range and optimization, or any combination thereof.

Another aspect provided herein is a method of treating or detecting in a subject in need thereof the method comprising administering a companion diagnostic agent, a photosensitizing agent, therapeutic agent or imaging agent, wherein such agent is detected by a systems or methods herein. In some embodiments, the agent comprises a chemical agent, a radiolabel agent, radiosensitizing agent, a photosensitizing agent, fluorophore, therapeutic agent, an imaging agent, a diagnostic agent, a protein, a peptide, a nanoparticle or a small molecule. In some embodiments, the system or method further incorporates radiology or fluorescence, including X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), surgical instrument, operating microscope, confocal microscope, fluorescence scope, exoscope, or a surgical robot, or a combination thereof. In some embodiments, the systems and methods are used to detect a therapeutic agent or to assess the agent's safety or physiologic effect, or both. In some embodiments, the agent's safety or physiologic effect is bioavailability, uptake, concentration, presence, distribution and clearance, metabolism, pharmacokinetics, localization, blood concentration, tissue concentration, ratio, measurement of concentrations in blood or tissues, therapeutic window, range and optimization, or any combination thereof. In some embodiments, the method is combined with or integrated into a surgical microscope, confocal microscope, fluorescence scope, exoscope, endoscope, or a surgical robot.

In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica ARveo, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, Retina Vue system (e.g., Retina Vue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPALI NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

One aspect provided herein is a imaging system for imaging an emission light emitted by a sample comprising a fluorophore, the system comprising: a laser emitting an excitation light; an excitation diffuser that diffuses the excitation light; a visible channel to receive and direct a visible light to the sample; an optical device directing the diffused excitation light to the sample and allowing the emission light and a reflected visible light to pass therethrough to an imaging assembly; and the imaging assembly comprising: a first notch filter; a longpass filter; a lens; a second notch filter; and an image sensor configured to detect both the emission light and the reflected visible light from the sample and configured to generate image frames based on the emission light and the reflected visible light.

In some embodiments, the emission light and the reflected visible light are directed from the sample through the notch beam splitter, the first notch filter, the longpass filter, the lens, and the second notch filter. In some embodiments, the emission light and the reflected visible light are directed from the sample and sequentially through the notch beam splitter, the first notch filter, the longpass filter, the lens, and the second notch filter.

In some embodiments, the excitation light has a wavelength of about 775 nm to about 792 nm. In some embodiments, the excitation light has a wavelength of about 775 nm to about 776 nm, about 775 nm to about 777 nm, about 775 nm to about 778 nm, about 775 nm to about 779 nm, about 775 nm to about 780 nm, about 775 nm to about 782 nm, about 775 nm to about 784 nm, about 775 nm to about 786 nm, about 775 nm to about 790 nm, about 775 nm to about 792 nm, about 775 nm to about 792 nm, about 776 nm to about 777 nm, about 776 nm to about 778 nm, about 776 nm to about 779 nm, about 776 nm to about 780 nm, about 776 nm to about 782 nm, about 776 nm to about 784 nm, about 776 nm to about 786 nm, about 776 nm to about 790 nm, about 776 nm to about 792 nm, about 776 nm to about 792 nm, about 777 nm to about 778 nm, about 777 nm to about 779 nm, about 777 nm to about 780 nm, about 777 nm to about 782 nm, about 777 nm to about 784 nm, about 777 nm to about 786 nm, about 777 nm to about 790 nm, about 777 nm to about 792 nm, about 777 nm to about 792 nm, about 778 nm to about 779 nm, about 778 nm to about 780 nm, about 778 nm to about 782 nm, about 778 nm to about 784 nm, about 778 nm to about 786 nm, about 778 nm to about 790 nm, about 778 nm to about 792 nm, about 778 nm to about 792 nm, about 779 nm to about 780 nm, about 779 nm to about 782 nm, about 779 nm to about 784 nm, about 779 nm to about 786 nm, about 779 nm to about 790 nm, about 779 nm to about 792 nm, about 779 nm to about 792 nm, about 780 nm to about 782 nm, about 780 nm to about 784 nm, about 780 nm to about 786 nm, about 780 nm to about 790 nm, about 780 nm to about 792 nm, about 780 nm to about 792 nm, about 782 nm to about 784 nm, about 782 nm to about 786 nm, about 782 nm to about 790 nm, about 782 nm to about 792 nm, about 782 nm to about 792 nm, about 784 nm to about 786 nm, about 784 nm to about 790 nm, about 784 nm to about 792 nm, about 784 nm to about 792 nm, about 786 nm to about 790 nm, about 786 nm to about 792 nm, about 786 nm to about 792 nm, about 790 nm to about 792 nm, about 790 nm to about 792 nm, or about 792 nm to about 792 nm. In some embodiments, the excitation light has a wavelength of about 775 nm, about 776 nm, about 777 nm, about 778 nm, about 779 nm, about 780 nm, about 782 nm, about 784 nm, about 786 nm, about 790 nm, about 792 nm, or about 792 nm. In some embodiments, the excitation light has a wavelength of at least about 775 nm, about 776 nm, about 777 nm, about 778 nm, about 779 nm, about 780 nm, about 782 nm, about 784 nm, about 786 nm, about 790 nm, or about 792 nm. In some embodiments, the excitation light has a wavelength of at most about 776 nm, about 777 nm, about 778 nm, about 779 nm, about 780 nm, about 782 nm, about 784 nm, about 786 nm, about 790 nm, about 792 nm, or about 792 nm.

In some embodiments, the visible light has a wavelength of about 400 nm to about 950 nm. In some embodiments, the visible light has a wavelength of about 400 nm to about 450 nm, about 400 nm to about 500 nm, about 400 nm to about 550 nm, about 400 nm to about 600 nm, about 400 nm to about 650 nm, about 400 nm to about 700 nm, about 400 nm to about 750 nm, about 400 nm to about 800 nm, about 400 nm to about 850 nm, about 400 nm to about 900 nm, about 400 nm to about 950 nm, about 450 nm to about 500 nm, about 450 nm to about 550 nm, about 450 nm to about 600 nm, about 450 nm to about 650 nm, about 450 nm to about 700 nm, about 450 nm to about 750 nm, about 450 nm to about 800 nm, about 450 nm to about 850 nm, about 450 nm to about 900 nm, about 450 nm to about 950 nm, about 500 nm to about 550 nm, about 500 nm to about 600 nm, about 500 nm to about 650 nm, about 500 nm to about 700 nm, about 500 nm to about 750 nm, about 500 nm to about 800 nm, about 500 nm to about 850 nm, about 500 nm to about 900 nm, about 500 nm to about 950 nm, about 550 nm to about 600 nm, about 550 nm to about 650 nm, about 550 nm to about 700 nm, about 550 nm to about 750 nm, about 550 nm to about 800 nm, about 550 nm to about 850 nm, about 550 nm to about 900 nm, about 550 nm to about 950 nm, about 600 nm to about 650 nm, about 600 nm to about 700 nm, about 600 nm to about 750 nm, about 600 nm to about 800 nm, about 600 nm to about 850 nm, about 600 nm to about 900 nm, about 600 nm to about 950 nm, about 650 nm to about 700 nm, about 650 nm to about 750 nm, about 650 nm to about 800 nm, about 650 nm to about 850 nm, about 650 nm to about 900 nm, about 650 nm to about 950 nm, about 700 nm to about 750 nm, about 700 nm to about 800 nm, about 700 nm to about 850 nm, about 700 nm to about 900 nm, about 700 nm to about 950 nm, about 750 nm to about 800 nm, about 750 nm to about 850 nm, about 750 nm to about 900 nm, about 750 nm to about 950 nm, about 800 nm to about 850 nm, about 800 nm to about 900 nm, about 800 nm to about 950 nm, about 850 nm to about 900 nm, about 850 nm to about 950 nm, or about 900 nm to about 950 nm. In some embodiments, the visible light has a wavelength of about 400 nm, about 450 nm, about 500 nm, about 550 nm, about 600 nm, about 650 nm, about 700 nm, about 750 nm, about 800 nm, about 850 nm, about 900 nm, or about 950 nm. In some embodiments, the visible light has a wavelength of about at least about 400 nm, about 450 nm, about 500 nm, about 550 nm, about 600 nm, about 650 nm, about 700 nm, about 750 nm, about 800 nm, about 850 nm, or about 900 nm. In some embodiments, the visible light has a wavelength of about at most about 450 nm, about 500 nm, about 550 nm, about 600 nm, about 650 nm, about 700 nm, about 750 nm, about 800 nm, about 850 nm, about 900 nm, or about 950 nm.

In some embodiments, the visible light has a wavelength of about 800 nm to about 950 nm. In some embodiments, the visible light has a wavelength of about 800 nm to about 825 nm, about 800 nm to about 850 nm, about 800 nm to about 875 nm, about 800 nm to about 900 nm, about 800 nm to about 925 nm, about 800 nm to about 950 nm, about 825 nm to about 850 nm, about 825 nm to about 875 nm, about 825 nm to about 900 nm, about 825 nm to about 925 nm, about 825 nm to about 950 nm, about 850 nm to about 875 nm, about 850 nm to about 900 nm, about 850 nm to about 925 nm, about 850 nm to about 950 nm, about 875 nm to about 900 nm, about 875 nm to about 925 nm, about 875 nm to about 950 nm, about 900 nm to about 925 nm, about 900 nm to about 950 nm, or about 925 nm to about 950 nm. In some embodiments, the visible light has a wavelength of about 800 nm, about 825 nm, about 850 nm, about 875 nm, about 900 nm, about 925 nm, or about 950 nm. In some embodiments, the visible light has a wavelength of at least about 800 nm, about 825 nm, about 850 nm, about 875 nm, about 900 nm, or about 925 nm. In some embodiments, the visible light has a wavelength of at most about 825 nm, about 850 nm, about 875 nm, about 900 nm, about 925 nm, or about 950 nm.

In some embodiments, the excitation diffuser is a circular excitation diffuser. In some embodiments, the excitation diffuser is a rectangular excitation diffuser.

In some embodiments, the circular excitation diffuser has a diffusion angle of about 4 degrees to about 25 degrees. In some embodiments, the circular excitation diffuser has a diffusion angle of about 4 degrees to about 6 degrees, about 4 degrees to about 8 degrees, about 4 degrees to about 10 degrees, about 4 degrees to about 12 degrees, about 4 degrees to about 14 degrees, about 4 degrees to about 16 degrees, about 4 degrees to about 18 degrees, about 4 degrees to about 20 degrees, about 4 degrees to about 22 degrees, about 4 degrees to about 25 degrees, about 6 degrees to about 8 degrees, about 6 degrees to about 10 degrees, about 6 degrees to about 12 degrees, about 6 degrees to about 14 degrees, about 6 degrees to about 16 degrees, about 6 degrees to about 18 degrees, about 6 degrees to about 20 degrees, about 6 degrees to about 22 degrees, about 6 degrees to about 25 degrees, about 8 degrees to about 10 degrees, about 8 degrees to about 12 degrees, about 8 degrees to about 14 degrees, about 8 degrees to about 16 degrees, about 8 degrees to about 18 degrees, about 8 degrees to about 20 degrees, about 8 degrees to about 22 degrees, about 8 degrees to about 25 degrees, about 10 degrees to about 12 degrees, about 10 degrees to about 14 degrees, about 10 degrees to about 16 degrees, about 10 degrees to about 18 degrees, about 10 degrees to about 20 degrees, about 10 degrees to about 22 degrees, about 10 degrees to about 25 degrees, about 12 degrees to about 14 degrees, about 12 degrees to about 16 degrees, about 12 degrees to about 18 degrees, about 12 degrees to about 20 degrees, about 12 degrees to about 22 degrees, about 12 degrees to about 25 degrees, about 14 degrees to about 16 degrees, about 14 degrees to about 18 degrees, about 14 degrees to about 20 degrees, about 14 degrees to about 22 degrees, about 14 degrees to about 25 degrees, about 16 degrees to about 18 degrees, about 16 degrees to about 20 degrees, about 16 degrees to about 22 degrees, about 16 degrees to about 25 degrees, about 18 degrees to about 20 degrees, about 18 degrees to about 22 degrees, about 18 degrees to about 25 degrees, about 20 degrees to about 22 degrees, about 20 degrees to about 25 degrees, or about 22 degrees to about 25 degrees. In some embodiments, the circular excitation diffuser has a diffusion angle of about 4 degrees, about 6 degrees, about 8 degrees, about 10 degrees, about 12 degrees, about 14 degrees, about 16 degrees, about 18 degrees, about 20 degrees, about 22 degrees, or about 25 degrees. In some embodiments, the circular excitation diffuser has a diffusion angle of at least about 4 degrees, about 6 degrees, about 8 degrees, about 10 degrees, about 12 degrees, about 14 degrees, about 16 degrees, about 18 degrees, about 20 degrees, or about 22 degrees. In some embodiments, the circular excitation diffuser has a diffusion angle of at most about 6 degrees, about 8 degrees, about 10 degrees, about 12 degrees, about 14 degrees, about 16 degrees, about 18 degrees, about 20 degrees, about 22 degrees, or about 25 degrees.

In some embodiments, the rectangular excitation diffuser has a first diffusion angle and a second diffusion angle perpendicular to the first diffusion angle. In some embodiments, the first diffusion angle, the second diffusion angle, or both are about 4 degrees to about 25 degrees. In some embodiments, the first diffusion angle, the second diffusion angle, or both are about 4 degrees to about 6 degrees, about 4 degrees to about 8 degrees, about 4 degrees to about 10 degrees, about 4 degrees to about 12 degrees, about 4 degrees to about 14 degrees, about 4 degrees to about 16 degrees, about 4 degrees to about 18 degrees, about 4 degrees to about 20 degrees, about 4 degrees to about 22 degrees, about 4 degrees to about 25 degrees, about 6 degrees to about 8 degrees, about 6 degrees to about 10 degrees, about 6 degrees to about 12 degrees, about 6 degrees to about 14 degrees, about 6 degrees to about 16 degrees, about 6 degrees to about 18 degrees, about 6 degrees to about 20 degrees, about 6 degrees to about 22 degrees, about 6 degrees to about 25 degrees, about 8 degrees to about 10 degrees, about 8 degrees to about 12 degrees, about 8 degrees to about 14 degrees, about 8 degrees to about 16 degrees, about 8 degrees to about 18 degrees, about 8 degrees to about 20 degrees, about 8 degrees to about 22 degrees, about 8 degrees to about 25 degrees, about 10 degrees to about 12 degrees, about 10 degrees to about 14 degrees, about 10 degrees to about 16 degrees, about 10 degrees to about 18 degrees, about 10 degrees to about 20 degrees, about 10 degrees to about 22 degrees, about 10 degrees to about 25 degrees, about 12 degrees to about 14 degrees, about 12 degrees to about 16 degrees, about 12 degrees to about 18 degrees, about 12 degrees to about 20 degrees, about 12 degrees to about 22 degrees, about 12 degrees to about 25 degrees, about 14 degrees to about 16 degrees, about 14 degrees to about 18 degrees, about 14 degrees to about 20 degrees, about 14 degrees to about 22 degrees, about 14 degrees to about 25 degrees, about 16 degrees to about 18 degrees, about 16 degrees to about 20 degrees, about 16 degrees to about 22 degrees, about 16 degrees to about 25 degrees, about 18 degrees to about 20 degrees, about 18 degrees to about 22 degrees, about 18 degrees to about 25 degrees, about 20 degrees to about 22 degrees, about 20 degrees to about 25 degrees, or about 22 degrees to about 25 degrees. In some embodiments, the first diffusion angle, the second diffusion angle, or both are about 4 degrees, about 6 degrees, about 8 degrees, about 10 degrees, about 12 degrees, about 14 degrees, about 16 degrees, about 18 degrees, about 20 degrees, about 22 degrees, or about 25 degrees. In some embodiments, the first diffusion angle, the second diffusion angle, or both are at least about 4 degrees, about 6 degrees, about 8 degrees, about 10 degrees, about 12 degrees, about 14 degrees, about 16 degrees, about 18 degrees, about 20 degrees, or about 22 degrees. In some embodiments, the first diffusion angle, the second diffusion angle, or both are at most about 6 degrees, about 8 degrees, about 10 degrees, about 12 degrees, about 14 degrees, about 16 degrees, about 18 degrees, about 20 degrees, about 22 degrees, or about 25 degrees. In some embodiments, the first diffusion angle is about 14 degrees, and wherein the second diffusion angle is about 8 degrees. In some embodiments, the optical device is a hot mirror, a dichroic mirror, a shortpass filter, or any combination thereof. In some embodiments, the hot mirror filters out the wavelength of the NIR light from the visible light. In some embodiments, the optical device directs the diffused excitation light to the sample in a first direction and allows the emission light and a reflected visible light to pass therethrough in a second direction opposite the first direction. In some embodiments, at least one of the first notch filter and the second notch filter block the excitation light from passing therethrough.

In some embodiments, at least one of the first notch filter and the second notch filter block light with a wavelength of about 775 nm to about 795 nm. In some embodiments, at least one of the first notch filter and the second notch filter block light with a wavelength of about 775 nm to about 780 nm, about 775 nm to about 785 nm, about 775 nm to about 790 nm, about 775 nm to about 795 nm, about 780 nm to about 785 nm, about 780 nm to about 790 nm, about 780 nm to about 795 nm, about 785 nm to about 790 nm, about 785 nm to about 795 nm, or about 790 nm to about 795 nm. In some embodiments, at least one of the first notch filter and the second notch filter block light with a wavelength of about 775 nm, about 780 nm, about 785 nm, about 790 nm, or about 795 nm. In some embodiments, at least one of the first notch filter and the second notch filter block light with a wavelength of at least about 775 nm, about 780 nm, about 785 nm, or about 790 nm. In some embodiments, at least one of the first notch filter and the second notch filter block light with a wavelength of at most about 780 nm, about 785 nm, about 790 nm, or about 795 nm.

In some embodiments, the imaging assembly further comprises a polarizer. In some embodiments, the emission light and the reflected visible light are directed through the long pass-filter, the polarizer, and the lens. In some embodiments, the emission light and the reflected visible light are directed sequentially through the long pass-filter, the polarizer and the lens. In some embodiments, the system further comprises a white light that emits the visible light. In some embodiments, the system further comprises a shortpass dichroic mirror between the imaging assembly and the sample and between the excitation diffuser and the sample.

In some embodiments, the shortpass dichroic mirror transmits wavelengths of about 400 nm to about 800 nm. In some embodiments, the shortpass dichroic mirror transmits wavelengths of about 400 nm to about 450 nm, about 400 nm to about 500 nm, about 400 nm to about 550 nm, about 400 nm to about 600 nm, about 400 nm to about 650 nm, about 400 nm to about 700 nm, about 400 nm to about 750 nm, about 400 nm to about 800 nm, about 450 nm to about 500 nm, about 450 nm to about 550 nm, about 450 nm to about 600 nm, about 450 nm to about 650 nm, about 450 nm to about 700 nm, about 450 nm to about 750 nm, about 450 nm to about 800 nm, about 500 nm to about 550 nm, about 500 nm to about 600 nm, about 500 nm to about 650 nm, about 500 nm to about 700 nm, about 500 nm to about 750 nm, about 500 nm to about 800 nm, about 550 nm to about 600 nm, about 550 nm to about 650 nm, about 550 nm to about 700 nm, about 550 nm to about 750 nm, about 550 nm to about 800 nm, about 600 nm to about 650 nm, about 600 nm to about 700 nm, about 600 nm to about 750 nm, about 600 nm to about 800 nm, about 650 nm to about 700 nm, about 650 nm to about 750 nm, about 650 nm to about 800 nm, about 700 nm to about 750 nm, about 700 nm to about 800 nm, or about 750 nm to about 800 nm. In some embodiments, the shortpass dichroic mirror transmits wavelengths of about 400 nm, about 450 nm, about 500 nm, about 550 nm, about 600 nm, about 650 nm, about 700 nm, about 750 nm, or about 800 nm. In some embodiments, the shortpass dichroic mirror transmits wavelengths of at least about 400 nm, about 450 nm, about 500 nm, about 550 nm, about 600 nm, about 650 nm, about 700 nm, or about 750 nm. In some embodiments, the shortpass dichroic mirror transmits wavelengths of at most about 450 nm, about 500 nm, about 550 nm, about 600 nm, about 650 nm, about 700 nm, about 750 nm, or about 800 nm. In some embodiments, the shortpass dichroic filter reflects wavelengths greater than about 720 nm, 725 nm, 730 nm, 735 nm, 740 nm, 750 nm, 755 nm, 760 nm, 770 nm, 780 nm, 800 nm, or more including increments therein. In some embodiments, the system further comprises a bottom window between the shortpass dichroic mirror and the sample. In some embodiments, the system further comprises a front window between the notch filter and the sample. In some embodiments, the excitation light is an infrared or a near-infrared excitation light. In some embodiments, the long pass filter comprises a visible light attenuator. In some embodiments, the visible light attenuator transmits near infrared wavelengths. In some embodiments, the system further comprises a laser monitor sensor comprising: an excitation light power gauge configured to measure a power of the excitation light; and a diffused beam shape sensor measuring a diffused beam shape, the diffused beam shape sensor comprising a first diffused beam shape gauge and a second diffused beam shape gauge; or both. In some embodiments, the system further comprises a reflector redirecting a portion of the excitation light to the excitation light power gauge. In some embodiments, the reflector is positioned between the excitation channel and the excitation diffuser. In some embodiments, the optical device allows a portion of the diffused excitation light to pass therethrough in a direction parallel to the diffused excitation light, and wherein the diffused beam shape sensor receives the portion of the diffused excitation light. In some embodiments, the first diffused beam shape gauge measures a power of the diffused beam at a center of the diffused beam shape and wherein the second diffused beam shape gauge measures the power of the diffused beam at an edge of the diffused beam shape. In some embodiments, the excitation light power gauge, the first diffused beam shape gauge, the second diffused beam shape gauge, or any combination thereof comprises a photodiode, a camera, a piezoelectric sensor, a linear sensor array, a CMOS sensor, or any combination thereof. In some embodiments, the laser monitor turns off the laser if: the measured power of the excitation light deviates from a set excitation light power by a first predetermined value; the diffused beam shape deviates from a set beam shape by a second predetermined value; or both. In some embodiments, the laser has an off mode and an on mode.

Another aspect provided herein is an imaging platform for imaging an emission light emitted by a sample comprising a fluorophore, the platform comprising: the imaging system herein; and an imaging station comprising: a non-transitory computer-readable storage media encoded with a computer program including instructions executable by a processor to receive the image frames from the image sensor; and an input device.

In some embodiments, the imaging station receives the image frames from the image sensor via an imaging cable, a wireless connection, or both. In some embodiments, the platform further comprises the imaging cable. In some embodiments, the imaging system further receives power from the image station via the imaging cable. In some embodiments, the imaging system comprises the laser monitor sensor, wherein the platform further comprises a laser monitor interlock receiving data from the laser monitor sensor, and wherein the laser monitor interlock turns off the laser if: the measured power of the excitation light deviates from a set excitation light power by a first predetermined value; the diffused beam shape deviates from a set beam shape by a second predetermined value; or both. In some embodiments, the wireless connection comprises a Bluetooth connection, a Wi-Fi connection, an RFID connection, or any combination thereof. In some embodiments, the input device comprises a mouse, a trackpad, a joystick, a touchscreen, a keyboard, a microphone, a camera, a scanner, an RFID reader, a Bluetooth device, a gesture interface, a voice interface, or any combination thereof.

In some embodiments, the NIR illumination source (laser) is in the imaging station and in some cases it is located in the imaging head. It could also be located in the imaging cable (‘shortstop’ solution).

In some embodiments, the microscope heats the imaging system above the ambient room temperature (e.g. through thermal conduction and/or radiation from the microscope's visible illumination source).

In some embodiments, the imaging system's design is optimized for operation at an elevated temperature (above ambient). Since laser emission wavelength shifts as the function of temperature, the optical filters are optimized for the thermally-shifted wavelength range.

In some embodiments, the temperature of the laser is controlled to reduce the range of the temperature-dependent emission wavelength shift. The laser temperature could be controlled using a thermo-electric cooler (TEC), a heater (e.g., resistive load)

In some embodiments, the temperature of the laser is not controlled.

In some embodiments, the imaging station is ‘cart based’. In other embodiments, the imaging station is contained in a small wheeled unit, is hung off of the microscope, is rested or hung elsewhere on the microscope, is placed on the floor next to the microscope, or is placed on a tray/pole/table. Or, the imaging station could be designed to be placed in multiple positions such as hanging from the microscope and hanging on a tray.

The imaging station may provide storage for the imaging system and/or imaging cable. Or, these components could be separated stored in some other container (e.g. a storage case or like container).

Another aspect provided herein is a method for imaging an emission light emitted by a sample comprising a fluorophore, the method comprising: emitting an excitation light; diffusing the excitation light; receiving and directing a visible light to the sample; directing the diffused excitation light to the sample; directing the emission light and a reflected visible light to an imaging assembly; and filtering the emission light and the reflected visible light; detecting both the emission light and the reflected visible light from the sample to generate image frames based on the emission light and the reflected visible light.

In some embodiments, filtering the emission light and the reflected visible light comprises directing the emission light and the reflected visible light from the sample through the notch beam splitter, the first notch filter, the longpass filter, the lens, and the second notch filter. In some embodiments, filtering the emission light and the reflected visible light comprises directing the emission light and the reflected visible light from the sample and sequentially through the notch beam splitter, the first notch filter, the longpass filter, the lens, and the second notch filter. In some embodiments, the excitation light has a wavelength of about 775 nm to about 795 nm. In some embodiments, the excitation light has a wavelength of about 785 nm. In some embodiments, the visible light has a wavelength of about 400 nm to about 700 nm. In some embodiments, the visible light has a wavelength of about 800 nm to about 950 nm. In some embodiments, the excitation light is diffused by a circular excitation diffuser. In some embodiments, the circular excitation diffuser has a diffusion angle of about 4 degrees to about 25 degrees. In some embodiments, the excitation light is diffused by a rectangular excitation diffuser. In some embodiments, the rectangular excitation diffuser has a first diffusion angle and a second diffusion angle perpendicular to the first diffusion angle.

In some embodiments, the first diffusion angle, the second diffusion angle, or both are about 4 degrees to about 25 degrees. In some embodiments, the first diffusion angle, the second diffusion angle, or both are about 4 degrees to about 6 degrees, about 4 degrees to about 8 degrees, about 4 degrees to about 10 degrees, about 4 degrees to about 12 degrees, about 4 degrees to about 14 degrees, about 4 degrees to about 16 degrees, about 4 degrees to about 18 degrees, about 4 degrees to about 20 degrees, about 4 degrees to about 22 degrees, about 4 degrees to about 25 degrees, about 6 degrees to about 8 degrees, about 6 degrees to about 10 degrees, about 6 degrees to about 12 degrees, about 6 degrees to about 14 degrees, about 6 degrees to about 16 degrees, about 6 degrees to about 18 degrees, about 6 degrees to about 20 degrees, about 6 degrees to about 22 degrees, about 6 degrees to about 25 degrees, about 8 degrees to about 10 degrees, about 8 degrees to about 12 degrees, about 8 degrees to about 14 degrees, about 8 degrees to about 16 degrees, about 8 degrees to about 18 degrees, about 8 degrees to about 20 degrees, about 8 degrees to about 22 degrees, about 8 degrees to about 25 degrees, about 10 degrees to about 12 degrees, about 10 degrees to about 14 degrees, about 10 degrees to about 16 degrees, about 10 degrees to about 18 degrees, about 10 degrees to about 20 degrees, about 10 degrees to about 22 degrees, about 10 degrees to about 25 degrees, about 12 degrees to about 14 degrees, about 12 degrees to about 16 degrees, about 12 degrees to about 18 degrees, about 12 degrees to about 20 degrees, about 12 degrees to about 22 degrees, about 12 degrees to about 25 degrees, about 14 degrees to about 16 degrees, about 14 degrees to about 18 degrees, about 14 degrees to about 20 degrees, about 14 degrees to about 22 degrees, about 14 degrees to about 25 degrees, about 16 degrees to about 18 degrees, about 16 degrees to about 20 degrees, about 16 degrees to about 22 degrees, about 16 degrees to about 25 degrees, about 18 degrees to about 20 degrees, about 18 degrees to about 22 degrees, about 18 degrees to about 25 degrees, about 20 degrees to about 22 degrees, about 20 degrees to about 25 degrees, or about 22 degrees to about 25 degrees. In some embodiments, the first diffusion angle, the second diffusion angle, or both are about 4 degrees, about 6 degrees, about 8 degrees, about 10 degrees, about 12 degrees, about 14 degrees, about 16 degrees, about 18 degrees, about 20 degrees, about 22 degrees, or about 25 degrees. In some embodiments, the first diffusion angle, the second diffusion angle, or both are at least about 4 degrees, about 6 degrees, about 8 degrees, about 10 degrees, about 12 degrees, about 14 degrees, about 16 degrees, about 18 degrees, about 20 degrees, or about 22 degrees. In some embodiments, the first diffusion angle, the second diffusion angle, or both are at most about 6 degrees, about 8 degrees, about 10 degrees, about 12 degrees, about 14 degrees, about 16 degrees, about 18 degrees, about 20 degrees, about 22 degrees, or about 25 degrees.

In some embodiments, the first diffusion angle is about 14 degrees, and wherein the second diffusion angle is about 8 degrees. In some embodiments, the diffused excitation light is directed to the sample by a hot mirror, a dichroic mirror, a shortpass filter, or any combination thereof. In some embodiments, the reflected visible light is directed to the imaging assembly by a hot mirror, a dichroic mirror, a shortpass filter, or any combination thereof. In some embodiments, the hot mirror filters out the wavelength of the NIR light from the visible light. In some embodiments, the diffused excitation light is directed to the sample in a first direction and wherein the emission light and the reflected visible light are directed in a second direction opposite the first direction. In some embodiments, filtering the emission light and the reflected visible light comprises blocking the excitation light.

In some embodiments, filtering the emission light and the reflected visible light comprises blocking light having a wavelength of about 775 nm to about 795 nm. In some embodiments, filtering the emission light and the reflected visible light comprises blocking light having a wavelength of about 775 nm to about 780 nm, about 775 nm to about 785 nm, about 775 nm to about 790 nm, about 775 nm to about 795 nm, about 780 nm to about 785 nm, about 780 nm to about 790 nm, about 780 nm to about 795 nm, about 785 nm to about 790 nm, about 785 nm to about 795 nm, or about 790 nm to about 795 nm. In some embodiments, filtering the emission light and the reflected visible light comprises blocking light having a wavelength of about 775 nm, about 780 nm, about 785 nm, about 790 nm, or about 795 nm. In some embodiments, filtering the emission light and the reflected visible light comprises blocking light having a wavelength of at least about 775 nm, about 780 nm, about 785 nm, or about 790 nm. In some embodiments, filtering the emission light and the reflected visible light comprises blocking light having a wavelength of at most about 780 nm, about 785 nm, about 790 nm, or about 795 nm.

In some embodiments, the method further comprises polarizing the emission light and the reflected visible light. In some embodiments, the method further comprises filtering the diffused excitation light. In some embodiments, filtering the diffused excitation light comprises filtering out wavelengths less than about 720 nm, 725 nm, 730 nm, 735 nm, 740 nm, 750 nm, 755 nm, 760 nm, 770 nm, 780 nm, 800 nm, or more including increments therein. In some embodiments, the excitation light is an infrared or a near-infrared excitation light. In some embodiments, filtering the emission light and the reflected visible light comprises attenuating the emission light and the reflected visible light. In some embodiments, attenuating the emission light and the reflected visible light comprises blocking all but near infrared wavelengths. In some embodiments, the method further comprises monitoring the laser by: measuring a power of the excitation light with an excitation light monitor; and measuring a diffused beam shape of the diffused excitation light with a first diffused beam shape gauge and a second diffused beam shape gauge; or both. In some embodiments, the excitation light monitor measures the power of the excitation light by receiving a redirected portion of the excitation light. In some embodiments, the first diffused beam shape gauge measures a power of the diffused beam at a center of the diffused beam shape and wherein the second diffused beam shape gauge measures the power of the diffused beam at an edge of the diffused beam shape. In some embodiments, the excitation light monitor, the first diffused beam shape gauge, the second diffused beam shape gauge, or any combination thereof comprises a photodiode, a camera, a piezoelectric sensor, a linear sensor array, a CMOS sensor, or any combination thereof. In some embodiments, the method further comprises turning off the laser if: the measured power of the excitation light deviates from a set excitation light power by a first predetermined value; the diffused beam shape deviates from a set beam shape by a second predetermined value; or both. In some embodiments, the laser has an off mode and an on mode. In some embodiments, the method further comprises receiving, by a non-transitory computer-readable storage media encoded with a computer program including instructions executable by a processor, the image frames from the image sensor. In some embodiments, receiving the image frames from the image sensor is performed by an imaging cable, a wireless connection, or both. In some embodiments, the wireless connection comprises a Bluetooth connection, a WIFI connection, an RFID connection, or any combination thereof.

One aspect provided herein is an imaging system for imaging an emission light emitted by a sample comprising a fluorophore, the system comprising: an excitation channel to receive an excitation light; an excitation diffuser that diffuses the excitation light; a visible channel to receive and direct a visible light to the sample; an optical device directing the diffused excitation light to the sample and allowing the emission light and a reflected visible light to pass therethrough to an imaging assembly; the imaging assembly comprising: a first notch filter; a longpass filter; a lens; a second notch filter; and an image sensor configured to detect both the emission light and the reflected visible light from the sample and configured to generate image frames based on the emission light and the reflected visible light.

In some embodiments, emission light and the reflected visible light are directed from the sample through the notch beam splitter, the first notch filter, the longpass filter, the lens, and the second notch filter. In some embodiments, emission light and the reflected visible light are directed from the sample and sequentially through the notch beam splitter, the first notch filter, the longpass filter, the lens, and the second notch filter. In some embodiments, the excitation light has a wavelength of about 700 nm to about 800 nm, about 800 nm to about 950 nm, about 775 nm to about 795 nm, or about 785. In some embodiments, the visible light source has a wavelength of about 400 nm to about 800 nm. In some embodiments, the excitation diffuser is a circular excitation diffuser. In some embodiments, the circular excitation diffuser has a diffusion angle of about 4 degrees to about 25 degrees, or about 8 to about 14 degrees. In some embodiments, the excitation diffuser is a rectangular excitation diffuser. In some embodiments, the rectangular excitation diffuser has a first diffusion angle and a second diffusion angle perpendicular to the first diffusion angle. In some embodiments, the first diffusion angle, the second diffusion angle, or both are about 4 degrees to about 25 degrees, or about 8 to about 14 degrees. In some embodiments, the first diffusion angle is about 14 degrees, and wherein the second diffusion angle is about 8 degrees. In some embodiments, the optical device is a hot mirror, a dichroic mirror, a shortpass filter, or any combination thereof. In some embodiments, the hot mirror filters out, reflects, or separates the wavelength of the NIR or IR light from the visible light. In some embodiments, the optical device directs the diffused excitation light to the sample in a first direction and allows the emission light and a reflected visible light to pass therethrough in a second direction opposite the first direction. In some embodiments, at least one of the first notch filter and the second notch filter block the excitation light from passing therethrough. In some embodiments, the width of the notch filter is greater than the spectral width of the source of excitation light. In some embodiments, at least one of the first notch filter and the second notch filter with a center blocking band of about 775 nm to about 795 nm from passing therethrough, wherein the center blocking bandwidth is of sufficient width to attenuate the excitation source. In some embodiments, at least one of the first notch filter and the second notch filter block light having a center blocking band of about 785 nm from passing therethrough, where the blocking bandwidth is of sufficient width to attenuate the excitation source. In some embodiments, the imaging assembly further comprises a polarizer. In some embodiments, the emission light and the reflected visible light are directed through the long pass-filter, the polarizer, and the lens in any order. In some embodiments, the emission light and the reflected visible light are directed sequentially through the long pass-filter, the polarizer and the lens. In some embodiments, the system further comprises a white light that emits the visible light. In some embodiments, the system further comprises a shortpass mirror between the imaging assembly and the sample and between the excitation diffuser and the sample. In some embodiments, the shortpass dichroic mirror transmits wavelengths of about 400 nm to about 720 nm, and wherein the shortpass dichroic mirror reflects wavelengths greater than about 720 nm. In some embodiments, the system further comprises a bottom window between the shortpass mirror and the sample. In some embodiments, the shortpass mirror comprises a pellicle mirror, a dichroic mirror, or any combination thereof. In some embodiments, the system further comprises a front window between the notch filter and the sample. In some embodiments, the excitation light is an infrared or a near-infrared excitation light. In some embodiments, the long pass filter comprises a visible light attenuator. In some embodiments, the visible light attenuator transmits near infrared or infrared wavelengths. In some embodiments, the system further comprises a laser monitor sensor comprising: an excitation light power gauge configured to measure a power of the excitation light (excitation power); and a diffused beam shape sensor measuring a diffused beam shape comprising at least one diffused beam shape gauge. In some embodiments, the system further comprises a first diffused beam shape gauge and a second diffused beam shape gauge. In some embodiments, the system further comprises a reflector redirecting a portion of the excitation light to the excitation light power gauge. In some embodiments, the reflector is positioned between the excitation channel and the excitation diffuser. In some embodiments, the system further comprises the optical device allows a portion of the diffused excitation light to pass therethrough in a direction parallel to the diffused excitation light, and wherein the diffused beam shape sensor receives the portion of the diffused excitation light. In some embodiments, the first diffused beam shape gauge measures a power of the diffused beam at a center of the diffused beam shape and wherein the second diffused beam shape gauge measures the power of the diffused beam at an edge of the diffused beam shape. In some embodiments, the system further comprises 2, 3, 4, 5, 6, 7, 8, 9, 10, or more additional beam shape gauges. In some embodiments, the first beam shape gauge, the second beam shape gauge, the additional beam shape gauges, or any combination thereof are arranged in a one-dimensional array. In some embodiments, the first beam shape gauge, the second beam shape gauge, the additional beam shape gauges, or any combination thereof are arranged in a two-dimensional array. In some embodiments, the excitation light power gauge, the first diffused beam shape gauge, the second diffused beam shape gauge, or any combination thereof comprises a photodiode, a camera, a piezoelectric sensor, a linear sensor array, a CMOS sensor, or any combination thereof. In some embodiments, the excitation light power gauge, the first diffused beam shape gauge, the second diffused beam shape gauge, or any combination thereof are positioned in the path of the excitation beam or behind an optical component. In some embodiments, the laser has an off mode and an on mode.

Since it is not possible to see NIR excitation and fluorescence with the naked eye, in some embodiments, the system includes a fluorescent imaging target that can be used to ensure that the system's infrared imaging is performing normally.

In some embodiments, the laser monitor system previously described can be used as part of a system that confirms that infrared imaging is performing normally, without the need for a separate fluorescent imaging target.

Another aspect provided herein is an imaging platform for imaging an emission light emitted by a sample comprising a fluorophore, the platform comprising: the imaging system; an imaging station comprising: a non-transitory computer-readable storage media encoded with a computer program including instructions executable by a processor to receive the image frames from the image sensor; and an input device. In some embodiments, the imaging station receives the image frames from the image system via an imaging cable, a wireless connection, or both. In some embodiments, further comprising the imaging cable. In some embodiments, the imaging system further receives power via the imaging cable. In some embodiments, the imaging platform further comprises an imaging system that receives power via the imaging cable. In some embodiments, the wireless connection comprises a Bluetooth connection, a Wi-Fi connection, a cellular data connection, an RFID connection, or any combination thereof. In some embodiments, the input device comprises a mouse, a trackpad, a joystick, a touchscreen, a keyboard, a microphone, a camera, a scanner, an RFID reader, a Bluetooth device, a gesture interface, a voice command interface, or any combination thereof. In some embodiments, the imaging system comprises the laser monitor sensor, wherein the platform further comprises a laser monitor electronics receiving data from the laser monitor sensor, and wherein the laser monitor electronics turns off the laser if: the measured power of the excitation light (i.e., excitation power) deviates from a set excitation light power by a first predetermined value; the diffused beam shape deviates from a set beam shape by a second predetermined value; or both. In some embodiments, the first predetermined value comprises excitation power as measured by a predetermined range value or a predetermined maximum magnitude of the rate of change value or both. In some embodiments, the first predetermined value has either exceeded the highest predetermined value in the range or is less than the lowest predetermined value in the range. In some embodiments, the second predetermined value comprises values that deviate from a set beam shape as measured by one or more diffused beam shape gauge In some embodiments, the second predetermined value determines that the diffused beam shape has deviated from the set beam shape based on the power of the diffused beam at least one point along the diffused beam shape as measured by a first diffused beam shape gauge as compared to the power of the diffused beam at least one other point along the diffused beam shape as measured by at least a second diffused beam shape gauge. In some embodiments, the laser monitor electronics turns off the laser by interrupting power supplied to the laser, or as a result of excitation power exceeding or being less than the set range value or a set range rate, or as a result of deviation as measured by one or more diffused beam shape gauge. In some embodiments, the laser is shut off within a millisecond, a microsecond, or a picosecond, or less, of when the laser monitor electronics determines that the magnitude of the rate of change of the excitation power relative to a predetermined maximum value has exceeded the highest predetermined rate, or that the beam shape has deviated from the set beam shape.

Another aspect provided herein is a method for imaging an emission light emitted by a sample comprising a fluorophore, the method comprising: emitting an excitation light; diffusing the excitation light; receiving and directing a visible light to the sample; directing the diffused excitation light to the sample; directing the emission light and a reflected visible light to an imaging assembly; filtering the excitation light and the reflected visible light from the emission light; detecting both the emission light and the reflected visible light from the sample to generate image frames based on the emission light and the reflected visible light.

In some embodiments, filtering the excitation light and the reflected visible light from the emission light comprises directing the emission light and the reflected visible light from the sample through the notch beam splitter, the first notch filter, the longpass filter, the lens, and the second notch filter. In some embodiments, filtering the excitation light and the reflected visible light comprises directing the emission light and the reflected visible light from the sample and sequentially through the notch beam splitter, the first notch filter, the longpass filter, the lens, and the second notch filter. In some embodiments, the excitation light has a wavelength of about 775 nm to about 795 nm. In some embodiments, the excitation light has a wavelength of about 785 nm. In some embodiments, the visible light source has a wavelength of about 400 nm to about 800 nm. In some embodiments, the excitation light has a wavelength of about 800 nm to about 950 nm. In some embodiments, the excitation light is diffused by a circular excitation diffuser. In some embodiments, the circular excitation diffuser has a diffusion angle of about 4 degrees to about 25 degrees. In some embodiments, the excitation light is diffused by a rectangular excitation diffuser. In some embodiments, the rectangular excitation diffuser has a first diffusion angle and a second diffusion angle perpendicular to the first diffusion angle. In some embodiments, the first diffusion angle, the second diffusion angle, or both are about 4 degrees to about 25 degrees. In some embodiments, the first diffusion angle is about 14 degrees, and wherein the second diffusion angle is about 8 degrees. In some embodiments, the diffused excitation light is directed to the sample by a hot mirror, a dichroic mirror, a shortpass filter, or any combination thereof. In some embodiments, the reflected visible light is directed to the imaging assembly by a hot mirror, a dichroic mirror, a shortpass filter, or any combination thereof. In some embodiments, the hot mirror filters out the wavelength of the NIR or IR light from the visible light. In some embodiments, the diffused excitation light is directed to the sample in a first direction and wherein the emission light and the reflected visible light are directed in a second direction opposite the first direction. In some embodiments, filtering the emission light and the reflected visible light comprises blocking light having a wavelength of about 775 nm to about 795 nm from passing therethrough. In some embodiments, filtering the emission light and the reflected visible light comprises blocking light having a wavelength of about 785 nm from passing therethrough. In some embodiments, the method further comprises polarizing the emission light and the reflected visible light. In some embodiments, the method further comprises filtering the diffused excitation light. In some embodiments, filtering the diffused excitation light comprises filtering out wavelengths less than about 720 nm, 725 nm, 730 nm, 735 nm, 740 nm, 750 nm, 755 nm, 760 nm, 770 nm, 780 nm, 800 nm, or more including increments therein. In some embodiments, the excitation light is an infrared or a near-infrared excitation light. In some embodiments, the method further comprises monitoring the laser by: measuring a power of the excitation light with an excitation light monitor; measuring a diffused beam shape of the diffused excitation light with a sensor system; or both. In some embodiments, the excitation light monitor measures the power of the excitation light by receiving a redirected portion of the excitation light. In some embodiments, the first diffused beam shape gauge measures a power of the diffused beam at a center of the diffused beam shape and wherein the second diffused beam shape gauge measures the power of the diffused beam at an edge of the diffused beam shape. In some embodiments, the excitation light monitor, the first diffused beam shape gauge, the second diffused beam shape gauge, or any combination or number of sensors/gauges thereof comprises a photodiode, a camera, a piezoelectric sensor, a linear sensor array, a CMOS sensor, or any combination thereof.

The method of any one of claims 79-82, further comprising measuring the power of the diffused beam by 2, 3, 4, 5, 6, 7, 8, 9, 10, or more additional beam shape gauges. In some embodiments, the first beam shape gauge, the second beam shape gauge, the additional beam shape gauges, or any combination thereof are arranged in a one-dimensional array. In some embodiments, the first beam shape gauge, the second beam shape gauge, the additional beam shape gauges, or any combination thereof are arranged in a two-dimensional array. In some embodiments, the excitation light power gauge, the first diffused beam shape gauge, the second diffused beam shape gauge, or any combination thereof comprises a photodiode, a camera, a piezoelectric sensor, a linear sensor array, a CMOS sensor, or any combination thereof. In some embodiments, the excitation light power gauge, the first diffused beam shape gauge, the second diffused beam shape gauge, or any combination thereof are positioned in the path of the excitation beam or behind an optical component. In some embodiments, the method further comprises turning off the laser if: the measured power of the excitation light deviates from a set excitation light power by a first predetermined value; the diffused beam shape deviates from a set beam shape by a second predetermined value; or both. In some embodiments, the laser has an off mode and an on mode. In some embodiments, the method further comprises receiving, by a non-transitory computer-readable storage media encoded with a computer program including instructions executable by a processor, the image frames from the image sensor. In some embodiments, receiving the image frames from the image sensor is performed by an imaging cable, a wireless connection, or both. In some embodiments, the wireless connection comprises a Bluetooth connection, a Wi-Fi connection, a cellular data connection, an RFID connection, or any combination thereof.

Another aspect provided herein is a computer-implemented method of forming a first overlaid image from laser induced fluorophore excitations, the method comprising: receiving a plurality of image frame sequences, each image frame sequence comprising: a VIS frame captured when the laser is in an off mode or in an on mode; and a primary quantity of NIR or IR frames captured when the laser is in an on mode; correcting each NIR or IR frame by subtracting one correcting VIS frame; generating a first NIR or IR image by adding a first corrected NIR or IR frame and N quantity of subsequent corrected NIR or IR frames; generating a first VIS image by adding a first VIS frame and a V quantity of VIS frames subsequent to the first VIS frame; and overlaying the NIR or IR image and the VIS image to form the first overlaid image.

In some embodiments, the correcting VIS_DRK frame is a VIS_DRK frame in the same frame sequence as the first NIR or IR frame, in a subsequent frame sequence to the frame sequence of the NIR frame, in a previous frame sequence to the frame sequence of the NIR frame, or combination thereof. In some embodiments, generating a first VIS image is achieved by either directly displaying a first VIS frame or adding a first VIS frame and a V quantity of VIS frames subsequent to the first VIS frame in an accumulator. In some embodiments, the overlaid images are obtained by overlaying the summed quantity of corrected NIR or IR image(s) and the summed quantity of VIS image(s) to form the first overlaid image. In some embodiments, the V quantity is zero or more. In some embodiments, a sequence comprises a primary quantity of NIR frames and a VIS frame, the correcting VIS_DRK frame for any given NIR or IR frame is the VIS_DRK frame that is temporally closest to the given NIR or IR frame, regardless of whether the correcting VIS frame is in the same, previous, or subsequent frame sequence relative to the given NIR or IR frame. In some embodiments, (N+1) is equal to or greater than the primary quantity. In some embodiments, generating the first NIR or IR image further comprises adding M quantity of corrected NIR or IR frames preceding the first corrected NIR or IR frame. In some embodiments, subtracting the temporally closest or nearest correcting VIS frame to each NIR frame reduces, or minimizes a motion artifact caused by movement between the capture of the VIS frame, the NIR or IR frame, or both. In some embodiments, each image frame sequence further comprises one or more VIS_DRK frames captured only under ambient light. In some embodiments, correcting each NIR or IR frame further comprises subtracting at least one of the one or more VIS_DRK frames from the NIR or IR frame. In some embodiments, a signal gain of each NIR or IR frame is equal to a signal gain of at least one of the one or more VIS_DRK frames multiplied by a captured input dynamic range. In some embodiments, each of the VIS frames, and each of the NIR or IR frames are captured by a sensor having visible and NIR or IR pixels. In some embodiments, one or more of the VIS frames and one or more of the NIR or IR frames are contained in a single frame. In some embodiments, subtracting the VIS_DRK frame comprises subtracting the VIS_DRK frame multiplied by a ratio between an exposure of the NIR or IR frame and an exposure of at least one of the one or more VIS_DRK frames. In some embodiments, at least one of the one or more VIS_DRK frames, the VIS frame of one or more sequences, and the NIR or IR frame of one or more sequences are captured by a sensor having visible and NIR or IR pixels. In some embodiments, at least one of the one or more VIS_DRK frames, the VIS frame of one or more sequences, and the NIR or IR frame of one or more sequences is contained in a single frame. In some embodiments, the VIS frame is captured when the laser is in the on mode. In some embodiments, at least one of the one or more VIS_DRK frames comprises the VIS frame. In some embodiments, at least one of the one or more VIS_DRK frames does not comprise the VIS frame. In some embodiments, the method further comprises: generating a second NIR or IR image by adding a (N+1)th or (N+2)th corrected NIR or IR frame and N quantity of subsequent corrected NIR or IR frames; generating a second VIS image by adding a second VIS frame and V quantity of VIS frames subsequent to the second VIS frame; overlaying the second NIR or IR image and the second VIS image to form the second overlaid image. In some embodiments, N+1 is equal to X times the primary quantity, wherein X is a whole number greater than 2, wherein the application further comprises a module generating a second NIR or IR image by adding a (N+primary quantity+1)th or (N+primary quantity+2)th corrected NIR or IR frame and N quantity of subsequent corrected NIR or IR frames In some embodiments, the method further comprises forming a display image from two or more overlaid images. In some embodiments, one display image is formed for each sequence. In some embodiments, one display image is formed from two or more sequences.

Another aspect provided herein is a computer-implemented system comprising: a digital processing device comprising: at least one processor, an operating system configured to perform executable instructions, a memory, and a computer program including instructions executable by the digital processing device to create an application for forming a first overlaid image from laser induced fluorophore excitations, the application comprising: a module receiving a plurality of image frame sequences, each image frame sequence comprising: a VIS frame captured when the laser is in an off mode or in an on mode; and a primary quantity of NIR or IR frames captured when the laser is in an on mode; a module correcting each NIR or IR frame by subtracting one correcting VIS frame; a module generating a first NIR or IR image by adding a first corrected NIR or IR frame and N quantity of subsequent corrected NIR or IR frames; a module generating a first VIS image by adding a first VIS frame and a V quantity of VIS frames subsequent to the first VIS frame; and a module overlaying the NIR or IR image and the VIS image to form the first overlaid image.

In some embodiments, the correcting VIS frame is a VIS frame in the same frame sequence as the first NIR or IR frame, in a subsequent frame sequence to the frame sequence of the NIR frame, in a previous frame sequence to the frame sequence of the NIR frame, or combination thereof. In some embodiments, generating a first VIS image is achieved by either directly displaying a first VIS frame or adding a first VIS frame and a V quantity of VIS frames subsequent to the first VIS frame in an accumulator. In some embodiments, the overlaid NIR or IR images are obtained by overlaying the summed quantity of NIR or IR image(s) and the summed quantity of VIS image(s) to form the first overlaid image. In some embodiments, the V quantity is zero or more. In some embodiments, a sequence comprises a primary quantity of NIR frames and a VIS frame, the correcting VIS frame for any given NIR or IR frame is the VIS frame that is temporally closest to the given NIR or IR frame, regardless of whether the correcting VIS frame is in the same, previous, or subsequent frame sequence relative to the given NIR or IR frame. In some embodiments, (N+1) is equal to or greater than the primary quantity. In some embodiments, generating the first NIR or IR image further comprises adding M quantity of corrected NIR or IR frames preceding the first corrected NIR or IR frame. In some embodiments, subtracting the temporally closest or nearest correcting VIS frame to each NIR frame reduces, minimizes, or corrects a motion artifact caused by movement between the capture of the VIS frame, the NIR or IR frame, or both. In some embodiments, each image frame sequence further comprises one or more VIS_DRK frames captured only under ambient light. In some embodiments, correcting each NIR or IR frame further comprises subtracting at least one of the one or more VIS_DRK frames from the NIR or IR frame. In some embodiments, a signal gain of each NIR or IR frame is equal to a signal gain of at least one of the one or more VIS_DRK frames multiplied by a captured input dynamic range. In some embodiments, each of the VIS frames, and each of the NIR or IR frames are captured by a sensor having visible and NIR or IR pixels. In some embodiments, one or more of the VIS frames and one or more of the NIR or IR frames are contained in a single frame. In some embodiments, subtracting the VIS_DRK frame comprises subtracting the VIS_DRK frame multiplied by a ratio between an exposure of the NIR or IR frame and an exposure of at least one of the one or more VIS_DRK frames. In some embodiments, at least one of the one or more VIS_DRK frames, the VIS frame of one or more sequences, and the NIR or IR frame of one or more sequences are captured by a sensor having visible and NIR or IR pixels. In some embodiments, at least one of the one or more VIS_DRK frames, the VIS frame of one or more sequences, and the NIR or IR frame of one or more sequences is contained in a single frame. In some embodiments, the VIS frame is captured when the laser is in the on mode. In some embodiments, at least one of the one or more VIS_DRK frames comprises the VIS frame. In some embodiments, at least one of the one or more VIS_DRK frames does not comprise the VIS frame. In some embodiments, the application further comprises a module generating a second NIR or IR image by adding a (N+1)th or (N+2)th corrected NIR or IR frame and N quantity of subsequent corrected NIR or IR frames; a module generating a second VIS image by adding a second VIS frame and V quantity of VIS frames subsequent to the second VIS frame; a module overlaying the second NIR or IR image and the second VIS image to form the second overlaid image. In some embodiments, N+1 is equal to X times the primary quantity, wherein X is a whole number greater than 2, wherein the application further comprises a module generating a second NIR or IR image by adding a (N+primary quantity+1)th or (N+primary quantity+2)th corrected NIR or IR frame and N quantity of subsequent corrected NIR or IR frames. In some embodiments, the application further comprises a module forming a display image from two or more overlaid images. In some embodiments, one display image is formed for each sequence. In some embodiments, one display image is formed from two or more sequences.

Another aspect provided herein is a non-transitory computer-readable storage media encoded with a computer program including instructions executable by a processor to create an application for forming a first overlaid image from laser induced fluorophore excitations, the application comprising: a module receiving a plurality of image frame sequences, each image frame sequence comprising: a VIS frame captured when the laser is in an off mode or in an on mode; and a primary quantity of NIR or IR frames captured when the laser is in an on mode; a module correcting each NIR or IR frame by subtracting one correcting VIS frame; a module generating a first NIR or IR image by adding a first corrected NIR or IR frame and N quantity of subsequent corrected NIR or IR frames; a module generating a first VIS image by adding a first VIS frame and a V quantity of VIS frames subsequent to the first VIS frame; and a module overlaying the NIR or IR image and the VIS image to form the first overlaid image.

In some embodiments, the correcting VIS frame is a VIS frame in the same frame sequence as the first NIR or IR frame, in a subsequent frame sequence to the frame sequence of the NIR frame, in a previous frame sequence to the frame sequence of the NIR frame, or combination thereof. In some embodiments, generating a first VIS image is achieved by either directly displaying a first VIS frame or adding a first VIS frame and a V quantity of VIS frames subsequent to the first VIS frame in an accumulator. In some embodiments, the overlaid NIR or IR images are obtained by overlaying the summed quantity of NIR or IR image(s) and the summed quantity of VIS image(s) to form the first overlaid image. In some embodiments, the V quantity is zero or more. In some embodiments, a sequence comprises a primary quantity of NIR frames and a VIS frame, the correcting VIS frame for any given NIR or IR frame is the VIS frame that is temporally closest to the given NIR or IR frame, regardless of whether the correcting VIS frame is in the same, previous, or subsequent frame sequence relative to the given NIR or IR frame. In some embodiments, (N+1) is equal to or greater than the primary quantity. In some embodiments, generating the first NIR or IR image further comprises adding M quantity of corrected NIR or IR frames preceding the first corrected NIR or IR frame. In some embodiments, subtracting the temporally closest or nearest correcting VIS frame to each NIR frame reduces, minimizes, or corrects a motion artifact caused by movement between the capture of the VIS frame, the NIR or IR frame, or both. In some embodiments, each image frame sequence further comprises one or more VIS_DRK frames captured only under ambient light. In some embodiments, correcting each NIR or IR frame further comprises subtracting at least one of the one or more VIS_DRK frames from the NIR or IR frame. In some embodiments, a signal gain of each NIR or IR frame is equal to a signal gain of at least one of the one or more VIS_DRK frames multiplied by a captured input dynamic range. In some embodiments, each of the VIS frames, and each of the NIR or IR frames are captured by a sensor having visible and NIR or IR pixels. In some embodiments, one or more of the VIS frames and one or more of the NIR or IR frames are contained in a single frame. In some embodiments, subtracting the VIS_DRK frame comprises subtracting the VIS_DRK frame multiplied by a ratio between an exposure of the NIR or IR frame and an exposure of at least one of the one or more VIS_DRK frames. In some embodiments, at least one of the one or more VIS_DRK frames, the VIS frame of one or more sequences, and the NIR or IR frame of one or more sequences are captured by a sensor having visible and NIR or IR pixels. In some embodiments, at least one of the one or more VIS_DRK frames, the VIS frame of one or more sequences, and the NIR or IR frame of one or more sequences is contained in a single frame. In some embodiments, the VIS frame is captured when the laser is in the on mode. In some embodiments, at least one of the one or more VIS_DRK frames comprises the VIS frame. In some embodiments, at least one of the one or more VIS_DRK frames does not comprise the VIS frame. In some embodiments, the application further comprises: a module generating a second NIR or IR image by adding a (N+1)th or (N+2)th corrected NIR or IR frame and N quantity of subsequent corrected NIR or IR frames; a module generating a second VIS image by adding a second VIS frame and V quantity of VIS frames subsequent to the second VIS frame; a module overlaying the second NIR or IR image and the second VIS image to form the second overlaid image. In some embodiments, N+1 is equal to X times the primary quantity, wherein X is a whole number greater than 2, wherein the application further comprises a module generating a second NIR or IR image by adding a (N+primary quantity+1)th or (N+primary quantity+2)th corrected NIR or IR frame and N quantity of subsequent corrected NIR or IR frames. In some embodiments, the application further comprises a module for forming a display image from two or more overlaid images. In some embodiments, one display image is formed for each sequence. In some embodiments, one display image is formed from two or more sequences.

Another aspect provided herein is a method of imaging a vasculature or structure in a sample from a subject, the method comprising producing an image of the vasculature or structure by imaging fluorescence using an imaging system, the system comprising: the imaging system or the imaging platform.

Another aspect provided herein is a method of imaging a vasculature or structure in a sample from a subject, the method comprising producing an image of the vasculature or structure by imaging fluorescence using an imaging system method. In some embodiments, the fluorescence imaged is autofluorescence, a contrast or imaging agent, chemical agent, a radiolabel agent, radiosensitizing agent, photosensitizing agent, fluorophore, therapeutic agent, an imaging agent, a diagnostic agent, a protein, a peptide, a nanoparticle or a small molecule, or any combination thereof. In some embodiments, the method further comprises administering a contrast or imaging agent to the subject.

Another aspect provided herein is a method of imaging a vasculature or structure in a sample from a subject, the method comprising: administering a contrast or imaging agent to the subject; producing an image of the vasculature or structure by imaging the contrast or imaging agent using an imaging system, the system comprising: the imaging system or the imaging platform

Another aspect provided herein is method of imaging a vasculature or structure in a sample from a subject, the method comprising: administering a contrast or imaging agent to the subject; producing an image of the vasculature or structure by imaging the contrast or imaging agent using an imaging system method. In some embodiments, the contrast or imaging agent comprises a dye, a fluorophore, a fluorescent biotin compound, a luminescent compound, a chemiluminescent compound, or any combination thereof. In some embodiments, the contrast or imaging agent absorbs a wavelength between from about 200 mm to about 900 mm. In some embodiments, the contrast or imaging agent comprises DyLight-680, DyLight-750, VivoTag-750, DyLight-800, IRDye-800, VivoTag-680, Cy5.5, or an indocyanine green (ICG) and any derivative of the foregoing; fluorescein and fluorescein dyes (e.g., fluorescein isothiocyanine or FITC, naphthofluorescein, 4′, 5′-dichloro-2′, 7′-dimethoxyfluorescein, 6-carboxyfluorescein or FAM, etc.), carbocyanine, merocyanine, styryl dyes, oxonol dyes, phycoerythrin, rythrosine, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin, coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), 7-aminocoumarin, a dialkylaminocoumarin reactive dye, 6,8-difluoro-7-hydroxycoumarin fluorophore, a hydroxycoumarin derivative, an alkoxycoumarin derivatives, a succinimidyl ester, a pyrene succinimidyl ester, a pyridyloxazole derivative, an aminonaphthalene-based dyes, dansyl chlorides, a dapoxyl dye, Dapoxyl sulfonyl chloride, amine-reactive Dapoxyl succinimidyl ester, carboxylic acid-reactive Dapoxyl (2-aminoethyl)sulfonamide), a bimane dye, bimane mercaptoacetic acid, an NBD dye, a QsY 35, or any combination thereof. In some embodiments, the administering comprises intravenous administration, intramuscular administration, subcutaneous administration, intraocular administration, intra-arterial administration, peritoneal administration, intratumoral administration, intradermal administration, or any combination thereof. In some embodiments, the imaging comprises tissue imaging, ex vivo imaging, intraoperative imaging, or any combination thereof. In some embodiments, the sample is in an in vivo sample, an in situ sample, an ex vivo sample, or an intraoperative sample. In some embodiments, the sample is an organ, an organ substructure, a tissue, or a cell. In some embodiments, the sample autofluoresces. In some embodiments, autofluorescence of the sample comprises an ocular fluorophore, tryptophan, or protein present in a tumor or malignancy. In some embodiments, the method is used to visualize vessel flow or vessel patency. In some embodiments, the vasculature or structure comprises a blood vessel, lymph vasculature, neuronal vasculature, or CNS structure. In some embodiments, the imaging is angiography, arteriography, lymphography, or cholangiography. In some embodiments, the imaging comprises detecting a vascular abnormality, vascular malformation, vascular lesion, organ or organ substructure, cancer or diseased region, tissue, structure or cell. In some embodiments, the vascular abnormality, vascular malformation, or vascular lesion is an aneurysm, an arteriovenous malformation, a cavernous malformation, a venous malformation, a lymphatic malformation, a capillary telangiectasia, a mixed vascular malformation, a spinal dural arteriovenous fistula, or a combination thereof. In some embodiments, the organ or organ substructure is brain, heart, lung, kidney, liver, or pancreas. In some embodiments, the method further comprises performing surgery on the subject. In some embodiments, the surgery comprises angioplasty, cardiovascular surgery, aneurysm repair, valve replacement, aneurysm surgery, arteriovenous malformation or cavernous malformation surgery, venous malformation surgery, lymphatic malformation surgery, capillary telangiectasia surgery, mixed vascular malformation surgery, or a spinal dural arteriovenous fistula surgery, repair or bypass, arterial bypass, organ transplant, plastic surgery, eye surgery, reproductive system surgery, stent insertion or replacement, plaque ablation, removing the cancer or the diseased region, tissue, structure or cell of the subject, or any combination thereof. In some embodiments, the imaging comprises imaging a vascular abnormality, cancer or diseased region, tissue, structure, or cell of the subject after surgery. In some embodiments, the method further comprises treating a cancer in the subject. In some embodiments, the method further comprises repair of an intracranial CNS vascular defect, a spinal CNS vascular defect; peripheral vascular defects; removal of abnormally vascularized tissue; ocular imaging and repair; anastomosis; reconstructive or plastic surgery; plaque ablation or treatment or restenosis in atherosclerosis; repair or resection (including selective resection), preservation (including selective preservation), of vital organs or structures such as nerves, kidney, thyroid, parathyroid, liver segments, or ureters; identification and management (sometimes preservation, sometimes selective resection) during surgery; diagnosis and treatment of ischemia in extremities; or treatment of chronic wounds. In some embodiments, the intracranial vascular defect and/or the spinal vascular defect comprises an aneurysm, an arteriovenous malformation, a cavernous malformation, a venous malformation, a lymphatic malformation, a capillary telangiectasia, a mixed vascular malformation, or a spinal dural arteriovenous fistula, or any combination thereof. In some embodiments, the peripheral vascular defect comprises an aneurysm, a coronary bypass, another vascular bypass, a cavernous malformation, an arteriovenous malformation, a venous malformation, a lymphatic malformation, a capillary telangiectasia, a mixed vascular malformation, a spinal dural arteriovenous fistula, or any combination thereof. In some embodiments, the abnormally vascularized tissue comprises endometriosis or a tumor. In some embodiments, the method further comprises radiology or fluorescence imaging using one or more of: an X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), microscope, operating microscope, confocal microscope, fluorescence scope, exoscope, surgical robot, surgical instrument, or any combination thereof. In some embodiments, the method comprises measuring and/or quantitating fluorescence using one or more of a microscope, a confocal microscope, a fluorescence scope, an exoscope, a surgical robot, a surgical instrument, or any combination thereof. In some embodiments, the system is combined with or integrated into: a microscope, a confocal microscope, a fluorescence scope, an exoscope, a surgical robot, a surgical instrument, or any combination thereof. In some embodiments, the system comprises a microscope, a confocal microscope, a fluorescence scope, an exoscope, a surgical robot, a surgical instrument, or any combination thereof. In some embodiments, at least one of the microscope, the confocal microscope, the fluorescence scope, the exoscope, the surgical instrument, the endoscope, or the surgical robot comprises a surgical microscope, confocal microscope, fluorescence scope, exoscope, endoscope, ophthalmoscope, retinal camera system, optical coherence tomography (OCT) system, surgical robot, or any combination thereof. In some embodiments, the system is configured to detect, image or assess a therapeutic agent; detect, image or assess a safety or a physiologic effect of the companion diagnostic agent; detect, image or assess a safety or a physiologic effect of the therapeutic agent; detect, image or assess a safety or a physiologic effect of the companion imaging agent; or any combination thereof. In some embodiments, the contrast or imaging agent's safety or physiologic effect is bioavailability, uptake, concentration, presence, distribution and clearance, metabolism, pharmacokinetics, localization, blood concentration, tissue concentration, ratio, measurement of concentrations in blood or tissues, therapeutic window, range and optimization, or any combination thereof. In some embodiments, the method comprises administering a companion diagnostic agent, an therapeutic agent, or an imaging agent, and wherein the imaging comprises detecting the companion diagnostic agent, the therapeutic agent, or the imaging agent. In some embodiments, the companion diagnostic agent, the therapeutic agent, or the imaging agent comprises a chemical agent, a radiolabel agent, radiosensitizing agent, photosensitizing agent, fluorophore, therapeutic agent, an imaging agent, a diagnostic agent, a protein, a peptide, a nanoparticle or a small molecule.

Another aspect provided herein is a method for imaging a sample, comprising: emitting, by a light source, an excitation light to induce fluorescence from the sample; emitting, by a plurality of sources, and excitation light or lights to induce fluorescence from that sample at multiple emission bands; directing, by plurality of optics, the excitation light to the sample; receiving, by plurality of optics, the fluorescence from the sample, wherein the emission light is directed to the sample substantially coaxially with fluorescence light received from the sample in order to decrease shadows; forming a fluorescence image of the sample and a visible light image of the sample on a detector; forming a fluorescence image of the sample and a visible light image of the sample on a plurality of detectors.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. A better understanding of the features and advantages of the present subject matter will be obtained by reference to the following detailed description that sets forth illustrative embodiments and the accompanying drawings of which:

FIG. 1A shows an image of the sterile drape placed over the microscope head and arm.

FIG. 1B shows an exemplary composite image of fluorescent and visible imaging in tissue acquired using the imaging platform and methods, in accordance with some embodiments;

FIG. 2 shows a schematic diagram of an exemplary dichroic filter, in accordance with some embodiments;

FIG. 3A shows a schematic diagram of an exemplary imaging system having non-coaxial illumination and imaging, in accordance with some embodiments;

FIG. 3B shows a schematic diagram of an exemplary imaging system having coaxial illumination and imaging, in accordance with some embodiments;

FIG. 4 shows a schematic diagram of an exemplary embodiment of the imaging system, in this case, a two-camera system that is attached to an operating microscope, in accordance with some embodiments;

FIG. 5 and FIG. 6 each show schematic diagrams of exemplary single camera imaging systems, in accordance various embodiments;

FIG. 5A shows a schematic diagram of an exemplary single camera imaging system, in accordance with some embodiments;

FIG. 5B shows another schematic diagram of an exemplary single camera imaging system, in accordance with some embodiments;

FIG. 5C shows yet another schematic diagram of an exemplary single camera imaging system, in accordance with some embodiments;

FIG. 5D shows yet another schematic diagram of an exemplary single camera imaging system, in accordance with some embodiments;

FIG. 6A shows a different schematic diagram of an exemplary single camera imaging system in communication with a computing device, in accordance with some embodiments;

FIG. 6B shows another schematic diagram of an exemplary single camera imaging system in communication with a computing device, in accordance with some embodiments;

FIG. 7 shows exemplary images captured using the imaging systems and methods herein, in accordance with some embodiments. Images show various aspects of the imaging systems and methods herein including NIR and VIS emission images, effect polarizer and dichroic filter thickness in reducing ghosting, and high magnification of ghosting reduction.

FIG. 7A shows exemplary images captured using the imaging systems and methods herein, in accordance with some embodiments;

FIG. 7B shows exemplary images of ghosting corrections due to a thickness of dichroic filter(s), in accordance with some embodiments;

FIG. 7C shows high magnification images of FIG. 7B;

FIG. 8 shows schematic diagrams of an exemplary imaging system and the path of the excitation light. In particular, FIG. 8A shows a schematic diagram of an exemplary imaging system and the path of the excitation light, in accordance with some embodiments, while FIG. 8B shows a high magnification of the schematic diagram of FIG. 8A, in accordance with some embodiments;

FIG. 9 shows an exemplary timing diagram showing the frame capture and laser on/off triggering for collection of infrared fluorescence images, near infrared (NIR) fluorescence images, and ambient light (dark background) images, in accordance with some embodiments;

FIG. 10 shows exemplary images of the fluorescent and/or visible light as described for imaging ex vivo tissue using the systems described herein. The superimposed composite images show tumor tissue (106a, 106b) and surrounding structures, wherein the tumor tissue 106a and 106b have different signal intensity. Such difference in signal intensity is caused by different level of tissue uptake of fluorescent dye.

FIG. 10A shows an exemplary image of the fluorescent imaging of an ex vivo tissue sample comprising a near infrared (NIR) fluorescent agent and one ex vivo tissue sample with less of the near infrared (NIR) fluorescent agent), in accordance with some embodiments;

FIG. 10B shows an exemplary image of the fluorescent and visible light imaging in ex vivo tissue from the samples in FIG. 10A, wherein the near infrared (NIR) image is displayed as a pseudo color, and wherein the visible light is displayed in true color, in accordance with some embodiments.

FIG. 10C shows an exemplary image of the fluorescent and visible light imaging in ex vivo tissue from the samples in FIG. 10A, wherein the near infrared (NIR) image is displayed as a pseudo color, and wherein the visible light is also displayed as a pseudo color, in accordance with some embodiments;

FIG. 11 shows an exemplary image of a lock and a key for an imaging system, in accordance with some embodiments;

FIG. 12 shows an exemplary illustration of a two-camera imaging system configured to attach to an operating microscope for simultaneous acquisition of near infrared (NIR) fluorescence and visible light; in this case, a, in accordance with some embodiments;

FIG. 13 shows an exemplary schematic diagram of the method steps of using the image systems, in accordance with some embodiments;

FIG. 14 shows a non-limiting schematic diagram of a digital processing device; in this case, a device with one or more CPUs, a memory, a communication interface, and a display, in accordance with some embodiments;

FIG. 15 shows exemplary images of the fluorescent and/or visible light as described for imaging in situ tissues using the systems described herein using visible, NIR, and VIS+NIR image of each tissue sample.

FIG. 15A shows an exemplary visible image of a first in situ tissue sample acquired using the imaging systems and methods herein, in accordance with some embodiments;

FIG. 15B shows an exemplary NIR or IR fluorescent image of the first in situ tissue sample acquired using the imaging systems and methods herein, in accordance with some embodiments;

FIG. 15C shows an exemplary composite visible and fluorescent image of the first in situ tissue sample acquired using the imaging systems and methods herein, in accordance with some embodiments;

FIG. 15D shows an exemplary visible image of a second in situ tissue sample acquired using the imaging systems and methods herein, in accordance with some embodiments;

FIG. 15E shows an exemplary NIR or IR fluorescent image of the second in situ tissue sample acquired using the imaging systems and methods herein, in accordance with some embodiments;

FIG. 15F shows an exemplary composite visible and fluorescent image of the second in situ tissue sample acquired using the imaging systems and methods herein, in accordance with some embodiments;

FIG. 16 shows a schematic diagram of an exemplary double camera imaging system capable of simultaneously acquiring both infrared or near infrared (NIR) fluorescence and visible light images, in accordance with some embodiments;

FIG. 17 shows a non-limiting example of a computing device; in this case, a device with one or more processors, memory, storage, and a network interface;

FIG. 18 shows a schematic diagram of another exemplary single camera imaging system, in accordance with some embodiments;

FIG. 19 shows a perspective view illustration of the optical ray paths within the exemplary single camera imaging system of FIG. 18, in accordance with some embodiments;

FIG. 20 shows an image of an exemplary single camera imaging system, in accordance with some embodiments;

FIG. 21A shows a perspective view illustration of an imaging platform, in accordance with some embodiments;

FIG. 21B shows a perspective view illustration of an imaging platform, in accordance with some embodiments;

FIG. 22 shows a schematic diagram of an imaging station of the imaging platform, in accordance with some embodiments;

FIG. 23 shows a schematic diagram of the time multiplexing of an exemplary single camera imaging system, in accordance with some embodiments;

FIG. 24 shows a schematic diagram of in imaging platform, in accordance with some embodiments

FIG. 25 shows a schematic diagram of another imaging platform, in accordance with some embodiments

FIG. 26 shows an image of an exemplary rectangular beam shape, in accordance with some embodiments;

FIG. 27 shows an image of an exemplary circular beam shape, in accordance with some embodiments;

FIG. 28 shows an exemplary graph of the placement of photodiodes within the beam shape, in accordance with some embodiments;

FIG. 29 shows exemplary images of the fluorescent and/or visible light as described for imaging in vivo or in situ tissues using the systems described herein using visible, NIR, and VIS+NIR image of each tissue sample.

FIG. 29A shows an exemplary visible light (VIS) image of a first in situ tissue sample, in accordance with some embodiments;

FIG. 29B shows an exemplary near-infrared (NIR) image of the first in situ tissue sample, in accordance with some embodiments;

FIG. 29C shows an exemplary overlaid (VIS+NIR) image of the first in situ tissue sample, in accordance with some embodiments;

FIG. 29D shows a near-infrared (NIR) image of a second in situ specimen. Fluorescence signal, corresponding to lighter and brighter areas in the NIR or IR images, is indicative of the presence of tozuleristide in the vascular lesion. Labeled arrows indicate non-fluorescent regions of normal blood vessels (“BV”) and normal brain tissue (“NB”). In contrast, fluorescence signal corresponding to lighter and brighter areas in the NIR or IR image, was indicative of the presence of tozuleristide on the abnormal vascular lesion (“VL”), and not in normal tissue.

FIG. 29E shows the white light image of the second in situ specimen corresponding to FIG. 29D that represents what the surgeon would normally see without fluorescence guidance. The arrows mark the same locations as shown in the NIR or IR image in FIG. 29D. The vascular lesion (“VL”) had a similar appearance to the normal blood vessels (“BV”) in this image.

FIG. 29F shows the NIR or IR fluorescence and white light composite image of the second in situ specimen of FIG. 29D and FIG. 29C, with arrows marking the same locations as shown in FIG. 29D and FIG. 29C. Fluorescence in the vascular lesion (“VL”) clearly differentiated it from the surrounding normal tissues, including normal blood vessels (“BV”).

FIG. 29G shows a near-infrared (NIR) another image of anin situ specimen showing a vascular lesion during the surgery. Arrows indicate the vascular lesion (labeled “VL”) and adjacent normal brain (labeled “NB”), which is non-fluorescent.

FIG. 29H shows the white light image of the in situ specimen corresponding to FIG. 29G. While the normal brain has a light tan to pink color (light gray in a gray scale image), it is perfused with normal blood vessels that are differentiated from the vascular lesion by the absence of fluorescence.

FIG. 29I shows the composite white light and NIR or IR image of the third in situ specimen shown in FIG. 29G and FIG. 29H.

FIG. 30 shows an exemplary diagram of the laser state and the respective frames captured, in accordance with some embodiments;

FIG. 31 shows an exemplary schematic diagram of a method for correcting the NIR/IR frames with a VIS_DRK frame that is subsequent to the NIR/IR frames, in accordance with some embodiments;

FIG. 32 shows an exemplary schematic diagram of a method acquiring NIR or IR and VIS images, correcting the NIR/IR frames with a VIS_DRK frame that is subsequent to the NIR/IR frames, and a first exemplary schematic of summing fluorescent NIR/IR frames and forming overlay images, in accordance with some embodiments;

FIG. 33 shows an exemplary schematic diagram of a method of summing NIR/IR and VIS frames, in accordance with some embodiments;

FIG. 34 shows an exemplary schematic diagram of a method of nearest neighbor correction, summing NIR/IR and VIS frames, in accordance with some embodiments;

FIG. 35 shows an exemplary schematic diagram of a method of nearest neighbor correction, summing NIR/IR and VIS frames, in accordance with some embodiments;

FIG. 36 shows another exemplary schematic diagram of a method of nearest neighbor correction, summing NIR/IR and VIS frames, in accordance with some embodiments;

FIG. 37 shows a first exemplary schematic diagram of a method of mitigating image saturation for multispectral cameras, in accordance with some embodiments;

FIG. 38 shows a second exemplary schematic diagram of a method of mitigating image saturation for multispectral cameras and a method of correcting the NIR/IR frame, in accordance with some embodiments; and

FIG. 39 shows a schematic diagram of a method for summing NIR/IR and VIS frames and forming an overlaid image, in accordance with some embodiments.

### DETAILED DESCRIPTION

Some systems for generating visible, infrared, and near infrared light require a greater control over visible lighting than is required for measurement of fluorescence signals such as infrared signals. However, in some cases, complete or partial control over the visible lighting is not readily available or ideal, for example in a surgical suite or other area where surgeons will adjust light for their needs to view tissue, which in some cases is less than ideal for measuring fluorescence signals. Additionally, in situations where the surgery is conducted using a surgical microscope, it is possible to control the illumination by repositioning the microscope in order to image the fluorescence signal from surgical tissues, and then replacing it to its original position to resume operating when the fluorescence imaging is complete. Moreover, with sources such as halogen lamps, the absorption of excitation light by the fluorophore is sub-optimum and thus such systems are not able achieve simultaneous recording in real time or at video rate without any perceivable lag (e.g., no more than about 100 ms). However, this process often disrupts the surgical techniques. For example, the surgeon is not able to use the microscope when the fluorescence is measured. One problem which often arises with prior systems is that the viewing angles of the fluorescence stimulation or emission wavelengths and the visible wavelengths of the operating microscope are less than ideally arranged, which often results in less than ideal optical signals and image registration resulting in sub-optimal, unclear or poor images. When the fluorescence excitation and the microscope's field of view are not optimally aligned (i.e., coaxial), the fluorescence signal exhibits “blind spots” in some prior systems, such that the tissue does not visibly fluoresce and appears normal and non-cancerous, resulting in failure to identify critical cancerous tissue during surgery in at least some instances.

In light of the above, there is a need for systems and methods that overcome at least some of the aforementioned disadvantages of the prior systems. Ideally such systems and methods would provide fluorescence and visible imaging together, for example simultaneously, with an operating microscope. Moreover, there is a need for systems that do not rely on repositioning the operating microscope to view fluorescence and visible images, and provide imaging of the surgical area together with the fluorescence imaging system during operations and/or pathological examination.

The systems and methods disclosed herein are well suited for combination with many types of surgical and other procedures with minimal disruption in workflow. For example, the presently disclosed methods and apparatus are well suited for incorporation with prior and future operating microscopes, and other imaging devices, such as cameras, monitors, exoscopes, surgical robots, endoscopes, in order to improve the surgical work flow. In some embodiments, the systems and methods disclosed herein are capable of simultaneous capture of visible light and infrared fluorescence and is either be used stand-alone (e.g. open field or endoscopic) or as an attachment to a surgical instrument, such as an operating microscope. For example, the methods and apparatus disclosed herein are well suited for combination and incorporation with commercially available operating microscopes known to one of ordinary skill in the art, such as those commercially available from such companies and sources including Zeiss, Leica, Intuitive Surgical, Olympus, and Haag-Streight, and each of their affiliates. The methods and apparatus, in some embodiments, are combined with commercially available surgical robotic systems and endoscopes known to one of ordinary skill in the art, such as, for example, those commercially available from Intuitive Surgical, and its affiliates.

### Imaging Systems

Provided herein are imaging systems and methods for detecting fluorophore emissions. In some embodiments, the imaging system comprises: a detector, a light source, and a plurality of optics. In some embodiments, the detector is configured to form a fluorescence image of the sample, to form a visible image of the sample, or both. In some embodiments, the light source is configured to emit an excitation light, a visible wavelength illumination, or both. In some embodiments, the excitation light induces fluorescence of the sample. In some embodiments, the visible light illuminates the sample for visible light imaging. In some embodiments, the plurality of optics is arranged to direct the excitation light toward the sample, direct a fluorescent light and a visible light from the sample to the detector, or both. In some embodiments, the illumination light, the excitation light, the fluorescence light, or any combination thereof is directed substantially coaxially.

Fluorophores can be conjugated or fused to another moiety as described herein and are used to home, target, migrate to, be retained by, accumulate in, and/or bind to, or be directed to specific organs, substructures within organs, tissues, targets or cells and used in conjunction with the systems and methods herein. In some embodiments, the fluorophore emission comprises an infrared, near infrared, blue or ultraviolet emission.

In some embodiments, the system is configured to detect fluorophores have an absorption wavelength of about 10 nm to about 200 nm. In some embodiments, the system is configured to detect fluorophores have an absorption wavelength of about 10 nm to about 20 nm, about 10 nm to about 30 nm, about 10 nm to about 40 nm, about 10 nm to about 50 nm, about 10 nm to about 75 nm, about 10 nm to about 100 nm, about 10 nm to about 125 nm, about 10 nm to about 150 nm, about 10 nm to about 200 nm, about 20 nm to about 30 nm, about 20 nm to about 40 nm, about 20 nm to about 50 nm, about 20 nm to about 75 nm, about 20 nm to about 100 nm, about 20 nm to about 125 nm, about 20 nm to about 150 nm, about 20 nm to about 200 nm, about 30 nm to about 40 nm, about 30 nm to about 50 nm, about 30 nm to about 75 nm, about 30 nm to about 100 nm, about 30 nm to about 125 nm, about 30 nm to about 150 nm, about 30 nm to about 200 nm, about 40 nm to about 50 nm, about 40 nm to about 75 nm, about 40 nm to about 100 nm, about 40 nm to about 125 nm, about 40 nm to about 150 nm, about 40 nm to about 200 nm, about 50 nm to about 75 nm, about 50 nm to about 100 nm, about 50 nm to about 125 nm, about 50 nm to about 150 nm, about 50 nm to about 200 nm, about 75 nm to about 100 nm, about 75 nm to about 125 nm, about 75 nm to about 150 nm, about 75 nm to about 200 nm, about 100 nm to about 125 nm, about 100 nm to about 150 nm, about 100 nm to about 200 nm, about 125 nm to about 150 nm, about 125 nm to about 200 nm, or about 150 nm to about 200 nm. In some embodiments, the system is configured to detect fluorophores have an absorption wavelength of about 10 nm, about 20 nm, about 30 nm, about 40 nm, about 50 nm, about 75 nm, about 100 nm, about 125 nm, about 150 nm, or about 200 nm. In some embodiments, the system is configured to detect fluorophores have an absorption wavelength of at least about 10 nm, about 20 nm, about 30 nm, about 40 nm, about 50 nm, about 75 nm, about 100 nm, about 125 nm, or about 150 nm. In some embodiments, the system is configured to detect fluorophores have an absorption wavelength of at most about 20 nm, about 30 nm, about 40 nm, about 50 nm, about 75 nm, about 100 nm, about 125 nm, about 150 nm, or about 200 nm.

In some embodiments, the systems and methods herein detect fluorophore emissions. In some embodiments, the fluorophores emissions comprise an ultraviolet emission. In some embodiments, the ultraviolet emissions have a wavelength from 10 nm to 400 nm, and up to 450 nm or 460 nm into the blue light spectrum, including fluorophores with absorption wavelengths in the ranges disclosed herein, including 10-20 nm, 20-30 nm, 30-40 nm, 40-50 nm, 50-60 nm, 60-70 nm, 70-80 nm, 80-90 nm, 90-100 nm, 100-110 nm, 110-120 nm, 120-130 nm, 130-140 nm, 140-150 nm, 150-160 nm, 160-170 nm, 170-180 nm, 180-190 nm, 190-200 nm, 200-210 nm, 210-220 nm, 220-230 nm, 230-240 nm, 240-250 nm, 250-260 nm, 260-270 nm, 270-280 nm, 280-290 nm, 290-300 nm, 300-310 nm, 310-320 nm, 320-330 nm, 330-340 nm, 340-350 nm, 350-360 nm, 360-370 nm, 370-380 nm, 380-390 nm, 390-400 nm, 400-410 nm, 410-420 nm, 420-430 nm, 430-440 nm, 440-450 nm, 450-460 nm, 300-350 nm, 325-375 nm, 350-400 nm, 400-450 nm, a wavelength in the range of 340 nm to 400 nm, 360 to 420 nm, 380 nm to 440 nm, 400 nm to 450 nm, 400 nm to 460 nm or any wavelength within any of these foregoing ranges.

In some embodiments, the fluorophores emissions comprise an NIR or IR emission. In some embodiments, NIR or IR emissions has a wavelength from about 750 nm to 3000 nm, or 800 nm to 1000 nm, including fluorophores with absorption wavelengths in the ranges disclosed herein. In some embodiments, the system is configured to detect fluorophores that have an absorption wavelength of about 200 nm to about 1,000 nm. In some embodiments, the system is configured to detect fluorophores have an absorption wavelength of about 200 nm to about 250 nm, about 200 nm to about 300 nm, about 200 nm to about 350 nm, about 200 nm to about 400 nm, about 200 nm to about 450 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 250 nm to about 300 nm, about 250 nm to about 350 nm, about 250 nm to about 400 nm, about 250 nm to about 450 nm, about 250 nm to about 500 nm, about 250 nm to about 600 nm, about 250 nm to about 700 nm, about 250 nm to about 800 nm, about 250 nm to about 900 nm, about 250 nm to about 1,000 nm, about 300 nm to about 350 nm, about 300 nm to about 400 nm, about 300 nm to about 450 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 350 nm to about 400 nm, about 350 nm to about 450 nm, about 350 nm to about 500 nm, about 350 nm to about 600 nm, about 350 nm to about 700 nm, about 350 nm to about 800 nm, about 350 nm to about 900 nm, about 350 nm to about 1,000 nm, about 400 nm to about 450 nm, about 400 nm to about 500 nm, about 400 nm to about 600 nm, about 400 nm to about 700 nm, about 400 nm to about 800 nm, about 400 nm to about 900 nm, about 400 nm to about 1,000 nm, about 450 nm to about 500 nm, about 450 nm to about 600 nm, about 450 nm to about 700 nm, about 450 nm to about 800 nm, about 450 nm to about 900 nm, about 450 nm to about 1,000 nm, about 500 nm to about 600 nm, about 500 nm to about 700 nm, about 500 nm to about 800 nm, about 500 nm to about 900 nm, about 500 nm to about 1,000 nm, about 600 nm to about 700 nm, about 600 nm to about 800 nm, about 600 nm to about 900 nm, about 600 nm to about 1,000 nm, about 700 nm to about 800 nm, about 700 nm to about 900 nm, about 700 nm to about 1,000 nm, about 800 nm to about 900 nm, about 800 nm to about 1,000 nm, or about 900 nm to about 1,000 nm. In some embodiments, the system is configured to detect fluorophores have an absorption wavelength of about 200 nm, about 250 nm, about 300 nm, about 350 nm, about 400 nm, about 450 nm, about 500 nm, about 600 nm, about 700 nm, about 800 nm, about 900 nm, or about 1,000 nm. In some embodiments, the system is configured to detect fluorophores have an absorption wavelength of at least about 200 nm, about 250 nm, about 300 nm, about 350 nm, about 400 nm, about 450 nm, about 500 nm, about 600 nm, about 700 nm, about 800 nm, or about 900 nm. In some embodiments, the system is configured to detect fluorophores have an absorption wavelength of at most about 250 nm, about 300 nm, about 350 nm, about 400 nm, about 450 nm, about 500 nm, about 600 nm, about 700 nm, about 800 nm, about 900 nm, or about 1,000 nm.

In some embodiments, the system is configured to detect fluorophores that have an absorption wavelength of about 1,000 nm to about 4,000 nm. In some embodiments, the system is configured to detect fluorophores have an absorption wavelength of about 1,000 nm to about 1,250 nm, about 1,000 nm to about 1,500 nm, about 1,000 nm to about 1,750 nm, about 1,000 nm to about 2,000 nm, about 1,000 nm to about 2,250 nm, about 1,000 nm to about 2,500 nm, about 1,000 nm to about 2,750 nm, about 1,000 nm to about 3,000 nm, about 1,000 nm to about 3,250 nm, about 1,000 nm to about 3,500 nm, about 1,000 nm to about 4,000 nm, about 1,250 nm to about 1,500 nm, about 1,250 nm to about 1,750 nm, about 1,250 nm to about 2,000 nm, about 1,250 nm to about 2,250 nm, about 1,250 nm to about 2,500 nm, about 1,250 nm to about 2,750 nm, about 1,250 nm to about 3,000 nm, about 1,250 nm to about 3,250 nm, about 1,250 nm to about 3,500 nm, about 1,250 nm to about 4,000 nm, about 1,500 nm to about 1,750 nm, about 1,500 nm to about 2,000 nm, about 1,500 nm to about 2,250 nm, about 1,500 nm to about 2,500 nm, about 1,500 nm to about 2,750 nm, about 1,500 nm to about 3,000 nm, about 1,500 nm to about 3,250 nm, about 1,500 nm to about 3,500 nm, about 1,500 nm to about 4,000 nm, about 1,750 nm to about 2,000 nm, about 1,750 nm to about 2,250 nm, about 1,750 nm to about 2,500 nm, about 1,750 nm to about 2,750 nm, about 1,750 nm to about 3,000 nm, about 1,750 nm to about 3,250 nm, about 1,750 nm to about 3,500 nm, about 1,750 nm to about 4,000 nm, about 2,000 nm to about 2,250 nm, about 2,000 nm to about 2,500 nm, about 2,000 nm to about 2,750 nm, about 2,000 nm to about 3,000 nm, about 2,000 nm to about 3,250 nm, about 2,000 nm to about 3,500 nm, about 2,000 nm to about 4,000 nm, about 2,250 nm to about 2,500 nm, about 2,250 nm to about 2,750 nm, about 2,250 nm to about 3,000 nm, about 2,250 nm to about 3,250 nm, about 2,250 nm to about 3,500 nm, about 2,250 nm to about 4,000 nm, about 2,500 nm to about 2,750 nm, about 2,500 nm to about 3,000 nm, about 2,500 nm to about 3,250 nm, about 2,500 nm to about 3,500 nm, about 2,500 nm to about 4,000 nm, about 2,750 nm to about 3,000 nm, about 2,750 nm to about 3,250 nm, about 2,750 nm to about 3,500 nm, about 2,750 nm to about 4,000 nm, about 3,000 nm to about 3,250 nm, about 3,000 nm to about 3,500 nm, about 3,000 nm to about 4,000 nm, about 3,250 nm to about 3,500 nm, about 3,250 nm to about 4,000 nm, or about 3,500 nm to about 4,000 nm. In some embodiments, the system is configured to detect fluorophores have an absorption wavelength of about 1,000 nm, about 1,250 nm, about 1,500 nm, about 1,750 nm, about 2,000 nm, about 2,250 nm, about 2,500 nm, about 2,750 nm, about 3,000 nm, about 3,250 nm, about 3,500 nm, or about 4,000 nm. In some embodiments, the system is configured to detect fluorophores have an absorption wavelength of at least about 1,000 nm, about 1,250 nm, about 1,500 nm, about 1,750 nm, about 2,000 nm, about 2,250 nm, about 2,500 nm, about 2,750 nm, about 3,000 nm, about 3,250 nm, or about 3,500 nm. In some embodiments, the system is configured to detect fluorophores have an absorption wavelength of at most about 1,250 nm, about 1,500 nm, about 1,750 nm, about 2,000 nm, about 2,250 nm, about 2,500 nm, about 2,750 nm, about 3,000 nm, about 3,250 nm, about 3,500 nm, or about 4,000 nm.

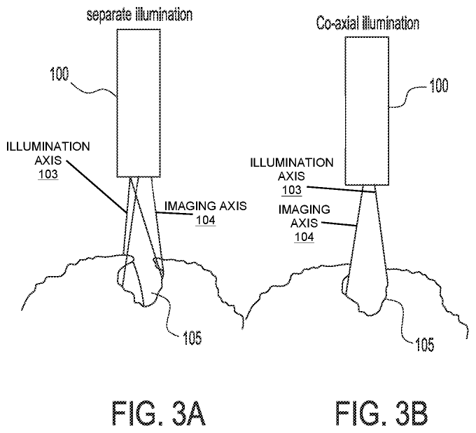

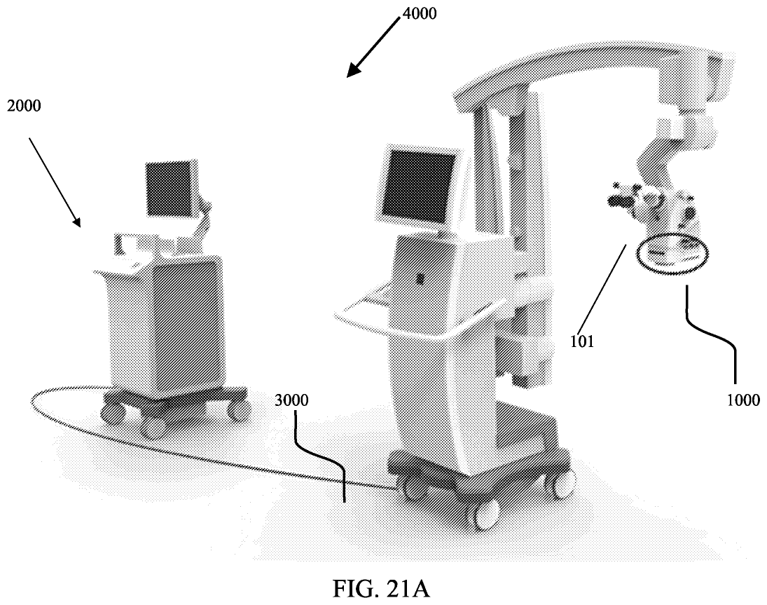

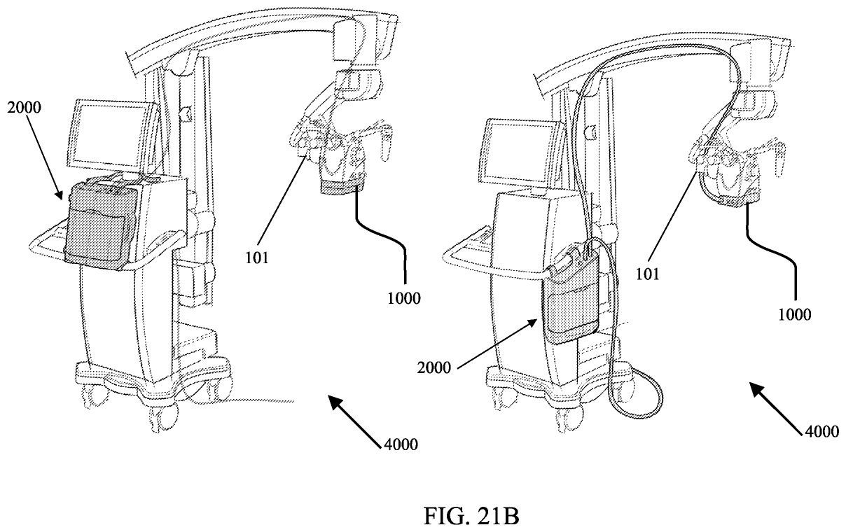

Referring to FIG. 21, in a particular embodiment, the imaging system 1000 herein is used with a microscope 101, e.g. a surgical microscope, for simultaneous imaging of fluorescence signal and visible light from the tissue 105. Referring to FIGS. 3A and 3B, in this embodiment, the illumination axis 103 of the fluorescence emission from the tissue is co-axial with the imaging axis 104. In other words, the excitation source's light is coaxial with an imaging axis of the imaging system 1000 and/or the operating microscope 101. In this embodiment, the microscope includes a visible light source for providing visible light to the imaging system.

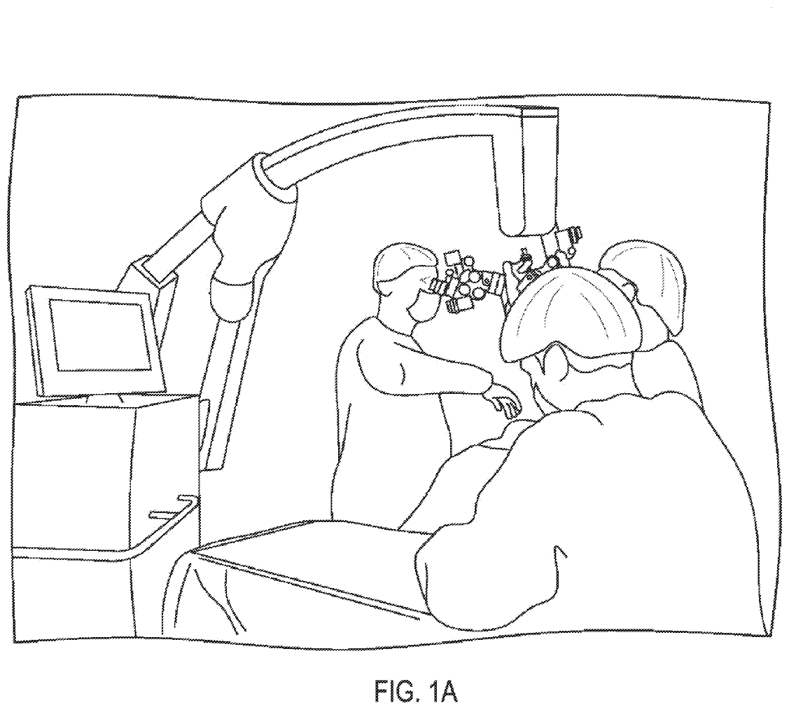

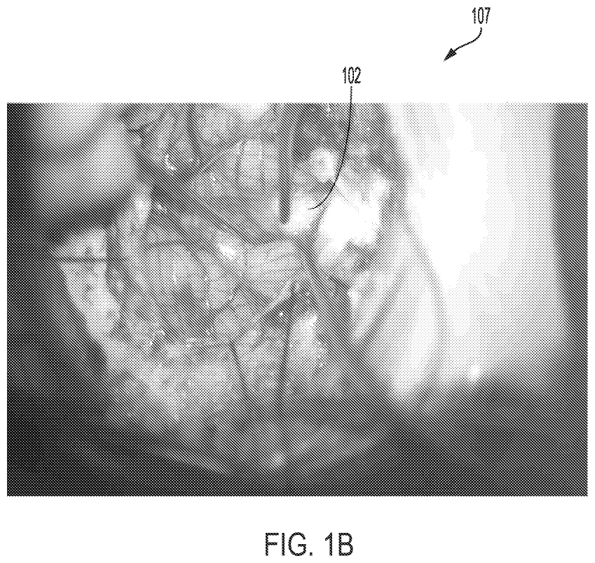

FIG. 1B shows an exemplary image generated using the imaging systems and methods herein. In this particular embodiment, the fluorescent tissue 102 is near the center of the field of view of the image display 107. In this embodiment, the fluorescent image is superimposed on visible image and the superimposed composite image is displayed on an external monitor. A digital processing device or a processor is used for processing and combining the images for display. In some embodiments, the surgeon directly views such visible and fluorescence images using the microscope. In some embodiments, the surgeon views such images from a heads-up display in the operation room or any other device capable of displaying images.

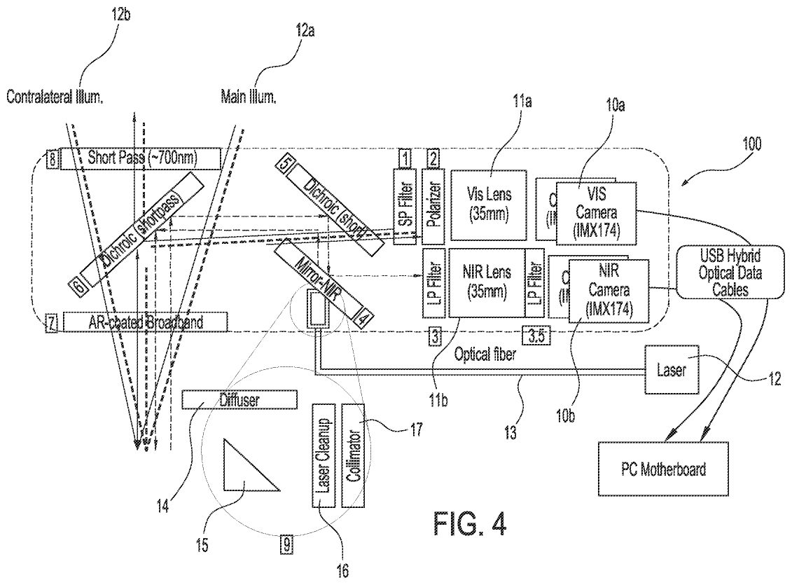

In some embodiments, the imaging system comprises a light source, one or more of an optical light guide, a shroud, a baffle, a light director, or any combination thereof. In some embodiments, the light source, the one or more optical light guide, the shroud, the baffle, and the light director are arranged to reduce the diffraction from the edges, and to reduce flooding of the NIR or IR sensor with the excitation light, the illumination light, or both. Exemplary arrangements of the light source and the optical light guide are shown in FIGS. 4, 5A-5D, 6A-6B, 16, and FIG. 18.

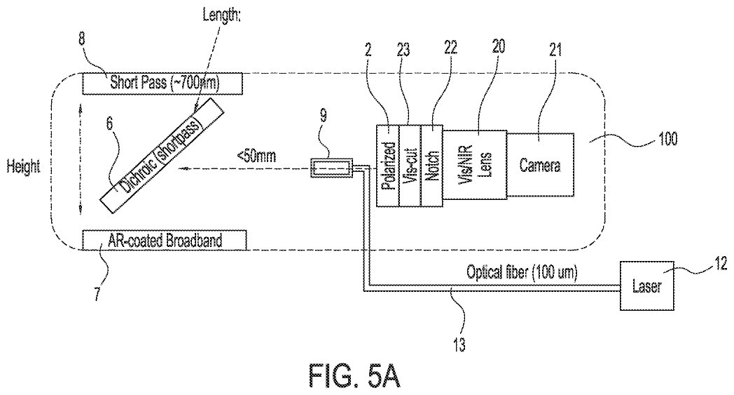

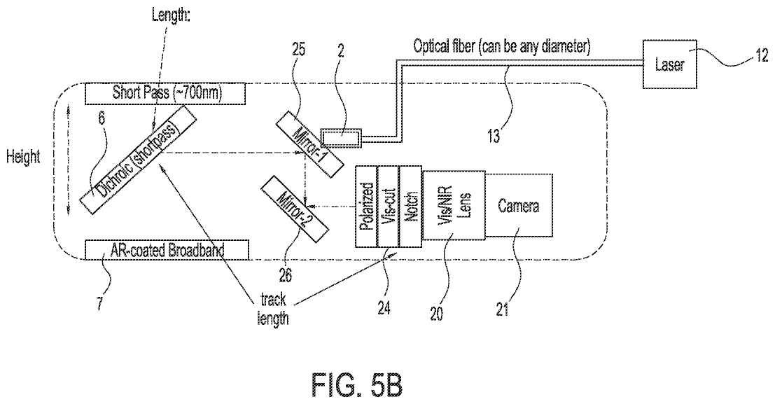

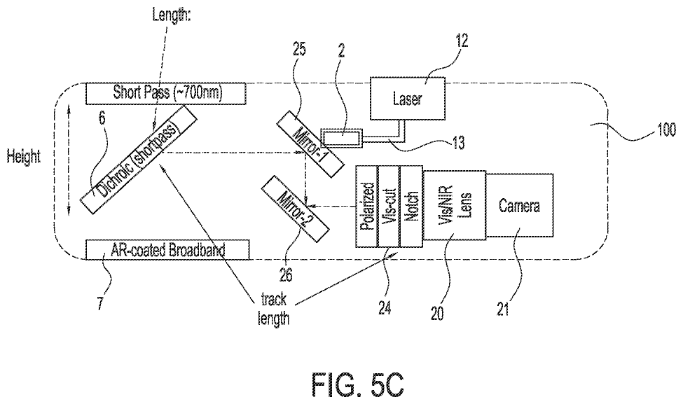

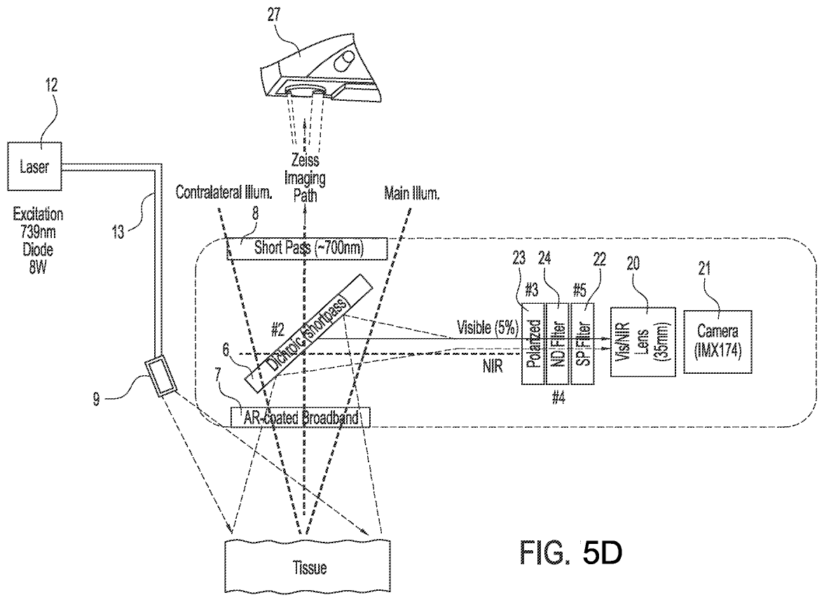

In some embodiments, the light source (e.g. laser and/or laser driver where light is emitted) is located internal to the imaging system 100, as shown in FIG. 5C. In some embodiments, the light source is adjacent to the imaging system, FIG. 5D. In some embodiments, the light source is located in close proximity to the imaging system. In some embodiments, the light source is located within about 5 mm to about 25 mm from the imaging system. In some embodiments the light source is external to the imaging system and light is introduced into the imaging system via an optical fiber.

In some embodiments, as shown in FIGS. 3A, 5D, and 16, the optical excitation assembly 9 is located proximal to the emitted light to reduce shadowing while imaging. In some embodiments, the optical excitation assembly 9 is about, 5 mm to about 25 mm from the light source 14. In some embodiments, the optical excitation assembly is within 0.5 mm, 1 mm, 3 mm, 5 mm, 10 mm, 13 mm, 15 mm, 18 mm, 20 mm, 23 mm, 25 mm, 28 mm, 30 mm up to 50 mm from where light is emitted 14.

Referring to FIGS. 4, 5A-5D, 6A-6B, FIG. 16 and FIG. 18 in a particular embodiment, the light source 12 generates an excitation light beam, whereby the excitation light beam has a wavelength in the ultraviolet, blue, visible, red, infrared, or NIR or IR range as described herein. In this embodiment, the light source 12 is coupled to an optical fiber 13. In some embodiments, the light from the optical fiber 13 is then be collimated using a collimator lens 17. Alternatively, the light source is directly coupled with a free space optic such as a mirror. In some embodiments the laser spectral characteristics correspond to the peak absorption value of the fluorophore.

After collimation, in some embodiments, the spectral bandwidth of the excitation light in some embodiments, is reduced using a band-pass filter, such as a laser clean up filter 16. In some embodiments, the laser clean up filter 16 is configured such that the excitation light spectrum is narrower than the notch filter. For example, optionally the notch reject is wider than the bandpass clean-up filter. In addition, the extra width required on the notch (i.e. reject filter) is related to the FOV and hence angle of incidence (AOI) on the filter(s) such that wider the AOI the larger the bandwidth required. A notch reject, or notch reject filter, also referred to as a band reject filter, passes all frequencies with the exception of those within a specified defined stop band which are greatly attenuated or not allowed to pass through the filter. In certain embodiments, the notch reject filter has a blocking band of greater than OD 6 at 785 nm with 39 nm notch bandwidth. In some embodiments, the transmission band is >93% transmission from 400-742 nm and >93% from 828-1600 nm. In some embodiments, the minimum blocking band is approximately double the transmission band of the clean-up filter. In some embodiments, for excitation light wavelengths outside 785 nm, the passband and blocking band of the respective filters should track the wavelength of the source used. In some embodiments, the notch blocking band is any width that does not block the bands of interest to the sensor, such as the visible band (for example, approximately 400-700 nm) and the fluorescence/emission band (for example, approximately 800-950 nm). In some embodiments, the notch filter is used to block reflected excitation source light from the target. In some embodiments, the laser cleanup filter comprises a filter with a full width half maximum that is less than a full width half maximum of the notch filter in order to inhibit cross talk between the excitation beam and the fluorescence beam emitted from the sample.

In some embodiments, the laser clean up filter and the notch filter both determine the spectral bandwidth that passes through to the respective filters and ultimately the sensor. For example, the spectrum of the excitation source and the specific clean up filter is configured such that the spectral width of the excitation beam emitted through the clean-up filter is narrower than the spectral width of the notch filter. In some embodiments, the spectral width of the notch filter as disclosed herein is a full width half maximum dimension of a beam transmitted through the filter. In some embodiments, the clean-up filter has a bandpass as described herein, depending on the excitation wavelength and fluorophore used. For example, in some embodiments, the clean-up filter has a bandpass of 15 nm (rejection of >4OD at 25 nm) depending on excitation wavelength and fluorophore used. In some embodiments, the laser energy is in the spectral bandwidth in the range of 5 nm with rest of the energy in wider spectral range up to but not limited to 15 nm.

In some embodiments, the laser cleanup filter narrows the bandwidth of the light source by about 1% to about 90%. In some embodiments, the laser cleanup filter narrows the bandwidth of the light source by about 1% to about 2%, about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 2% to about 5%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some embodiments, the laser cleanup filter narrows the bandwidth of the light source by about 1%, about 2%, about 5%, about 10%, about 20%, about 30%, about 40%, about 50%, about 60%, about 70%, about 80%, or about 90%. In some embodiments, the laser cleanup filter narrows the bandwidth of the light source by at least about 1%, about 2%, about 5%, about 10%, about 20%, about 30%, about 40%, about 50%, about 60%, about 70%, or about 80%. In some embodiments, the laser cleanup filter narrows the bandwidth of the light source by at most about 2%, about 5%, about 10%, about 20%, about 30%, about 40%, about 50%, about 60%, about 70%, about 80%, or about 90%.

In some embodiments, the laser cleanup filter narrows the bandwidth of the light source by about 1 nm to about 100 nm. In some embodiments, the laser cleanup filter narrows the bandwidth of the light source by about 1 nm to about 2 nm, about 1 nm to about 5 nm, about 1 nm to about 10 nm, about 1 nm to about 20 nm, about 1 nm to about 30 nm, about 1 nm to about 40 nm, about 1 nm to about 50 nm, about 1 nm to about 60 nm, about 1 nm to about 70 nm, about 1 nm to about 80 nm, about 1 nm to about 100 nm, about 2 nm to about 5 nm, about 2 nm to about 10 nm, about 2 nm to about 20 nm, about 2 nm to about 30 nm, about 2 nm to about 40 nm, about 2 nm to about 50 nm, about 2 nm to about 60 nm, about 2 nm to about 70 nm, about 2 nm to about 80 nm, about 2 nm to about 100 nm, about 5 nm to about 10 nm, about 5 nm to about 20 nm, about 5 nm to about 30 nm, about 5 nm to about 40 nm, about 5 nm to about 50 nm, about 5 nm to about 60 nm, about 5 nm to about 70 nm, about 5 nm to about 80 nm, about 5 nm to about 100 nm, about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 100 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 100 nm, about 40 nm to about 50 nm, about 40 nm to about 60 nm, about 40 nm to about 70 nm, about 40 nm to about 80 nm, about 40 nm to about 100 nm, about 50 nm to about 60 nm, about 50 nm to about 70 nm, about 50 nm to about 80 nm, about 50 nm to about 100 nm, about 60 nm to about 70 nm, about 60 nm to about 80 nm, about 60 nm to about 100 nm, about 70 nm to about 80 nm, about 70 nm to about 100 nm, or about 80 nm to about 100 nm. In some embodiments, the laser cleanup filter narrows the bandwidth of the light source by about 1 nm, about 2 nm, about 5 nm, about 10 nm, about 20 nm, about 30 nm, about 40 nm, about 50 nm, about 60 nm, about 70 nm, about 80 nm, or about 100 nm. In some embodiments, the laser cleanup filter narrows the bandwidth of the light source by at least about 1 nm, about 2 nm, about 5 nm, about 10 nm, about 20 nm, about 30 nm, about 40 nm, about 50 nm, about 60 nm, about 70 nm, or about 80 nm. In some embodiments, the laser cleanup filter narrows the bandwidth of the light source by at most about 2 nm, about 5 nm, about 10 nm, about 20 nm, about 30 nm, about 40 nm, about 50 nm, about 60 nm, about 70 nm, about 80 nm, or about 100 nm.

In some embodiments, the cleaned up light is then reflected by a dielectric mirror. The cleaned light is reflected at an angle of about 0 degrees to about 180 degrees. In some embodiments, the cleaned up light is reflected by the dielectric mirror. In some embodiments, the cleaned light is reflected at an angle of about 60 degrees to about 120 degrees. In some embodiments, the cleaned up light is reflected by the dielectric mirror at an angle of at least about 10 degrees, 20 degrees, 30 degrees, 40 degrees, 50 degrees, 60 degrees, 70 degrees, 80 degrees, 90 degrees, 100 degrees, 110 degrees, or 120 degrees, including increments therein. In some embodiments, the cleaned up light is reflected by the dielectric mirror at an angle of at most about 10 degrees, 20 degrees, 30 degrees, 40 degrees, 50 degrees, 60 degrees, 70 degrees, 80 degrees, 90 degrees, 100 degrees, 110 degrees, or 120 degrees, including increments therein. In some embodiments, the cleaned light is reflected at an angle of about 90 degrees. In some embodiments, the reflected light is then diffused at calculated angle(s) through a hole in the NIR mirror 4 to match the cone of imaging light using a diffuser. In some embodiments, the diffuser also ensures that the excitation source's light is evenly distributed to produce a flat or relatively homogenous illumination profile on the target tissue.

A non-limiting example of the laser is a BWT 8 W diode laser. Non-limiting example of the optical fiber is a 105 um core optical fiber with a cladding of 125 um, with a buffer of 250 um and 0.22NA, and a length of 100 cm=/−10 cm. Non-limiting example of the diffuser is Thorlabs 20 degree Circle engineered diffuser (RPC) #ED1-C20. Non-limiting example of the collimator lens is Thorlabs A110TM-B, f=6.24 mm, NA=0.40, Rochester Aspheric. Non-limiting example of the laser clean-up filter is DiodeMax 785 Semrock-LD01-785/10-12.5. In some embodiments, the laser or other infrared light source has a power of about 1 watt to about 8 watts. In some embodiments, the power of the laser or other infrared light source is at least partially determined based on the location. In some embodiments, if the laser or other infrared light source is located in the Imaging System, rather than the Imaging Station, fewer optical couplers are required, and the optical guide distance will be shorter. In some embodiments, as optical couplers and fiber length contributes to optical power loss, a relatively lower power laser or infrared light source are employed if it is located in the Imaging Head.

In some embodiments, the excitation light source includes one or more elements in the assembly, which includes one or more of but is not limited to collimator, clean up filter, dielectric mirror, and diffuser. In some embodiments, this cleaned up light is reflected at any angle, for example, between 45 degrees and 90 degrees, or between 90 degrees and 135 degrees, using a dielectric mirror. Moreover, in other embodiments although the cleaned up light is reflected at any arbitrary angle, with or without dielectric mirror. As shown in FIG. 4, the dichroic shortpass filter 6 accepts light perpendicular to the plane of the paper.

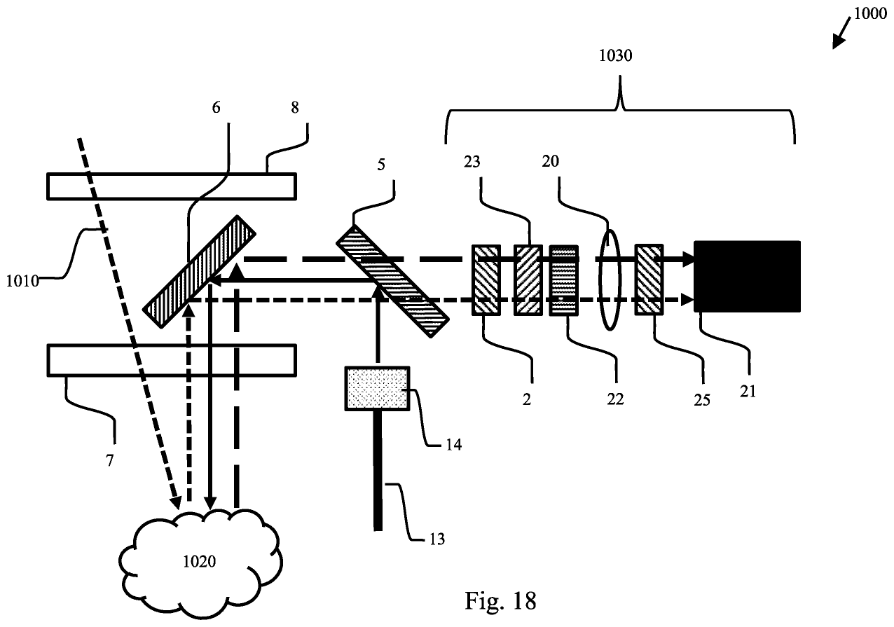

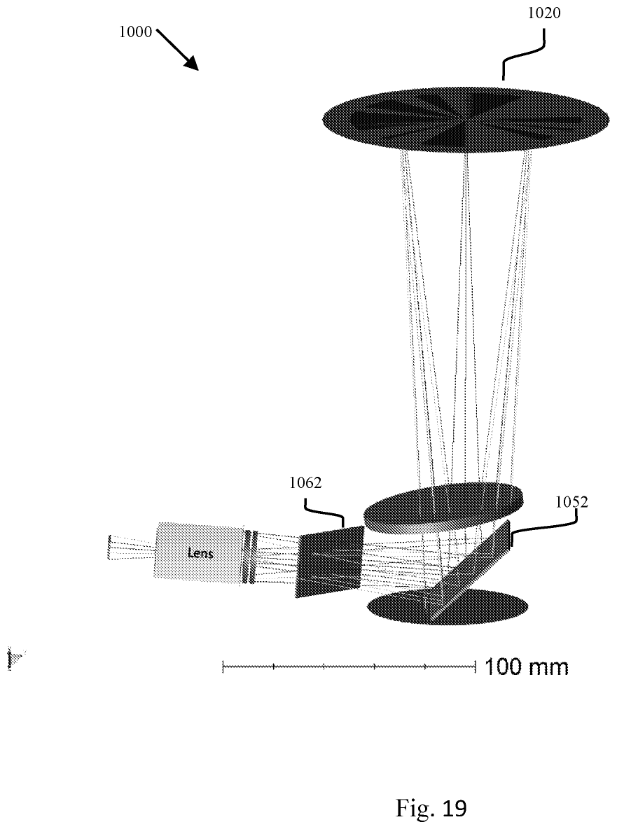

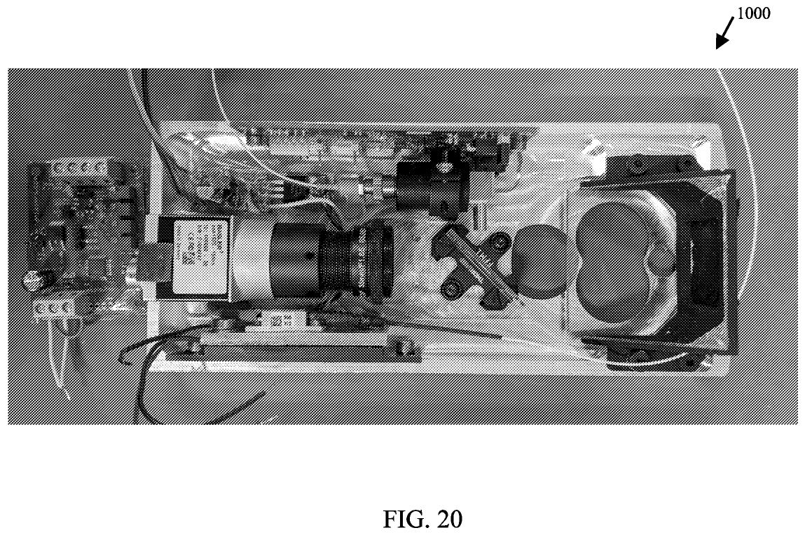

Further provided herein, per FIGS. 18-20, is an imaging system 1000 for imaging an emission light emitted by a tissue sample 1020 comprising a fluorophore. As shown in FIG. 18, the system 1000 comprises an optical fiber 13, a diffuser 14, a visible channel 1010, an optical device 1052, that may be but is not limited to the shortpass dichroic mirror 5, and an imaging assembly 1030. FIG. 19 provides a ray diagram illustrating exemplary light paths of the imaging system 1000 shown in FIG. 18, while FIG. 20 is a photograph of an example imaging system according to one embodiment.

In some embodiments, at least a portion of the excitation light is emitted by a laser. In some embodiments, the imaging system 1000 comprises the laser. In some embodiments, the imaging system 1000 does not comprise the laser. In some embodiments, the laser has an off mode and an on mode. In some embodiments, the excitation light has a wavelength of about 775 nm to about 795 nm. In some embodiments, the excitation light has a wavelength of about 785 nm. In some embodiments, the excitation light is an infrared or a near-infrared excitation light.

In some embodiments, the excitation diffuser 14 diffuses at least a portion of the excitation light. In some embodiments, the excitation diffuser 14 is a circular excitation diffuser 14. In some embodiments, the circular excitation diffuser 14 has a diffusion angle of about 4 degrees to about 25 degrees. In some embodiments, the excitation diffuser 14 is a rectangular excitation diffuser 14. In some embodiments, the rectangular excitation diffuser 14 has a first diffusion angle and a second diffusion angle perpendicular to the first diffusion angle. In some embodiments, the first diffusion angle, the second diffusion angle, or both are about 4 degrees to about 25 degrees. In some embodiments, the first diffusion angle is about 14 degrees, and wherein the second diffusion angle is about 8 degrees. In some embodiments, the diffuser comprises a glass diffuser, a ground diffuser, a holographic diffuser, an engineered diffuser, or any combination thereof. In some embodiments, the engineered diffuser comprises an etched plastic, an etched film bonded to a glass substrate, or both.

In some embodiments, the diffused light forms a shape comprising a line, a triangle, a circle, a square, a rectangle, a polygon, or any combination thereof. In some embodiments, the diffused light forms a shape comprising a line, a triangle, a circle, a square, a rectangle, a polygon, or an array with mirrors that scan at least the field of view (FOV). In some embodiments, the shape of the diffused light illuminates the field of view (FOV) of the camera. In some embodiments, the shape of the diffused light does not illuminate any area beyond the FOV of the camera to conserve laser power. In some embodiments, the shape of the diffused light only illuminates the FOV of the camera to conserve laser power. In some embodiments, conserving and/or minimizing the required laser power reduces excess heat within the system and thus reduces the amount of cooling required n some embodiments, conserving and/or minimizing the required laser power reduces the amount of required current and the wire size necessary to transmit such current.

In some embodiments, the diffuser uniformly illuminates the FOV of the camera. In some embodiments, the circular diffuser provides the most uniform coverage. In some embodiments, the circular diffuser is arranged with respect to the camera, such that it illuminates a circle that circumscribes the FOV of the camera. In some embodiments, the rectangular diffuser reduces the required laser power by about half because it optimally fills the FOV of the camera. In some embodiments, the shape of the diffuser depends on the required FOV of the fluorescence area, and should be matched appropriately to increase the efficiency and avoid overfilling the FOV. In some embodiments, the shape of the diffuser cis modified or adjusted to correspond to the shape of the FOV in order to reduce the overfilling of the FOV, as well increase efficiency and conserve power usage as described herein. Types of diffusers include, for example, coated or not coated ground glass, holographic, white diffusing glass, and engineered diffusers amongst other substrates.

In some embodiments, the visible channel 1010 receives and directs at least a portion of a visible light to the sample 1020. In some embodiments, at least a portion of the visible light is received by a microscope, or an endoscope, and sourced by a light bulb, a light emitting diode (LED), or any combination thereof. In some embodiments, the imaging system 1000 comprises the visible light. In some embodiments, the imaging system 1000 does not comprise the visible light. In some embodiments, the visible light has a wavelength of about 400 nm to about 700 nm, while extending into the NIR band from 700 to 950 nm.

In some embodiments, the optical device 1052 directs at least a portion of the diffused excitation light to the sample 1020. In some embodiments, the optical device 1052 allows at least a portion of the emission light and a reflected visible light to pass therethrough to an imaging assembly 1030. In some embodiments, the optical device 1052 filters at least a portion of the emission light, the excitation light, or both. In some embodiments, the optical device 1052 does not filter the emission light, the excitation light, or both. In some embodiments, the optical device 1052 directs at least a portion of the diffused excitation light to the sample 1020 in a first direction and allows at least a portion of the emission light and a reflected visible light to pass therethrough in a second direction opposite the first direction. In some embodiments, the optical device 1052 allows at least a portion of the diffused excitation light to pass therethrough in a direction parallel to the diffused excitation light. In some embodiments, the optical device 1052 blocks and/or reflects at least 5%, 10%, 15%, 20%, 25%, 30%, 35%, 40%, 45%, 50%, 55%, 60%, 65%, 70%, 75%, 80%, 85%, 90%, 95% or more of diffused excitation light, including increments therein. In some embodiments, the optical device 1052 blocks and/or reflects at least 96%, 97%, 98%, 99% or more, up to 100% or all of the diffused excitation light from passing therethrough in a direction parallel to the diffused excitation light, including increments therein.

In some embodiments, the optical device 1052 is a hot mirror, a dichroic mirror, a shortpass filter, or any combination thereof. In some embodiments, the hot mirror transmits visible light while blocking NIR or IR light. In cases where the fluorescence is shorter in wavelength (e.g., in the ultraviolet spectrum), the optical device functions as a long-pass filter, to reflect shorter wavelengths in the UV spectrum. In some embodiments, the optical device transmits UV light while blocking visible light.

In some embodiments, the system 1000 further the optical device 1052 is a hot mirror 6 in the path of the visible light. In some embodiments, the hot mirror 6 filters out at least a portion of the wavelength of the NIR or IR light from the visible light.

Further, as shown in FIG. 18, the imaging assembly 1030 comprises a first notch filter 2, a longpass filter 23, a lens 20, a second notch filter 25, and an image sensor 21. As shown, in some embodiments, the emission light and the reflected visible light are consecutively directed from the sample 1020 through the notch beam splitter, the first notch filter 2, the longpass filter 23, the lens 20, and the second notch filter 25. In some embodiments, the emission light and the reflected visible light are directed from the sample 1020 and sequentially through the notch beam splitter, the first notch filter 2, the longpass filter 23, the lens 20, and the second notch filter 25 in any order. In some embodiments, the imaging assembly 1030 does not comprise the second notch filter 25. In some embodiments, the imaging assembly 1030 does not comprise one or more of the first notch filter 2, the longpass filter 23, the lens 20, and the second notch filter 25.

In some embodiments, at least one of the first notch filter 2 and the second notch filter 25 block at least a portion of the excitation light from passing therethrough. In some embodiments, at least one of the first notch filter 2 and the second notch filter 25 block at least 5%, 10%, 15%, 20%, 25%, 30%, 35%, 40%, 45%, 50%, 55%, 60%, 65%, 70%, 75%, 80%, 85%, 90%, 95% or more of the excitation light from passing therethrough. In some embodiments, at least one of the first notch filter 2 and the second notch filter 25 block at least 96%, 97%, 98%, 99% or more, up to 100% or all of the excitation light from passing therethrough. In some embodiments, at least one of the first notch filter 2 and the second notch filter 25 block at least a portion of light with a wavelength of about 775 nm to about 795 nm from passing therethrough. In some embodiments, at least one of the first notch filter 2 and the second notch filter 25 block at least a portion of light with a wavelength of about 785 nm from passing therethrough.

In some embodiments, the longpass filter 23 comprises a vis-cut longpass filter 23. In some embodiments, the longpass filter 23 at least partially reduces transmission of the visible light therethrough. In some embodiments, the longpass filter 23 transmits a majority of the NIR or IR light. In some embodiments, the longpass filter 23 transmits at least about 85%, 86%, 87%, 88%, 89%, 90%, 91%, 92%, 93%, 94%, 95%, 96%, 97%, 98%, or 99% of the NIR or IR light, including increments therein. In some embodiments, the long pass filter comprises a visible light attenuator. In some embodiments, the visible light attenuator transmits near infrared wavelengths. In some embodiments, the second notch filter 25 reduces any scatter that passes through the first notch filter 2. In some embodiments, the second notch filter 25 reflects back at least a portion of the excitation light . . . .

In some embodiments, the image sensor 21 is configured to detect both the emission light and the reflected visible light from the sample 1020. In some embodiments, the image sensor 21 is configured to generate image frames based on the emission light and the reflected visible light.

In some embodiments, the imaging assembly 1030 further comprises a polarizer 22. In some embodiments, the imaging assembly 1030 does not comprise the polarizer 22. In some embodiments, at least a portion of the emission light and the reflected visible light are directed through the long pass-filter, the polarizer 22, and the lens 20. In some embodiments, at least a portion of the emission light and the reflected visible light are directed sequentially through the longpass filter 23, the polarizer 22 and the lens 20. In some embodiments, the polarizer 22 reduces a ghosting effect from reflections of the front/back surfaces of the shortpass dichroic 6. In some embodiments, the polarizer 22 is removable from the imaging assembly 1030.

In some embodiments, the system 1000 further comprises a white light that emits at least a portion of the visible light. In some embodiments, the system 1000 further comprises a laser that emits at least a portion of the excitation light. In some embodiments, the system 1000 does not comprise the white light that emits the visible light. In some embodiments, the system 1000 does not comprise the laser that emits the excitation light. In some embodiments, the system 1000 further comprises a shortpass dichroic mirror 6 between the imaging assembly 1030 and the sample 1020 and between the excitation diffuser 14 and the sample 1020. In some embodiments, the shortpass dichroic mirror 6 transmits wavelengths of about 400 nm to about 800 nm. In some embodiments, the shortpass dichroic filter 6 reflects wavelengths greater than about 720 nm. In some embodiments, a size of a clear aperture of the shortpass dichroic mirror 6 is sufficiently small such that the shortpass dichroic mirror 6 does not block at least a portion of the visible light transmitted to the sample 1020. In some embodiments, the visible light is not transmitted through the shortpass dichroic mirror 6.

In some embodiments, the shortpass dichroic mirror 6 has a shape comprising a circle, a triangle, a rectangle, a square, or any polygon. In some embodiments, the shape of the shortpass dichroic mirror 6 is configured to mechanically fit within the system 1000. In some embodiments, the shape of the shortpass dichroic mirror 6 is configured based on the microscope, the endoscope, or both. In some embodiments, the shortpass dichroic mirror 6 is ground/cut and shaped to fit into the system 1000. In some embodiments, the shape of the shortpass dichroic mirror 6 is configured to avoid interference with the visible light channel, a microscope's illumination light path, or both. In some embodiments, the shape of the shortpass dichroic mirror 6 is configured to avoid interference with the visible light channel, a microscope's illumination light path, or both while maintaining coincidence with the imaging path of the microscope, the optical device, or both. In some embodiments, the shortpass dichroic mirror 6 is formed of glass, structural metallic-glass composites, plastic, pellicle mirror, or any combination thereof. In some embodiments, the shortpass dichroic mirror 6 is shaped to reduce wavefront error. In some embodiments, the shortpass dichroic mirror 6 comprises a concave surface, a convex surface, a flat surface, or any combination thereof to reduce wavefront error.

In some embodiments, an angle between a reflective plane of the shortpass dichroic mirror 6 and an imaging axis normal to the image sensor 21 is about 40 degrees to about 60 degrees. In some embodiments, the angle between a reflective plane of the shortpass dichroic mirror 6 and the imaging axis normal to the image sensor 21 is about 45 degrees to about 50 degrees. In some embodiments, an angle between a reflective plane of the shortpass dichroic mirror 6 and the path of the excitation light is about 0 degrees to about 90 degrees, or between 0 and 90 degrees (not inclusive). In some embodiments, the angle between the reflective plane of the shortpass dichroic mirror 6 and the path of the excitation light is about 30 degrees to about 55 degrees. In some embodiments, the angle between a reflective plane of the shortpass dichroic mirror 6 and the path of the excitation light is about 40 degrees to about 50 degrees. In some embodiments, the angle between a reflective plane of the shortpass dichroic mirror 6 and the path of the excitation light is 45 degrees+/−10 degrees. In some embodiments, the angle between a reflective plane of the shortpass dichroic mirror 6 and the path of the excitation light is 5°, 10°, 15°, 20°, 25°, 30°, 35°, 45°, 50°, 55°, 60°, 65°, 70°, 75°, 80°, 85° or any other angle between 0 and 90 degrees. In some embodiments, the angle between a reflective plane of the shortpass dichroic mirror 6 and the path of the excitation light is 5°, 10°, 15°, 20°, 25°, 30°, 35°, 45°, 50°, 55°, 60°, 65°, 70°, 75°, 80°, 85° or any other angle between 0 and 90 degrees. In some embodiments, the angle between a reflective plane of the shortpass dichroic mirror 6 and the path of the excitation light is about 30° to about 55°, about 30° to about 60°, about 40° to about 50°, about 40° to about 60°, about 45° to about 50°, about 45° to about 60°.

In some embodiments, the angle between a reflective plane of the shortpass dichroic mirror 6 and the path of the excitation light is achieved by using one or more additional mirrors relative to the shortpass dichroic mirror 6. In some embodiments, the angle between a reflective plane of the shortpass dichroic mirror 6 and the one or more additional mirrors is 5°, 10°, 15°, 20°, 25°, 30°, 35°, 45°, 50°, 55°, 60°, 65°, 70°, 75°, 80°, 85° or any other angle between 0 and 90 degrees, 45 degrees+/−10 degrees, about 30° to about 55°, about 30° to about 60°, about 40° to about 50°, about 40° to about 60°, about 45° to about 50°, about 45° to about 60°.

In some embodiments, the system 1000 further comprises a bottom window 7 between the shortpass dichroic mirror 6 and the sample 1020. In some embodiments, the bottom window 7 is at least partially transparent. In some embodiments, the bottom window 7 is fully transparent. In some embodiments, the system 1000 further comprises a top window 8 at the interface of the system 1000 and microscope 101.

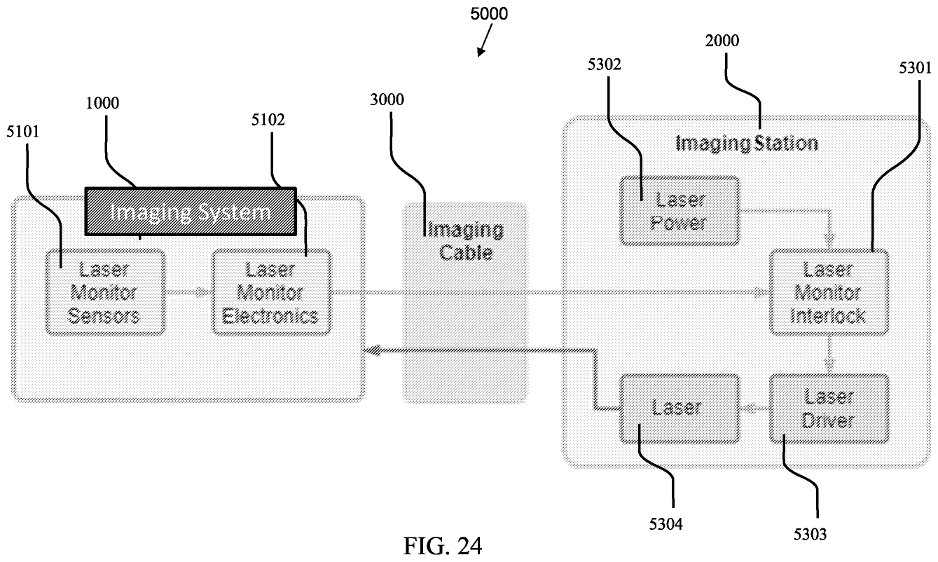

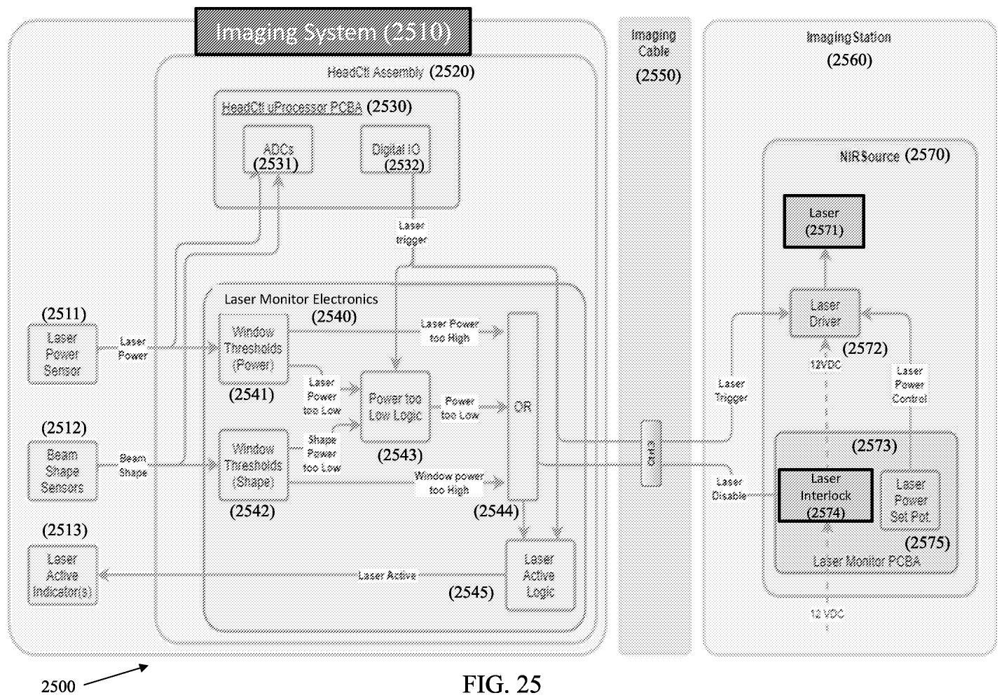

In some embodiments, per FIGS. 24-25, the system 1000 further comprises a laser monitor sensor. In some embodiments, the laser monitor sensor comprises an excitation light power gauge, a diffused beam shape sensor, or both. In some embodiments, the excitation light power gauge is configured to measure a power of the excitation light. In some embodiments, the diffused beam shape sensor measures a diffused beam shape. In some embodiments, the system 1000 further comprises one or more diffused beam shape sensor, one or more diffused beam shape gauges, or both. In some embodiments, the diffused beam shape sensor comprises a first diffused beam shape gauge, a second diffused beam shape gauge, or both. In some embodiments, the diffused beam shape sensor comprises 2, 3, 4, 5, 6, 7, 8, 9, 10 or more beam shape gauges. In some embodiments, the two or more diffused beam shape gauges measure a strength of the diffused beam at multiple locations with the shape of the diffused beam. In some embodiments, at least one diffused beam sensor measures at least one edge of the beam shape, and/or incrementally along at least one of 10%, 20%, 30%, 40%, 50%, 60%, 70%, 80% or up to 90% inclusive within diffused beam shape or at one or more locations on the beam, and at least one other diffused beam sensor measures at least one other point within diffused beam shape or at one or more other locations on the beam. In some embodiments, the two or more diffused beam shape gauges measure a sample at one-or-more locations on the beam. In some embodiments, each measurement along the beam shape is compared to high and/or low limits, or could be compared with one-or-more other measurements along the beam shape to calculate a relative measurement between two or more points along the beam, which would then be tested against high and/or low limits. In some embodiments, the diffused beam shape gauges or sensors monitor and detect a positive or negative rate-of-change of the monitored values or relative measurements, and if the rate-of-change or the measured value or relative measurements changes too rapidly, laser or imaging system malfunction is indicated and the laser is shut off. This safety mechanism is used to monitor the beam and performance of the laser to serve as a safety switch or shutoff to reduce fluence (an indication of the amount of energy delivered over the area) in the event the laser or imaging system malfunctions. In some embodiments, the laser is shut off within a millisecond of malfunction, within a microsecond of malfunction, or a picosecond of malfunction, or less. Shutting off the laser is critical to avoid burning of tissue in the event the imaging system is used in applications that are in situ in human or animals, for example in open field surgical applications or endoscopically. As shown in FIG. 24, in some embodiments, one or more of the laser monitor sensors 5101 and the laser monitor electronics 5102 communicate with a laser monitor interlock 5301. In some embodiments, the laser monitor sensors 5101, the laser monitor electronics 5102, or both are located within the imaging system 1000. In some embodiments, one or more of the laser monitor sensors 5101 and the laser monitor electronics 5102 communicate with a laser monitor interlock 5301 via an imaging cable 3000. In some embodiments, the laser monitor interlock 5301 transmits power from a laser power source 5301 to the laser driver 5303 based on the data received by the laser monitor sensors 5101, the laser monitor electronics 5102, or both. In some embodiments, the laser driver 5303 directs the laser 5304. In some embodiments, one or more of the laser monitor interlock 5301, the laser power source 5302, the laser driver 5303, and the laser 5304 are located within an imaging station 2000. In some embodiments, a laser beam output by the laser 5304 is transmitted through the imaging cable 3000 to the imaging system 1000. In other embodiments, the laser 5304, laser driver 5303, laser interlock 5301, and laser monitor electronics 5102 are all located in the Imaging System 1000. In some embodiments, any of the components are located at the Imaging Station 2000 or Imaging System 1000

In some embodiments, the system 1000 further comprise a reflector redirecting at least a portion of the excitation light to the excitation light power gauge. In some embodiments, the reflector is positioned between the optical fiber and the excitation diffuser 14. In some embodiments, an optical device 1062 that includes the shortpass dichroic filter 6, allows at least a portion of the diffused excitation light to pass therethrough in a direction parallel to the original axis from diffuser 14. In some embodiments, the diffused beam shape sensor receives the portion of the diffused excitation light. In some embodiments, the first diffused beam shape gauge measures a power of the diffused beam at a center of the diffused beam shape and wherein the second diffused beam shape gauge measures the power of the diffused beam at an edge of the diffused beam shape. In some embodiments, the excitation light power gauge, the first diffused beam shape gauge, the second diffused beam shape gauge, or any combination thereof comprises a photodiode, a camera, a piezoelectric sensor, a linear sensor array, a CMOS sensor, or any combination thereof. In some embodiments, one or more of the beam shape sensors are located near, in the plane of, or behind the notch dichroic mirror 5. In some embodiments, one or more of the beam shape sensors are located in the plane of, or behind the shortpass dichroic filter 6. In other embodiments, the sensors are located anywhere between the diffuser in 14 to the bottom window 7. In other embodiments, one or more of the beam shape sensors are located at any location within the imaging system directly in the path of the beam.

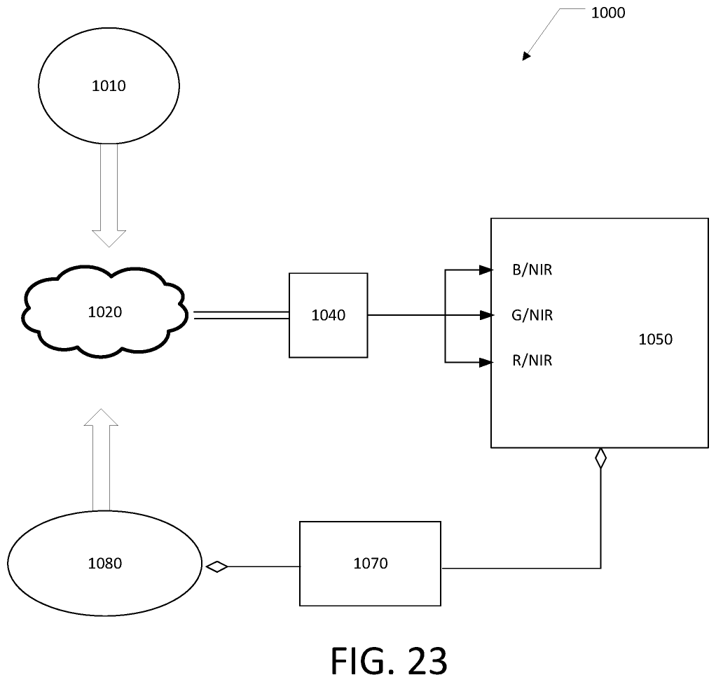

FIG. 23 shows a schematic diagram of the time multiplexing assembly of an exemplary single camera imaging system. Exemplary single camera imaging systems are shown in FIGS. 5A-D, 6A-B, and 18. In some embodiments, the white light 1010 and the laser 1080 are directed towards a tissue sample 1020, wherein excitation light emitted by the fluoresced sample 1020 is filtered at filter 1040 and sent to B/NIR, G/NIR, and R/NIR ports in an RGB sensor 1050. According to various embodiments, the RGB sensor 1050 is a portion of the sensor or camera 21. The assembly also includes a laser driver 1070 that receives the timing or clock data from the camera for triggering the excitation laser 1080.

In some aspects, the imaging system is configured to detect, image or assess a therapeutic agent; detect, image or assess a safety or a physiologic effect of the companion diagnostic agent; detect, image or assess a safety or a physiologic effect of the therapeutic agent; detect, image or assess a safety or a physiologic effect of the companion imaging agent; or any combination thereof. In some aspects, the contrast or imaging agent's safety or physiologic effect is bioavailability, uptake, concentration, presence, distribution and clearance, metabolism, pharmacokinetics, localization, blood concentration, tissue concentration, ratio, measurement of concentrations in blood or tissues, therapeutic window, range and optimization, or any combination thereof. In some aspects, the method comprises administering a companion diagnostic agent, a therapeutic agent, or an imaging agent, and wherein the imaging comprises detecting the companion diagnostic agent, the therapeutic agent, or the imaging agent. In some aspects, the companion diagnostic agent, the therapeutic agent, or the imaging agent comprises a chemical agent, a radiolabel agent, radiosensitizing agent, photosensitizing agent, fluorophore, therapeutic agent, an imaging agent, a diagnostic agent, a protein, a peptide, nanoparticle or a small molecule.

The systems and methods of the present disclosure can be used alone or in combination with a companion diagnostic, therapeutic or imaging agent (whether such diagnostic, therapeutic or imaging agent is a fluorophore alone, or conjugated, fused, linked, or otherwise attached to a chemical agent or other moiety, small molecule, therapeutic, drug, chemotherapeutic, protein, peptide, nanoparticle, antibody protein or fragment of the foregoing, and in any combination of the foregoing; or used as a separate companion diagnostic, therapeutic or imaging agent in conjunction with the fluorophore or other detectable moiety is alone, conjugated, fused, linked, or otherwise attached to a chemical agent or other moiety, small molecule, therapeutic, drug, chemotherapeutic, peptide, nanoparticle, antibody protein or fragment of the foregoing, and in any combination of the foregoing). Such companion diagnostics utilize agents including chemical agents, radiolabel agents, radiosensitizing agents, photosensitizing agents, fluorophores, imaging agents, diagnostic agents, protein, peptide, nanoparticle or small molecule such agent intended for or having diagnostic or imaging effect. Agents used for companion diagnostic agents and companion imaging agents, and therapeutic agents, include the diagnostic, therapeutic and imaging agents described herein or other known agents. Diagnostic tests are used to enhance the use of therapeutic products, such as those disclosed herein or other known agents. The development of therapeutic products with a corresponding diagnostic test, such as a test that uses diagnostic imaging (whether in vivo, in situ, ex vivo or in vitro) aid in diagnosis, treatment, identify patient populations for treatment, and enhance therapeutic effect of the corresponding therapy. The systems and methods of the present disclosure are also be used to detect therapeutic products, such as those disclosed herein or other known agents, to aid in the application of a therapy and to measure it to assess the agent's safety and physiologic effect, e.g. to measure bioavailability, uptake, distribution and clearance, metabolism, pharmacokinetics, localization, blood concentration, tissue concentration, ratio, measurement of concentrations in blood and/or tissues, assessing therapeutic window, range and optimization, and the like of the therapeutic agent. Such The systems and methods are employed in the context of therapeutic, imaging and diagnostic applications of such agents. Tests also aid therapeutic product development to obtain the data FDA uses to make regulatory determinations. For example, such a test may identify appropriate subpopulations for treatment or identify populations who should not receive a particular treatment because of an increased risk of a serious side effect, making it possible to individualize, or personalize, medical therapy by identifying patients who are most likely to respond, or who are at varying degrees of risk for a particular side effect. Thus, the present disclosure, in some embodiments, includes the joint development of therapeutic products and diagnostic devices, including the systems and methods herein (used to detect the therapeutic and/or imaging agents themselves, or used to detect the companion diagnostic or imaging agent, whether such diagnostic or imaging agent is linked to the therapeutic and/or imaging agents or used as a separate companion diagnostic or imaging agent linked to the peptide for use in conjunction with the therapeutic and/or imaging agents) that are used in conjunction with safe and effective use of the therapeutic and/or imaging agents as therapeutic or imaging products. Non-limiting examples of companion devices include a surgical instrument, such as an operating microscope, confocal microscope, fluorescence scope, exoscope, endoscope, or a surgical robot and devices used in biological diagnosis or imaging or that incorporate radiology, including the imaging technologies of X-ray radiography, magnetic resonance imaging (MRI), medical ultrasonography or ultrasound, endoscopy, elastography, tactile imaging, thermography, medical photography and nuclear medicine functional imaging techniques as positron emission tomography (PET) and single-photon emission computed tomography (SPECT). In some embodiments, the companion diagnostics and devices comprise tests that are conducted ex vivo, including detection of signal from tissues or cells that are removed following administration of the companion diagnostic to the subject, or application of the companion diagnostic or companion imaging agent directly to tissues or cells following their removal from the subject and then detecting signal. Examples of devices used for ex vivo detection include fluorescence microscopes, flow cytometers, and the like. Moreover, in some embodiments, the systems and methods herein for such use in companion diagnostics are used alone or alongside, in addition to, combined with, attached to or integrated into an existing surgical microscope, confocal microscope, fluorescence scope, exoscope, endoscope, or a surgical robot. In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica ARveo Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., Retina Vue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPALI NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

Moreover, in some embodiments, the imaging, diagnostic, detecting and therapeutic methods herein are performed using the systems described herein alongside, in addition to, combined with, attached to, or integrated into such an existing surgical microscope, confocal microscope, fluorescence scope, exoscope, endoscope, surgical robot, microscope, exoscope, or endoscope as described above.

In some embodiments, the components of the system herein are positioned and coupled using fasteners such as, for example, a screw, a nut and a bolt, clamps, vices, adhesives, bands, ties, or any combination thereof.

In some embodiments, the systems 1000 and the components therein are configured to minimize its the overall size. In some embodiments, the compactness of the systems 1000 herein improves its operability, maintains sensitivity, improves portability, storage, ease of use, and affordability. In some embodiments, the compactness of the systems 1000 herein improves a caregiver's ability and speed to employ and manipulate the system 1000. In addition, by minimizing the overall size of the systems 1000 and the components therein avoiding large system volume, storage, cost, integration, improving human factors and general usability for commercial applicability of imaging systems.

### Imaging Platforms

Another aspect provided herein, per FIGS. 21A-25, is an imaging platform for imaging an emission light emitted by a sample comprising a fluorophore. FIGS. 21A-B, illustrate example imaging platforms 4000 where the imaging system is operatively engaged to the surgical microscope 101. As shown in FIGS. 21A and 21B, the platform 4000 comprises the imaging system 1000 herein and an imaging station 2000. The platform 4000 further comprises an imaging cable 3000 communicatively coupling the imaging system 1000 herein and an imaging station 2000. In some embodiments, the imaging system 1000, the imaging station 2000, and the imaging cable 3000 are each individual components. In some embodiments, at least two of the imaging system 1000, the imaging station 2000, and the imaging cable 3000 are combined into a single component. In some embodiments, the imaging station 2000 comprises a non-transitory computer-readable storage media encoded with a computer program including instructions executable by a processor to receive the image frames from the image sensor and an input device. In some embodiments, the imaging station is ‘cart based’ as shown in FIG. 21A. In other embodiments, the imaging station is contained in a small wheeled unit, is hung off of the microscope, is rested or hung elsewhere on the microscope, is placed on the floor next to the microscope, or is placed on a tray/pole/table. Or, the imaging station could be designed to be placed in multiple positions such as hanging from the microscope and hanging on a tray as shown in FIG. 21B.

In some embodiments, the imaging station 2000 receives the image frames from the imaging system 1000 via an imaging cable, a wireless connection, or both. In some embodiments, the wireless connection comprises a Bluetooth connection (e.g., short wave wireless, short-wavelength UHF radio waves), a Wi-Fi connection (e.g., wireless local area networks (LANs), wireless broadband Internet), an radio-frequency identification (RFID) connection (e.g., whereby digital data encoded in tags or smart technology labels are captured by a reader via radio wave, and where an RFID comprises a transponder; a radio receiver and transmitter), or any combination thereof. In some embodiments, the platform 4000 further comprises the imaging cable. In some embodiments, the imaging system 1000 further receives power from the image station 2000 via the imaging cable 3000.

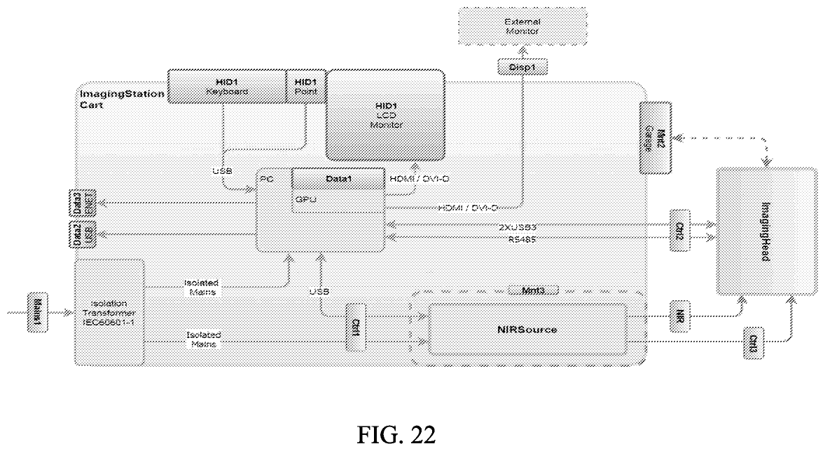

In some embodiments, the input device comprises a mouse, a trackpad, a joystick, a touchscreen, a keyboard, a microphone, a camera, a scanner, an RFID reader, a Bluetooth device, a gesture interface, a voice interface, or any combination thereof. FIG. 22, shows an exemplary schematic diagram of the imaging station 2000. In some embodiments, the platform 4000 further comprises a laser configured to emit the excitation light, a white light configured to emit the visible light or both. In some embodiments, the imaging station 2000 comprises a power system, a CPU, an interface for a display (e.g. HDMI, DP), and an interface to connect the imaging system 1000. In some embodiments, the imaging station 2000 only comprises a power system, a CPU, an interface for a display (e.g. HDMI, DP), and an interface to connect the imaging system 1000.

In some embodiments, per FIGS. 24-28, the platform 4000 further comprises a laser monitor interlock 5301. In some embodiments, the laser monitor interlock 5301 comprises a relay capable of cutting power to the laser driver. In some embodiments, for example as shown in FIG. 24, the laser monitor electronics 5102 receives data from the laser monitor sensor(s) 5101. In some embodiments, the laser monitor sensor(s) 5101 comprises excitation light power gauge, the diffused beam shape sensor 5101 or both. In some embodiments, the laser monitor electronics 5102 receives data from the excitation light power gauge, the diffused beam shape sensor 5101, or both. In some embodiments, the laser monitor electronics 5102 receives data from the excitation light power gauge, the diffused beam shape sensor 5101, or both, in real time. In some embodiments, the laser monitor electronics 5102 receives data from the excitation light power gauge, the diffused beam shape sensor 5101, or both only when the laser is in an on mode. In some embodiments, the laser monitor electronics 5102 receives data from first diffused beam shape gauge, the second diffused beam shape gauge, or both. In some embodiments, the laser monitor electronics 5102 receives data from 2, 3, 4, 5, 6, 7, 8, 9, 10 or more beam shape gauges. In some embodiments, the system further comprising 2, 3, 4, 5, 6, 7, 8, 9, 10, or more additional beam shape gauges. In some embodiments, the first beam shape gauge, the second beam shape gauge, the additional beam shape gauges, or any combination thereof are arranged in a one-dimensional array. In some embodiments, the first beam shape gauge, the second beam shape gauge, the additional beam shape gauges, or any combination thereof are arranged in a two-dimensional array. In some embodiments, the first beam shape gauge, the second beam shape gauge, the additional beam shape gauges, or any combination thereof are positioned within about 5% to about 50% of the width of the diffused beam shape from the center of the diffused beam shape.

In some embodiments, the laser monitor electronics 5102 is configured to turn off the laser 5304. In some embodiments, the laser monitor electronics 5102 is configured to turn off the laser 5304 if: the measured power of the excitation light (“power of the excitation” light also referred to as “excitation power” herein) deviates from a set excitation light power by a first predetermined value, the diffused beam shape deviates from a set beam shape by a second predetermined value, or both. In some embodiments, the laser monitor electronics 5102 is configured to turn off the laser 5304 within less than 1 second, 0.9 seconds, 0.8 seconds, 0.7 seconds, 0.6 seconds, 0.5 seconds, 0.25 seconds, 0.1 seconds, 0.05 seconds, 0.01 seconds, 0.005 seconds, 0.001 seconds, 0. seconds, 0. seconds, 0.5 seconds, 0.1 seconds, 0.05 seconds, 0.01 seconds, or less, including increments therein if the measured power of the excitation light deviates from a set excitation light power by a first predetermined value, the diffused beam shape deviates from a set beam shape by a second predetermined value, or both. In some embodiments, the laser monitor electronics 5102 receives data from 2, 3, 4, 5, 6, 7, 8, 9, 10 or more beam shape gauges wherein the laser monitor electronics 5102 is configured to turn off the laser 5304 within less than 1 second, 0.9 seconds, 0.8 seconds, 0.7 seconds, 0.6 seconds, 0.5 seconds, 0.25 seconds, 0.1 seconds, 0.05 seconds, 0.01 seconds, 0.005 seconds, 0.001 seconds, 0. seconds, 0. seconds, 0.5 seconds, 0.1 seconds, 0.05 seconds, 0.01 seconds, or less, including increments therein if the measured power of the excitation light deviates measured by each of the beam shape gauges differs from a set excitation light power by a predetermined value. In some embodiments, the predetermined value is a positive value, wherein the laser monitor electronics 5102 is configured to turn off the laser 5304 if the measured power of the excitation light deviates measured by each of the beam shape gauges is greater than the set excitation light power plus the predetermined value. In some embodiments, the predetermined value is a negative value, wherein the laser monitor electronics 5102 is configured to turn off the laser 5304 if the measured power of the excitation light deviates measured by each of the beam shape gauges is less than the set excitation light power plus the negative predetermined value. In some embodiments, the predetermined value comprises both a negative predetermined value and a positive predetermined value, wherein the negative predetermined value is greater than the positive predetermined value. In some embodiments, the predetermined value comprises both a negative predetermined value and a positive predetermined value, wherein the negative predetermined value is less than the positive predetermined value. In some embodiments, the positive predetermined value, the negative predetermined value, or both are based on laser class power, a desired illumination shape, or both.

In some embodiments, the laser monitor electronics 5102 determines whether the excitation power relative to a predetermined range (e.g., such range to set high and low limits for the excitation power) has either exceeded the highest predetermined value in the range or is less than the lowest predetermined value in the range. In the event the laser monitor electronics 5102 detects excitation power that exceeds of the highest predetermined value (i.e., “too high” value) or detects excitation power less than the lowest predetermined value (i.e., “too low” value) relative to the predetermined range, the laser is shut off. Similarly, a predetermined range resulting in laser shutoff can be assessed using the rate of change of the excitation power, for example, by comparing the magnitude of the time derivative of the power measurement against a maximum allowed value, rather than testing the measured power against a range of power values. In some embodiments, the laser monitor electronics 5102 determines whether the magnitude of the rate of change of the excitation power relative to a predetermined maximum value (e.g., high limit for the magnitude of the rate of change of the excitation power) has exceeded the highest predetermined rate. In the event the laser monitor electronics 5102 detects excitation power change that exceeds of the highest predetermined rate (i.e., “too high” rate), the laser is shut off. Exceeding the predetermined rate is potentially detrimental in applications relating to in vivo or in situ applications in humans or animals, and a laser shutoff responding to exceeding the highest predetermined rate is an important safety feature of the imaging system in such applications. In some embodiments, the laser monitor electronics 5102 determines whether the excitation power relative to a predetermined range can combine the concepts of a predetermined range of excitation power value and/or excitation power rate as described herein. In some embodiments, the laser is shut off within a millisecond, a microsecond, or a picosecond or less, of when the laser monitor electronics 5102 determines that the magnitude of the rate of change of the excitation power relative to a predetermined maximum value (e.g., high limit for the magnitude of the rate of change of the excitation power) has exceeded the highest predetermined rate.

In some embodiments, the laser monitor electronics 5102 determines that the beam shape has deviated from the set beam shape based on the power of the diffused beam at one position in the diffused beam shape as measured by a first diffused beam shape gauge as compared to the power of the diffused beam at least one other position in the diffused beam shape as measured by at least a second diffused beam shape gauge. It is understood that at least two diffused beam shape gauges measure along the diffused beam shape at different positions along the diffused beam shape.

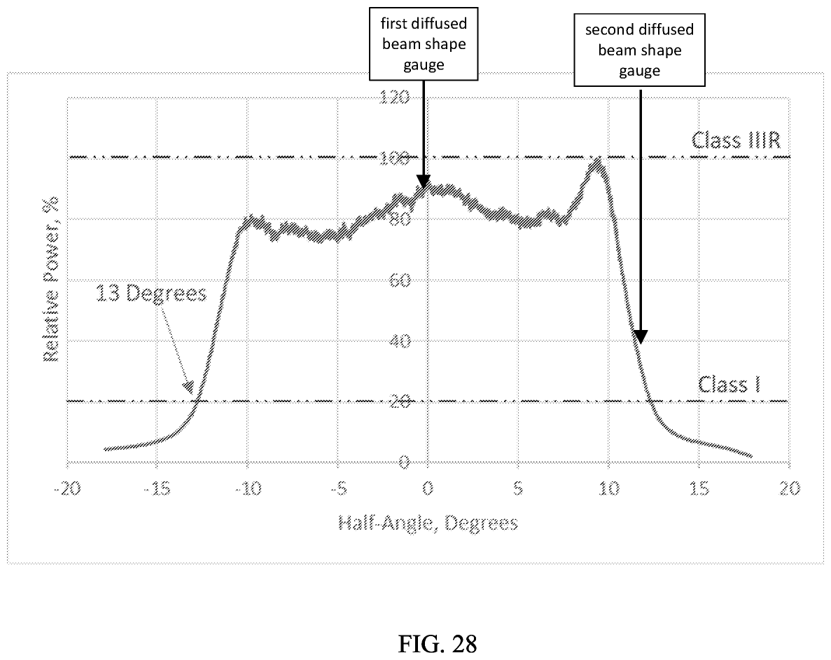

FIG. 28 illustrates an embodiment where a first diffused beam shape gauge is positioned at the center of the beam profile, and a second diffused beam shape gauge is positioned where the beam shape should be approximately half of the maximum value. It is further understood that at least two and up to multiple beam shape gauges may be used. It further understood that multiple power measurements can be taken at any positions along the diffused beam shape and compared. For example, such positions measured include measuring any two or more positions including the edge of the diffused beam shape (whether the edge is a position where the beam shape is designed to be less than the maximum value of the set beam shape) or anywhere else along the diffused beam shape, provided that the two or more positions measured include at least two separate and distinct positions along the beam shape. In some embodiments, the laser monitor electronics 5102 determines that the beam shape has deviated from the set beam shape based on the power of the diffused beam at least one position along diffused beam shape as measured by a single diffused beam shape gauge that compares to the power of the diffused beam at one or more positions or area along of the diffused beam shape. Such single diffused beam gauge can measure same position multiple times or be a diffused beam gauge that measures anywhere in an area along the diffused beam shape. In some embodiments, the laser monitor electronics 5102 determines that the beam shape has deviated from the set beam shape based on the power of the diffused beam at a center of the diffused beam shape as measured by the first diffused beam shape gauge, the power of the diffused beam at the edge of the diffused beam shape as measured by the second diffused beam shape gauge, or both. In some embodiments, the laser monitor electronics 5102 determines that the beam shape has deviated from the set beam shape by comparing the power of the diffused beam at a center of the diffused beam shape as measured by the first diffused beam shape gauge with the power of the diffused beam at the edge of the diffused beam shape as measured by the second diffused beam shape gauge. In some embodiments, the laser monitor electronics 5102 determines that the beam shape has deviated from the set beam shape when the power of the diffused beam at a center of the diffused beam shape as measured by the first diffused beam shape gauge with the power of the diffused beam at the edge of the diffused beam shape as measured by the second diffused beam shape gauge diffused beam shape deviates from a set beam shape by at least the second predetermined value. In some embodiments, the laser monitor electronics 5102 determines that the beam shape has deviated from the set beam shape when a difference in power between the two or more of the diffused beam shape gauges differs sufficiently (e.g. by the second predetermined value). In some embodiments, the laser is shut off within a millisecond, a microsecond, or a picosecond or less, of when the laser monitor electronics 5102 determines that the beam shape has deviated from the set beam shape.

In some embodiments, a measured power of the excitation light (i.e., excitation power) below the set excitation light power by at least the first predetermined value, is an indication that the laser 5304 is not performing correctly, that at least a portion of the optical path is damaged, that at least a portion of the NIR or IR light is leaking from the system, or any combination thereof. As such, turning off the laser 5304 by the laser monitor electronics 5102 if the measured power of the excitation light below the set excitation light power by at least the first predetermined value prevents the system from capturing image frames of samples that are not fully excited, prevents damage to the system, or both. In some embodiments, a measured power of the excitation light above the set excitation light power by at least the first predetermined value, is an indication that the laser 5304 is exceeding design and/or safety limits and not performing correctly. As such, turning off the laser 5304 by the laser monitor electronics 5102 if the measured power of the excitation light above the set excitation light power by at least the first predetermined value prevents damage to the system, a patient, a system user, or any combination thereof. In some embodiments, a beam shape that deviates from a set beam shape by at least or at most a second predetermined value indicates that the diffuser has failed, that the laser 5304 is being emitted as a collimated beam, or both. In some embodiments, a laser 5304 emitted as a collimated beam is capable of damaging the components of the system and/or harming users of the system.

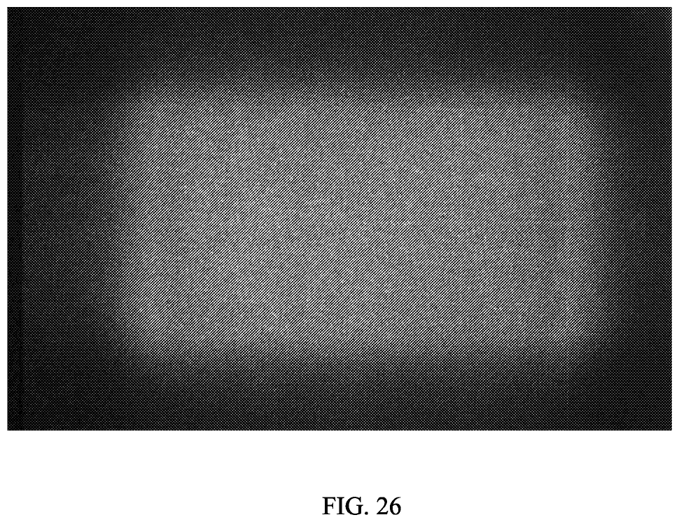

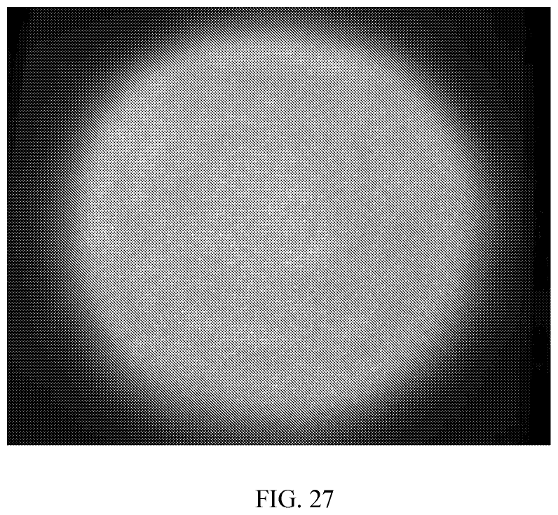

FIG. 24 shows a first schematic diagram of one embodiment of a laser monitoring system 5000. As shown therein, in some embodiments, the laser monitor sensors transmit the sensor data to a laser monitor electronics 5102, which transmits at least a portion of the sensor data to the laser monitor interlock 5301 in the imaging station via the imaging cable 3000. Further as shown, in some embodiments, the laser monitor interlock 5301 acts as an intermediary between a laser power source 5302 and the laser driver 5303, wherein the laser driver 5303 directs the laser 5304, and wherein the laser beam is transmitted to the imaging system. In some embodiments, the sensor data comprises the measured power of the excitation light, the diffused beam shape, the power of the diffused beam at the edge of the diffused beam shape, the power of the diffused beam at the edge of the diffused beam shape, or any combination thereof. In some embodiments, the laser monitor electronics 5102 determine the diffused beam shape. In some embodiments, the laser monitor electronics 5102 determine if the diffused beam shape deviates from a set beam shape by at least the second predetermined value. FIG. 26 shows an image of an exemplary rectangular beam shape. FIG. 27 shows an image of an exemplary circular beam shape.

In some embodiments, the first predetermined value, the second predetermined value, or both are determined by a classification rating of one or more of the laser safety guidelines in the Code of Federal Regulations (CFR), IEC 60825, or both. In some embodiments, the first predetermined value, the second predetermined value or both are determined by the regulations regarding class 1, 1M, 2, 2M, 34, 3B, or 4 lasers of IEC 60825. In some embodiments, the laser monitor electronics 5102 is configured to turn off the laser, through the laser monitor interlock 5301, before the laser safety guidelines for one or more safety limits for one or more of the classification ratings are exceeded. In some embodiments, the time required for the laser monitor interlock 5301 depends on the incident laser power, the laser decay time, the driver delay time, or any combination thereof. FIG. 28, shows an exemplary graph of the placement of photodiodes within the beam shape. Shown therein are the relative power percentages vs half-angle, for a 20-degree diffuser, where 100% power corresponds to the maximum power that is output for a class IIIR laser. Further, as shown, the first diffused beam shape gauge measures a power of the diffused beam at a center of the diffused beam shape, wherein the second diffused beam shape gauge measures a power of the diffused beam at an edge of the diffused beam shape.

FIG. 25 shows a schematic diagram of another embodiment of a laser monitoring system 2500. As shown therein, in some embodiments, the laser monitoring design comprises the imaging system 2510, an imaging cable 2550, and an imaging station 2560. In some embodiments, as shown, the imaging system 2510 comprises a head control assembly 2520, a laser power sensor 2511, a beam shape sensor 2512, and a laser active indicator 2513. In some embodiments, the head control assembly 2520 comprises a head control processor PCBA 2530 and a laser monitor electronics 2540. In some embodiments, the head control assembly 2520 comprises a head control process printed circuit board assembly (PCBA) 2530, and a laser monitor electronics 2540. In some embodiments, the head control PCBA 2520 comprises a Digital IO 2532 and an ADC. In some embodiments, the laser monitor electronics comprises a window threshold for power circuit 2541, a window threshold for shape circuit 2542, a power too low logic circuit 2543, and an OR operator 2544. In some embodiments, the imaging station 2560 comprises an NIR source 2570 comprising a laser 2571, a laser driver 2572, and a laser monitor PCBA 2573. In some embodiments, the laser monitor PCBA 2573 comprises a laser interlock relay 2574 and a laser power set pot 2575.

In some embodiments, the Digital IO 2532 sends a laser trigger to the power too low logic component 2543. In some embodiments, an ADC 2531 monitors the sensor outputs. In some embodiments, the ADC 2531 is used to supply digital values to a CPU, which is monitored and/or logged. In some embodiments, the ADC 2531 is used for diagnostics. In some embodiments, the Laser Monitor Electronics 2540 do not comprise any software, so that a software bug is not able to compromise the performance of the laser monitor system. In some embodiments, at least one of the window threshold for power circuit 2541 and a window threshold for shape circuit 2542 receives a power of the excitation light (i.e., excitation power) from the laser power sensor 2511, the measured beam shape from the beam shape sensor 2512, or both. In some embodiments, if the laser power is too low, the window threshold for power circuit 2541 transmits a notification to the power too low logic 2543. In some embodiments, if the laser shape power is too low, the window threshold for shape circuit 2542 transmits a notification to the power too low logic 2543. In some embodiments, if the laser power is too high, the window threshold for power 2541 transmits a notification to an (OR) operator 2544. In some embodiments, if the laser shape power is too high, the window threshold for shape 2542 transmits a notification to the (OR) operator 2544. In some embodiments, the (OR) operator 2544 transmits a laser disable signal to the laser interlock 2574. In some embodiments, the laser interlock 2574 is a solid-state relay, a mechanical relay, a ‘disable’ input to the laser 2571, the laser driver 2572 or both. In some embodiments, the laser interlock 2574 is a circuit that shuts off power to the laser 2541. In some embodiments, the laser interlock 2574 acts as a ‘disable’ input to the laser 2571, laser driver 2572, or both. In some embodiments, the laser interlock 2574 comprises a circuit that shuts off power to the laser 2571. in some embodiments, the laser interlock 2574 is in the imaging station 2560 or the imaging system 2510. In some embodiments, a laser active logic 2545 provides a laser active signal to one or more laser active indicators 2513. In some embodiments, the laser active logic 2545 receives a signal from the (OR) operator 2544, the laser trigger from the digital IO device 2532, or both. In some embodiments, the laser driver 2572 further receives a power set control from the laser power set potentiometer 2575 in the laser monitor PCBA 2573. In some embodiments, the laser interlock 2574 transmits or does not transmit power to the laser driver 2572 based on the (OR) operator 2544. In some embodiments, the laser driver 2572 powers the laser 2571.

### Methods of Imaging an Emission Light

Another aspect provided herein is a method for imaging an emission light emitted by a sample comprising a fluorophore, the method comprising: emitting an excitation light; diffusing the excitation light; receiving and directing a visible light to the sample; directing the diffused excitation light to the sample; directing the emission light and a reflected visible light to an imaging assembly; and filtering the emission light and the reflected visible light; detecting both the emission light and the reflected visible light from the sample to generate image frames based on the emission light and the reflected visible light.

In some embodiments, filtering the emission light and the reflected visible light comprises directing at least a portion of the emission light and the reflected visible light from the sample through the notch beam splitter, the first notch filter, the longpass filter, the lens, and the second notch filter. In some embodiments, filtering the emission light and the reflected visible light comprises directing at least a portion of the emission light and the reflected visible light from the sample and sequentially through the notch beam splitter, the first notch filter, the longpass filter, the lens, and the second notch filter. In some embodiments, the longpass filter comprises a vis-cut longpass filter. In some embodiments, the longpass filter at least partially reduces transmission of at least a portion of the visible light therethrough. In some embodiments, the longpass filters a majority of the NIR or IR light. In some embodiments, the longpass filter transmits at least about 85%, 86%, 87%, 88%, 89%, 90%, 91%, 92%, 93%, 94%, 95%, 96%, 97%, 98%, or 99% of the NIR or IR light, including increments therein. In some embodiments, the polarizer reduces a ghosting effect from reflections of the front/back surfaces of the shortpass dichroic. In some embodiments, the method does not comprise directing the emission light and the reflected visible light through the polarizer. In some embodiments, the method further comprises removing the polarizer from the imaging assembly. In some embodiments, the method further comprises adding the polarizer to the imaging assembly.

In some embodiments, the laser has an off mode and an on mode. In some embodiments, the excitation light has a wavelength of about 775 nm to about 795 nm. In some embodiments, the excitation light has a wavelength of about 785 nm. In some embodiments, the excitation light has a wavelength of about 400 nm to about 800 nm. In some embodiments, the excitation light has a wavelength of about 800 nm to about 950 nm.

In some embodiments, for example as shown in FIG. 27, the excitation light is at least a portion of light diffused by a circular excitation diffuser. In some embodiments, the circular excitation diffuser has a diffusion angle of about 4 degrees to about 25 degrees. In some embodiments, for example as shown in FIG. 26, the excitation light is at least a portion of diffused by a rectangular excitation diffuser. In some embodiments, the rectangular excitation diffuser has a first diffusion angle and a second diffusion angle perpendicular to the first diffusion angle. In some embodiments, the first diffusion angle, the second diffusion angle, or both are about 4 degrees to about 25 degrees. In some embodiments, the first diffusion angle is about 14 degrees, and wherein the second diffusion angle is about 8 degrees. In some embodiments, at least a portion of diffused excitation light is directed to the sample by a hot mirror, a dichroic mirror, a shortpass filter, or any combination thereof. In some embodiments, the reflected visible light is directed to the imaging assembly by a notch beam splitter. In some embodiments, the notch beam splitter blocks and reflects a notch band. In some embodiments, the reflected visible light is directed to the imaging assembly by a notch beam splitter, a hot mirror or both. In some embodiments, which only reflects NIR or IR, not VIS. In some embodiments the hot mirror filters out the wavelength of the NIR or IR light from the visible light. In some embodiments, at least a portion of the diffused excitation light is directed to the sample in a first direction and wherein the emission light and the reflected visible light are directed in a second direction opposite the first direction.

In some embodiments, filtering the emission light and the reflected visible light comprises blocking at least a portion of the excitation light. In some embodiments, filtering the emission light and the reflected visible light comprises blocking light having a wavelength of about 775 nm to about 795 nm from passing therethrough. In some embodiments, filtering the emission light and the reflected visible light comprises blocking light having a wavelength of about 785 nm from passing therethrough. In some embodiments, the method further comprises polarizing at least a portion of the emission light and the reflected visible light. In some embodiments, the method further comprises filtering at least a portion of the diffused excitation light. In some embodiments, filtering the diffused excitation light comprises filtering out wavelengths less than about 720 nm, less than about 720 nm, 725 nm, 730 nm, 735 nm, 740 nm, 750 nm, 755 nm, 760 nm, 770 nm, 780 nm, 800 nm, or more including increments therein. In some embodiments, at least a portion of the excitation light is an infrared or a near-infrared excitation light. In some embodiments, filtering at least a portion of the emission light and the reflected visible light comprises attenuating the emission light and the reflected visible light. In some embodiments, attenuating at least a portion of the emission light and the reflected visible light comprises blocking all but near infrared wavelengths.

In some embodiments, the method further comprises monitoring the laser. In some embodiments, monitoring the laser comprises: measuring a power of at least a portion of the excitation light, measuring a diffused beam shape, or both. In some embodiments, the power of the excitation light (i.e., excitation power) is measured by an excitation light monitor. In some embodiments, the diffused beam shape of the diffused excitation light is measured by a first diffused beam shape gauge, a second diffused beam shape gauge, or both. In some embodiments, the diffused beam shape sensor comprises 2, 3, 4, 5, 6, 7, 8, 9, 10 or more beam shape gauges, arranged in a 1-d or 2-d array. In some embodiments, the two or more diffused beam shape gauges measure a strength of the diffused beam at multiple locations with the shape of the diffused beam. In some embodiments, the excitation light monitor measures the power of the excitation light by receiving a redirected portion of the excitation light. In some embodiments, the first diffused beam shape gauge measures a power of the diffused beam at a center of the diffused beam shape and wherein the second diffused beam shape gauge measures the power of the diffused beam at an edge of the diffused beam shape. In some embodiments, the excitation light monitor, the first diffused beam shape gauge, the second diffused beam shape gauge, or any combination thereof comprise a photodiode, a camera, a piezoelectric sensor, a linear sensor array, a CMOS sensor, or any combination thereof.

In some embodiments, the method further comprises turning off the laser. In some embodiments, the method further comprises turning off the laser by a laser monitor. In some embodiments, the method further comprises turning off the laser if: the measured power of the excitation light deviates from a set excitation light power by a first predetermined value; the diffused beam shape deviates from a set beam shape by a second predetermined value; or both. In some embodiments, the method further comprises turning off the laser within less than 1 second, 0.9 seconds, 0.8 seconds, 0.7 seconds, 0.6 seconds, 0.5 seconds, 0.25 seconds, 0.1 seconds, 0.05 seconds, 0.01 seconds, 0.005 seconds, 0.001 seconds, 0. seconds, 0. seconds, 0.5 seconds, 0.1 seconds, 0.05 seconds, 0.01 seconds, or less, or less, including increments therein if the measured power of the excitation light deviates from a set excitation light power by a first predetermined value, the diffused beam shape deviates from a set beam shape by a second predetermined value, or both. In some embodiments, the method comprises turning off the laser based on a comparison between the power of the diffused beam at a center of the diffused beam shape as measured by the first diffused beam shape gauge and the power of the diffused beam at the edge of the diffused beam shape as measured by the second diffused beam shape gauge. In some embodiments, the method comprises turning off the laser if beam shape deviates from the set beam shape by at least the second predetermined value, based on the power of the diffused beam at a center of the diffused beam shape as measured by the first diffused beam shape gauge with the power of the diffused beam at the edge of the diffused beam shape as measured by the second diffused beam shape gauge. In some embodiments, the diffused beam shape sensor comprises 2, 3, 4, 5, 6, 7, 8, 9, 10 or more beam shape gauges. In some embodiments, the two or more diffused beam shape gauges measure a strength of the diffused beam at two or more locations with the shape of the diffused beam.

In some embodiments, a measured power of the excitation light below the set excitation light power by at least the first predetermined value, is an indication that the laser is not performing correctly, that at least a portion of the optical path is damaged, that at least a portion of the NIR or IR light is leaking from the system, or any combination thereof. As such, turning off the laser by the laser monitor electronics if the measured power of the excitation light below the set excitation light power by at least the first predetermined value prevents the system from capturing image frames of samples that are not fully excited, prevents damage to the system, or both. In some embodiments, a measured power of the excitation light above the set excitation light power by at least the first predetermined value, is an indication that the laser is exceeding design and/or safety limits and not performing correctly. As such, turning off the laser by the laser monitor if the measured power of the excitation light above the set excitation light power by at least the first predetermined value prevents damage to the system, a patient, a system user, or any combination thereof.

In some embodiments, turning off the laser serves as a fail-safe against any software errors. In some embodiments, the power of the excitation light, the diffused beam shape, or both are only measured when the laser is turned on to prevent a false positive of the excitation light being below the first predetermined value when the laser is off. In some embodiments, the first predetermined value, the second predetermined value, or both are determined by a classification rating of one or more of the laser safety guidelines in the Code of Federal Regulations (CFR), IEC 60825, or both. In some embodiments, the first predetermined value, the second predetermined value, or both are determined by the regulations regarding class 1, 1M, 2, 2M, 34, 3B, or 4 lasers of IEC 60825. In some embodiments, the method comprises turning off the laser before the laser safety guidelines for one or more safety limits for one or more of the classification ratings are exceeded. In some embodiments, the time required for the laser monitor interlock depends on the incident laser power, the laser decay time, the driver delay time, or any combination thereof.

In some embodiments, the method further comprises receiving, by a non-transitory computer-readable storage media encoded with a computer program including instructions executable by a processor, the image frames from the image sensor. In some embodiments, receiving the image frames from the image sensor is performed by an imaging cable, a wireless connection, or both. In some embodiments, the wireless connection comprises a Bluetooth connection, a Wi-Fi connection, an RFID connection, or any combination thereof.

In some embodiments, the method further comprises cleaning the bottom window.

### Illumination and Excitation Sources

In some embodiments, the system comprises one or more excitation sources configured to generate an excitation beam to excite a fluorophore or excite fluorescence tagged tissue or stimulate fluorescence in the region of tissue imaged. In some embodiments, the system comprises one or more illumination light sources configured to emit visible light in order to enable a user such as a surgeon to view the sample and non-fluorescent aspects.

The one or more illumination sources can act as an excitation light source. The one or more excitation sources can act as an illumination light source. At least one of the illumination source and the excitation source can comprise a visible light source. Visible light can be generated by a number of white light or visible light spectrum sources. At least one of the illumination source and the excitation source can comprise a broadband source, a narrowband laser, a wide band source, narrow-band light source, or any combination thereof. At least one of the illumination source and the excitation source can be an incoherent light or a coherent light.

At least one of the illumination source and the excitation source can comprise an incandescent lamp, a gas discharge lamp, a xenon lamp, an LED, a halogen lamp, or any combination thereof. The broadband source can emit NIR or IR spectrum light. The wide band source can comprise a light emitting diode (LED) coupled to a notch filter.

At least one of the illumination source and the excitation source can be a visible, red, infrared (IR), near-infrared (NIR), ultraviolet, or blue light. The excitation light can comprise red light having a wavelength within a range from about 620 to 700 nm, red light having a wavelength of about 650 to about 700 nm, near infrared or infrared light having a wavelength of about 710 to about 800 nm, near infrared or infrared light having a wavelength of about 780 to about 850 nm, ultraviolet light having a wavelength of about 10 to 400 nm, ultraviolet light having a wavelength of about 200 to about 400 nm, blue light having a wavelength of about 380 to 460 nm, or blue light having a wavelength from about 400 to 450 nm.

At least one of the illumination source and the excitation source can be controlled by the imaging system, or be uncontrolled. The uncontrolled source can be, for example, a microscope light source, an ambient light source, or both. The excitation light source can comprise a laser or a wide band source (e.g., light emitting diode (LED)) coupled to a band pass filter.

In some embodiments, the excitation source has a wavelength of about 720, 750, 785, 790, 792, or 795 nm. In some embodiments, the excitation source has a wavelength in the infrared spectrum including light wavelengths the IR-A (about 800-1400 nm), IR-B (about 1400 nm-3 um) and IR-C (about 3 um-1 mm) spectrum. In some embodiments, the excitation source has a wavelength that is in the near infrared (NIR) spectrum from about 650 nm to 4000 nm, 700 nm to 3000 nm, 700-800 nm, 750 nm to 950 nm, 760 nm 825 nm, 775 nm to 795 nm, 780 nm to 795 nm, 785 nm to 795 nm, 780 nm to 790 nm, 785 nm to 792 nm, 790 nm to 795 nm, or any wavelength within any of these foregoing NIR or IR ranges.

In some embodiments, the excitation source comprises a laser to cause the target (e.g., tissue tagged with fluorescence dye) to fluoresce and generate a fluorescence emission. The excitation source can alternate between on and off status. The visible light can or cannot be present to illuminate the target tissue in addition to the excitation source. In some embodiments, if there is a visible light source present in the system and method herein, it can have on and off status such that the light can be synchronously turned on/off with the excitation source. In some embodiments, external visible light such as from an operating microscope or surgical or examination light can be used. In some embodiments, the external light has an on and off status but is not synchronized with the excitation source's light. In other embodiments the external light source can be continuously on or continuously off.

In some aspects, the laser monitor system previously described can be used as part of a system that confirms that infrared imaging is performing normally, without the need for a separate fluorescent imaging target. In these aspects, embodiments of the laser monitoring 5000, as shown in FIG. 24, or the laser monitoring 2500, as shown in FIG. 25, aid in accurately illuminating the target tissue by monitoring the laser beam shape parameters. Form the beam shape parameters the operation and accuracy of the excitation source may be inferred.

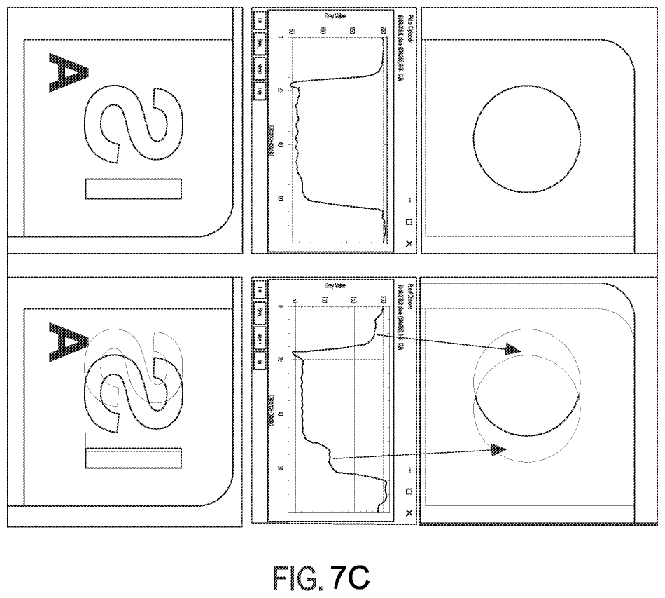

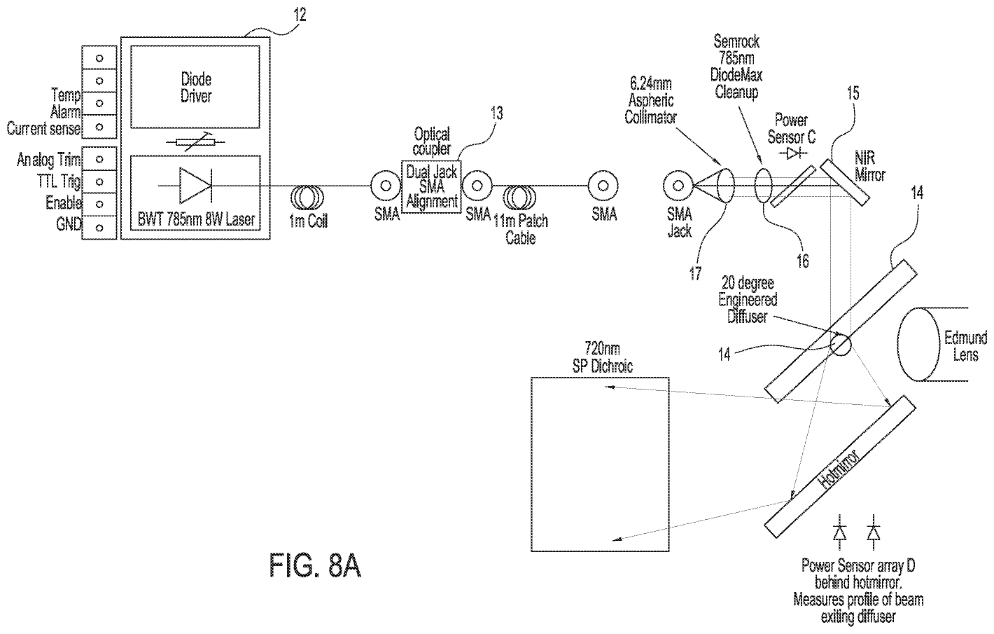

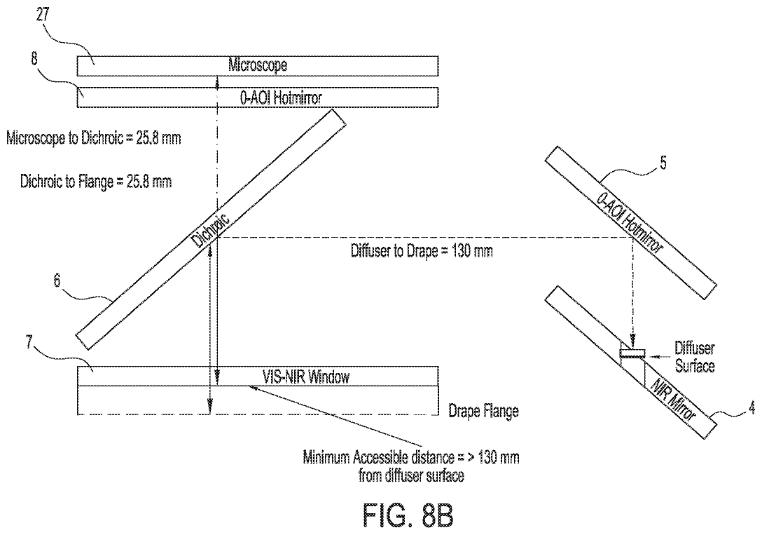

FIG. 8A shows an exemplary embodiment of the illumination opto-electrical system of the light source. In some embodiments, the systems and methods herein include one or more beam splitters, dichroic filters, dichroic mirrors, or use of the same. In some embodiments, the systems and methods include a primary dichroic mirror, and a secondary dichroic mirror. In some embodiments, the systems and methods include one or more shortpass dichroic mirrors and/or one or more longpass dichroic mirrors. In some embodiments, the beam splitters or dichroic mirrors, herein are configured to enable longpass-passing long wavelength while reflecting short wavelength (e.g. longpass filter or cold mirror) or shortpass-passing short wavelength while reflecting long wavelength (e.g., shortpass filter hot mirror). In some embodiments, the visible light herein is considered short wavelengths (e.g., shorter than 700 nm, or shorter than 800 nm) while the NIR or IR light are long wavelength (e.g., longer than 780 nm). In some embodiments, a mirror or filter herein includes filtering function (i.e., selective transmitting function) and/or or mirroring function (i.e., selective reflecting function).

The human eye can see color in the “visible light” spectrum in wavelengths from about 350 nm up to about 750 nm, although a person of ordinary skill in the art will recognize variations depending on the intensity of light used. The light provided to the user with surgical microscope's eyepieces and the visible light imaging system will typically comprise wavelengths within this visible range. In some embodiments, the excitation beam comprises wavelengths shorter than at least some of the wavelengths transmitted with the eyepieces and used with the visible imaging system and detector, for example wavelengths ranging from 300 to 400 nm. In some embodiments, the excitation beam comprises wavelengths longer than at least some of the wavelengths transmitted with the eyepieces and used with the visible imaging system and detector, for example wavelengths longer than about 750 nm. In some embodiments, the excitation wavelengths comprise wavelengths greater than about 750 nm. For example, the dichroic mirror/filter can comprise a transition wavelength of about-700 nm. (This optical element can also be referred to as 700 nm SP dichroic filter, for example.) By way of example, the shortpass (SP) dichroic filter can be configured to allow light with a wavelength of less than the transition frequency of about 700 to pass through the filter. This filter can be used to transmit more than 90% of the visible light, such that images seen by the user are substantially free of chromatic distortion, hue imbalance, or both. The filter can be designed to show very little attenuation of the images seen through the eyepieces as compared with a microscope without this filter, which creates a better user experience and allows a surgeon to better visualize the surgical field with decreased amounts of light that might otherwise interfere with the fluorescence measurement, in accordance with some embodiments. It is understood that the shortpass filter can alternatively be a bandpass or notch filter. For example, one approximately “˜” 700 nm SP dichroic filter can comprise a FF720-SDi01 filter that has a transmission band Tavg=>90% for VIS (visible light), meaning that a 720 nm SP dichroic filter transmits>90% of visible light between 400 nm and 700 nm while reflecting>99% in the fluorescence emission band. The ˜700 nm SP dichroic filter allows most of the light (e.g., greater than 90%) shorter than about 700 nm through the dichroic filter, while reflecting almost all the light above about 700 nm. In some embodiments, these SP dichroic filters are very efficient in visible light filtering and are 99% efficient or greater with a transmission band Tavg=>99% for VIS (visible light) (e.g., when the incident light, e.g., visible light, or NIR or IR light, on the filter is at a 45° angle). In other embodiments, the SP dichroic filters comprise >50%, >60%, >65%, >75%, >80%, >85%, >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% efficiency or greater with a transmission band Tavg=>50%, >60%, >65%, >75%, >80%, >85%, >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% for VIS (visible light). Moreover, in some embodiments, the ˜700 nm SP dichroic filter, while allowing transmission light to pass through at efficiencies comprising any of the foregoing, can also reflect>75%, >80%, >85%, >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% in the fluorescence emission band.

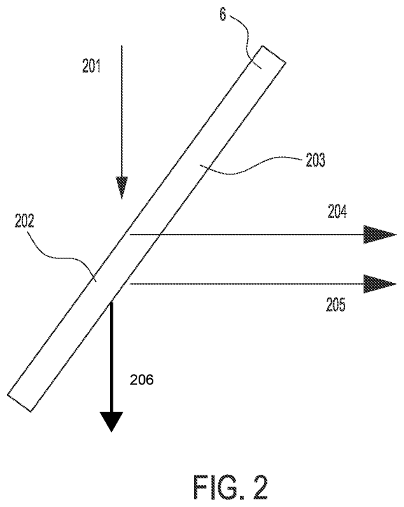

FIG. 2 shows an exemplary embodiment of a dichroic filter 6 having an anti-reflective or other coating 203 or other coating to balance front coating 202 and a dichroic reflecting or other coating 202. As seen, in this embodiment, the dichroic filter 6 is placed so that the incident light 201 is at 45°. The incident light 201 can have a wavelength of less than about 700 nm and transmit through both surfaces, 202 and 203, to result in ray 206. Less than 1% of the incoming light 201 is reflected by the anti-reflective coating 203, shown by ray 205. Light exiting from a front surface of the dichroic filter 205 having the dichroic reflecting coating 202 can have an intensity of greater than about 99% of the intensity of the incident light 201 and a wavelength greater than about 700 nm. In some embodiments, the anti-reflective or other coatings may be selected to transmit light within a specific wavelength range. . . . It is understood that one or more coatings with varied spectral properties can be applied.

In some embodiments, the dichroic filter 6 is placed at 10°, 15°, 20°, 25°, 30°, 35°, 41°, 45°, 50°, 55°, 60°, 65°, 70°, or 75° relative to the incident visible/NIR or IR light path. In some embodiments, the dichroic filter 6 is placed at 35°, 36°, 37°, 38°, 39°, 40°, 41°, 42°, 43°, 44°, 45°, 46°, 47°, 48°, 49°, 50°, 51°, or 52°, relative to the incident visible/NIR or IR light path. In some embodiments, the reflection primarily happens on the front-coated surface 202 of the filter. In order to get better separation of light by wavelengths, the back side of the filter is coated with anti-reflection coating 203, thus further reducing reflection of the light<700 nm. In some embodiments, still a small amount (5-10%) of visible light (<about 700 nm) is reflected from the front as well as back of the filter. In some embodiments, 1%-5%, 3%-10%, 5%-12%, 10%-15%, up to 20% or less of visible light (<about 700 nm) is reflected from the front as well as back of the filter. In some embodiments, such a small amount, i.e., leaked visible light, is advantageous when used in the systems and methods herein for visible light imaging.

### Imaging Control

Imaging parameters controlled by the user/surgeon include the following; focus, magnification, sensitivity (for visible and NIR images), NIR imaging on/off, and displayed view (e.g. overlay, side-by-side, mosaic). This list is not exhaustive.

The imaging parameters may be controlled by an operator using the GUI on the imaging station.

The imaging parameters may also be controlled by the surgeon. Controls for the surgeon could be located on the imaging system, or on a control ‘pad’ connected to the imaging head, or on a control pad connected to the imaging station. The connection could be wired or wireless (e.g. Bluetooth)

Some imaging parameters may be read directly from the microscope; e.g. via an electronic data interchange (EDI) interface. In this case, for example, when the microscope's focus and/or magnification settings are changed, the new settings are communicated to the imaging station which subsequently updates the settings for the imaging system.

This EDI interface could be an existing interface, e.g., used for communicating with a surgical navigation system. It could also be a new/novel/dedicated interface (e.g. wired or wireless)

### Samples

The sample can comprise an ex vivo biological sample, such as a tissue sample. Alternatively, the sample can comprise in vivo or in situ tissue of a subject undergoing surgery.

The sample can include a marking dye. The marking dye can comprise an ultraviolet (UV) dye, a blue dye, or both. Exemplary UV and blue dyes for fluorophores include: ALEXA FLUOR 350 and AMCA dyes (e.g., AMCA-X Dyes), derivatives of 7-aminocoumarin dyes, dialkylaminocoumarin reactive versions of ALEXA FLUOR 350 dyes, ALEXA FLUOR 430 (and reactive UV dyes that absorb between 400 nm and 450 nm have appreciable fluorescence beyond 500 nm in aqueous solution), Marina Blue and Pacific Blue dyes (based on the 6,8-difluoro-7-hydroxycoumarin fluorophore), exhibit bright blue fluorescence emission near 460 nm, hydroxycoumarin and alkoxycoumarin derivatives, Zenon ALEXA FLUOR 350, Zenon ALEXA FLUOR 430 and Zenon Pacific Blue, succinimidyl ester of the Pacific Orange dye, Cascade Blue acetyl azide and other pyrene derivatives, ALEXA FLUOR 405 and its derivatives, pyrene succinimidyl esters, Cascade Yellow dye, PyMPO and pyridyloxazole derivatives, aminonaphthalene-based dyes and dansyl chlorides, dapoxyl dyes (e.g., Dapoxyl sulfonyl chloride, amine-reactive Dapoxyl succinimidyl ester, carboxylic acid-reactive Dapoxyl (2-aminoethyl)sulfonamide), bimane dyes (e.g., bimane mercaptoacetic acid) and its derivatives, NBD dyes and its derivatives, QsY 35 dyes and its derivatives, fluorescein and its derivatives. The marking dye can comprise an infrared dye, near infrared dye or both. Exemplary infrared and near infrared dyes for fluorophores include: DyLight-680, DyLight-750, VivoTag-750, DyLight-800, IRDye-800, VivoTag-680, Cy5.5, or an indocyanine green (ICG) and any derivative of the foregoing, cyanine dyes, acradine orange or yellow, ALEXA FLUORs and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FLASH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′, 7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are known and described in international patent application no. PCT/US2014/77.

The marking dyes used for detection of a sample by the systems and methods herein can comprise one or more dyes, two or more, three, four five and up to ten or more such dyes in a given sample using any class of dye (e.g., ultraviolet (UV) dye, a blue dye, an infrared dye, or near infrared dye) in any combination.

### Cameras and Sensor

The system can comprise one or more imaging sensors to capture the fluorescence light and the visible light.

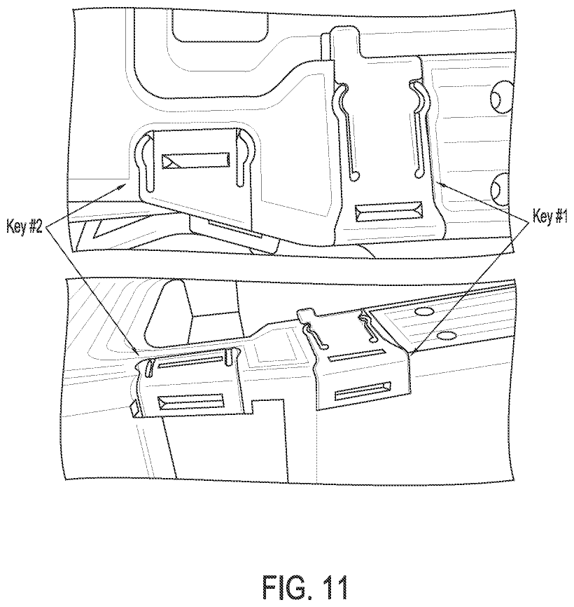

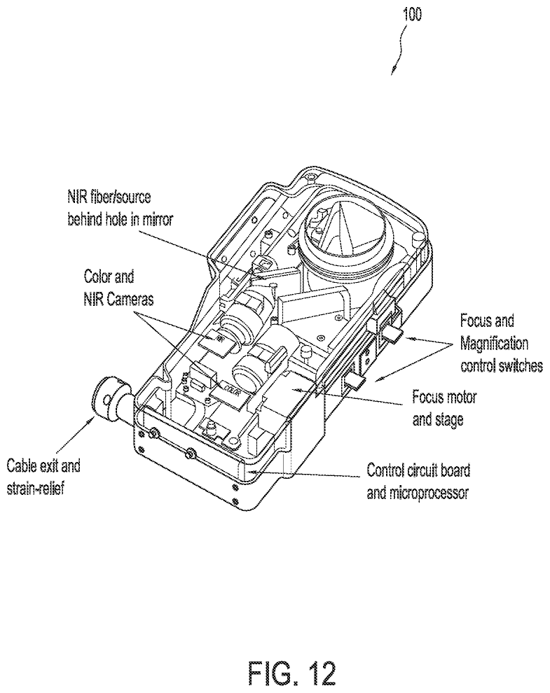

Referring to FIG. 12, in a particular embodiment, the imaging system 100 includes two separate cameras for substantially simultaneous acquisition of near infrared (NIR) fluorescence and visible light. In this embodiment, the imaging system can be attached to an operating microscope.

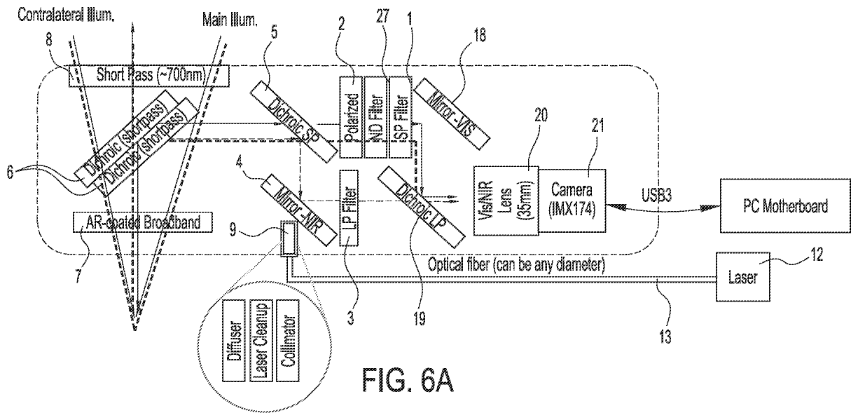

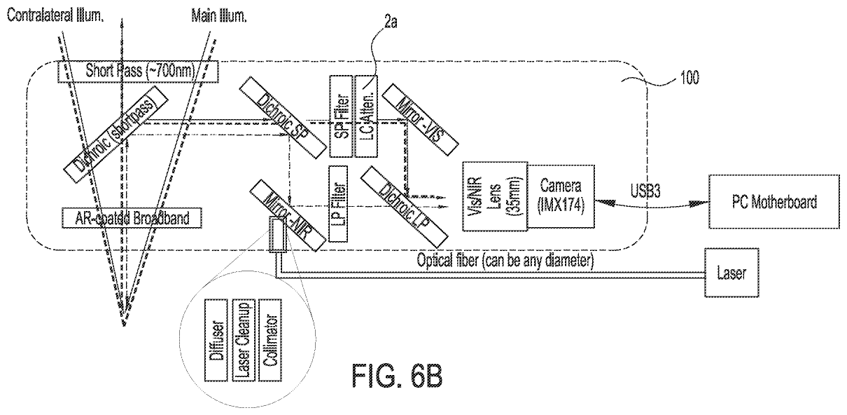

Referring to FIGS. 5A-D, FIGS. 6A-B, 18-20, embodiments of the imaging system 100 may include a single camera for acquisition of near infrared (NIR) fluorescence and visible light. In some embodiments, the excitation light has a wavelength of about 700 to about 800 nm. In some embodiments, the excitation light has a wavelength of about 775 nm to about 795 nm. In this embodiment, the imaging system can be attached to an operating microscope. In some embodiments, the shortpass filter 8 only allows a wavelength of about 400 nm to about 800 nm to pass through. In some embodiments, the shortpass filter 8 only allows a wavelength of about 400 nm to 720 nm to pass through. In some embodiments, the shortpass filter prevents leakage of the excitation laser back into the microscope oculars. In some embodiments, the shortpass filter 8 filters the illumination from the microscope 27, reducing the NIR or IR component. In some embodiments, the shortpass filter is a dichroic filter configured to remove the NIR or IR component from the microscope imaging path. In some embodiments the Notch filter 2 removes excitations having a wavelength of about 785 nm. In some embodiments, the VIS-cut 23 and Notch filters 2 are combined into a single filter. In some embodiments, the VIS-cut 23 or Notch filter 2 have blocking between 650-800 nm for color correction of the single camera image. This additional blocking band is required for a single camera, since the red, green and blue pixels are sensitive to light past 650 nm. Therefore, there is mixing of the colors and generally a magenta hue. In some embodiments, the polarizer 22 reduces ghosting while simultaneously attenuating to some extent the light incident on the polarizer. The filters as shown in FIG. 5D can be arranged in any alternative order.

In some embodiments, the systems and methods herein include one or more image sensors detectors, lenses, or cameras. In some embodiments, the detector herein includes one or more image sensors, lenses, and camera(s) herein. In some embodiments, the systems and methods herein are use a single camera, two cameras, or two or more cameras. In further embodiments, at least one camera is an infrared or NIR camera. In further embodiments, at least one camera is a VIS/NIR camera or a VIS/IR camera.

In some embodiments, the systems and methods herein is a single camera imaging system which only includes a VIS/NIR camera that is configured to sense both visible and NIR or IR signals, as in FIGS. 5A-5D, 6A-6B, and FIG. 18.

Referring to FIGS. 6A-6B, in a particular embodiment, the filtered visible light is reflected at a mirror 18 to a longpass dichroic filter 19 where it gets reflected again and combines with the filtered fluorescence signal to the single VIS/NIR lens 20 and camera 21 of the imaging system.

In some embodiments, two camera imaging systems herein advantageously allow one or more of: complete isolation of the VIS and NIR or IR imaging paths, allowing filtering that is not wavelength or temporally dependent; reduction in temporal artifacts from visible light subtraction (e.g., with high ambient light, a dark frame (DRK) can be of a significant higher brightness level relative to the infrared or NIR signal); ghosting reduction from a dichroic filter without a corresponding loss in sensitivity in the infrared or NIR channel (e.g., the polarizer is only in the visible light path, not in the NIR or IR light path); and there are no constraints on the brightness of the white light from the microscope, or other source of illumination of the surgical field.

In some embodiments, for a single camera design, a visible light filter, neutral density filter or LCD filter or any other optical element which passively or actively reduce the total amount of light passing through) e.g., 23 in FIG. 5D, is required to step down the intensity of the white light, while passing the NIR or IR. In some embodiments, a shutter (e.g. LCD shutter, or ‘filter wheel,’ electronic variable optical attenuator (EVOA), an optical ‘chopper’, or a combination of polarizers can be synchronized to the excitation signal in order to selectively attenuate the visible light, but not the NIR or IR. In some embodiments, a filter that physically moves can be used to selectively attenuate the visible light, but not the NIR or IR. In some embodiments, such a filter sets the relative intensity of the VIS and infrared or NIR images and the dynamic range of the corresponding fluorescence signal.

In some embodiments, the two camera imaging system herein advantageously allows one or more of: a reduction in the required frame rates of the cameras, allowing the use of multiple, smaller, longer data cables from the cameras; a decrease in the bandwidth over a given cable, since there can be one or more data cables; a reduction in system cost by eliminating an expensive high-speed camera and frame grabber cards; allowing independent apertures on each of the VIS and infrared or NIR cameras for large depth of field on the VIS camera while not reducing the sensitivity in the NIR or IR camera; not requiring the use of an apochromatic lens (corrected for infrared or NIR and VIS wavelengths to focus at the same imaging plane) and broadband coatings for optimal transmission in VIS and NIR or IR as in the single camera imaging system. In some embodiments, only one cable is used, wherein a hub multiplexes data from the two cameras data onto one communication channel.

In some embodiments, a single camera or a two-camera image system is selected at least partly based on specifics in applications. In some embodiments, the two-camera imaging system herein advantageously allows different sensitivity (e.g., very high sensitivity for infrared or NIR and normal sensitivity for visible which can be useful in applications when the tissue can take up the dye but not in high concentration). The sensitivity range is defined by exposure time, which is related to the frames per second (fps) displayed. A ‘real time’ frame rate is approximately 25 fps. In some cases, for example, when viewing tissues, samples or tumors with high uptake of a fluorescent compound or drug, the associated exposure time for 25 fps will be sufficiently sensitive to detect the fluorescence emission. Higher sensitivity can be achieved by taking a longer exposure, leading to a slower frame rate. In some applications, frame rate as slow as 2 frames per second or any exposure longer than about 25 fps may be used to capture the autofluorescence in the tissues, or sample. In general, the frame rate is a function of the reciprocal of the exposure time. The exposure times, and the subsequent fps rate can be adjusted in real time to meet the sensitivity needs for the application. The two-camera image system herein can allow for varying each camera's exposures for optimal sensitivity of the infrared or NIR images, without saturating the visible images. In some embodiments, the imaging system is used as a microscope attachment or an exoscope. For purposed herein, an exoscope is a system that collects images from a position outside the body, as opposed to an endoscope, which captures images while positioned within body structures. In some embodiments, an exoscope is a video telescope operating monitor system to perform microsurgery, or surgical robot attachment or as a stand-alone imaging system for open field application(s). In some embodiments, a single camera imaging system advantageously includes the ability to miniaturize the entire setup, e.g., for endoscopes. The single camera imaging system or the two-camera imaging system can be attached in front of a flexible or rigid endoscope (e.g., the optics and sensor of the endoscope are at the distal end towards the target while the body of the endoscope will carry the electrical signal from the sensor instead of optical as in other endoscopes. An electrical sensor located at the end or tip of the endoscope (rather than a light guide on the end of the endoscope) can also be used to carry the image to camera. In some embodiments, the single-camera or two-camera imaging systems herein is used in minimally invasive surgical approaches with endoscopes.

In some embodiments, the image sensors herein include a charge-coupled device (CCD) or complementary metal-oxide semiconductor (CMOS) image sensor.

A non-limiting exemplary embodiment of the sensor used herein is the Sony IMX 174 CMOS chip in a Basler acA1920-155 camera. In this particular embodiment, the camera includes a 1/1.2 inch area sensor, a pixel size of about 5.86 um, and a resolution of 1936×1216 (2.3 MP).

In some embodiments, the camera being used is a standard CMOS or CCD camera. In some embodiments, the CMOS and CCD cameras have a High Definition (HD) resolution of about 1920×1080p. In some embodiments, the camera CMOS and CCD cameras have a resolution below 1920×1080p. In some embodiments, the camera CMOS and CCD cameras have a resolution greater than 1920×1080p. In some embodiments, the camera resolution is lower than HD, e.g., fewer than 1080 pixels. In some embodiments, the camera resolution High Definition (HD) resolution or higher, e.g., 1920-4000 pixels, 4K (Ultra HD/UHD), 8K, or higher pixel numbers. In some embodiments, the systems and methods here do not require specialized cameras such as EMCCD, ICCD etc. In some embodiments, the specialized cameras can be used to increase sensitivity, resolution, or other parameters associated with imaging. Table 1 shows information of exemplary embodiments of visible light and NIR or IR cameras herein.

TABLE 1

| Camera Manufacturer | Basler AG | |
|---|---|---|
| Camera Model: | VIS: acA1920-155uc | NIR: acA1920-155um |
| Sensor Pixel Resolution: | 1936 × 1216 | |
| Sensor Type | Sony IMX174LLJ-C, Progressive Scan CMOS, | |
| | Global Shutter | |
| Sensor active size | 11.3 × 7.1 mm | |
| Effective sensor | 13.4 mm | |
| diagonal size | | |
| Pixel Size (H × V) | 5.86 × 5.86 microns | |
| Max frame rate | Approximately 155 fps | |
| Data transport | USB 3.1 Gen 2, 10 Gbit/s | |
| Pixel formats | Mono 8 | Mono 8 |
| | Mono 12p | Bayer RG 8 |
| | Mono 12 | Bayer RG 12 |
| | | Bayer RG 12p |
| | | RGB 8 |
| | | BGR 8 |
| | | YCbCr422 8 |
| Filter | None | Hot mirror |

| | |
|---|---|
| Size (L × W × H) | 48.2 × 29 × 29 mm |
| Weight | 80 g |
| Conformity | CE, UL in preparation, FCC |

In some embodiments, the systems and methods herein include one or more light sensor(s) (e.g., photodiode, or other appropriate sensor). In some embodiments, the light sensors are configured for safety calculations and monitoring in the systems and methods. In some embodiments, light sensor(s) are located at the prism after the collimation lens, behind the dichroic filter 6, at the proximal end of excitation fiber and/or anywhere in the excitation path for total and relative power measurements. In some embodiments, two or any other number of photodiodes are located behind a hot mirror to monitor the shape of excitation source's illumination, thereby ensuring NIR or IR source and/or diffuser performance.

In some embodiments, a one- or two-dimensional sensor array, or alternatively a CMOS array, is located behind a hot mirror to monitor the excitation source's illumination thereby ensuring diffuser performance.

### Optical Light Guides

The plurality of optics can be configured to illuminate the tissue and to collect the visible light and fluorescence light emitted therefrom. In some embodiment, the optical guide is not present and the laser travels in free space.

The plurality of optics can comprise a component selected from a list including but not limited to: a filter, an optical transmission mechanism, a lens, a mirror, and a diffuser. The filter can be configured to block light from the excitation source. The filter can comprise a band pass filter, a cleanup filter, or both. The band pass filter can be configured to control a wavelength of light. The cleanup filter can allow light with a certain wavelength and/or a certain angle of incidence to pass through. The cleanup filter can comprise a narrow-band bandpass filter. The mirror can comprise a dielectric mirror.

The optical transmission mechanism can comprise free space, or a light guide. The optical light guide can comprise an optical fiber, a fiber optic cable, a liquid light guide, a waveguide, a solid light guide, a plastic light guide, or any combination thereof. In some embodiments the optical fiber comprises silicate glass, plastic, quartz or any other material capable of transmitting excitation laser light. In some embodiments at least one of the plurality of optics comprises a coaxial light injection mechanism configured to provide additional coaxial light to the system. The coaxial light injection mechanism can comprise a through hole in one or more of the plurality of optics. It is understood that any type of optical transmission mechanism can be used in any of the embodiments of this system. The optical transmission mechanism can be configured to transmit infrared or near infrared light. The optical light can comprise a spliced or unspliced optical fiber. The diameter of the optical fiber can depend on the amount of power and the number of emitters in the excitation source, including the physics of collection optics.

In some embodiments, the optical fiber has a cross-sectional diameter of about 10 um to about 1,000 um. In some embodiments, the optical fiber has a cross-sectional diameter of about 10 um to about 25 um, about 10 um to about 50 um, about 10 um to about 75 um, about 10 um to about 100 um, about 10 um to about 200 um, about 10 um to about 300 um, about 10 um to about 400 um, about 10 um to about 500 um, about 10 um to about 600 um, about 10 um to about 800 um, about 10 um to about 1,000 um, about 25 um to about 50 um, about 25 um to about 75 um, about 25 um to about 100 um, about 25 um to about 200 um, about 25 um to about 300 um, about 25 um to about 400 um, about 25 um to about 500 um, about 25 um to about 600 um, about 25 um to about 800 um, about 25 um to about 1,000 um, about 50 um to about 75 um, about 50 um to about 100 um, about 50 um to about 200 um, about 50 um to about 300 um, about 50 um to about 400 um, about 50 um to about 500 um, about 50 um to about 600 um, about 50 um to about 800 um, about 50 um to about 1,000 um, about 75 um to about 100 um, about 75 um to about 200 um, about 75 um to about 300 um, about 75 um to about 400 um, about 75 um to about 500 um, about 75 um to about 600 um, about 75 um to about 800 um, about 75 um to about 1,000 um, about 100 um to about 200 um, about 100 um to about 300 um, about 100 um to about 400 um, about 100 um to about 500 um, about 100 um to about 600 um, about 100 um to about 800 um, about 100 um to about 1,000 um, about 200 um to about 300 um, about 200 um to about 400 um, about 200 um to about 500 um, about 200 um to about 600 um, about 200 um to about 800 um, about 200 um to about 1,000 um, about 300 um to about 400 um, about 300 um to about 500 um, about 300 um to about 600 um, about 300 um to about 800 um, about 300 um to about 1,000 um, about 400 um to about 500 um, about 400 um to about 600 um, about 400 um to about 800 um, about 400 um to about 1,000 um, about 500 um to about 600 um, about 500 um to about 800 um, about 500 um to about 1,000 um, about 600 um to about 800 um, about 600 um to about 1,000 um, or about 800 um to about 1,000 um. In some embodiments, the optical fiber has a cross-sectional diameter of about 10 um, about 25 um, about 50 um, about 75 um, about 100 um, about 200 um, about 300 um, about 400 um, about 500 um, about 600 um, about 800 um, or about 1,000 um. In some embodiments, the optical fiber has a cross-sectional diameter of at least about 10 um, about 25 um, about 50 um, about 75 um, about 100 um, about 200 um, about 300 um, about 400 um, about 500 um, about 600 um, or about 800 um. In some embodiments, the optical fiber has a cross-sectional diameter of at most about 25 um, about 50 um, about 75 um, about 100 um, about 200 um, about 300 um, about 400 um, about 500 um, about 600 um, about 800 um, or about 1,000 um.

In some embodiments, the optical light guide has a length of about 0.005 m to about 10 m. In some embodiments, the optical light guide has a length of about 0.005 m to about 0.01 m, about 0.005 m to about 0.05 m, about 0.005 m to about 0.1 m, about 0.005 m to about 0.5 m, about 0.005 m to about 1 m, about 0.005 m to about 2 m, about 0.005 m to about 3 m, about 0.005 m to about 4 m, about 0.005 m to about 6 m, about 0.005 m to about 8 m, about 0.005 m to about 10 m, about 0.01 m to about 0.05 m, about 0.01 m to about 0.1 m, about 0.01 m to about 0.5 m, about 0.01 m to about 1 m, about 0.01 m to about 2 m, about 0.01 m to about 3 m, about 0.01 m to about 4 m, about 0.01 m to about 6 m, about 0.01 m to about 8 m, about 0.01 m to about 10 m, about 0.05 m to about 0.1 m, about 0.05 m to about 0.5 m, about 0.05 m to about 1 m, about 0.05 m to about 2 m, about 0.05 m to about 3 m, about 0.05 m to about 4 m, about 0.05 m to about 6 m, about 0.05 m to about 8 m, about 0.05 m to about 10 m, about 0.1 m to about 0.5 m, about 0.1 m to about 1 m, about 0.1 m to about 2 m, about 0.1 m to about 3 m, about 0.1 m to about 4 m, about 0.1 m to about 6 m, about 0.1 m to about 8 m, about 0.1 m to about 10 m, about 0.5 m to about 1 m, about 0.5 m to about 2 m, about 0.5 m to about 3 m, about 0.5 m to about 4 m, about 0.5 m to about 6 m, about 0.5 m to about 8 m, about 0.5 m to about 10 m, about 1 m to about 2 m, about 1 m to about 3 m, about 1 m to about 4 m, about 1 m to about 6 m, about 1 m to about 8 m, about 1 m to about 10 m, about 2 m to about 3 m, about 2 m to about 4 m, about 2 m to about 6 m, about 2 m to about 8 m, about 2 m to about 10 m, about 3 m to about 4 m, about 3 m to about 6 m, about 3 m to about 8 m, about 3 m to about 10 m, about 4 m to about 6 m, about 4 m to about 8 m, about 4 m to about 10 m, about 6 m to about 8 m, about 6 m to about 10 m, or about 8 m to about 10 m. In some embodiments, the optical light guide has a length of about 0.005 m, about 0.01 m, about 0.05 m, about 0.1 m, about 0.5 m, about 1 m, about 2 m, about 3 m, about 4 m, about 6 m, about 8 m, or about 10 m. In some embodiments, the optical light guide has a length of at least about 0.005 m, about 0.01 m, about 0.05 m, about 0.1 m, about 0.5 m, about 1 m, about 2 m, about 3 m, about 4 m, about 6 m, or about 8 m. In some embodiments, the optical light guide has a length of at most about 0.01 m, about 0.05 m, about 0.1 m, about 0.5 m, about 1 m, about 2 m, about 3 m, about 4 m, about 6 m, about 8 m, or about 10 m. The length of the optical light guide can be measured as a minimum, average, or maximum distance between an input side and an output side of the optical light guide when the optical light guide is straightened.

In some embodiments, a laser module generates the excitation light, which is directed into an optical light guide. In some embodiments, an infrared source generates the excitation light, which is directed into an optical light guide. In some embodiments, a near-infrared source generates the excitation light, which is directed into an optical light guide.

In some embodiments, at least a portion of the diffuser fits within a hole in the NIR mirror, for example, as shown in FIGS. 8A-8B. In this particular embodiment, one or more of the optical elements of the light source (e.g., collimator 17, clean up filter 16, mirror 15, diffuser 14, excitation assembly, optical excitation assembly, or optical scaffold) can be located outside the hole of the NIR mirror. In other embodiments, one or more of the optical elements of the light source (e.g., collimator 17, clean up filter 16, mirror 15, and diffuser 14) can be located inside the hole of the NIR mirror. In other embodiments, one or more of the optical elements of the light source (e.g., collimator 17, clean up filter 16, mirror 15, and diffuser 14) can be located inside the surface of the NIR Mirror (e.g., mirror 4), or directly proximal to the mirror. In some embodiments, a distance from the diffuser to the drape is about 130 mm. According to various aspects of the system 1000, the mirror 15 can be a dielectric, NIR mirror, hot mirror, turn mirror, metal-coated mirrors, dielectric mirrors, Bragg mirrors, crystalline mirrors, first surface mirrors, parabolic mirrors, variable reflectivity mirrors, deformable mirrors, laser mirrors, laser line mirrors, fiber loop mirrors, semiconductor saturable absorber mirrors, supermirrors or other suitable mirror.

In some embodiments, the optical light guide includes an optical scaffold for introduction of the excitation light into the imaging system. In some embodiments, such a scaffold includes a hot mirror, dielectric mirror, silvered mirror, gold mirror or the like, such as a NIR dielectric mirror 4. The excitation light can be inserted into the imaging system through a hole within the mirror

In some embodiments, the system comprises one or more illumination sources. The one or more illumination sources can comprise a fluorescence excitation light source such as a narrowband laser configured to generate an excitation beam to stimulate fluorescence in the region of tissue imaged. Alternatively, or in combination, the fluorescence excitation source can comprise a wide band source such as a light emitting diode (LED) coupled to a notch filter to generate the fluorescence excitation wavelengths. In some embodiments, the system comprises multiple excitation light sources. The one or more illumination sources can comprise a visible light illumination source to illuminate the region of tissue imaged with visible light. Moreover, a broadband source can be used as an illumination source. The broadband source can comprise a white light, an infrared light an incandescent lamp, a gas discharge lamp, a xenon lamp, an LED, or any combination thereof. The broadband source can emit NIR or IR spectrum light for fluorescence excitation and visible light for illumination. A plurality of optics can be configured to illuminate the target and collect the visible light and fluorescence emission light. The plurality of optics can comprise filters to remove the light from the excitation source. The system can comprise one or more imaging sensors to capture the fluorescence emission light and the visible illumination light reflected from the target.

Referring to FIGS. 4 & 6A, in a particular embodiment, the target or sample is illuminated by the main illumination 12a and/or contra-later illumination 12b. The visible light from the target or sample is filtered by the primary dichroic shortpass filter 6, and only a small amount (i.e., leaked visible light), for example, 5-10% of the incident light at the shortpass filter 6 goes through a secondary dichroic filter 5 and reaches the visible lens 11a and camera 10a. In some embodiments, 1%-5%, 3%-10%, 5%-12%, 10%-15%, up to 20% or more of the incident light at the shortpass filter 6 goes through a secondary dichroic filter 5 and reaches the visible lens 11a and camera 10a. Non-limiting exemplary embodiment of the visible camera is Basler acA1920-155uc. Non-limiting exemplary embodiment of the NIR or IR camera is acA1920-155 um. In some embodiments, 1%-5%, 3%-10%, 5%-12%, 10%-15%, up to 20% or more of the incident light at the shortpass filter 6 goes through a secondary dichroic filter 5 and is then filtered using a polarizer to remove ghosting, neutral density filter (optional) and a shortpass filter (to remove any traces of excitation light and fluorescence emission and gets further reflected by mirror FIG. 6A.

In some embodiments, the primary dichroic shortpass filter 6 and the secondary dichroic filter 5 is any beam splitter, prism, filter, mirror, or other optical component that is configured to perform similar shortpass function as the dichroic filter.

Continue to refer to FIG. 4, in the same embodiment, almost all of the fluorescence light from the target or sample gets reflected by the primary dichroic shortpass filter 6 and then the secondary dichroic shortpass filter 5, thus separated from the majority of visible light at the primary dichroic filter and then separated from the leaked visible light at the secondary dichroic filter. In this embodiment, the fluorescence light gets reflected at the NIR mirror 4 and further filtered by a longpass filter 3 before it reaches the NIR lens 11b and NIR camera 10b. An additional NIR longpass filter 3.5 can be included between the NIR lens and the camera. In some embodiments, there is no additional NIR longpass filter between the NIR lens and the camera. In some embodiments, the aforementioned filters are infrared filters. Non-limiting exemplary embodiment of the longpass filter 3 is Edmund UV/VIS cut imaging filter. Non-limiting exemplary embodiment of the NIR longpass filter 3.5 is 808 nm longpass Semrock Edge Basic.

In some embodiments, the dichroic filter/mirror e.g., 5, 6, and/or 8 herein includes an angle of incidence (AOI). The angle of incidence is 0 degree, 45 degrees, or any other angles. In some embodiments, the angle of incidence is 10°, 15°, 20°, 25°, 30°, 35°, 45°, 50°, 55°, 60°, 65°, 70°, 75°, or any other angle. Non-limiting exemplary embodiment of dichroic filter 5, 6is Edmund 45AOI hot mirror and 720 nm SP filter from Semrock, FF720-SDi01-55×55, respectively.

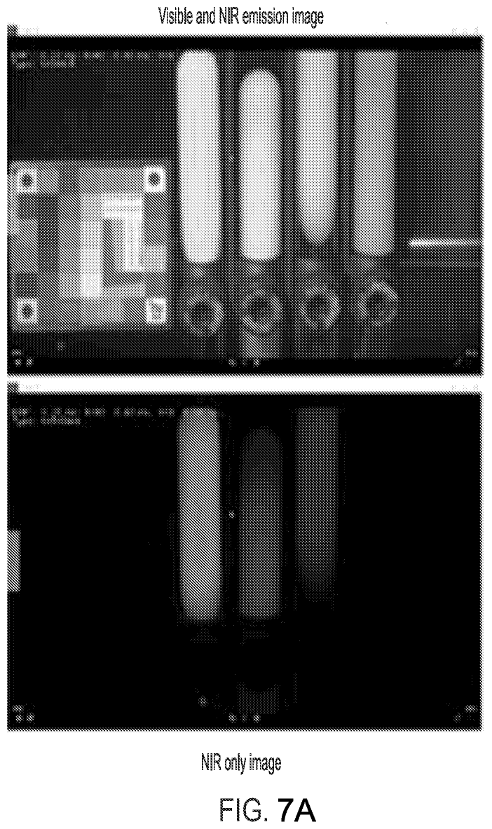

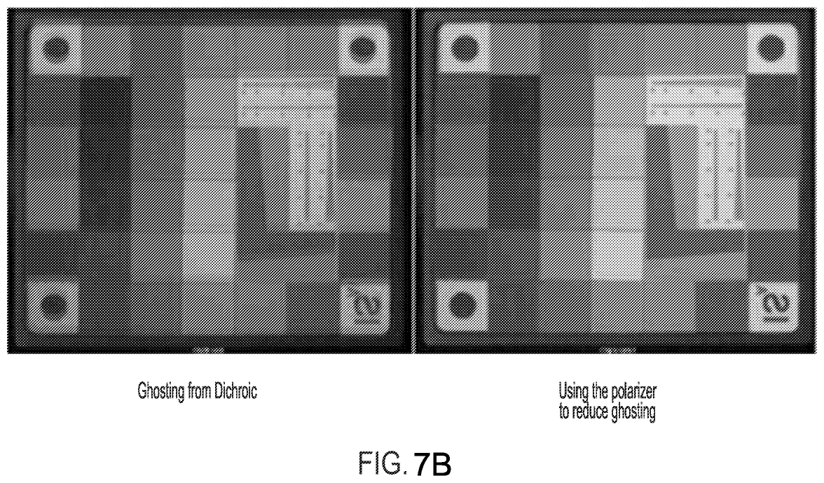

In some embodiments, the dichroic filter 6 is a filter that is specifically configured to allow the specified amount of VIS reflection, with high surface quality to reduce reflections from the excitation source, and a short enough wavelength edge to allow reflection of the large cone-angle for the excitation that reflects at AOI of 45+/−10 degrees. In some embodiments, the dichroic filter allows the reflection of the large cone-angle for the excitation that reflects at an AOI of 10°, 15°, 20°, 25°, 30°, 35°, 45°, 50°, 55°, 60°, 65°, 70°, 75°, or any other angle+/−10 degrees. In some embodiments, the dichroic filter 6 causes ghosting (FIGS. 7B-7C) in the visible light image due to secondary reflection of the leaked visible light from back surface. This light has different polarization than the light emitted by first surface. This allows the use of polarizer to eliminate (ghosting) images from a selected surface. In some embodiments, the polarizer only works if one surface (generally the primary reflective surface) has a substantial polarizing effect. The AR coating on the back will not show any discretion when polarized, other than equal attenuation. FIG. 7C show exploded views of top and bottom right corners of FIG. 7B. In this embodiment, ghosting is significantly reduced or even removed by the use of polarizer, LC attenuator, or other optical elements of similar functions.

In some embodiments, the dichroic filter 5 has various functions including but not limited to: reflecting the excitation beam; 2) reflecting the infrared or NIR fluorescence; 3) transmitting the visible image to the VIS camera. In some embodiments, this element is used for the splitting of the infrared or NIR and VIS paths.

FIG. 8B shows an exemplary embodiment of the path of light followed by the illumination from the light source. In this embodiment, the system includes a 0-AOI hot mirror 8 which is positioned between a 45 AOI hot mirror 6 and the microscope 27. In this embodiment, the hot mirror 8 is configured as a safety filter for reducing excitation from leaking into the microscope (e.g., 785 nm) and eliminates NIR or IR illumination from the microscope light, of the tissue that will be mixed in the VIS_DRK frame (a frame captured when the excitation source is OFF that may include ambient light or other light present in the imaging environment) and requires subtraction from the actual NIR or IR fluorescence. In some embodiments, the aforementioned functionalities are as applied to infrared light. In some embodiments, the aforementioned functionalities are as applied to excitation source's light in the infrared range or NIR range. In some embodiments, the aforementioned functionalities are as applied to an infrared source (e.g., a wide band source (e.g., light emitting diode (LED)) with a band pass filter) in the infrared range or NIR range.

In some embodiments, one or more of the dichroic filters or dichroic mirrors herein functions as a wavelength-specific beam splitter. In some embodiments, the dichroic filter herein is any optical element that is configured to perform passive wavelength-specific beam splitting or beam separation.

Referring to FIG. 4, in a particular embodiment, the NIR or IR imaging path includes a longpass (LP) filter 3 (e.g., a dielectric-coated filter, with 0-degree angle of incidence) that reflects all light shorter in wavelength than 800 nm (greater than OD6 blocking for <800 nm). The primary function of this LP filter is to eliminate the excitation light reflected off the sample and thus enable the sensor to image the fluorescence signal. In some embodiments, the LP filter further filters VIS light out of the fluorescence light. In some embodiments, with a single camera the long pass filter can be replaced by a notch filter (broader in spectral band than the band pass laser clean up filter) which will block only the excitation light while letting both the visible image as well as fluorescence image on the sensor.

In some embodiments, little or no fluorescence reaches the VIS camera, since >90% is reflected by the dichroic filter 5. The shortpass filter 1, in some embodiments, is to reduce excitation leakage into the VIS camera. The VIS camera can have an additional hot mirror placed in front of the sensor (not shown in FIG. 4).

In some embodiments, dichroic filter 5 is the primary splitting agent for the VIS and NIR or IR imaging paths. In some embodiments, one or more SP and LP dielectric filters herein are primarily for attenuation of the excitation into the imaging lens.

In some embodiments, fluorescence signal from tissue is reflected by a dichroic shortpass filter while visible light passes through as if it is completely transparent. The reflected fluorescent light can be further reflected by a second shortpass dichroic before it is reflected again on a mirror and passes through a longpass filter unchanged (e.g., “unchanged” meaning with less than 1%, 2%, 3%, 4%, or 5% of attenuation while rejecting unwanted excitation) to reach the lens and sensor.

In some embodiments, 95% or even more of the visible light just passes through the dichroic shortpass filter 6, only a tiny amount is reflected (leaked by) the filter. The leaked visible light can pass unchanged through a secondary dichroic filter 5 before a normal mirror reflects it. The visible light then can get reflected again by a dichroic longpass filter before it is received at the lens and imaging sensor, as shown in FIGS. 6A-6B.

In some embodiments, a small portion of visible light is reflected from both the front and back surfaces of a dichroic mirror. Due to the thickness of the dichroic mirror, the back surface reflection has a longer optical path length, registering as an offset on the sensor, leading to a ghosting effect where the image appears doubled, as shown in FIGS. 7B-7C. In some embodiments, the light from the front surface is 90° rotated in polarization compared to light reflected from the back surface. In some embodiments, one surface may be polarized, the other not, or one reflection can be blocked, but the other is not blocked. Thus, this ghosting effect can be eliminated using a polarizer 2 as shown in FIG. 6A. Alternatively, a liquid crystal attenuator 2a in FIG. 6B can be used for variable attenuation of the visible light. In this embodiment, in FIG. 6B, the LC attenuator polarizes (e.g., accepts linearly polarized light, rejecting other axis, as the LC is sandwiched between two polarizers) the incoming light, therefore reducing ghosting. In some embodiments, the systems and methods herein include a polarizer positioned in front of or behind the LC for reducing ghosting. In some embodiments, each member of crossed polarizers is placed on a side of the LC. In some embodiments, the systems and methods herein include no polarizer additional to the LC for reducing ghosting. In some embodiments, the LC attenuator herein is inherently polarized and thus by controlling the polarization of LC, front or the back reflection of the dichroic mirror can be eliminated thereby removing ghosting. But there can be a significant drawback in using a polarizer or a similar device in the systems and methods herein if the polarizer is in front of reflected near infrared light. In some embodiments, a polarizer or similar element reduces the photons from the infrared fluorescence signal, which causes undesired fluorescence signal loss. In order to reduce ghosting without affecting or reducing the infrared fluorescence signal, in some embodiments, the polarizer or similar device is used only on visible light but not the infrared or NIR light. In some embodiments, the positioning of the polarizer is in a separate image path from infrared or NIR signal, in order to minimize ghosting. In some embodiments, the polarizer is placed in front of the lens, camera or mirror without any additional optical elements there between. In some embodiments, the polarizer is placed at least behind the primary and/or the secondary dichroic filter/mirror. In some embodiments, the polarizer is placed in front of the lens, camera or mirror with only a notch filter and/or a VIS-Cut filter there between. Referring to FIGS. 4, 6A-6B, in a particular embodiment, the polarizer 2, attenuator 2a, or similar device is placed so that mixed visible and infrared light is split using a hot mirror 5 (which is a shortpass (SP) dichroic filter) in which the visible light (blue arrows) goes through filter 5 and then the polarizer 2 and onto a secondary visible light lens 11a, and visible camera 10a, or onto a mirror 18 which again reflects in back on a single sensor 21, with another longpass dichroic filter 19 which reflects the visible light on the sensor.

Referring to FIG. 5A, in one embodiment, the visible light directly reaches the VIS/NIR lens 20 and camera 21 after it is filtered by a polarizer 2 to remove ghosting, an optional VIS-Cut filter (neutral density filter or LCD filter or any other optical element which passively or actively reduce the total amount of light passing through) 23 to selectively further attenuate the visible light if needed but not the IR or NIR light Alternatively, a synchronized ‘shutter’ (e.g. LCD, or ‘filter wheel’, or optical ‘chopper’, electronic variable optical attenuator (EVOA)) can be used to provide such attenuation. (e.g., 1% visible light transmission and about 100% NIR or IR transmission in the range of 800-950 nm), and a notch filter 22 to remove light from the excitation source. The fluorescence light, in the same embodiment, after getting reflected at the primary dichroic mirror 6, is attenuated by the polarizer 2, transmitted through the VIS-Cut filter 23, and notch filter 22 to reach the single VIS/NIR camera 21. In some embodiments, the primary dichroic mirror 6 has a length of about 35 mm to about 40 mm, or about 23 mm to about 54 mm. In some embodiments, the primary dichroic mirror 6 has a height of about 29 mm to about 35 mm, or about 23 mm to about 38 mm. In some embodiments, a distance from the dichroic shortpass 6 mirror to the VIS or NIS lens is less than about 50 mm. In some embodiments, a distance from the dichroic shortpass mirror to the VIS or NIS lens is less than about 1,000 mm. In various other embodiments, the dichroic mirror 6 may have smaller or larger dimensions, while miniaturization of the mirror 6 is preferred.

As shown in FIGS. 5B-5C, a pair of mirrors 25, 26 can be used to allow coaxial illumination through a hole at mirror-1 25, and both the visible light and the fluorescence light are twice reflected at the pair of mirrors before they reach the polarizer 22.

In some embodiments, the systems and methods herein is a two-camera imaging system that are configured to sense either visible or NIR or IR signals, separately, as in FIG. 4. In some embodiments, the systems and methods herein is a single-camera imaging system that are configured to sense both visible or NIR or IR signals, as in FIGS. 6A & 6B. In some embodiments, a two-camera imaging system is capable of providing both infrared or NIR and visible light images when high levels of visible ambient light are present in the imaging environment (without adverse imaging artifacts or the use of a VIS-Cut filter). Non-limiting examples of such high level of ambient light include: windows in the operating room, high intensity surgical lamps, and lights in the operation room that are required to be ON during the imaging. In some embodiments, at least one of the components shown in FIG. 4 can be aligned perpendicular to the page in displayed orientation. In some embodiments, the NIR mirror 4 is a dielectric mirror. In some embodiments, the optical fiber 13 is bent. In some embodiments, the optical fiber 13 is unbent.

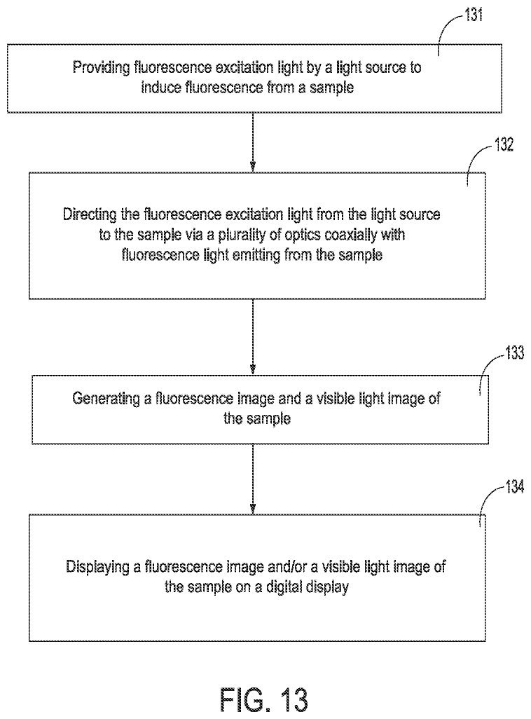

FIG. 13 shows an exemplary schematic diagram of one or more method steps for simultaneous visible light and fluorescence imaging using the imaging systems herein. In this particular embodiment, fluorescence excitation light, e.g., infrared light is provided by a light source to induce fluorescence from a sample 131. In some embodiments, the light source can be transmitted or “injected” through a hole in a dielectric mirror along the optical path of fluorescent light for NIR or IR imaging. In this embodiment, the infrared or NIR light from the light source is directed to the sample via a plurality of optics 132, the infrared light to the sample is substantially coaxial with fluorescence light received from the sample in order to decrease shadows in fluorescence image(s). The plurality of optics herein includes but is not limited to one or more of: a dichroic filter, a hot mirror, a beam splitter, a dielectric mirror, a polarizer, an attenuator, a notch filter, a neutral-density filter, a shortpass filter (e.g., wavelength shorter than 700 nm or 780 nm, or any wavelength between 700 nm and 800 nm), and a longpass filter (e.g., wavelength longer than 700 nm or 780 nm). In this embodiment, a plurality of optics collects the target's fluorescence signal and the reflected visible light image. The imaging system herein then captures a fluorescence image and a visible light image of the sample 133. The fluorescence image and the visible light image are not necessarily captured at the same frame rate. The fluorescence image(s) and the visible light image(s) can be processed by a processor to form a composite image. The composite image, the fluorescence image and/or the visible light image of the sample can be displayed to a user using a digital display 134.

FIGS. 4, 5A-5D, and 6A-6B show non-limiting exemplary positions of the polarizer or attenuator with respect to the lens, camera and other elements of the image systems. In some embodiments, the polarizer or attenuator here can include one or more polarizer or attenuator that can be placed in other positions of the optical train.

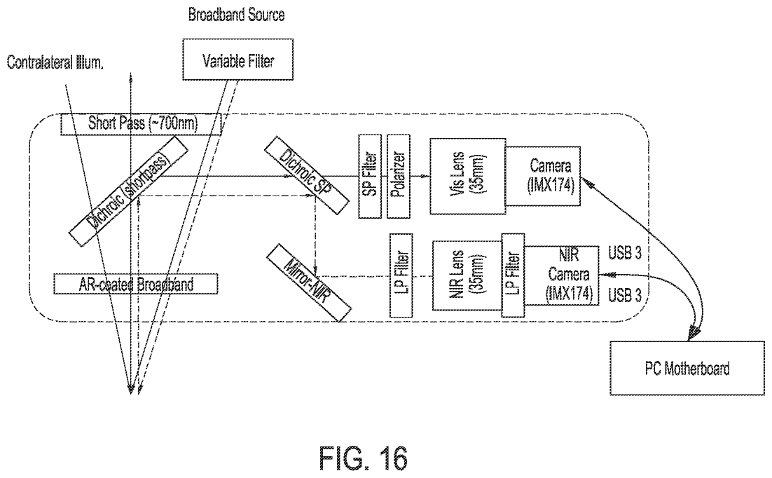

In some embodiments, the systems and methods described herein include a notch filter, for example the notch filter (22) as shown in FIG. 5A. In some embodiments, the notch filter is in the optical path between a dichroic mirror and the imaging sensor. As shown in FIGS. 5A-5D, and optionally in FIG. 4, FIGS. 6A and 6B, and FIG. 16, in some embodiments, the notch filer is in between a primary dichroic mirror and the imaging sensor. In some embodiments, the notch filter is in between a polarizer and an imaging sensor. In some embodiments, the notch filter is configured to filter out at least a part of the excitation source's light (e.g., >90%, >90.5%, >91%, >91.5%, >92%, >92.5%, >93%, >93.5%, >94%, >94.5%, >95%, >95.5%, >96%, >96.5%, >97%, >97.5%, >98%, >98.5%, >99%, >99.5%, >99.6%, >99.7%, >99.8%, or >99.9% or more) and a lens can be used to focus the remaining fluorescence light on the sensor. In some embodiment the notch filter always has wider spectral band width than the band pass filter such as laser clean up filter. In some embodiments, the notch filter includes a spectrum width of about 20 nm at 0 degree AOI and 10 nm at 10 degree AOI. In some embodiments the notch filter is >OD3 for 770-800 nm for 0 degree AOI. In some embodiments, i.e., for non-zero AOI, the filter notch bandstop shifts to a shorter wavelength whereby each 10 degrees it shifts by 5 nm. In some embodiments, the angle of incidence relative to the notch filter is 10°, 15°, 20°, 25°, 30°, 35°, 45°, 50°, 55°, 60°, 65°, 70°, 75°, 80°, 85°, or 90° or any other angle. It is understood that, depending on the AOI, the wavelength bandstop shifts accordingly.

In some embodiments, the working distance from an objective lens of the optical system to the tissue being imaged is less than 0.1 cm (1 mm), less than 0.2 cm (2 mm), less than 0.3 cm (3 mm), less than 0.4 cm (4 mm), less than 0.5 cm (5 mm), less than 0.6 cm (6 mm), less than 0.7 cm (7 mm), less than 0.8 cm (8 mm), less than 0.9 cm (9 mm), less than 1 cm, less than 2 cm, less than 3 cm, less than 4 cm, less than 5 cm, less than 6 cm, less than 7 cm, less than 8 cm, less than 9 cm, less than 10 cm, less than 20 cm, less than 30 cm, less than 40 cm, less than 50 cm, or more.

In some embodiments, the working distance is about 0.1 cm to about 50 cm. In some embodiments, the working distance is about 0.1 cm to about 0.2 cm, about 0.1 cm to about 0.5 cm, about 0.1 cm to about 0.7 cm, about 0.1 cm to about 0.9 cm, about 0.1 cm to about 1 cm, about 0.1 cm to about 5 cm, about 0.1 cm to about 10 cm, about 0.1 cm to about 20 cm, about 0.1 cm to about 30 cm, about 0.1 cm to about 40 cm, about 0.1 cm to about 50 cm, about 0.2 cm to about 0.5 cm, about 0.2 cm to about 0.7 cm, about 0.2 cm to about 0.9 cm, about 0.2 cm to about 1 cm, about 0.2 cm to about 5 cm, about 0.2 cm to about 10 cm, about 0.2 cm to about 20 cm, about 0.2 cm to about 30 cm, about 0.2 cm to about 40 cm, about 0.2 cm to about 50 cm, about 0.5 cm to about 0.7 cm, about 0.5 cm to about 0.9 cm, about 0.5 cm to about 1 cm, about 0.5 cm to about 5 cm, about 0.5 cm to about 10 cm, about 0.5 cm to about 20 cm, about 0.5 cm to about 30 cm, about 0.5 cm to about 40 cm, about 0.5 cm to about 50 cm, about 0.7 cm to about 0.9 cm, about 0.7 cm to about 1 cm, about 0.7 cm to about 5 cm, about 0.7 cm to about 10 cm, about 0.7 cm to about 20 cm, about 0.7 cm to about 30 cm, about 0.7 cm to about 40 cm, about 0.7 cm to about 50 cm, about 0.9 cm to about 1 cm, about 0.9 cm to about 5 cm, about 0.9 cm to about 10 cm, about 0.9 cm to about 20 cm, about 0.9 cm to about 30 cm, about 0.9 cm to about 40 cm, about 0.9 cm to about 50 cm, about 1 cm to about 5 cm, about 1 cm to about 10 cm, about 1 cm to about 20 cm, about 1 cm to about 30 cm, about 1 cm to about 40 cm, about 1 cm to about 50 cm, about 5 cm to about 10 cm, about 5 cm to about 20 cm, about 5 cm to about 30 cm, about 5 cm to about 40 cm, about 5 cm to about 50 cm, about 10 cm to about 20 cm, about 10 cm to about 30 cm, about 10 cm to about 40 cm, about 10 cm to about 50 cm, about 20 cm to about 30 cm, about 20 cm to about 40 cm, about 20 cm to about 50 cm, about 30 cm to about 40 cm, about 30 cm to about 50 cm, or about 40 cm to about 50 cm. In some embodiments, the working distance is about 0.1 cm, about 0.2 cm, about 0.5 cm, about 0.7 cm, about 0.9 cm, about 1 cm, about 5 cm, about 10 cm, about 20 cm, about 30 cm, about 40 cm, or about 50 cm. In some embodiments, the working distance is at least about 0.1 cm, about 0.2 cm, about 0.5 cm, about 0.7 cm, about 0.9 cm, about 1 cm, about 5 cm, about 10 cm, about 20 cm, about 30 cm, or about 40 cm. In some embodiments, the working distance is at most about 0.2 cm, about 0.5 cm, about 0.7 cm, about 0.9 cm, about 1 cm, about 5 cm, about 10 cm, about 20 cm, about 30 cm, about 40 cm, or about 50 cm.

### Coaxial Illumination

In some embodiments, as the illumination signal is injected though a hole in a mirror in the imaging path, the systems and methods herein enable coaxial illumination and light collection. Unlike prior imaging systems, the coaxial illumination of the imaging systems herein enable visualization of organs, substructures of organs, targets, tissue and cells without casting a shadow on the sample being viewed. Avoiding shadows is beneficial to prevent obstruction from both the visible, infrared, and near infrared light within the images of the organs, substructures of organs, targets, tissue and cells. Further, such shadows can obstruct fluorescent signals from the tissue and cause false negatives. In some embodiments, the systems and methods herein utilize coaxial illumination to avoid this problem. FIG. 3B shows the coaxial illumination and imaging axes, in comparison to separate illumination and imaging axes, FIG. 3A. In this particular embodiment, coaxial illumination improves the visibility of the tissue by reducing shadows, thus false negatives (no fluorescence), thereby improving the imaging of a tissue cavity, organ, and substructure of organs, target, tissue or cell that is under observation by the system.

In some embodiments, the illumination signal is injected into the imaging axis by a notch dichroic filter 5 as shown in FIG. 18. This optical filter transmits the visible and fluorescent signals while reflecting the excitation, and thereby efficiently injecting the excitation coaxially. The angle of incidence on the filter 5 for example, can be 22.5 degrees, 45 degrees.

In some embodiments, the imaging axis of the microscope, the imaging axis of the imaging system herein, and the excitation axis are all coaxial with each other. In some embodiments, the image axis and the excitation axis share the same common axis.

In some embodiments, the imaging axis is aligned to the center of the right ocular axis or aligned to the left ocular axis, thus enabling a concentric field of view with the right ocular axis or the left ocular axis, for example. Alternatively, the light beam corresponding to excitation can extend toward the tissue from a location between the left and right objective lenses, and the imaging axis of the fluorescence camera can extend coaxially with the excitation axis from the tissue toward the sensor. The images may not necessarily comprise the same image size, and can comprise the same or different image sizes. The center point of each coaxial beam can be aligned so both beams are within an appropriate tolerance of each other so as to be considered coaxial as would be understood by one of ordinary skill in the art. In some embodiments, coaxial imaging as described herein corresponds to the illumination and excitation axes (e.g., visible and NIR/IR) substantially overlapping or being substantially parallel with the imaging axis of image sensors (e.g. of camera), or other imaging axis of the imaging systems disclosed herein such as the left and right eyepieces and objective lenses of the microscope. The imaging axes can be configured for visible and/or fluorescence imaging, such as NIR/IR light imaging. For example, systems disclosed herein can comprise: 1) an imaging axis for visible light corresponding to an image as seen by the user through an eyepiece of the microscope, 2) the fluorescent light imaging axis such as infrared or NIR light received from the sample, and 3) the excitation light beam axis directed to the sample, are all coaxial with each other (i.e., they share the same common axis, or at least within an appropriate tolerance as disclosed herein).

In some embodiments, substantially overlapping or parallel includes an intersecting angle between two axes to be less than 30 degrees, 20 degrees, 10 degrees, less than 5 degrees, less than 2 degrees, less than 1 degree, less than 0.1 degree, or less than 0.01 degree or about 0 degrees. Substantially overlapping can correspond to beams that are coaxial to within an acceptable tolerance of each other, e.g. to within 1 mm, 0.5 mm, 0.25 mm or 0.1 mm of each other. In some embodiments, substantially overlapping or parallel includes an intersecting angle between two axes to be less than 10 degrees, less than 5 degrees, less than 2 degrees, less than 1 degree, less than 0.1 degree, or less than 0.01 degree or about 0 degrees. The working distance from an objective lens of the optical system to the tissue being imaged can be within a range from about few millimeters (less than 1 cm) (e.g., endoscope) to 200-500 mm (e.g., microscope) or longer (e.g., open field imaging system).

In some embodiments, coaxial imaging does not include stereoscopic imaging. In some embodiments, coaxial imaging as disclosed herein includes overlap of two or more optical paths, at least one for illumination, and at least one other for imaging. Moreover, in some embodiments, two or more optical paths can be coaxially aligned to enable coaxial visualization of multiple infrared or near infrared wavelengths, for example from two or more fluorophores that home, target, migrate to, are retained by, accumulate in, and/or bind to, or are directed to an organ, organ substructure, tissue, target, cell or sample. In some embodiments, two or more, three or more, four or more, or five or more such paths are coaxially positioned. In some embodiments, the infrared or near infrared light is delivered to the sample along an infrared or near infrared optical path and the fluorescent light received from the sample is received along a fluorescence optical path and wherein the fluorescence optical path overlaps with the infrared optical path at a beam splitter. In some embodiments, the intersecting angle between two axes comprises no more than 10 degrees, no more than 5 degrees, no more than 2 degrees, no more than 1 degree, no more than 0.1 degree, or no more than 0.01 degree or about 0 degrees.

In some embodiments, coaxial imaging herein includes concentric fields of view (not necessarily the same image size, but the center point of imaging systems (e.g., microscope, imaging system, etc. are aligned). In a coaxial imaging system, there is no user perceptible parallax as the working distance changes. In a coaxial imaging system, the imaging shift due to variation in the accuracy of coaxiality is less than about 0.3 degrees. In some embodiments of a coaxial imaging system, the imaging shift due to variation in the accuracy of coaxiality is less than about 0.05, 0.1, 0.05, 0.2, 0.25, 0.3, 0.35, 0.4, 0.45, 0.5, 0.55, or 0.6 degrees. In some embodiments, the imaging axis of the imaging system herein is aligned to the center of the right/left ocular axes, for example with reference to endoscopic applications.

### Eliminating Unwanted Light

In some embodiments, stray light arriving at the camera or sensor arrives from outside the imaging system, from one or more of the light channels, from ambient light (e.g. room lights, windows), or other light-emitting equipment (e.g. operating room equipment, neuronavigation equipment, endoscopes, exoscopes, microscopes, headlamps, and loupes). In some embodiments, the stray light is emitted continually or in a pattern of pulses. In some embodiments, the stray light is visible, infrared, or both.

Such unwanted light reduces the contrast of the fluorescent image. Additionally, visual illumination by the device interferes with fluorescence excitation. For example, the visible light illumination by the device may excite fluorophore and cause fluorescence in the VIS light image, turning the laser on and off is used when subtracted to see the difference. Ambient light isolation greatly improves image quality by reducing interference from non-visible wavelengths, visible wavelengths, infrared wavelengths, or any combination thereof. Many current devices, however, lack light isolation components to shield against such stray lighting, and must be used in a dark room to eliminate or diminish such ambient light.

In some embodiments, the systems herein further comprise an attenuator to block, filter or attenuate stray light. In some embodiments, the attenuator comprises a filter, a shield, a hood, a sleeve, a light shroud, a drape port, a baffle, or any combination thereof. In some embodiments, the physical attenuator blocks and/or filters out such stray or ambient light. In some embodiments, the attenuator is external or affixed to the systems herein. In some embodiments, the attenuator blocks light at angles of entrance greater than the field of view (FOV). In some embodiments, a drape port at an entrance aperture is sized so to block at least a portion of the exterior FOV of the imaging system. In some embodiments, the housing and/or optomechanical mounts are blackened to prevent reflection of light within the imaging system. In some embodiments, the light channels of the systems herein employ a light filter. In some embodiments, the light channels of the systems herein do not employ a baffle, which would eliminate the signal to be measured. In some embodiments, scatter within the imaging Head when the source has an angle greater than the clear aperture of the optical path prior to exit of the Imaging Head. In some embodiments, the optical path comprises a baffle that absorbs incident radiation. In some embodiments, the systems and methods herein eliminate interference between visual and fluorescence lights through synchronization and optimization of laser ON/OFF rates. In some embodiments, a power of the laser sufficiently high to be absorbed by the sample to cause fluorescence, while being minimized to reduce stray excitation within the device.

One or more stray light shrouds or baffles can be used between the camera sensor and lens assembly. The optical system is focused by moving the camera sensor relative to the lens (fixed). This requires a variable gap between the sensor and lens which is particularly sensitive to any stray light in the imaging system enclosure. A simple concentric tube design where one tube screws onto the camera C-mount and the other tube onto the lens support may be used to shroud this gap from stray light. The shroud surfaces may be painted with highly absorptive paint and overlap even when the sensor is at maximum extent of the focus range. Other embodiments can include a shield, hood, sleeve, light shroud, baffle, boot or other physical attenuator to block, filter or attenuate such light to enhance the methods and systems of the disclosure. Such a shield, hood, sleeve, light shroud, baffle, boot or other physical attenuator can be external or affixed to the systems of the disclosure.

Stray light can be inadvertently admitted into the imaging system enclosure through a gap between the sensor and lens necessary for focusing the system by moving the camera sensor relative to the fixed lens. For example, any of the systems described in FIGS. 4, 5, 6, 7, and 16 and throughout the disclosure can be used as described above or throughout the disclosure to eliminate the problems with stray light or ambient light. As such, the system can further comprise a light shroud between the camera sensor and lens assembly. The light shroud can comprise a tray, a cover, a baffle, a sleeve, a hood, or any combination thereof. The light shroud can block, filter or attenuate such stray or ambient light to enhance the methods and systems of the disclosure. The light shroud can be external or be affixed to the systems of the disclosure. The light shroud can be internal or be integrated within the systems of the disclosure. In some embodiments, the light shroud comprises a first tube and a second tube, wherein the first tube attaches to the camera, and wherein the second tube attaches to the lens support. The first tube and the second tube can be concentric. The first tube and the second tube can overlap when the sensor is at maximum extent of the focus range. The light shroud can attach to the camera via the c-mound of the camera. The light shroud can attach to the first tube, the second tube, or both via a fastener. The fastener can comprise an adhesive, a screw, a bolt, a nut, a clamp, a tie, or any combination thereof. The surfaces of the light shroud can be painted with or be formed of a highly absorptive paint. Any number of materials and types of shield, hood, sleeve, light shroud, baffle or other physical attenuator can be used for eliminating or reducing stray light.

### Microscopes

In some embodiments, the imaging system herein is stereoscopic. In some embodiments, the imaging system herein is not stereoscopic. In some embodiments, the imaging system herein is a surgical microscope, confocal microscope, fluorescence scope, exoscope, endoscope, or a surgical robot.

In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica ARveo, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., Retina Vue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPALI NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

Combining or integrating a system herein into an existing surgical microscope, confocal microscope, fluorescence scope, exoscope, endoscope, or a surgical robot can be accomplished by: co-housing (in whole or in part), combining one or more aspect or component of the disclosed systems into the existing system, or integrating one or more aspect or component of the disclosed systems into the existing system. Such a combination can reduce shadowing and/or ghosting, utilize confocal improvements, enhance coaxial imaging, increase image clarity, optimize imaging, enable overlapping of optical paths, and improve surgical work flow, amongst other features of the systems and methods disclosed herein. Further such a combination or integration can utilize beam splitters, dichroic filters, dichroic mirrors, polarizers, attenuators, a lens shuttering, frame rate, or any other feature of the systems disclosed herein, or any combination thereof. Additionally, such combinations or integrations can reduce leakiness (imperfection) of one or more filters, utilize ON/OFF rates of visible and fluorescent light sources, or both.

Further, the lighting external to the systems herein, e.g., from the microscope, can be very bright (e.g., ˜400 W), which means that the difference between the intensity of visible light reflection compared to the intensity of fluorescence emission can be substantial. In the embodiments with a single sensor, for example, as shown in FIG. 5D, this can be a disadvantage as the increased sensitivity settings such as higher gain of the sensor or longer exposure can lead to saturation of the light in visible spectrum, and thus, a small reflection of the visible light off the dichroic filter or the dichroic mirror for imaging using an efficient sensor to get a visible image so as to fill around half of the dynamic range of the sensor. In some embodiments, an efficient sensor has a quantum efficiency of more than about 40%, 45%, 50%, 55%, 60%, 65%, 70%, 75%, 80%, or more. In some embodiments, an efficient sensor has the dynamic range of about 60 dB to about 90 dB. In some aspects, a the sensor range is between about 60 dB to about 73 dB or a range between about (73 dB to about 90 dB. In another aspect, the sensor may have a dynamic range of about 73 dB+/−10 dB, 73 dB+/−5 dB. Or 73 dB+/−3 dB. In some embodiments, the dichroic filter, the dichroic mirror, or both transmits a majority of the incident visible light so as to not dim the light seen in the ocular by the surgeon. In some embodiments, at least a portion of the incident visible light is reflected from the dichroic filter, the dichroic mirror, or both. In some embodiments, about 0.5% to about 8% of the incident visible light is reflected from the dichroic filter, the dichroic mirror, or both. In some embodiments, at least about 0.5% of the incident visible light is reflected from the dichroic filter, the dichroic mirror, or both. In some embodiments, at most about 8% of the incident visible light is reflected from the dichroic filter, the dichroic mirror, or both. In some embodiments, the quantity of reflected incident visible light correlates directly with a power of the light.

In some embodiments, the optical light guide is a liquid light guide or other light guide. In some embodiments, the optical light guide couples to a lens which collimates the diverging output light from the fiber. The collimated light from the collimating lens can then pass through a band pass filter which can be a laser cleanup filter to further reduce the spectral bandwidth of the excitation source light. In some embodiments, the light is then diffused using a diffuser. This diffused light is then illuminated on the tissue in such a way as to match the field of view of the microscope and/or the field of view of the operating field.

In some embodiments, the diffuser is configured to match the illumination cone to the imaging field of view of the visible light (VIS), the imaging field of view of the near infrared (NIR) or infrared fluorescence, the microscope imaging field of view, or any combination thereof. In some embodiments, the hole in the NIR mirror 4 is sized, shaped, and/or positioned in to match the imaging axis of the visible light (VIS), the imaging axis of the near infrared (NIR) or infrared fluorescence, the microscope imaging axis, or any combination thereof. Such configurations ensure that the tissue which the surgeon is operating on through the operating microscope's ocular is completely illuminated and captured by the imaging system.

In some embodiments, the illumination path of the surgical microscope is independent of the dichroic filters, hot mirrors herein. In some embodiments, for example as shown in FIG. 4 the diffuser 14 determines the shape of the light beam exiting the hole in the mirror 4. In other embodiments, the size of the hole is governed by the selection of diffusers capable of diffusing the light in a cone of a certain angles. In other embodiments, the hole in the mirror is sized and positioned to achieve coaxial illumination, whereby the imaging axis is incident on the mirror angle and the illumination passes through the hole in the mirror. The hole size can be determined by one or more of: 1) a numerical aperture (NA) and/or core size of fiber which determines the final size of collimated beam incident on diffuser; 2) a feature size on diffuser (a minimum number of features (i.e., 1, 2, 3, 4, or 5 features or less, less than 10, 15, 20, 25, 30 features) can be illuminated to yield a good beam quality); 3) an f/# and focal length of the NIR lens-which can directly determine the maximum hole size so as to not visually obstruct the NIR or IR imaging path and a corresponding reduction in the sensitivity as seen at the detector; or 4) a laser class level and maximum permissible exposure are based on the area of the retina for thermal hazard, where the smaller the beam on the diffuser, the smaller the area illuminated on the back of the retina and therefore the lower the laser power at the tissue for a given classification (e.g., such laser classification, for example in accordance with the ANSI Z136.1 Standard (Z136.1-2000) which assigns lasers into one of four broad hazard classes (1, 2, 3a, 3b and 4) depending on the potential for causing biological damage).

As shown in FIG. 4, the dichroic filter or dichroic mirror (5) can be positioned such that the visible and infrared images from the sample are coaxial, to allow the imaging system to superimpose the visible and infrared images on the display. In addition, the dichroic filter or dichroic mirror (6) can be positioned such that the imaging field of view of the microscope is coaxial with the visible and infrared images captured by the imaging system. Such alignment allows the imaging system to display the same field of view as is seen by the surgeon through the microscope.

In some embodiments, the white or visible light illumination from the microscope cannot be controlled or strobed by the imaging system herein. In some embodiments, the two-camera imaging system advantageously allows a non-multiplexed imaging path (e.g., NIR and visible images are not superimposed) in cases where they cannot be demultiplexed in time. In some embodiments, the imaging system allows strobing of the visible light for demultiplexing, thus a single camera system or a two-camera can both be used. In some embodiments, where control is available on the illumination and ambient light levels, a single camera imaging system can be used.

In some embodiments, the image system herein includes a hatch for servicing the imaging system (e.g., for allowing field reprogramming of the microcontroller firmware). In some embodiments, the hatch is located on the head of the imaging system. In some embodiments, the hatch is located on the back panel.

In some embodiments, the images, for example those shown in FIGS. 1B, 10A-10C, 15, and 29, may be generated by the systems and methods herein are displayed on a separate monitor. In some embodiments, the surgeon is able to select the type of images displayed: visible light image along with fluorescent image overlaid on top; or visible light image displayed in pseudo color, e.g., gray or red, and the fluorescent image displayed in different pseudo color, e.g., teal (blue+green) to achieve high contrast while maintaining the context of surrounding non-fluorescent tissue. In some embodiments, only visible or only fluorescent images can be displayed. In some embodiments, the images of different display types can be placed side by side for display. In some embodiments, the visible only and only fluorescent and overlaid visible and fluorescent images are simultaneously displayed. In some embodiments, the image display is not restricted to a monitor. In some embodiments, the images or videos can be just as easily displayed in surgeon's microscope, or augmented reality glasses, virtual reality glasses, or even used to display remotely for applications such as robotic surgery.

In some embodiments, if an infrared or a NIR frame is not ready, visible frames can take one or more previous NIR or IR frame from the memory/buffer.

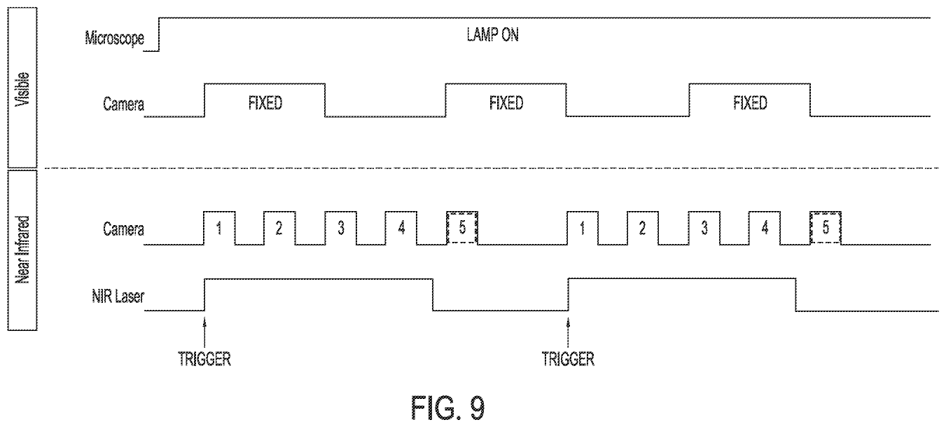

In a non-limiting exemplary embodiment, the systems and methods herein include two cameras. On some embodiments, the system displays both visible and IR or NIR frame simultaneously even if the capture rate is not the same. In some embodiments, the infrared camera captures fluorescence light generated from the tissue when the tissue is excited by the excitation source's light. In some embodiments, the excitation source's light, as can be seen in FIG. 9, is not continuously “ON”. The excitation source's light can be turned on/off rapidly, or strobed either automatically or manually, using a digital processing device. In some embodiments, the excitation source's light can be modulated on/off using a mechanical means; e.g. a shutter or filter wheel, electronic variable optical attenuator (EVOA), or optical ‘chopper’, or a combination of polarizers. In some embodiments, in synchronization with the capture of each frame in the camera. The time when the excitation source is ON or OFF can be dynamically controlled and in real time. In an exemplary embodiment, the excitation source is ON for 1 to 10, 1-2, 1-4, 1-5, 1-6, 1-8, 1-20, 1-50, 1-60, 1-100 or any other frame ranges for NIR or IR fames (i.e., frames captured by the infrared camera). The excitation light can be turned off for one or more of the VIS_DRK frames. The VIS_DRK frame is captured when the excitation source is OFF, the sensor/camera captures all the light which is not from the tissue but is usually ambient light in the operation room or other imaging environment. In some embodiments, the VIS_DRK frame is subtracted from all the NIR or IR frames to remove the artifacts from the ambient or stray light. Afterward, in this particular embodiment, all the first frames are added and displayed as a single frame. In some embodiments, such image frame processing (subtract and/or addition) herein provides the user a great control over the frame capture. In one exemplary embodiment, 4 frames of NIR or IR image corresponds to 1 dark frame (FIG. 9). In other embodiment, any number of 1 or more NIR or IR frames can be followed by 1 VIS_DRK frame.

In some embodiments, the visible (VIS) and NIR or IR excitation are provided by the same broadband source. FIG. 16 shows an alternate illumination pathway that is external to the imaging system. The system can comprise a broadband source, an AR-coated broadband filter, a first shortpass filter, a second shortpass filter, a first filter, a second lowpass filter, a polarizer, a variable filter, a NIR mirror, a VIS lens, a NIR lens, a VIS sensor, a NIR sensor, and a PC motherboard.

As shown in FIG. 4, the NIR or IR fluorescent signal is directed through the window, is redirected by the first shortpass filter, is further redirected by the second shortpass filter and the NIR mirror, where it passes through the first lowpass filter, the NIR lens, the second lowpass filter and arrives at the NIR sensor. Additionally, reflected visible light, and to the first shortpass filter, wherein a portion of the, reflected visible light passes through the first shortpass filter to and through the first shortpass filter, and wherein a portion of the reflected visible light is redirected by the first shortpass filter to the second shortpass filter to and through the second shortpass filter, the polarizer, and the VIS lens to arrive at the VIS sensor. Additionally, in another aspect, a portion of the visible light incident on the first shortpass filter 6 is reflected and transmitted through the second filter 5, through the shortpass filter (VIS-CUT) 23, through the polarizer 22, VIS lens 20, and finally to the VIS camera 21. The VIS sensor and the NIR or IR sensor can then communicate with the PC motherboard based on the received light. The VIS sensor and the NIR or IR sensor can communicate with the PC via a USB3 cable, a serial coax cable such as CoaXPress, an optical fiber, a serial cable, a USB C cable, parallel cable such as Camera Link, or any combination thereof, as shown in FIGS. 6A-6B.

The window can serve as a protection from dust particles and other foreign objects. The window can be fully transparent, and allow all or most wavelengths to pass. The window can have an anti-reflective coating. The window can have a filter. The filter can be a broadband filter. In some embodiments, the window is an AR-coated broadband filter. Additionally, this window can include notch filtering to reduce interference by other surrounding systems emitting wavelengths in the fluorescence band.

In some embodiments, at least one of the first shortpass filter and the second shortpass filter comprise a dichroic filter, an interference filter, a hot mirror, or dielectric mirror or pellicle type mirror. Such filters can include dielectric mirrors, hot mirrors (a type a dielectric mirror), interference filters (e.g., a dichroic mirror or filter). In some embodiments, the system does not comprise the second shortpass filter. The first shortpass filter and the second shortpass filter can be congruent, whereas both filters allow the same band of wavelengths to pass. The first shortpass filter and the second shortpass filter can be incongruent, whereas both filters allow different bands of wavelengths to pass, whereby the different bands of wavelengths does or does not overlap. At least one of the first shortpass filter and the second shortpass filter can be custom made or can be selected from a commercially available filter. In some embodiments, the second shortpass filter includes power monitoring of the transmitted light behind the filter. One or more photodiodes or an array of photodiodes can be used to monitor beam shape and/or beam power. In other embodiments, the photodiodes are placed behind the hot mirror to enable monitoring of transmission of light through the hot mirror.

In some embodiments, the polarizer comprises an absorptive polarizer, a beam-splitting polarizer, a birefringent polarizer, a Nicol prism, a Wollaston prism, a thin film polarizer, a wire-grid polarizer, a circular polarizer, a linear polarizer, or any combination thereof.

In some embodiments, the variable filter comprises an attenuator, a cross polarizer, filter wheel, a liquid crystal, an optical chopper, or a shutter or any other optical component that actively selects or transmits/blocks light of desired wavelengths. The variable filter selectively blocks or attenuates one wavelength band while transmitting another. The variable filter selectively blocks the visible light or dims it as required while not obscuring the NIR or IR fluorescent signal. In some embodiment, the system does not comprise a variable filter.

In some embodiments, the NIR mirror comprises a dielectric mirror, a silver mirror, a gold mirror, an aluminum mirror, a hot mirror, or any combination thereof. The NIR mirror can comprise a dichroic mirror. The NIR mirror can comprise a coated mirror. The NIR mirror can comprise a hole to allow transmission of a laser from behind the NIR mirror. The NIR mirror can comprise a filter which reflects the fluorescence signal while transmitting the excitation wavelength(s), eliminating the physical hole in the optic. Additionally, the NIR mirror can comprise different coatings applied to different areas of the optic that optimize the area of reflection for the fluorescence signal while minimizing the area required for the “hole” that transmits the excitation wavelength(s). The small area for transmission is optimized for maximum transmission at one or more wavelengths while still allowing substantial reflection in the fluorescence band.

In some embodiments, at least one of the VIS lens, the NIR lens, and the VIS/NIR lens comprises a fixed focal length lens. At least one of the VIS lens and the NIR lens can have a focal length of about 10 mm to about 70 mm. In some embodiments, at least one of the VIS lens and the NIR lens comprises a 35 mm lens. Alternatively, at least one of the VIS lens and the NIR lens comprises a variable focal length. The size of the lens can directly correlate with the field of view of the system. The size of the lens can also determine an optimal size of the sensor. At least one of the VIS lens and the NIR lens can have a fixed F-number. Alternatively, at least one of the VIS lens and the NIR lens can have a variable F-number. The VIS lens and the NIR lens can have the same F-number. The VIS lens and the NIR lens can have different F-numbers. The VIS lens can have a greater F-number than the NIR lens. The NIR lens can have a greater F-number than the VIS lens. At least one of the VIS lens and the NIR lens can have an F-number of about 0.5 to about 11. In one exemplary embodiment, the VIS lens has an F-number of about 5.6 and the NIR lens has an F-number of about 1.65. In some cases, higher F-numbers enable higher image quality. In some cases, lower F-numbers enable higher image quality, depending on the applicability of the higher or lower F-number to the VIS or NIR lens, respectively. Unique f/#'s of NIR and VIS lenses can enable system offsets and optimization while maintaining focus. Anti-reflection coatings on the NIR and VIS lenses can be of the same broadband coating or can be individually optimized for NIR or IR or VIS transmission. Optionally, both NIR and VIS lenses can be color corrected specifically for VIS and NIR or IR, respectively, or can be optimized for both VIS and NIR or IR correction, reducing volume and cost.

In some embodiments, at least one of the VIS sensor, the NIR sensor, and the NIR/VIS sensor comprises a visible sensor, a Complementary Metal Oxide Semiconductor (CMOS) sensor, or a Charge-Coupled Device (CCD) sensor. In some embodiments, at least one of the VIS sensor and the NIR sensor comprises an IMX174 sensor, a CMV2000 sensor, or an IMX134 sensor, high-resolution back plane sensor, or cell phone sensor. In some embodiments, at least one of the VIS sensor and the NIR sensor comprise a component within a commercially available camera. The pixel size and form factor of the sensor can be determined by the optical volume and the field-of-view required by the system. The pixel size and form factor of the sensor can be driven by system design specifications. Other embodiments can include any CCD or CMOS sensor, either operating as a complete camera or at the board level, integrated at the imaging station or prior to data transmission. Such processing can be formed at the imaging system via FPGA or by other means. The VIS camera can also include a Bayer filter mosaic or other color filter array to decode the RGB color information. Additionally, the color filter array can include the fluorescent band(s) for additional encoding beyond the pixel sensor array. Other examples of sensors can include back illuminated sensors, multiple sensor arrays (with or without filter arrays, e.g. monochrome), or cooled arrays. In some cases, the NIR sensor is a monochrome sensor. In some cases, the NIR sensor has a color filter array. Additional designs can include a filter array that selects different fluorescent band(s) or reduces interference from other emitting devices. Additionally, certain pixels can be filtered for either alignment to the VIS camera, enhancing resolution, and decoding spectral information.

In some embodiments, the PC motherboard comprises a commercially available PC motherboard. In one example, the commercially available is a PC ASUS ROG STRIX Z370-G micro-ATX motherboard, or an MSI Pro Solution Intel 170A LGA 1151 ATX motherboard.

In some embodiments, the broadband source emits visible through NIR or IR spectrum is a Xenon lamp, a Xenon bulb, an LED light, a laser, a halogen lamp, a halogen bulb, sunlight, fluorescent lighting, or any combination thereof. The broadband source should be configured to provide balanced white light and should have sufficient power in the absorption band of the fluorophore to emit detectable fluorescence. In some instances, the broadband source is unfiltered. In some instances, the broadband source is non-blocked. The broadband light source can be naked, unhindered or non-controlled. In some cases, the broadband light source does not contain a shutter or a filter. Any of the systems and methods of the present disclosure can be used with such a broadband source, including, for example, the systems shown in FIGS. 4, 5A-D, 6A-B, and 16, 18. In other embodiments, the broadband source is filtered or shuttered or otherwise the input/output from the source is synchronized to capture various images. For example, the optical components in a filter or shutter ensure that the resultant VIS and NIR or IR illumination is coaxial and within the same field of view. Any of the systems and methods of the present disclosure can be used with such a filtered or shuttered broadband source, including, for example, the systems shown in FIGS. 4, 5A-D, 6A-B, and 16, 18.

In some embodiments, such filtered or shuttered broadband sources can include a filter, a filter wheel, an electronic variable optical attenuator (EVOA), an optical ‘chopper’, a polarizing shutter, modulator. Such filtering or shuttering enables passages of only certain wavelengths of light from the broadband source. Such filtering or shuttering can code image frames as either: 1) NIR or IR only, where no visible light is emitted but non-visible light in the absorption band is passed, 2) visible only, with minimal inside the absorption band, or 3) ambient only (shutter or “off”). In such embodiments, the light source can be external to the imaging system. In such embodiments, the light source can be within an operating microscope. In such embodiments, the light source can be synchronized with the imaging system sync OUT, the light source sync IN, the imaging system sync IN, the light source sync OUT, or any combination thereof. In some embodiments, the synchronization between the filtered light and camera frame capture can comprise a master/slave relationship. In such cases, the light source can act as a master based on a filter in front of the light source. In such cases, the light source can act as a master based on a shutter state (e.g., ON/OFF, sync IN/OUT, etc.). In such cases, the light source can send signal to camera to start and stop frame capture. Alternatively, per the illumination pattern in FIG. 9, each frame captured by the camera can be communicated to the light source/filter/shutter via a protocol. The protocol can comprise TTL (Transistor Logic). This arrangement can also be implemented in the optical designs shown in FIGS. 4-6 and 7. This arrangement can be further implemented with respect to the placement of the illumination path axis shown in FIG. 16. In general, the visible and fluorescence images can be captured by many acquisition schemes, including a 1-camera or a 2-camera scheme.

In other embodiments, the VIS and NIR or IR excitation is provided by a gas discharge lamp, a Xenon lamp, an LED, a LASER, or any combination thereof. In some instances, such broad excitation source is unfiltered and non-blocked so that the broadband excitation source is naked, unhindered or non-controlled (i.e., does not contain a shutter or filter). Any of the systems and methods of the present disclosure can be used with such a broadband source, including, for example, the systems shown in FIGS. 4, 5A-D, 6A-B, 16 and 18.

In some embodiments, the system further comprises a filter, a bandpass-filter, a filter wheel, an electronic variable optical attenuator (EVOA), an optical ‘chopper’, a polarizer shutter, a modulator, or any combination thereof to selectively filter VIS and NIR or IR excitation wavelengths from the broadband source. For example, a filter wheel might have a shortpass filter, a longpass filter, or both, wherein the shortpass filter allows visible illumination to pass while blocking IR wavelengths. Alternatively, the longpass filter can allow IR wavelengths to pass while blocking visible wavelengths. Moreover, a shortpass filter can be used to block IR light in conjunction with a neutral density (ND) filter, to allow both VIS and NIR or IR to pass from the broadband excitation source. Any of the systems and methods of the present disclosure can be used with such a broadband excitation source, including, for example, the systems shown in FIGS. 4, 5A-D, 6A-B, 16 and 18. In some cases, all VIS and NIR or IR excitation wavelengths can be blocked where the system employs a single-camera which cannot decipher NIR or IR and VIS channels. Blocking all VIS and NIR or IR excitation wavelengths can cause a light flickering that can distract the surgeon. In some embodiments, the system does not comprise a filter, a sync to the light/camera, or both. In such cases, stray light can be emitted by the system.

The broadband source can be used “as is” or as a shuttered or filtered broadband source depending on the source of fluorophore or tissue or cells being detected. The illumination optics which form the beam or path of detection can be optimized or selected based on the field of view (FOV) of the microscope

In some embodiments, the system further comprises an imaging cable strain relief. The imaging cable strain relief can be attached to the imaging system, the imaging station, the imaging cable, or any combination thereof. The imaging cable strain relief can comprise a two-part component. The imaging cable strain relief can comprise a clamp over the imaging cable during manufacture of the imaging system or imaging station. The imaging cable strain relief can comprise a sleeve over an existing terminated cable during manufacture of the imaging system. The imaging cable strain relief can be 3D printed. The imaging cable strain relief can comprise a commercially available strain relief. A sleeve around the imaging cable can be employed to increase the grip of a commercial or custom strain relief. The sleeve can be made of rubber, silicone, plastic, wood, carbon fiber, fiberglass, thermoplastic elastomer, fabric, other polymer, or any combination thereof.

The imaging cable strain relief can further comprise a stop configured to prevent the imaging cable strain relief from translating along the imaging cable. The stop can comprise a grommet, a screw, a tie, a clamp, a string, an adhesive, an O-ring, or any combination thereof. Alternatively, the imaging cable can comprise an integrated strain relief. The imaging cable can have a set flex rating. The imaging cable strain relief can be configured to prevent, minimize or prevent and minimize binding against the any parts of the surgical microscope during imaging system translation, microscope translation, or both. The imaging cable strain relief can be configured to allow and limit twisting of the image cable to prevent cable damage and increase component lifetime. The internal surface of the strain relief can be smooth so as to not puncture the cables. Auto-balance of the microscope can accommodate the additional weight of the imaging cable strain relief.

Image data from one or more of the cameras can be transmitted using optical serial communication rather than passive or active copper wires. Optical serial communication generally allows for greater cable flexibility and longer cable lengths. In further embodiments, such cables can enable electrical transmission, optical transmission, or both. In addition, passive cables with right angle connectors and high-flexibility to accommodate focus stage movement can be included.

The imaging system can comprise one or more locking keys. The locking keys can be configured to securely lock the imaging system onto the microscope. The locking keys can be configured to securely lock the imaging system onto the microscope without requiring any tools. The locking keys can be permanently fixed via one or more lanyards to the imaging system to prevent loss of the locking keys. FIG. 11 shows an exemplary embodiment for the lock and key of the imaging system. The imaging system of the imaging system herein locks onto the microscope by two independent keys, where each key can be sufficient for restraint of the head to the scope. In some cases this key mechanism does not require tools for removing of any existing hardware on the microscope, allowing quick and easy insertion or removal of the device prior or after surgical procedures. The locking mechanism can also consist of a lever system or other mechanical system to secure the imaging head to the microscope.

The systems herein can further comprise a photodiode. The systems herein can further comprise a plurality of photodiodes. The photodiode can continuously monitor and directly trip the interlock on the laser for both an underpower and overpower event. The photodiode can detect beam shape discrepancy that could indicate a diffuser failure. The photodiode can be placed at one, two, three or more locations in the laser beam path. The photodiode can be placed prior to the diffuser. The photodiodes can be placed after the diffuser to detect beam shape discrepancy that could indicate a diffuser failure. Laser classification requires a specific laser beam spot size of the diffuser. While larger beam spot sizes enable a high laser power while maintaining safe emission levels, smaller beam spot sizes reduce the obstruction required to direct the beam into the imaging pathway and provide increased sensitivity to fluorescence. Baffles reduce reflections or stray light. A crescent shaped baffle on the dichroic 16 may be used to prevent microscope illumination light from reflecting back into VIS or NIR cameras. Other baffles may be used to reduce reflections from the optical excitation assembly. The systems herein can further comprise a baffle, a hood or both attached to the diffuser or optical excitation assembly. The baffle, hood, or both can reduce stray light from the optical excitation assembly received by the notch filter, or LP filter on camera lens. The baffle for the VIS light from scope can have a moon shape. The baffle, hood, or both can further prevent the long tails of the top-hat diffuser profile from illuminating the filter on the camera lens at a large angle of incidence, and being transmitted through the filter, whereby the stray light could reach the imaging detector. The baffles, hood or both prevent exceeding the design angle of incidence (AOI) on the filter.

The system shown in FIG. 4 can employ objective lenses with different f-numbers. Optimizing NIR or IR sensitivity allows greater depth of field in the visible camera images. Further, such configurations allow for lower cost lenses with smaller optical volumes. The NIR or IR resolution requirement can be low compared to the visible and chromatic correction from 400-1000 nm are not required. In some embodiments, the system's NIR or IR resolution is less than or equal to the VIS resolution. Such reduced resolution can enable optimal design of volume. Typically, as VIS light is more abundant than NIR or IR light, the system can be designed to maximize capture of photons of light in the NIR, IR or other range to obtain a better NIR, IR, or other signal to noise ratio, respectively. Increasing the NIR or IR signal to noise ratio can be done in a number of ways including lowering the resolution of the NIR sensors (i.e., the use of a lower resolution sensor has larger pixel size to optimize collection of NIR or IR photons which is more efficient (better signal to noise). Alternatively, the NIR or IR signal to noise ratio can be increased using a faster lens (smaller F-number). Generally, the NIR or IR resolution can be less than or equal to VIS resolution in such embodiments, however if the NIR sensor is sensitive enough, smaller pixel sizes can be used and still obtain a sufficient NIR or IR signal to noise ratio. Consequently, in some embodiments, the system NIR or IR resolution is greater than the VIS resolution. It is recognized that focal length and F-number can further affect NIR or IR resolution or VIS resolution in the system, and such can be adjusted and optimized accordingly.

The systems herein can further comprise an ex-vivo docking station configured to allow use of the imaging system without the microscope. The ex-vivo docking station can comprise an optomechanical tub/tray/frame separate from enclosure, to enable safe illumination, imaging and control of visible and NIR or IR illumination. The ex-vivo docking station enables controlled imaging for, in one example, imaging ex vivo tissue samples or fluorescence reference or calibration targets.

The systems herein can further comprise a drape 150 as shown in FIG. 1A. The drape can be configured to surround at least a portion of the microscope head to maintain sterility therein. The drape can comprise a transparent window for viewing the sample. The drape 150 can be compatible with current operating rooms draping systems. The drape can be a commercially available sterile drape; such as the Zeiss OPMI Sterile Drape with VisionGuard optical lens (REF: 3 6- -000). In some embodiments, stray excitation is prevented from being reflected towards the microscope from the drape window. In some embodiments, the systems herein comprise rounded outer edges to prevent the drape from being punctured. In some embodiments, the drape maintains a sterility boundary between the surgical field and the imaging systems described herein. In some embodiments, the drape comprises a sterile circular window that covers at least a portion of a bottom window of the microscope, the imaging system, or both.

In some cases, the imaging system on the microscope further comprises one or more of a flange, a rib, a guide, a clamp configured to enable easy and precise attachment to the head to the microscope. In some cases, the imaging system on the microscope has a shape, a contour, or both that enable smooth integration and minimal cable interference during attachment of the imaging system and the microscope. In some cases, the imaging system can further comprise an arrow, a symbol, a text or any combination thereof to describe or annotate proper connection of the imaging system to the microscope. The arrow, symbol, text or any combination thereof, can be adhered to or directly machined onto the imaging system. In further embodiments, the shape of the imaging system, the imaging cable, or both can be configured for efficient movement and reduced drag. Further, the imaging system can comprise a seal enhancing the sealability of the connections of the head to the scope (e.g., the top/bottom windows) and aids in maintaining smooth operation and cleanliness of the device.

In some cases, the imaging system further comprises a mount adapted to the specific microscope, the confocal microscope, the fluorescence scope, the exoscope, the surgical instrument, the endoscope, or the surgical robot to facilitate ease of attachment and removal. Such mounts can be separate adaptors (or integral to the imaging system) that enable fitting the main body of the imaging system to a variety of such scopes and instruments, such that the imaging system can be adapted to one or more of any microscope, the confocal microscope, the fluorescence scope, the exoscope, the surgical instrument, the endoscope, or the surgical robot such as one or more of those as described herein.

In some embodiments, the system comprises one or more excitation source active indicators. In some embodiments, one excitation source active indicator is in the front of the device and another excitation source active indicator is at the bottom of the device.

In some embodiments, contralateral illumination is automatically disabled when the head is inserted onto the microscope. The systems herein can comprise a second source of illumination to prevent formations of shadows within valleys, depressions and uneven surfaces in the imaging bed, such as the tissue viewed or exposed during surgery. However, in some cases, the second source of illumination is periodically dimmed or turned off to prevent interference with additional optical components.

In order to view the sample without fluorescence, a VIS_DRK frame can be subtracted from any fluorescence caused by the microscope illumination. The VIS_DRK frame can be applied mechanically, electronically, or by an image processing software.

In some embodiments, the systems and methods herein only include a VIS/NIR or a VIS/IR camera that is configured to sense both visible and NIR or IR signals. In some embodiments, the sensitivity for visible and NIR or IR signal is different.

In some embodiments, there are two cameras on a single stage. In some embodiments, both cameras are looking at the same area and focus together. In some embodiments, the field of view, aperture, focal length, depth of field, or any other parameters of both cameras are identical. In some embodiments the field of view, aperture, focal length, depth of field, or any other parameters of both cameras are not the same (e.g. aperture). In some embodiments, the systems and methods herein only include a NIR or IR camera. In some embodiments, the capture of visible frame, trigger frames (or NIR or IR frames), and VIS_DRK frames can be in the same sequence.

In some embodiments, there can be additional pair(s) of excitation sources and notch filters for illuminating the source with different excitation wavelengths. For example, frames 1, 2, 3, 4, and 5 (such that each frame is excited by a different wavelength—e.g., exciting different fluorophores per frame, and also one visible (white) and one VIS_DRK frame)—thus the sequence of 1, 2, 3, 4, and 5 enables visualization of 3 different fluorophores simultaneously (and one white, one DRK) in a single frame. With this flexibility, any number of frames and fluorophores can be imaged to allow detection of multiple fluorophores emitting at different wavelengths (e.g., on the same molecule and/or in the same sample being tested). Thus, the systems and methods herein not only apply to dyes that are NIR or IR fluorophores, but a variety of sources that emit light (e.g., dyes which emit in green, red and infrared wavelengths). For example, various dyes that could be conjugated to peptides can be imaged with the systems and methods herein. In some embodiments, how a sample can be imaged (e.g., with or without use of a non-specific dye in normal tissue (contrast) with a different dye on targeting molecule that homes, targets, migrates to, is retained by, accumulates in, and/or binds to, or is directed to an organ, organ substructure, tissue, target, cell or sample) can be adjusted or tested using the systems and methods herein.

Using the systems and methods herein, autofluorescence in an organ, organ substructure, tissue, target, cell or sample can be detected. Moreover, based on their autofluorescence profile, different biological structures (e.g., organ, organ substructure, tissue, target, cell or sample) can be distinguished at various wavelengths. Such autofluorescence can be enhanced and further distinguished by introducing an exogenous contrast or imaging agent, or any combination thereof. Moreover, using the systems and methods herein, fluorophores that home, target, migrate to, are retained by, accumulate in, and/or bind to, or are directed to an organ, organ substructure, tissue, target, cell or sample can be detected, whether such fluorophore is alone, conjugated, fused, linked, or otherwise attached to a chemical agent or other moiety, nanoparticle, small molecule, therapeutic, drug, chemotherapeutic, peptide, antibody protein or fragment of the foregoing, and in any combination of the foregoing. For example, human serum albumin (HSA) can be conjugated to a fluorophore and thereby increase its retention within the vasculature and its half-life. Peptides, antibodies, or antibody fragments can be engineered to target specific tissues of interest, for example vascular endothelium or nerves, so that these structures are stably labeled for the duration of a surgical or diagnostic procedure. Conjugates can be created that are non-fluorescent until they are activated in the presence of the diseased tissue or other condition to be detected.

Examples include peptide moieties that are cleaved by cathepsins or matrix metalloproteinases that can be used to detect atherosclerotic plaques, tumor microenvironment, or other areas of abnormal tissue or inflammation. For example, the fluorophore is a fluorescent agent emitting at a wavelength between 650 nm and 4000 nm, such emissions being used to detect such agent in an organ, organ substructure, tissue, target, cell or sample using the systems and methods herein. In some embodiments the fluorophore is a fluorescent agent that is selected from the group consisting of non-limiting examples of fluorescent dyes that could be used as a conjugating molecule (or each class of molecules) in the present disclosure include DyLight-680, DyLight-750, VivoTag-750, DyLight-800, IRDye-800, VivoTag-680, Cy5.5, or an indocyanine green (ICG) and any derivative of the foregoing. In some embodiments, near infrared dyes often include cyanine dyes. Additional non-limiting examples of fluorescent dyes for use as a conjugating molecule in the present disclosure include acradine orange or yellow, ALEXA FLUORs and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FLASH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, methylene blue and any derivative thereof, nile dyes and any derivative thereof, OS680, OS750, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, topotecan, TSQ, umbelliferone, violanthrone, yellow fluorescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′, 7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in international patent application no. PCT/US2014/77.

Moreover, using the systems and methods herein, fluorescent biotin conjugates that can act both as a detectable label and an affinity handle can be used to detect such agent in an organ, organ substructure, tissue, or sample using the systems and methods herein. Non limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, ALEXA FLUOR 488 biocytin, ALEXA FLUOR 546, ALEXA FLUOR 549, lucifer yellow cadaverine biotin-X, Lucifer yellow biocytin, Oregon green 488 biocytin, biotin-rhodamine and tetramethylrhodamine biocytin. In some other examples, the conjugates could include chemiluminescent compounds, colloidal metals, luminescent compounds, enzymes, radioisotopes, and paramagnetic labels. In some embodiments, the peptide-active agent fusions described herein can be attached to another molecule. For example, the peptide sequence also can be attached to another active agent (e.g., small molecule, nanoparticle, peptide, polypeptide, polynucleotide, antibody, aptamer, cytokine, growth factor, neurotransmitter, an active fragment or modification of any of the preceding, fluorophore, radioisotope, radionuclide chelator, acyl adduct, chemical linker, or sugar, etc.). In some embodiments, the peptide can be fused with, or covalently or non-covalently linked to an active agent.

The systems and methods of the present disclosure can be used alone or in combination with a companion diagnostic, therapeutic or imaging agent (whether such diagnostic, therapeutic or imaging agent is a fluorophore alone, or conjugated, fused, linked, or otherwise attached to a chemical agent or other moiety, small molecule, nanoparticle, therapeutic, drug, chemotherapeutic, peptide, antibody protein or fragment of the foregoing, and in any combination of the foregoing; or used as a separate companion diagnostic, therapeutic or imaging agent in conjunction with the fluorophore or other detectable moiety is alone, conjugated, fused, linked, or otherwise attached to a chemical agent or other moiety, small molecule, nanoparticle, therapeutic, drug, chemotherapeutic, peptide, antibody protein or fragment of the foregoing, and in any combination of the foregoing). Such companion diagnostics can utilize agents including chemical agents, radiolabel agents, radiosensitizing agents, photosensitizing agents, fluorophores, imaging agents, diagnostic agents, protein, peptide, a nanoparticle or small molecule such agent intended for or having diagnostic or imaging effect. Agents used for companion diagnostic agents and companion imaging agents, and therapeutic agents, can include the diagnostic, therapeutic and imaging agents described herein or other known agents. Diagnostic tests can be used to enhance the use of therapeutic products, such as those disclosed herein or other known agents. The development of therapeutic products with a corresponding diagnostic test, such as a test that uses diagnostic imaging (whether in vivo, in situ, ex vivo or in vitro) can aid in diagnosis, treatment, identify patient populations for treatment, and enhance therapeutic effect of the corresponding therapy. The systems and methods of the present disclosure can also be used to detect therapeutic products, such as those disclosed herein or other known agents, to aid in the application of a therapy and to measure it to assess the agent's safety and physiologic effect, e.g. to measure bioavailability, uptake, distribution and clearance, metabolism, pharmacokinetics, localization, blood concentration, tissue concentration, ratio, measurement of concentrations in blood and/or tissues, assessing therapeutic window, range and optimization, and the like of the therapeutic agent. Such The systems and methods can be employed in the context of therapeutic, imaging and diagnostic applications of such agents. Tests also aid therapeutic product development to obtain the data FDA uses to make regulatory determinations. For example, such a test can identify appropriate subpopulations for treatment or identify populations who should not receive a particular treatment because of an increased risk of a serious side effect, making it possible to individualize, or personalize, medical therapy by identifying patients who are most likely to respond, or who are at varying degrees of risk for a particular side effect. Thus, the present disclosure, in some embodiments, includes the joint development of therapeutic products and diagnostic devices, including the systems and methods herein (used to detect the therapeutic and/or imaging agents themselves, or used to detect the companion diagnostic or imaging agent, whether such diagnostic or imaging agent is linked to the therapeutic and/or imaging agents or used as a separate companion diagnostic or imaging agent linked to the peptide for use in conjunction with the therapeutic and/or imaging agents) that are used in conjunction with safe and effective use of the therapeutic and/or imaging agents as therapeutic or imaging products. Non-limiting examples of companion devices include a surgical instrument, such as an operating microscope, confocal microscope, fluorescence scope, exoscope, endoscope, or a surgical robot and devices used in biological diagnosis or imaging or that incorporate radiology, including the imaging technologies of X-ray radiography, magnetic resonance imaging (MRI), medical ultrasonography or ultrasound, endoscopy, elastography, tactile imaging, thermography, medical photography and nuclear medicine functional imaging techniques as positron emission tomography (PET) and single-photon emission computed tomography (SPECT). Companion diagnostics and devices can comprise tests that are conducted ex vivo, including detection of signal from tissues or cells that are removed following administration of the companion diagnostic to the subject, or application of the companion diagnostic or companion imaging agent directly to tissues or cells following their removal from the subject and then detecting signal. Examples of devices used for ex vivo detection include fluorescence microscopes, flow cytometers, and the like. Moreover, the systems and methods herein for such use in companion diagnostics can be used alone or alongside, in addition to, combined with, attached to or integrated into an existing surgical microscope, confocal microscope, fluorescence scope, exoscope, endoscope, or a surgical robot.

In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica ARveo, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, Retina Vue system (e.g., Retina Vue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPALI NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

Moreover, in some embodiments, the imaging, diagnostic, detecting and therapeutic methods herein are performed using the systems described herein alongside, in addition to, combined with, attached to, or integrated into such an existing surgical microscope, confocal microscope, fluorescence scope, exoscope, endoscope, surgical robot, microscope, exoscope, or endoscope as described above.

Any additional surgical microscope, confocal microscope, fluorescence scope, exoscope, endoscope, or surgical robot systems can be used. The surgical microscope, confocal microscope, fluorescence scope, exoscope, endoscope, or surgical robot systems can be provided by, for example, Carl Zeiss A/G, Leica Microsystems, Leica Biosystems, Haag-Streit (5-1000 or 3-1000 systems) or Intuitive Surgical (e.g.: da Vinci surgical robot system), or any other manufacturer of such systems.

Combining or integrating a system herein into an existing surgical microscope, confocal microscope, fluorescence scope, exoscope, endoscope, or a surgical robot can be accomplished by: co-housing (in whole or in part), combining one or more aspect or component of the disclosed systems into the existing system, or integrating one or more aspect or component of the disclosed systems into the existing system. Such a combination can reduce shadowing and/or ghosting, utilize confocal improvements, enhance coaxial imaging, increase image clarity, optimize imaging, enable overlapping of optical paths, and improve surgical work flow, amongst other features of the systems and methods disclosed herein. Further such a combination or integration can utilize beam splitters, dichroic filters, dichroic mirrors, polarizers, attenuators, a lens shuttering, frame rate, or any other feature of the systems disclosed herein, or any combination thereof. Additionally, such combinations or integrations can reduce leakage (imperfection) of one or more filters, utilize ON/OFF rates of visible and fluorescent light sources, or both.

In some aspects, at least one of the microscope, the confocal microscope, the fluorescence scope, the exoscope, the surgical instrument, the endoscope, or the surgical robot comprises a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, OPMI Sensera, OPMI Movena, OPMI 1 FC, EXTARO 300, TREMON 3DHD system, CIRRUS system (e.g., CIRRUS 6000 and CIRRUS HD-OCT), CLARUS system (e.g., CLARUS 500 and CLARUS 700), PRIMUS 200, PLEX Elite 9000, AngioPlex, VISUCAM 524, VISUSCOUT 100, ARTEVO 800, (and any other surgical microscope, confocal microscope, fluorescence scope, exoscope, endoscope, ophthalmoscope, retinal camera system, optical coherence tomography (OCT) system, and surgical robot systems from Carl Zeiss A/G,); PROVido system, ARvido system, GLOW 800 system, Leica ARveo Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica M844 system, Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Leica EnFocus, Leica Proveo 8, Leica Envisu C2300, Leica PROvido, and any other surgical microscope, confocal microscope, fluorescence scope, exoscope, endoscope, ophthalmoscope, retinal camera system, OCT system, and surgical robot systems from Leica Microsystems or Leica Biosystems; Haag-Streit 5-1000 system, Haag-Streit 3-1000 system, Haag-Streit HI-R NEO 900, Haag-Streit Allegra 900, Haag-Streit Allegra 90, Haag-Streit EIBOS 2, and any other surgical microscope, confocal microscope, fluorescence scope, exoscope, endoscope, and, surgical robot systems from Haag-Strait; Intuitive Surgical da Vinci surgical robot systems, and any other surgical microscope, confocal microscope, fluorescence scope, exoscope, endoscope, ophthalmoscope, retinal camera system, OCT system, and surgical robot systems from Intuitive Surgical; Heidelberg Engineering Spectralis OCT system, and any other surgical microscope, confocal microscope, fluorescence scope, exoscope, endoscope, ophthalmoscope, retinal camera system, OCT system, and surgical robot systems from Heidelberg Engineering; Topcon 3D OCT 2000, DRI OCT Triton, TRC system (e.g., TRC 50DX, TRC-NW8, TRC-NW8F, TRC-NW8F Plus, TRC-NW400), IMAGEnet Stingray system (e.g., Stingray, Stingray Pike, Stingray Nikon), IMAGEnet Pike system (e.g., Pike, Pike Nikon), and any other surgical microscope, confocal microscope, fluorescence scope, exoscope, endoscope, ophthalmoscope, retinal camera system, OCT system, and surgical robot systems from Topcon; Canon CX-1, CR-2 AF, CR-2 PLUS AF, and any other surgical microscope, confocal microscope, fluorescence scope, exoscope, endoscope, ophthalmoscope, retinal camera system, OCT system, and surgical robot systems from Canon; Welch Allyn 3.5 V system (e.g., 3.5V, 3.5V Autostep), CenterVue DRS, Insight, PanOptic, RetinaVue system (e.g., Retina Vue 100, Retina Vue 700), Elite, Binocular Indirect, PocketScope, Prestige coaxial-plus, and any other surgical microscope, confocal microscope, fluorescence scope, exoscope, endoscope, ophthalmoscope, retinal camera system, OCT system, and surgical robot systems from Welch Allyn; Metronic INVOS system, and any other surgical microscope, confocal microscope, fluorescence scope, exoscope, endoscope, ophthalmoscope, retinal camera system, OCT system, and surgical robot systems from Medtronic; Karl Storz ENDOCAMELEON, IMAGE1 system (e.g., IMAGE1 S, IMAGE1 S 3D, with or without the OPALI NIR imaging module), SILVER SCOPE series instrument (e.g., gastroscope, duodenoscope, colonoscope) and any other surgical microscope, confocal microscope, fluorescence scope, exoscope, endoscope, ophthalmoscope, retinal camera system, OCT system, and surgical robot systems from Karl Storz, or any combination thereof.

The systems and methods herein can be used to detect one or more detectable agents, affinity handles, fluorophores, or dyes, two or more, three, four five and up to ten or more such detectable agents, affinity handles, fluorophores, or dyes in a given sample (e.g., organ, organ substructure, tissue, or sample).

### Image Processing

In some embodiments, the systems and methods herein allow for reinforcement and dropping of NIR or IR frames as required based on the signal strength. In some embodiments, it can be determined how many NIR or IR frames need to be captured before performing the above-mentioned processing. If the fluorescence light from the tissue is very bright, only 2 or 3 frames instead of 4 or more frames may need to be added for every displayed frame. Conversely, if the signal is very low, 6-9 or more NIR or IR frames may need be captured before the NIR or IR fluorescence image intensity is sufficient. Thus, the system may reinforce or drop NIR or IR frames as required and dynamically change the sensitivity of the imaging system.

Referring to FIG. 9, in a particular embodiment, the visible light from the surgical microscope's illumination is always on (i.e., continuous wave (CW)) while the NIR or IR laser is switched between on and off regularly. In this embodiment, the laser light is on for every 4 frames of NIR or IR frames, so that fluorescence light from such 4 frames is added for an NIR or IR image displayed, the excitation sources light is then turned off for a VIS_DRK frame to provide baseline ambient light image to be removed from the NIR or IR image.

In some embodiments, the VIS_DRK frame exposure time and gain values match those of the NIR or IR frame. There is flexibility in the VIS_DRK frame exposure relative to the NIR or IR frame exposure. Mathematically, it can be an exact match. In other cases, the VIS_DRK frame can be of a different exposure and digitally matched to the NIR or IR frame's exposure by scaling. In some embodiments, the NIR or IR frame's exposure can be a multiple of the VIS_DRK frame exposure (either longer or shorter) and can be scaled to match the NIR or IR frame exposure mathematically during image processing. In some embodiments, the exposure time for each frame can be dynamically changed.

In some embodiments, the visible camera captures the frames at a fixed frame rate and optionally after each visible image is captured, the NIR or IR frame buffer is checked, if the buffer is updated with the latest captured NIR or IR image, the NIR or IR image is added to the visible light image. In some embodiments, when an older NIR or IR image (as the case can be) is in the buffer, the older NIR or IR image is used for display with the new VIS image, thus there can be asynchronous frame capture between visible and infrared fluorescence images. In some embodiments, this is advantageous to allow independence of the frame capture rates of the fluorescence image and visible images, which can be faster or slower, while the frame rate of the displayed image (visible and fluorescence image) is consistently full video rate. In some embodiments, the video rate provided by the systems and methods herein advantageously enables the user to fine tune or simply adjust the image to maximize its visibility, clarity, operation and use in real time.

In some embodiments, the systems and methods herein use a transistor-transistor-logic (TTL) trigger signal for camera frame capture. In some embodiments, the duty cycle of the TTL trigger for camera frame capture is used to drive the excitation source's illumination. In some embodiments, one or more TTL triggers from the camera frame capture is used to drive the excitation source's illumination.

In some embodiments, various image processing technologies can be used on the NIR or IR images and/or visible light images, thereby facilitating display of color maps or contour images.

In some embodiments, images herein are processed by a digital processing device, a processor, or the like. In some embodiments, image processing herein includes: image reconstruction, image filtering, image segmentation, addition of two or more images, subtraction of one or more images from image(s), image registration, pseudo coloring, image masking, image interpolation, or any other image handling or manipulation.

In some embodiments, images herein are displayed to a digital display and controlled by a digital processing device, a processor, or the like. In some embodiments, a digital processing device, a processor, or the like herein enable the surgeon or other users to select image type(s) to be displayed. In some embodiments, image processing is performed by an application specific integrated circuit (ASIC), located within one or more of the cameras in the imaging system, providing for the fully-processed composite image to be transmitted from the imaging system. Use of the ASIC for image processing reduces the bandwidth requirements for the cable, and the subsequent processing requirements on the ‘display side’.

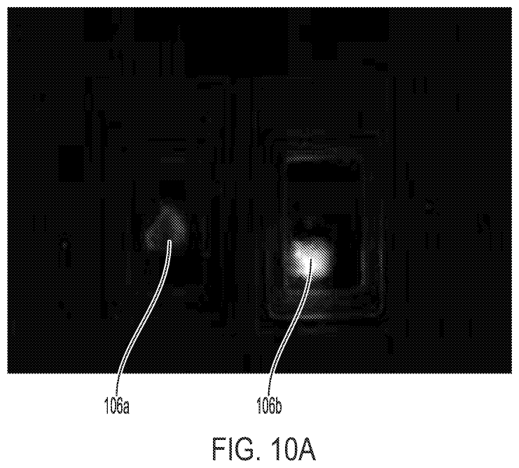

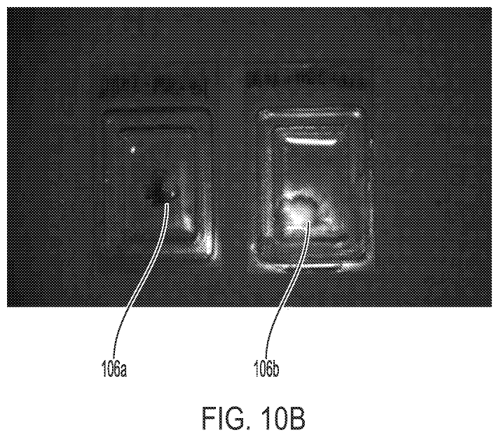

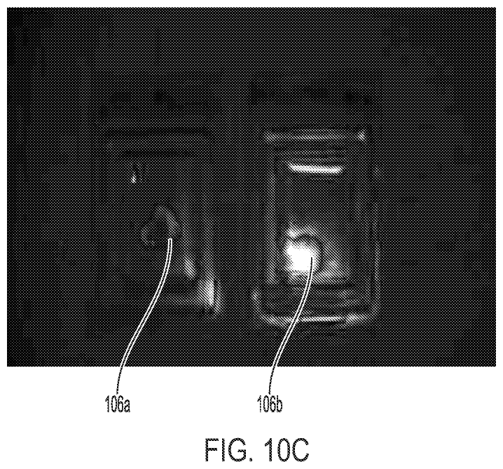

In some embodiments, false or pseudo coloring is used on the NIR or IR images or visible light images. Referring to FIGS. 10A-10C, in a particular embodiment, the visible light image is colored differently, for example in black (FIG. 10A), as a true color (FIG. 10B) or as an alternative color (e.g., red) (FIG. 10C), while the NIR or IR image includes false coloring to increase the contrast on the images over the background visible light. In these embodiments, the superimposed composite image with both fluorescent light and visible light shows the tumor tissue 106a and 106b. Each tumor tissue sample 106a and 106b are shown having differing signal intensities. Such difference in signal intensity is caused by different level of tissue uptake of fluorescent dye(s).

Referring to FIG. 7A, the systems and methods provide the option to view the fluorescence image superimposed on the visible image or the fluorescence image alone, or view the visible and NIR or IR images side-by-side thus providing the user flexibility with image visualization. In some embodiments, the images, visible or fluorescent images are two-dimensional image frames that can be stacked to make three-dimensional volumetric image(s).

In some embodiments, the tumor is automatically, semi-automatically, or manually contoured in visible light and/or NIR or IR image during image processing so that the tumor and the tumor boundary can be better visualized by a surgeon or any other medical professional. In some embodiments, the NIR or IR image is integrated along x axis and/or y axis so that a one-dimensional fluorescence signal profile is generated.

### Methods, Systems, and Media for Forming Images of Fluorophore Excitations

Provided herein are computer-implemented methods of forming a first overlaid image from laser induced fluorophore excitations. Also, provided herein are computer-implemented systems comprising a digital processing device comprising at least one processor, an operating system configured to perform executable instructions, a memory, and a computer program including instructions executable by the digital processing device to create an application for forming a first overlaid image from laser induced fluorophore excitations. Further, provided herein are a non-transitory computer-readable storage media encoded with a computer program including instructions executable by a processor to create an application for forming a first overlaid image from laser induced fluorophore excitations.

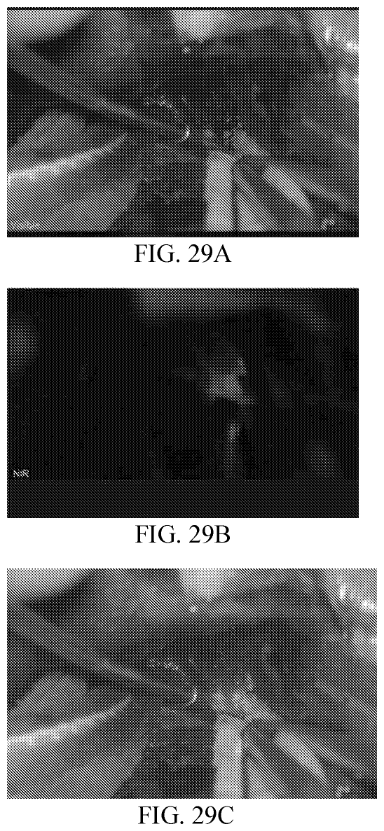

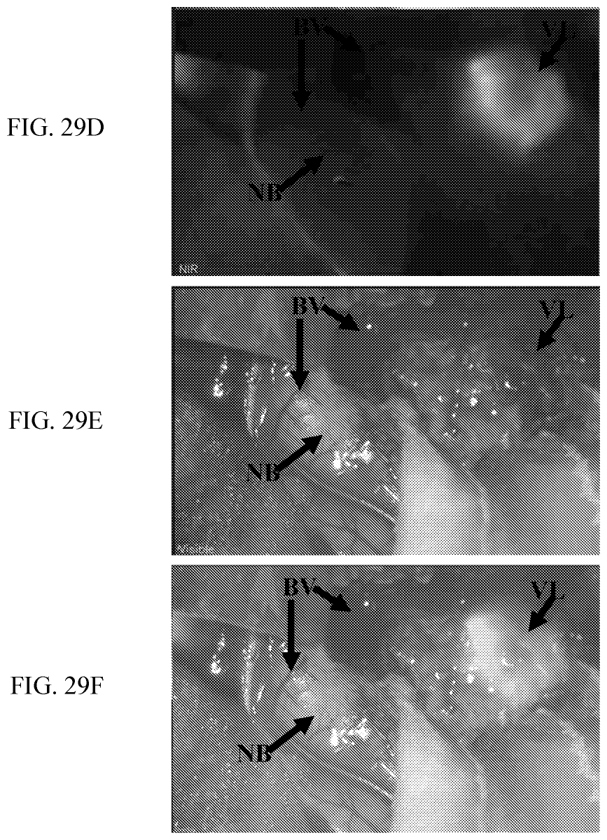

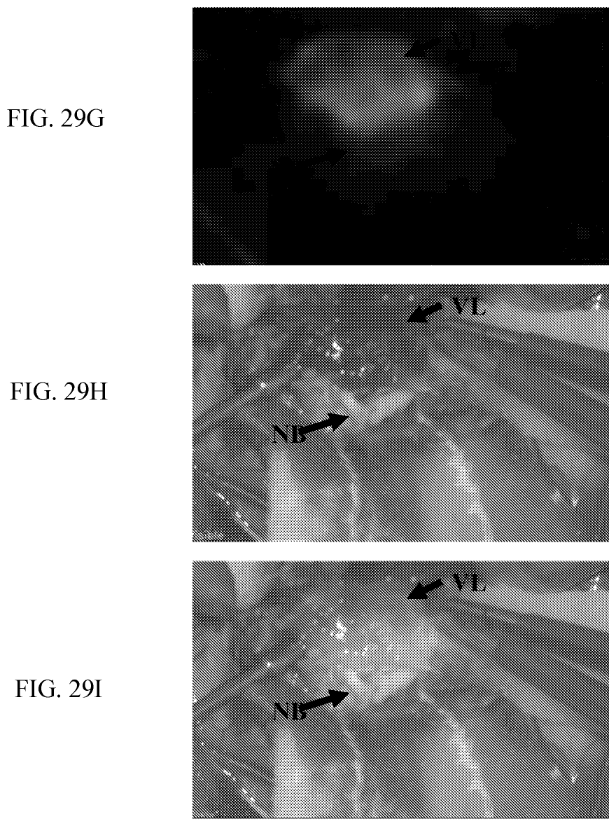

In some embodiments, the method comprises receiving a plurality of image frame sequences, correcting each NIR or IR frame, generating a first NIR or IR image, generating a first VIS image, and overlaying the first NIR or IR image and the first VIS image to form the first overlaid image. In some embodiments, the application receives a plurality of image frame sequences, corrects each NIR or IR frame, generates a first NIR or IR image, generates a first VIS image, and overlays the first NIR or IR image and the first VIS image to form the first overlaid image. Several examples of such images are shown in FIGS. 29A-I. According to various aspects, these images are exemplary of what a surgeon may see while using while performing surgery and using the system in in vivo or in situ applications. In particular, FIG. 29A-C show exemplary visible images of an in situ tissue sample of a tumor or abnormality in a patient. s FIG. 29A hows an exemplary visual light (VIS) image. FIG. 29B shows an exemplary near-infrared (NIR) image. FIG. 29C shows an exemplary overlaid image.

FIG. 29D-F and FIG. 29G-I show exemplary visible images of in situ tissue samples of a vascular lesion, vascular malformation or vascular abnormality acquired using the imaging systems and methods herein. In particular, the set of images in FIGS. 29D-F and the set of images in FIGS. 29G-I, are representative images of in situ or intra-operative tissue during surgery on a vascular lesion in a subject, wherein 22 mg of tozuleristide was administered.

FIG. 29D shows a near-infrared (NIR) image of the in situ specimen. Fluorescence signal, corresponding to lighter and brighter areas in the NIR or IR images, is indicative of the presence of tozuleristide in the vascular lesion. Labeled arrows indicate non-fluorescent regions of normal blood vessels (“BV”) and normal brain tissue (“NB”). In contrast, fluorescence signal corresponding to lighter and brighter areas in the NIR or IR image, was indicative of the presence of tozuleristide on the abnormal vascular lesion (“VL”), and not in normal tissue.

FIG. 29E shows the white light image corresponding to FIG. 29D that represents what the surgeon would normally see without fluorescence guidance. The arrows mark the same locations as shown in the NIR or IR image in FIG. 29D. The vascular lesion (“VL”) had a similar appearance to the normal blood vessels (“BV”) in this image.

FIG. 29F shows the NIR or IR fluorescence and white light composite image of FIG. 29D and FIG. 29E, with arrows marking the same locations as shown in FIG. 29D and FIG. 29E. Fluorescence in the vascular lesion (“VL”) clearly differentiated it from the surrounding normal tissues, including normal blood vessels (“BV”).

FIG. 29G shows a near-infrared (NIR) image of the vascular lesion during the surgery. Arrows indicate the vascular lesion (labeled “VL”) and adjacent normal brain (labeled “NB”), which is non-fluorescent.

FIG. 29H shows the white light image corresponding to FIG. 29G. While the normal brain has a light tan to pink color (light gray in a gray scale image), it is perfused with normal blood vessels that can be differentiated from the vascular lesion by the absence of fluorescence.

FIG. 29I shows the composite white light and NIR or IR image shown in FIG. 29G and FIG. 29H.

The images that are acquired during one laser cycle are referred to as a “sequence”. A sequence will generally contain at least one VIS_DRK frame and one NIR frame, but any other combination of VIS_DRK and NIR frames is possible. Typically in application, an imaging situation has relatively much more visible light than NIR fluorescence present. The ratio of NIR to VIS_DRK frames is shown as NIR:(VIS_DRK).

The gains used when the VIS_DRK (DRKgain) and NIR frames (NIRgain) are acquired may be different. Generally, they will be the same, which allows for direct subtraction of the VIS_DRK from the NIR. However, if the gains are different, a scaling function must be used to correct for the different gains. This scaling function is the function that represents the difference between the VIS_DRK and NIR camera gains across sensor's input dynamic range (also described here as an input dynamic range): K(signal)_{gain}, such that for an equivalent input signal, the following is true.

$${NIR ( signal ) = K ( signal )}_{gain} \times VIS\_DRK ( signal )$$

Thus, in simple terms, the gain corrected dark frame (DRK*) can be calculated as follows;

$$DRK *= {K}_{gain} ( VIS \_ DRK )$$

Note that this function may not be linear, it could be a polynomial, exponential, or another function. This correction should be applied before any correction for different exposure times, as described herein. The exposures for the VIS_DRK frames (DRKₑₓₚ) and the NIR frames (NIRₑₓₚ) may be different. Generally, the ratio of NIRₑₓₚ to DRKₑₓₚ will be an integer greater than 1: EXP_RATIO_{NIR-DRK}=1, 2, 3, etc. However, fractional ratios are also possible. Fractions greater than 1 may be useful; e.g. 1.5, 2.5, etc. However, fractions less than 1 (which indicate that the VIS_DRK frame's exposure is longer than the NIR exposure) are less likely to be useful in situations where there is relatively much more visible light than NIR fluorescence. The raw NIR frame contains the following data:
1. Visible light reflection from the target
2. Ambient NIR from the environment
3. Fluorescence from the target fluorophore

Further, the VIS_DRK frame contains the following data:
1. Visible light reflection from the target
2. Ambient NIR from the environment
3. In some cases, the fluorophore may be excited by stray visible light (intentionally or unintentionally provided when the Excitation illumination off) and fluorescence from the target fluorophore will be present.

In some embodiments the VIS_DRK frame is also referred to as the VIS frame. While the VIS_DRK frame or VIS frame also contains the ambient NIR from the environment, this signal is relatively low compared to the intensity of the reflected visible light.

A final NIR image should contain only the NIR fluorescence from the target fluorophore. Note that the raw NIR frames contain this information, as well as the reflected visible light and the ambient NIR from the environment, and that the VIS_DRK frame contains the later two components: the reflected visible light and the ambient NIR from the environment. In some embodiments, subtracting the VIS_DRK frame (which may be referred to as “DRK” frame for this purpose) from the raw NIR frame yields an image containing only the fluorescence from the fluorophore, referred to as the dark-corrected NIR image (i.e., NIR*). The following formula describes this process, using the terms previously described for correcting for different gains and exposures:

$${NIR}^{*} = NIR - ( {DRK}^{*} \times {EXP\_RATIO}_{NIR - DRK} )$$

If a sequence contains only one VIS_DRK and one NIR frame (NIR-ratio: VISratio=1), subsequent image processing is simple:
The Visible image (VIS) is the VIS_DRK frame
NIR image (NIR*) is the dark-corrected NIR frame according to the previous formula
The Overlay image (OVL) is the sum of the VIS and NIR* images

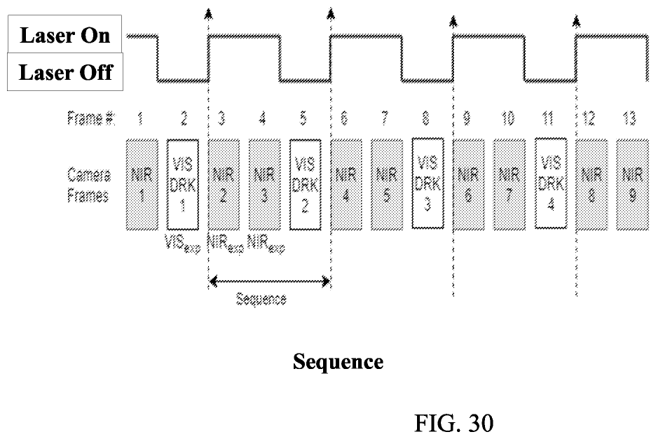

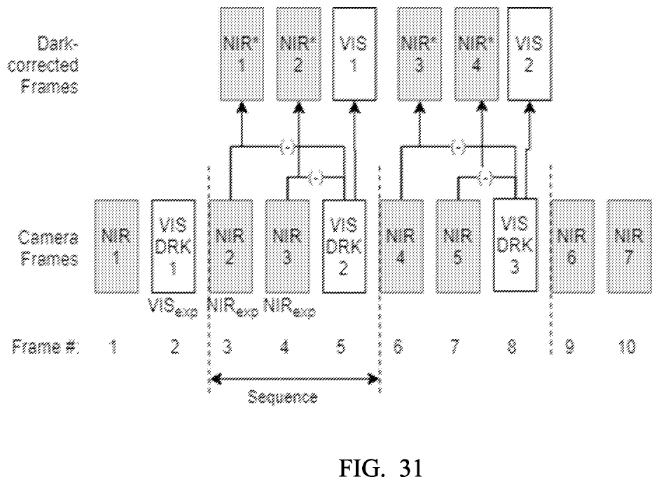

In some embodiments, the NIR: VIS_DRK is greater than 1 to allow us to sum the NIR data to enhance sensitivity to the NIR fluorescence signal. Determining which VIS_DRK frame to use for the NIR dark correction is described throughout. Moreover, when a sequence comprises a plurality of both NIR frames and VIS_DRK frames, a primary quantity for both the NIR frames and the VIS_DRK frames in the sequence may be quantified for subsequent image processing methods disclosed herein. For example, the sequences shown in FIGS. 30-35 includes a primary quantity of 2 NIR frames and a single VIS_DRK frame in each sequence. As disclosed herein, various imaging processing systems and methods may use NIR and/or VIS_DRK frames from preceding and/or subsequent sequences.

In some embodiments, as shown in FIG. 30 for example, when laser is in an off mode, a frame is captured which contains the visible light reflection from the target as well as the ambient NIR or IR signal. Thus, this frame is used as both the VIS and the VIS_DRK frame in this embodiment. When the laser is in an on mode, one or more NIR or IR frames are captured. Note that the NIR or IR frames contain all of the following: the reflected visible illumination, the ambient NIR or IR image, and the NIR or IR fluorescence from the tissue. In some embodiments, each NIR or IR frame is corrected by subtracting one correcting VIS_DRK frame, resulting in a corrected NIR or IR frame, which contains only the NIR or IR fluorescence from the tissue. In some embodiments, each NIR or IR image is generated by adding a first corrected NIR or IR frame and N quantity of subsequent corrected NIR or IR frames. In some embodiments, the VIS image is generated by adding a first VIS frame and V quantity of subsequent VIS frames. In some embodiments, N quantity and V quantity are positive whole numbers, while in other embodiments one or both of N and V may be zero.

In some embodiments, an exposure time for the VIS_DRK frame is equal to the exposure time for the NIR or IR frames. In some embodiments, the exposure time for the VIS_DRK frame is greater than the exposure time for the NIR or IR frame. In some embodiments, the exposure time for the VIS_DRK frame is less than the exposure time for the NIR or IR frame. In some embodiments, each VIS_DRK frame has the same exposure time. In some embodiments, at least a portion of the VIS_DRK frames have different exposure times. In some embodiments, each NIR or IR frame has the same exposure time. In some embodiments, at least a portion of the NIR or IR frames have different exposure times. In some embodiments, an exposure time when capturing the VIS_DRK image result in a VIS_DRK pixel value less than a maximum pixel value divided by the ratio between NIR or IR and VIS_DRK frames in a Sequence (FIG. 30). In one example, for an 8-bit image taken with a maximum pixel value in the VIS_DRK frame and in some embodiments the raw image value does not exceed maximum of about 254/4 or 255/4 (or about 63 counts). In some embodiments, the exposure time is reduced if the resulting VIS_DRK, NIR, or IR image is too bright. In some embodiments, reducing the count number reduces a motion artifact caused by subtraction. In some embodiments, the count number is reduced by decreasing the maximum pixel value, decreasing the ration between NIR or IR and VIS_DRK frames in a sequence, or both. In some embodiments, a saturation of one or more summed NIR images is less critical than saturation in the raw images.

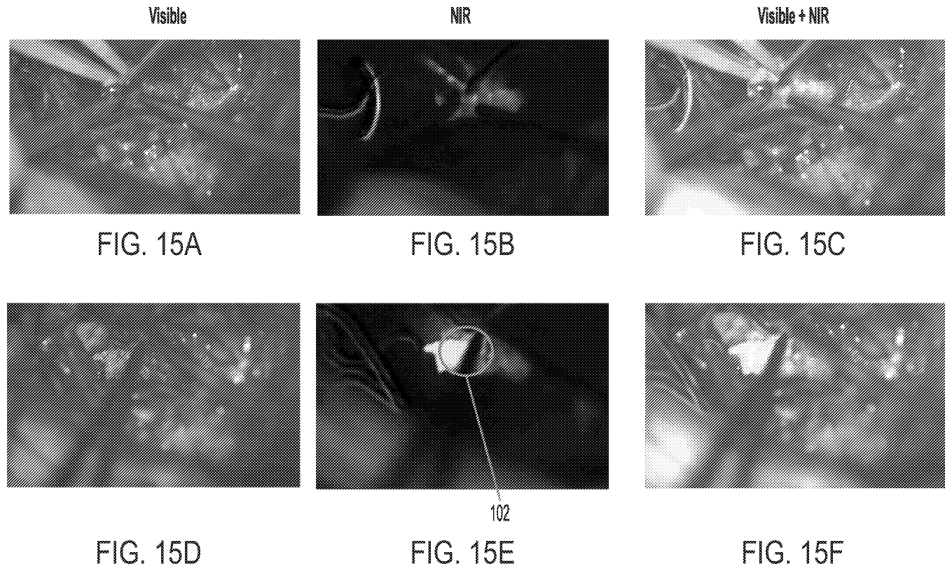

One image artifact is called ‘motion artifact’. One cause of motion artifacts occurs when two images are subtracted and something in the field of view moves between the times the two frames were captured. The systems and methods herein can reduce, minimize, or correct a motion artifact. An example of a subtraction-related motion artifact can be seen in FIG. 15 (i.e., FIGS. 15E and 15F). FIGS. 15E and 15F show exemplary depiction or what a motion artifact 1501 looks like based on VIS_DRK frame subtraction methods. As shown, the field of view contains several threads (attached to surgical sponges) which moved during the imaging sequence showing a multiple or repeated image of the threads as a result of such movement. The systems and methods described herein reduce, or minimize such a motion artifact. In some embodiments, the correcting VIS_DRK frame is a VIS_DRK frame in the same frame sequence as the first NIR or IR frame. In this case, however, the first NIR or IR frame is separated from the VIS_DRK frame used for correction by at least (N−1) times the NIR or IR frame exposure time. If the target tissue (or an object in the frame) moves during this period, the subtraction operation for dark correction will result in a motion artifact. To address this issue, in some embodiments, the VIS_DRK frame used to correct each NIR or IR frame can be chosen to be the VIS_DRK frame that's temporally closest to the given NIR or IR frame. In some embodiments, the correcting VIS_DRK frame is a VIS_DRK frame in a subsequent frame sequence to the frame sequence of the NIR or IR frame. In some embodiments, the correcting VIS_DRK frame for a first portion e of the primary quantity of the NIR or IR frames is a VIS_DRK frame in the same frame sequence as the first NIR or IR frame, wherein the correcting VIS_DRK frame for a second portion of the of the primary quantity of the NIR or IR frames is a VIS_DRK frame in a subsequent frame sequence to the frame sequence of the NIR or IR frame. In some embodiments, (N+1) is equal to or greater than the primary quantity. In some embodiments, generating the first NIR or IR image further comprises adding M quantity of corrected NIR or IR frames preceding the first corrected NIR or IR frame.

In some embodiments, the method further comprises generating a second NIR or IR image, overlaying the first NIR or IR image and the first VIS image to form the first overlaid image, and overlaying the second NIR or IR image and the second VIS image to form the second overlaid image. In some embodiments, the application further generates a second NIR or IR image, overlays the first NIR or IR image and the first VIS image to form the first overlaid image, and overlays the second NIR or IR image and the second VIS image to form the second overlaid image. In some embodiments, the second NIR or IR image is generated by adding a (N+1)th or (N+2)th corrected NIR or IR frame and N quantity of subsequent corrected NIR or IR frames. In some embodiments, the second VIS image is generated by adding a second VIS frame and V quantity of VIS frames subsequent to the second VIS frame.

In some embodiments, the correcting VIS frame is a VIS frame in the same frame sequence as the first NIR or IR frame. In some embodiments, the correcting VIS frame is a VIS frame in a subsequent frame sequence to the frame sequence of the NIR or IR frame. In some embodiments, the correcting VIS frame for a first portion of the of the primary quantity of the NIR or IR frames is a VIS frame in the same frame sequence as the first NIR or IR frame. In some embodiments, the correcting VIS frame for a second portion of the primary quantity of the NIR or IR frames is a VIS frame in a subsequent frame sequence to the frame sequence of the NIR or IR frame. In some embodiments, (N+1) is equal to or greater than the primary quantity. In some embodiments, generating the first NIR or IR image further comprises adding M quantity of corrected NIR or IR frames preceding the first corrected NIR or IR frame. In some embodiments, the application further comprises generating a second NIR or IR image, and overlaying the second NIR or IR image and the second VIS image to form the second overlaid image.

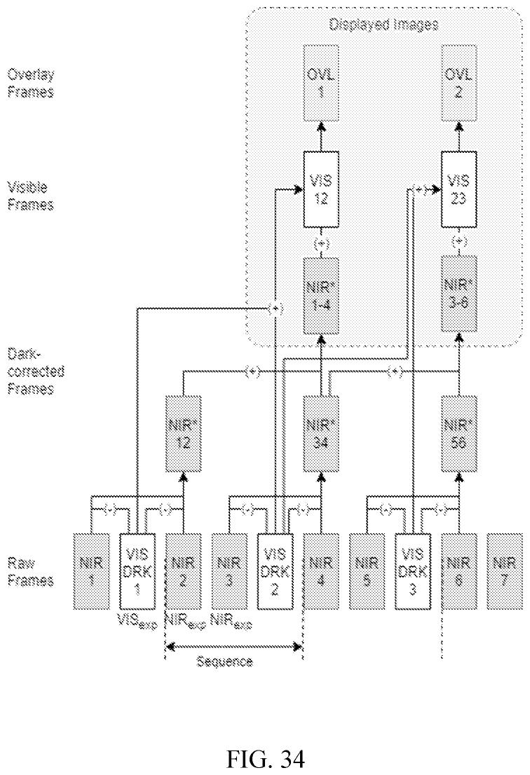

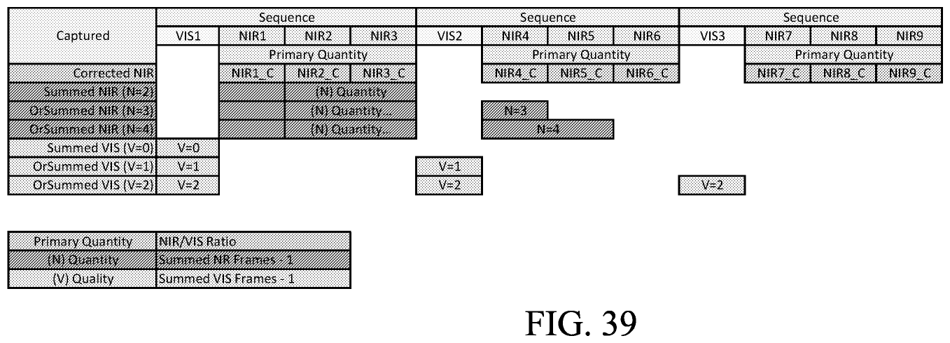

In some embodiments, the second image is generated by adding a (N+1)th or (N+2)th corrected NIR or IR frame and N quantity of subsequent corrected NIR or IR frames. N quantity is the total number of additional NIR frames summed with an original or primary NIR frame minus 1, consequently, the N quantity can be zero or more. In some embodiments, the second VIS image is generated by adding a second VIS frame and V quantity of VIS frames subsequent to the second VIS frame. The N quantity and V quantity is graphically represented in FIG. 39, wherein the V quantity is the total number of VIS frames summed with an original or primary VIS frame minus 1, consequently, the V quantity can be zero or more. In some embodiments, the VIS frame is the last frame in the sequence. In some embodiments, the VIS frame is the first frame in the sequence. In some embodiments, the VIS frame is anywhere in the sequence. In some embodiments, the VIS frame is the first frame in the sequence, last frame in the sequence or the VIS frame is anywhere in the sequence, or in any combination. In some embodiments, the display rate is the same as the sequence rate wherein the image made from a number of frames that exceed the number in the sequence are summed using a ‘boxcar’ accumulator as exemplified in FIG. 34 and Example 21.

The display rate describes the rate at which images are displayed to the user, as commonly measured in frames per unit time (e.g., frames per second, per millisecond, per microsecond, etc.) or as measured in hertz or cycles per unit time (e.g., cycles per second, per millisecond, per microsecond, etc. The sequence rate describes the rate at which the total number of frames are acquired in a sequence. A display rate that is the same as or equal to sequence rate means that the display images are being acquired and displayed at same rate as the sequence of fames are acquired.

### Computing System

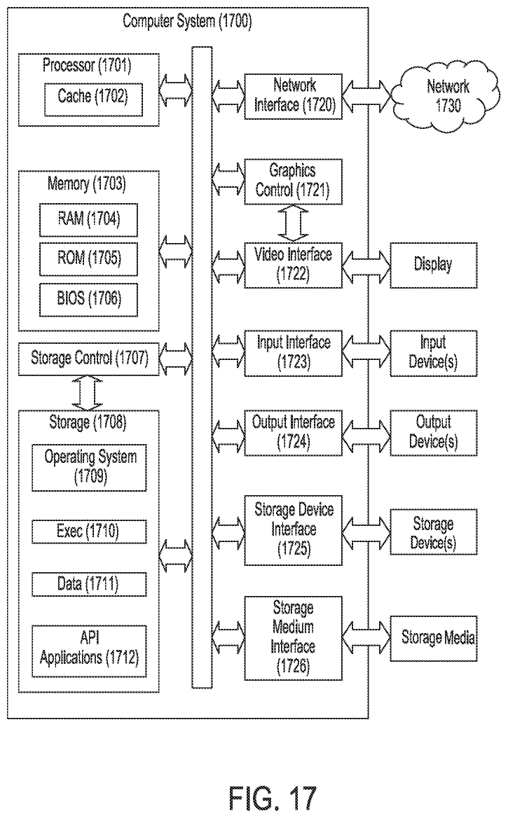

Referring to FIG. 17, a block diagram is shown depicting an exemplary machine that includes a computer system 1700 (e.g., a processing or computing system) within which a set of instructions can execute for causing a device to perform or execute any one or more of the aspects and/or methodologies for static code scheduling of the present disclosure. The components in FIG. 17 are examples only and do not limit the scope of use or functionality of any hardware, software, embedded logic component, or a combination of two or more such components implementing particular embodiments.

Computer system 1700 can include one or more processors 1701, a memory 1703, and a storage 1708 that communicate with each other, and with other components, via a bus 1740. The bus 1740 can also link a display 1732, one or more input devices 1733 (which can, for example, include a keypad, a keyboard, a mouse, a stylus, etc.), one or more output devices 1734, one or more storage devices 1735, and various tangible storage media 1736. All of these elements can interface directly or via one or more interfaces or adaptors to the bus 1740. For instance, the various tangible storage media 1736 can interface with the bus 1740 via storage medium interface 1726. Computer system 1700 can have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1700 includes one or more processor(s) 1701 (e.g., central processing units (CPUs) or general purpose graphics processing units (GPGPUs)) that carry out functions. Processor(s) 1701 optionally contains a cache memory unit 1702 for temporary local storage of instructions, data, or computer addresses. Processor(s) 1701 are configured to assist in execution of computer readable instructions. Computer system 1700 can provide functionality for the components depicted in FIG. 17 as a result of the processor(s) 1701 executing non-transitory, processor-executable instructions embodied in one or more tangible computer-readable storage media, such as memory 1703, storage 1708, storage devices 1735, and/or storage medium 1736. The computer-readable media can store software that implements particular embodiments, and processor(s) 1701 can execute the software. Memory 1703 can read the software from one or more other computer-readable media (such as mass storage device(s) 1735, 1736) or from one or more other sources through a suitable interface, such as network interface 1720. The software can cause processor(s) 1701 to carry out one or more processes or one or more steps of one or more processes described or illustrated herein. Carrying out such processes or steps can include defining data structures stored in memory 1703 and modifying the data structures as directed by the software.

The memory 1703 can include various components (e.g., machine readable media) including, but not limited to, a random access memory component (e.g., RAM 1704) (e.g., static RAM (SRAM), dynamic RAM (DRAM), ferroelectric random access memory (FRAM), phase-change random access memory (PRAM), etc.), a read-only memory component (e.g., ROM 1705), and any combinations thereof. ROM 1705 can act to communicate data and instructions unidirectionally to processor(s) 1701, and RAM 1704 can act to communicate data and instructions bidirectionally with processor(s) 1701. ROM 1705 and RAM 1704 can include any suitable tangible computer-readable media described below. In one example, a basic input/output system 1706 (BIOS), including basic routines that help to transfer information between elements within computer system 1700, such as during start-up, can be stored in the memory 1703.

Fixed storage 1708 is connected bidirectionally to processor(s) 1701, optionally through storage control unit 1707. Fixed storage 1708 provides additional data storage capacity and can also include any suitable tangible computer-readable media described herein. Storage 1708 can be used to store operating system 1709, executable(s) 1710, data 1711, applications 1712 (application programs), and the like. Storage 1708 can also include an optical disk drive, a solid-state memory device (e.g., flash-based systems), or a combination of any of the above. Information in storage 1708 can, in appropriate cases, be incorporated as virtual memory in memory 1703.

In one example, storage device(s) 1735 can be removably interfaced with computer system 1700 (e.g., via an external port connector (not shown)) via a storage device interface 1725. Particularly, storage device(s) 1735 and an associated machine-readable medium can provide non-volatile and/or volatile storage of machine-readable instructions, data structures, program modules, and/or other data for the computer system 1700. In one example, software can reside, completely or partially, within a machine-readable medium on storage device(s) 1735. In another example, software can reside, completely or partially, within processor(s) 1701.

Bus 1740 connects a wide variety of subsystems. Herein, reference to a bus can encompass one or more digital signal lines serving a common function, where appropriate. Bus 1740 can be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1700 can also include an input device 1733. In one example, a user of computer system 1700 can enter commands and/or other information into computer system 1700 via input device(s) 1733. Examples of an input device(s) 1733 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1733 can be interfaced to bus 1740 via any of a variety of input interfaces 1723 (e.g., input interface 1723) including, but not limited to, serial, parallel, game port, USB, FIREWIRE, THUNDERBOLT, or any combination of the above.

In particular embodiments, when computer system 1700 is connected to network 1730, computer system 1700 can communicate with other devices, specifically mobile devices and enterprise systems, distributed computing systems, cloud storage systems, cloud computing systems, and the like, connected to network 1730. Communications to and from computer system 1700 can be sent through network interface 1720. For example, network interface 1720 can receive incoming communications (such as requests or responses from other devices) in the form of one or more packets (such as Internet Protocol (IP) packets) from network 1730, and computer system 1700 can store the incoming communications in memory 1703 for processing. Computer system 1700 can similarly store outgoing communications (such as requests or responses to other devices) in the form of one or more packets in memory 1703 and communicated to network 1730 from network interface 1720. Processor(s) 1701 can access these communication packets stored in memory 1703 for processing.

Examples of the network interface 1720 include, but are not limited to, a network interface card, a modem, and any combination thereof. Examples of a network 1730 or network segment 1730 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1730, can employ a wired and/or a wireless mode of communication. In general, any network topology can be used.

Information and data can be displayed through a display 1732. Examples of a display 1732 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1732 can interface to the processor(s) 1701, memory 1703, and fixed storage 1708, as well as other devices, such as input device(s) 1733, via the bus 1740. The display 1732 is linked to the bus 1740 via a video interface 1722, and transport of data between the display 1732 and the bus 1740 can be controlled via the graphics control 1721. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1732, computer system 1700 can include one or more other peripheral output devices 1734 including, but not limited to, an audio speaker, a printer, a storage device, and any combinations thereof. Such peripheral output devices can be connected to the bus 1740 via an output interface 1724. Examples of an output interface 1724 include, but are not limited to, a serial port, a parallel connection, a USB port, a FIREWIRE port, a THUNDERBOLT port, and any combinations thereof.

In addition or as an alternative, computer system 1700 can provide functionality as a result of logic hardwired or otherwise embodied in a circuit, which can operate in place of or together with software to execute one or more processes or one or more steps of one or more processes described or illustrated herein. Reference to software in this disclosure can encompass logic, and reference to logic can encompass software. Moreover, reference to a computer-readable medium can encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, computer-implemented aspects, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein can be embodied directly in hardware, in a software module executed by one or more processor(s), or in a combination of the two. A software module can reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX® VX Works®, an embedded Linux (e.g. RT Linux, QNX, LynxOS), and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, AppleRiOS®, Research In Motion® BlackBerry OS®, Google® Android®, Microsoft® Windows Phone® OS, Microsoft® Windows Mobile® OS, Linux®, Palm® WebOS®, VX Works®, or an embedded Linux (e.g. RT Linux, QNX, LynxOS). Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, Ouya®, VX Works®, or an embedded Linux (e.g. RT Linux, QNX, LynxOS).

### Digital Processing Device

In some embodiments, the systems and methods described herein include a digital processing device, a processor, or use of the same. In further embodiments, the digital processing device includes one or more hardware central processing units (CPUs) and/or general-purpose graphics processing units (GPGPUs), or special purpose GPGCUs that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. In addition, in accordance with the description herein, devices include also partitioning the signal processing and computation between a unit located proximally to the imaging optics (e.g. a FPGA or DSP), and a ‘back end’ PC. It is understood that distribution of the processing can be performed between various locations.

In some embodiments, the digital processing device includes an operating system configured to perform executable instructions. The operating system is, for example, software, including programs and data, which manages the device's hardware and provides services for execution of applications.

In some embodiments, the device includes a storage and/or memory device. The storage and/or memory device is one or more physical apparatuses used to store data or programs on a temporary or permanent basis.

In some embodiments, the digital processing device includes a display to send visual information to a user.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

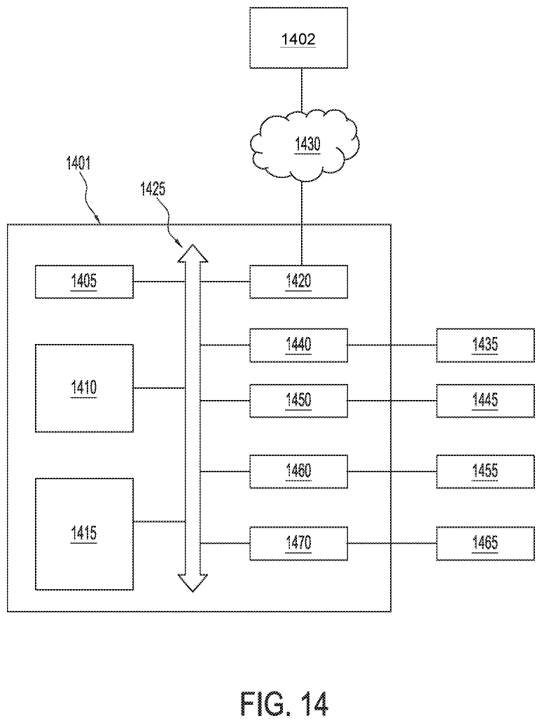

Referring to FIG. 14, in a particular embodiment, an exemplary digital processing device 1401 is programmed or otherwise configured to control imaging and image processing aspects of the systems herein. In this embodiment, the digital processing device 1401 includes a central processing unit (CPU, also “processor” and “computer processor” herein) 1405, which can be a single core or multi core processor, or a plurality of processors for parallel processing. The digital processing device 1401 also includes memory or memory location 1410 (e.g., random-access memory, read-only memory, flash memory), electronic storage unit 1415 (e.g., hard disk), communication interface 1420 (e.g., network adapter, network interface) for communicating with one or more other systems, and peripheral devices, such as cache, other memory, data storage and/or electronic display adapters. The peripheral devices can include storage device(s) or storage medium 1465 which communicate with the rest of the device via a storage interface 1470. The memory 1410, storage unit 1415, interface 1420 and peripheral devices are in communication with the CPU 1405 through a communication bus 1425, such as a motherboard. The storage unit 1415 can be a data storage unit (or data repository) for storing data. The digital processing device 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some embodiments is a telecommunication and/or data network. The network 1430 can include one or more computer servers, which can enable distributed computing, such as cloud computing. The network 1430, in some embodiments with the aid of the device 1401, can implement a peer-to-peer network, which can enable devices coupled to the device 1401 to behave as a client or a server.

Continuing to refer to FIG. 14, the digital processing device 1401 includes input device(s) 1445 to receive information from a user, the input device(s) in communication with other elements of the device via an input interface 1450. The digital processing device 1401 can include output device(s) 1455 that communicates to other elements of the device via an output interface 1460.

Continuing to refer to FIG. 14, the memory 1410 can include various components (e.g., machine readable media) including, but not limited to, a random-access memory component (e.g., RAM) (e.g., a static RAM “SRAM”, a dynamic RAM “DRAM, etc.), or a read-only component (e.g., ROM). The memory 1410 can also include a basic input/output system (BIOS), including basic routines that help to transfer information betweF5-10en elements within the digital processing device, such as during device start-up, can be stored in the memory 1410.

Continuing to refer to FIG. 14, the CPU 1405 can execute a sequence of machine-readable instructions, which can be embodied in a program or software. The instructions can be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and write back. The CPU 1405 can be part of a circuit, such as an integrated circuit. One or more other components of the device 1401 can be included in the circuit. In some embodiments, the circuit is an application specific integrated circuit (ASIC) or a field programmable gate array (FPGA), or other like circuit or array.

Continuing to refer to FIG. 14, the storage unit 1415 can store files, such as drivers, libraries and saved programs. The storage unit 1415 can store user data, e.g., user preferences and user programs. The digital processing device 1401 in some embodiments can include one or more additional data storage units that are external, such as located on a remote server that is in communication through an intranet or the Internet. The storage unit 1415 can also be used to store operating system, application programs, and the like. Optionally, storage unit 1415 can be removably interfaced with the digital processing device (e.g., via an external port connector (not shown)) and/or via a storage unit interface. Software can reside, completely or partially, within a computer-readable storage medium within or outside of the storage unit 1415. In another example, software can reside, completely or partially, within processor(s) 1405.

Continuing to refer to FIG. 14, the digital processing device 1401 can communicate with one or more remote computer systems 1402 through the network 1430. For instance, the device 1401 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. In some embodiments, the remote computer system is configured for image and signal processing of images acquired using the image systems herein. In some embodiments, the imaging systems herein allows partitioning of image and signal processing between a processor in the imaging system (e.g. based on a MCU, DSP or FPGA) and a remote computer system, i.e., a back-end server, or other like signal processor.

Continuing to refer to FIG. 14, information and data can be displayed to a user through a display 1435. The display is connected to the bus 1425 via an interface 1440, and transport of data between the display other elements of the device 1401 can be controlled via the interface 1440.

Methods as described herein can be implemented by way of machine (e.g., computer processor) executable code stored on an electronic storage location of the digital processing device 1401, such as, for example, on the memory 1410 or electronic storage unit 1415. The machine executable or machine-readable code can be provided in the form of software. During use, the code can be executed by the processor 1405. In some embodiments, the code can be retrieved from the storage unit 1415 and stored on the memory 1410 for ready access by the processor 1405. In some situations, the electronic storage unit 1415 can be precluded, and machine-executable instructions are stored on memory 1410.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some embodiments, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions can be implemented as program modules, such as functions, objects, Application Programming Interfaces (APIs), data structures, and the like, that perform particular tasks or implement particular abstract data types. In light of the disclosure provided herein, those of skill in the art will recognize that a computer program can be written in various versions of various languages.

The functionality of the computer readable instructions can be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Indications and Methods

The systems and methods herein can be used to identify presence of health or disease, in diagnosis, imaging, health monitoring and the like in a given sample (e.g., organ, organ substructure, tissue, or sample, whether ex-vivo or in situ). The systems and methods herein can be used to aid surgery and other medical procedures in the treatment of the foregoing.

In some aspects, the systems and methods herein can be used after administering a detectable agent, such as a therapeutic or imaging agent comprising a fluorophore, comprises intravenous administration, intramuscular administration, subcutaneous administration, intraocular administration, intra-arterial administration, peritoneal administration, intratumoral administration, intradermal administration, or any combination thereof. In some aspects, the imaging comprises tissue imaging, ex vivo imaging, intraoperative imaging, or any combination thereof. In some aspects, the sample is in an in vivo sample, an in situ sample, an ex vivo sample, or an intraoperative sample. In further aspects, the sample is an organ, an organ substructure, a tissue, a tumor, or a cell. In some aspects, the sample autofluoresces. In some aspects, autofluorescence of the sample comprises an ocular fluorophore, tryptophan, or protein present in a tumor or malignancy. In some aspects, the method is used to visualize vessel flow or vessel patency.

In some aspects, the abnormal tissue, cancer, tumor, vasculature or structure comprises a blood vessel, lymph vasculature, neuronal vasculature, or CNS structure. In some aspects, the imaging is angiography, arteriography, lymphography, or cholangiography. In some aspects, the imaging comprises detecting a vascular abnormality, vascular malformation, vascular lesion, organ or organ substructure, cancer or diseased region, tissue, structure or cell. In some aspects, the vascular abnormality, vascular malformation, or vascular lesion is an aneurysm, an arteriovenous malformation, a cavernous malformation, a venous malformation, a lymphatic malformation, a capillary telangiectasia, a mixed vascular malformation, a spinal dural arteriovenous fistula, or a combination thereof. In some aspects, the organ or organ substructure is brain, heart, lung, kidney, liver, or pancreas. In further aspects, the method further comprises performing surgery on the subject. In some aspects, the surgery comprises angioplasty, cardiovascular surgery, aneurysm repair, valve replacement, aneurysm surgery, arteriovenous malformation or cavernous malformation, a venous malformation surgery, a lymphatic malformation surgery, a capillary telangiectasia surgery, a mixed vascular malformation surgery, or a spinal dural arteriovenous fistula surgery, repair or bypass, arterial bypass, organ transplant, plastic surgery, eye surgery, reproductive system surgery, stent insertion or replacement, plaque ablation, removing the cancer or the diseased region, tissue, structure or cell of the subject, or any combination thereof. In some aspects, the imaging comprises imaging a vascular abnormality, cancer or diseased region, tissue, structure, or cell of the subject after surgery. In further aspects, the method further comprises treating a cancer in the subject.

In some aspects, the method further comprises repair of an intracranial CNS vascular defect, a spinal CNS vascular defect; peripheral vascular defects; removal of abnormally vascularized tissue; ocular imaging and repair; anastomosis; reconstructive or plastic surgery; plaque ablation or treatment or restenosis in atherosclerosis; repair or resection (including selective resection), preservation (including selective preservation), of vital organs or structures such as nerves, kidney, thyroid, parathyroid, liver segments, or ureters; identification and management (sometimes preservation, sometimes selective resection) during surgery; diagnosis and treatment of ischemia in extremities; or treatment of chronic wounds. In some aspects, the intracranial vascular defect and/or the spinal vascular defect comprises an aneurysm, an arteriovenous malformation, a cavernous malformation, a venous malformation, a lymphatic malformation, a capillary telangiectasia, a mixed vascular malformation, or a spinal dural arteriovenous fistula, or any combination thereof. In some aspects, the peripheral vascular defect comprises an aneurysm, a coronary bypass, another vascular bypass, a cavernous malformation, an arteriovenous malformation, a venous malformation, a lymphatic malformation, a capillary telangiectasia, a mixed vascular malformation, a spinal dural arteriovenous fistula, or any combination thereof. In some aspects, the abnormally vascularized tissue comprises endometriosis or a tumor.

For example, fluorescence angiography is useful during certain neurosurgical procedures in the brain and spinal cord. Repair of blood vessel defects such as a vascular lesion, vascular malformation, vascular abnormality, aneurysm, arteriovenous malformation, cavernous malformation, venous malformation, lymphatic malformation, capillary telangiectasia, mixed vascular malformation, or spinal dural arteriovenous fistula, and the like, requires imaging of the defect architecture, confirmation that the defect is successfully isolated prior to repair, and confirmation that repaired vessels have restored proper blood flow and patency. Vessel patency is particularly crucial in the CNS to avoid neurologic damage or death that can result from undetected bleeding into these tissues. Neurosurgical microscopes, neuroendoscopes, endovascular endoscopes, and robotic surgical systems including the systems and methods described herein may all be used in this setting. Removal of CNS tumors such as pituitary adenoma is another setting in which fluorescence angiography can be applied to improve safety and efficacy of treatment. The visualization of vascular flow to the tumor and verification that the tumor has been removed without residual bleeding are both important uses for this technology.

Fluorescence angiography, cholangiography, lymphography, and the like are useful in support of a variety of surgical interventions. The systems and methods described herein can be used in various cardiovascular surgeries, including aneurysm repair, valve replacement, arteriovenous malformation, cavernous malformation or a venous malformation, a lymphatic malformation, a capillary telangiectasia, a mixed vascular malformation, or a spinal dural arteriovenous fistula, repair or bypass, arterial bypass, and the like for visualization of blood flow and vessel patency. The systems and methods described herein can be used in plastic surgery, trauma surgery, reconstructive surgery, and the like for vascular mapping and for assessment of tissue perfusion. Tissue perfusion is of particular importance in flap reconstructions and in anastomoses of the gastrointestinal tract, for example following colorectal cancer surgery or esophagectomy, as tissue ischemia following such surgeries can result in loss of tissue and graft failure or leaking anastomosis. Fluorescence lymphography using systems and methods described herein is useful for demonstrating flow of the lymphatic vessels, for example to support re-routing of lymphatic drainage to treat lymphedema.

The systems and methods described herein are useful for visualization of organs or organ segments in a variety of surgical procedures. Liver segments can be imaged following intra-arterial dye injection during partial hepatectomy. Perfusion and bile production can be assessed following partial or total liver transplantation. Other hepatobiliary surgeries, including resection of liver cancers or metastases, are also supported by angiography or cholangiography. Contrast between the kidney and adrenal gland can be achieved using fluorescent dyes or conjugates that are cleared through renal filtration. This procedure can help in differentiating the adrenal gland from the kidney, for example to avoid kidney damage during removal of the adrenal glands. The ureters can also be identified using these methods to avoid damage to them during uro-abdominal surgeries. Abnormally vascularized tissues, such as endometriosis or tumors, can be identified and removed using these methods.

Coupled with a targeting moiety that binds specifically to nerves, fluorescence imaging systems and methods described herein can be used to visualize nerves during surgery to avoid damage. This is important during surgeries in highly innervated areas, particularly where damage to the nerves can result in significant morbidity. Examples include facial nerves, visceral nerves, and cavernous nerves. For example, cavernous nerves are important for penile and clitoral erection, and thus important for erectile function. Moreover, vascular injury and diseases affect blood vessels and blood flow. For example, affected cavernous nerves or decreased blood flow to the penis can cause erectile dysfunction. Moreover the cavernous nerves of the penis are frequently damaged during prostate cancer surgery. Vascular reconstructive surgery is one way to improve blood flow to the penis to help a man with erectile dysfunction (ED).

Coupled with a targeting moiety that binds or accumulates specifically in abnormal vascular tissue, fluorescence imaging systems and methods described herein can be used to identify vascular abnormalities during surgery. For example, a cavernoma is a cluster of abnormal blood vessels, usually found in the brain and spinal cord. They are sometimes known as cavernous angiomas, cavernous hemangiomas, or cerebral cavernous malformation (CCM). A typical cavernoma looks like a raspberry. Cavernomas are treated by microsurgical resection or stereotactic radiosurgery if the patient is experiencing severe symptoms, such as intractable seizures, progressive neurological deterioration, one severe hemorrhage in a noneloquent region of the brain, or at least two severe hemorrhages in eloquent brain. The systems and methods herein can be used to detect, image and treat cavernous malformation, cavernous angiomas, cavernous hemangiomas, or cerebral cavernous malformation (CCM) including via surgery.

Similarly, the systems and methods herein can be used to detect, image and treat arteriovenous malformation including via surgery. An arteriovenous malformation (AVM) is an abnormal tangle of blood vessels connecting arteries and veins, which disrupts normal blood flow and oxygen circulation. Arteries are responsible for taking oxygen-rich blood from the heart to the brain. Veins carry the oxygen-depleted blood back to the lungs and heart. AVM treatment sometimes requires a combination of treatments, including surgery, embolization and radiation.

Disruptions of the ophthalmic vasculature occur as a result of diseases such as diabetes, glaucoma, or Susac's syndrome, secondary to trauma, or spontaneously. The systems and methods described herein are useful in diagnosis and in treatment targeting and/or monitoring of such disruptions. These can include macular edema, macular ischemia, age-related macular degeneration, retinal tear, retinal degeneration, retinal artery occlusion, retinal vein occlusion, and the like. Treatment of tumors in the eye often require intraocular injection of chemotherapeutics, which requires careful monitoring to ensure accurate delivery to the tumor while minimizing damage to normal structures within or surrounding the eye. In some instances, endogenous fluorescence of the chemotherapeutic, such as topotecan, can be monitored. In other instances, a tracer dye can be administered with the chemotherapeutic to facilitate imaging.

Certain types of cancer, such as head and neck cancer or sarcomas of the extremities, may be treated using super selective intra-arterial chemotherapy. This method can improve prognosis and spare normal organ function but requires precise identification of the vessels supplying blood to the cancer tissue. The systems and methods described herein are useful for fluorescence imaging and identification of appropriate arteries prior to administration of chemotherapy.

Such systems can be useful for endovascular imaging for diagnosis and treatment monitoring in cardiovascular diseases such as atherosclerosis. Examination of features such as lumen dimensions, plaque burden, remodeling, lipid components, cap thickness, neo-angiogenesis, and inflammation are used to diagnose plaque instability; fluorescence imaging in combination with other technologies can improve these assessments. Following stent placement, fluorescence angiography can be used to detect vessel restenosis.

The systems and methods described herein are useful in non-invasive diagnosis and monitoring of tissue perfusion, for example in chronic wounds or limb/extremity ischemia.

The systems and methods described herein are useful in microvasculature imaging. For example, oxyhemoglobin and deoxyhemoglobin have sequential two-color, two-photon absorption properties that can serve as endogenous contrasts in microvasculature imaging. Using a sensitive modulation transfer technique, the systems and methods described herein can image hemoglobin in red blood cells with micrometer resolution, with or without labeling using a fluorophore or other detectable compound.

The systems and methods described herein can use multispectral images to identify subcutaneous vasculature, with improved contrast in the near infrared spectrum, including detection and methods involving infrared and near-infrared imaging of superficial blood vessels.

The systems and methods described herein can be used in angiography and coronary catheterization. For example, a coronary angiogram is a procedure that uses imaging to see the heart's blood vessels. The test is generally done to visualize any restrictions in blood flow going to the heart. Coronary angiograms are part of a general group of procedures known as heart (cardiac) catheterizations. Cardiac catheterization procedures can both diagnose and treat heart and blood vessel conditions. A coronary angiogram, which can help diagnose heart conditions, is the most common type of cardiac catheterization procedure. Similarly, such systems and methods described herein can be applied to other vasculature including lymph, cerebral vasculature, organ vasculature, arteries, capillaries, veins, and the like.

The systems and methods described herein can be used in imaging and detecting cancers, e.g., for detecting and imaging angiogenesis, (i.e., the formation of new blood vessels) associated with tumors.

The systems and methods described herein can be used to diagnose, image, and detect blood vessel derived tumors and aid in their treatment through surgery and improve the health of patients through monitoring. Vascular tumors may be benign or malignant. Benign tumors form recognizable vascular channels filled with blood or lymphatic fluid. Malignant tumors are usually more solid and cellular without well-formed vascular channels. Similarly, such systems and methods described herein can be applied to other vasculature including lymph, cerebral vasculature, organ vasculature, arteries, capillaries, veins, and the like. Exemplary vessel derived tumors include those of endothelial cells, including hemangiomas, lymphangiomas, angiosarcomas, or cells supporting or surrounding blood vessels including glomus tumors, or hemangiopericytomas.

The systems and methods described herein can be used to diagnose, image, monitor and determine the outcome of heart surgery, including heart valve surgery, and treatment through surgery and improve the health of patients through monitoring.

In some applications the systems and methods disclosed herein can be used to diagnose, image, and monitor intrinsic fluorescence or autofluorescence in tissues with or without the administration of a fluorescent dye or other fluorescent agent as a contrast agent or an imaging agent per se. Intrinsic protein fluorescence, predominantly derived from tryptophan (ΔEX˜280 nm, λEM˜350 nm), as well as other aromatic amino acids tyrosine and phenylalanine, in proteins can be used with the systems and methods herein, for example in label-free Förster resonance energy transfer (FRET) techniques. For example, in terms of wavelength and intensity, tryptophan fluorescence is strongly influenced by its (or the protein's) local environment, which, in addition to fluorescence quenching, has been applied to study protein conformational changes. Intrinsic FRET utilizes the intrinsic fluorescence of tryptophan in conjunction with target-specific fluorescent probes as FRET donors and acceptors, respectively, for real time detection of native proteins. For example, fluorescence intensity profiles measured along the optical axis of human eye lenses can correlate with age-related nuclear cataract showing increasing concentration of fluorescent post-translational modification (PTM) towards the lens center in accord with the increased optical density in the lens nucleolus. The imaging systems and methods herein can provide spatiotemporal information of PTMs with little perturbation to the cellular environment. Significant differences between fluorescence lifetimes of “free” Trp derivatives hydroxytryptophan (OH-Trp), N-formylkynurenine (NFK), kynurenine (Kyn), hydroxykynurenine (OH-Kyn) and their residues can be measured and used to image, monitor, and diagnose disease in the eye. In addition, fundus autofluorescence (FAF) is a non-invasive retinal imaging modality used in clinical practice to provide a density map of lipofuscin, the predominant ocular fluorophore, in the retinal pigment epithelium. The imaging systems and methods herein can be used to evaluate, image, diagnose, and monitor various retinal diseases, including age related macular degeneration, macular dystrophies, retinitis pigmentosa, white dot syndromes, retinal drug toxicities, and various other retinal disorders. Moreover, autofluorescence depends on endogenous fluorophores in the tissue, which undergo a change associated with malignant transformation. This change (malignancy) can be detected as an alteration in the spectral profile and intensity of autofluorescence. Consequently, autofluorescence of tumors can be detected using the systems and methods described herein, making the systems and methods herein useful for imaging, diagnosing, and monitoring a variety of cancers. For example, bladder cancer is an exemplary cancer that autofluoresces. Fluorescence excitation wavelengths varying from 220 to 500 nm were used to induce tissue autofluorescence, and emission spectra can be measured in the 280-700 nm range. These spectra are combined to construct 2-dimensional fluorescence excitation-emission matrices (EEMs). Significant changes in fluorescence intensity of EEMs observed between normal and tumor bladder tissues are indicative of disease, the most marked differences being at the excitation wavelengths of 280 and 330 nm. Addition of contrast, fluorescent imaging agents, or target-specific fluorescent agents, can be used to further exemplify the application of the systems and methods in the detection, imaging, diagnosis, and monitoring of intrinsic tissue autofluorescence and tissue autofluorescence various applications.

Table 2 shows information of exemplary embodiments of indications and applicable organ vasculature for use with the systems and methods herein.

TABLE 2
Use of Systems and Methods in Vascular Intervention

| Indication | Intervention type | Organ System |
|---|---|---|
| Arteriovenous malformation | Neurosurgery | CNS |
| Cavernous malformation | Neurosurgery | CNS |
| Intracranial aneurysm | Neurosurgery | CNS |
| Pituitary adenoma | Neurosurgery | CNS |
| Spinal dural arteriovenous | Surgery | CNS |
| fistula | | |
| Adrenal surgery | Surgery | Endocrine |
| Thyroid surgery | Surgery | Endocrine |
| (parathyroid preservation) | | |
| Critical limb ischemia | Diagnostic | Extremities |
| Retinoblastoma | Chemotherapy | Eye |
| Diabetic macular edema | Diagnostic | Eye |
| Diabetic macular ischemia | Diagnostic | Eye |
| Diabetic retinopathy | Diagnostic | Eye |
| Macular degeneration | Diagnostic | Eye |
| Retinal artery occlusion | Diagnostic | Eye |
| Retinal vein occlusion | Diagnostic | Eye |
| Susac's syndrome | Diagnostic | Eye |
| Glaucoma | Diagnostic | Eye |
| Retinal surgery | Surgery | Eye |
| Kidney transplant | Surgery | Genitourinary |
| Ureter visualization | Surgery | Genitourinary |
| (any uro-abdominal surgery) | | |
| Kidney stones | Surgery | Genitourinary |
| Colorectal surgery | Surgery | GI |
| Esophageal anastomosis | Surgery | GI |
| Craniomaxillofacial trauma | Surgery | Head and neck |
| Liver cancer | Surgery | Hepatobiliary |
| Partial hepatectomy | Surgery | Hepatobiliary |
| Partial liver transplantation | Surgery | Hepatobiliary |
| Hepatobiliary surgery | Surgery | Hepatobiliary |
| Chronic wounds | Diagnostic | Soft tissue |
| Plastic surgery | Surgery | Soft tissue |
| Reconstructive surgery | Surgery | Soft tissue |
| Intra-arterial chemotherapy | Chemotherapy | Tumor |
| Lymphedema | Diagnostic | Vasculature |
| Atherosclerosis | Diagnostic | Vasculature |
| Endometriosis | Surgery | Viscera |

### Terms and Definitions

For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein.

As used herein A and/or B encompasses one or more of A or B, and combinations thereof such as A and B. It will be understood that although the terms “first,” “second,” “third” etc. can be used herein to describe various elements, components, regions and/or sections, these elements, components, regions and/or sections should not be limited by these terms. These terms are merely used to distinguish one element, component, region or section from another element, component, region or section. Thus, a first element, component, region or section discussed below could be termed a second element, component, region or section without departing from the teachings of the present disclosure.

The terminology used herein is for the purpose of describing particular embodiments only and is not intended to limit the present disclosure. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

As used in this specification and the claims, unless otherwise stated, the term “about,” and “approximately,” or “substantially” refers to variations of less than or equal to +/−0.1%, +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

As used herein, “LP” refers to longpass filters. LP filters transmit wavelengths longer than the transition wavelength and reflect a range of wavelengths shorter than the transition wavelength, as will be understood by one of ordinary skill in the art.

As used herein “SP” refers to shortpass filters. SP filters transmit wavelengths shorter than the transition wavelength and reflect a range of wavelengths longer than the transition wavelength, as will be understood by one of ordinary skill in the art.

As used herein “infrared” means any light in the infrared spectrum including light wavelengths in the IR-A (about 800-1400 nm), IR-B (about 1400 nm-3 μm) and IR-C (about 3 μm-1 mm) ranges, and near infrared (NIR) spectrums from 700 nm to 3000 nm. Generally, NIR or IR light comprises light in the infrared spectrum including light wavelengths from about 750 nm to 3000 nm.

As used herein “visible” means any light typically in the human visible spectrum including light wavelengths from about 300 nm to 750 nm. Visible is also used to depict something that is generally seen by the human eye whether or not as seen directly or through an instrument such as a microscope or other optical instrument.

As used herein, “coaxial” means that two or more light beam paths substantially overlap or are substantially parallel to each other within appropriate tolerances. That is, the axis along which a cone of light used for excitation extends along the imaging axis.

As used herein, “hot mirror”, “shortpass dichroic filter”, and “shortpass dichroic mirror” have the meaning as would be understood by one of ordinary skill in the art.

As used herein, “cold mirror”, “long pass dielectric filter”, and “longpass dichroic mirror” as used herein have the same meaning as would be understood by one of ordinary skill in the art.

As used herein, “dielectric filter”, and “dielectric mirror” as used herein can refer to a same physical element. A “dielectric filter” can refer to a device for selective transmitting. A “dielectric filter” can refer to a device for selective reflecting.

As used herein, “filter”, and “mirror” as used herein can refer to a same physical element.

Unless otherwise defined, all technical terms used herein have the same meaning as commonly understood by one of ordinary skill in the art to which this invention belongs.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein can be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

### EXAMPLES

The following illustrative examples are representative of embodiments of the software applications, systems, and methods described herein and are not meant to be limiting in any way.

### Example 1. Use of System During Pediatric Brain Tumor Resection

This example describes use of the imaging system and/or method disclosed herein for coaxial illumination and visualization of tozuleristide fluorescence during surgical resection of a pediatric brain tumor. The imaging system of the present invention was used to image brain tissue to detect a cancer using fluorescence imaging. Surgery was performed to remove cancer from the subject.

Subject T613 was diagnosed with a Grade 4 Atypical Teratoid Rhabdoid Tumor (ATRT) in the posterior fossa/brain stem. Tozuleristide which is a peptide-fluorophore detectable agent (15 mg/m2 dose), was given by intravenous (IV) bolus injection about 13.5 hours prior to the start of surgery. The imaging system was attached to the Zeiss Pentero surgical microscope prior the start of surgery, and the microscope head and arm and the imaging system and proximal imaging cable were enclosed in the microscope manufacturer's standard sterile drape. The drape's cover glass was secured over the bottom window aperture of the imaging system.

After the tumor was exposed, the imaging platform was initialized and used continuously. The imaging platform enabled the surgeon to view fluorescence and visible imaging separately and together on the imaging station display, simultaneously with viewing the surgical site in visible light through the operating microscope's eyepieces. The surgeon noted that the imaging system was unobtrusive and easy to use, and its use did not burden or hinder surgical routine practice. Moreover, there was no need to reposition the operating microscope to view the imaging platform's fluorescence and visible images, thus allowing normal imaging of the surgical area through the microscope's eyepieces together with the fluorescence imaging system during the operation, which decreased disruption to the surgical workflow.

Video was captured for the duration of the tumor resection, and still images were captured of the exposed tumor. Tozuleristide fluorescence was observed in situ in the exposed tumor. FIGS. 15A-15F show images taken from the tumor resection with the near-infrared (NIR) fluorescence images of the tumor using the imaging system (FIGS. 15B and 15E) and the overlay image with the NIR or IR fluorescence overlaid with the white light or visible light spectrum illumination (FIGS. 15C and 15F). The tumor appeared to the surgeon as a bright blue-green mass 102 in the NIR or IR fluorescence image and in the overlay image (shown as a bright white mass in grey-scale), while the non-tumor brain tissue did not show any fluorescence in the fluorescence image (FIGS. 15B and 15E) and appeared normal in the visible (FIGS. 15A and 15D). and overlay images appeared darker than the tumor mass in the NIR or IR fluorescence image indicating no discernable background fluorescence in non-tumor or normal brain tissue. In the overlay image, the normal brain tissue appeared red, as it does under normal visible light or white light as shown the visible light images of the tumor (FIGS. 15C and 15F). The surgeon noted that only tumor tissue appeared fluorescent. The surgeon also noted that under normal visible light it was “somewhat difficult to distinguish tumor from normal tissue,” but with NIR or IR fluorescence using the imaging system there was “very good distinction between tumor and normal tissue fluorescence.” The fluorescent tissue samples were demonstrated and confirmed to be viable tumor by histopathology.

This case demonstrated that the imaging system could be used continuously in an intraoperative setting to capture images and video of visible light and NIR or IR fluorescence, without disrupting the normal surgical flow. The data further demonstrated that the coaxial illumination and imaging system enabled the surgeon to visualize and precisely localize fluorescence in tumor tissues during surgery and use this information to remove tumor tissue during resection.

### Example 2. Example of Creating an Overlay Image-First Example of Forming First and Second Overlaid Images

In the example is provided per Table 2 below of a method of forming an overlaid image, wherein the primary quantity (N+1) is 3, wherein N=2 and wherein V=0

TABLE 2

| | Sequence 1 | | | | Sequence 2 | | | | |
|---|---|---|---|---|---|---|---|---|---|
| Frame Name | VIS1 | NIR1 | NIR2 | NIR3 | VIS2 | NIR4 | NIR5 | NIR6 | VIS3 |
| Frame # | 1 | 2 | 3 | 4 | 5 | 6 | 7 | 8 | 9 |

The NIR or IR frames (NIR1, NIR2, NIR3) are corrected by subtracting the previous Visible frame (VIS1) to yield the following corrected NIR or IR frames: NIR1−VIS1; NIR2−VIS1; and NIR3−VIS1. As N=2, the first NIR or IR image is generated by the sum of the first 3 corrected NIR or IR frames, the first NIR or IR image=(NIR1−VIS1)+(NIR2−VIS1)+(NIR3−VIS1). As V=0, the first VIS image is equal to VIS1. Thereafter, the first NIR image and the first VIS image are overlaid.

To form a second overlaid image, the NIR4, NIR5, and NIR 6 frames are corrected by subtracting VIS2 frame as the correcting frame, wherein the second NIR or IR image=(NIR4−VIS2)+(NIR5−VIS2)+(NIR6−VIS2). A second VIS image is VIS2, whereafter the second NIR or IR image and the second VIS image are overlaid to form the second overlaid image. This example shows correction of the NIR or IR frames in a sequence by subtraction of the VIS frame from the same sequence.

It is understood that the sequences described herein are obtained in a continuum throughout the imaging process. It is also understood that the first frame in a sequence can be an NIR frame or a VIS frame, and that one or more VIS frames may be present in any order within a sequence by applying these concepts. Other aspects are exemplified in FIGS. 30-37 and described herein.

### Example 3. Example of Creating an Overlay Image-Second Example of Forming First and Second Overlaid Images

In the example is provided per Table 3 below of a method of forming an overlaid image, wherein the primary quantity (N+1) is 2, wherein N=1, and wherein V=1.

TABLE 3

| | Sequence | | | Sequence | | | Sequence | | |
|---|---|---|---|---|---|---|---|---|---|
| Frame Name | VIS1 | NIR1 | NIR2 | VIS2 | NIR3 | NIR4 | VIS3 | NIR5 | NIR6 |
| Frame # | 1 | 2 | 3 | 5 | 6 | 7 | 9 | 10 | 11 |

The NIR or IR frames (NIR1, NIR2) are corrected by subtracting the subsequent Visible frame from the next Sequence (VIS2) to yield the following corrected NIR or IR frames: NIR1−VIS2; NIR2−VIS2. As N=1, the first NIR or IR image is generated by the sum of the first 2 corrected NIR or IR frames, the first NIR or IR image=(NIR1−VIS2)+(NIR2−VIS2). As V=1, the first VIS image is equal to VIS1+VIS2. Thereafter, the first NIR or IR image and the first VIS image are overlaid.

To form a second overlaid image, the NIR or IR frames (NIR3, NIR4), are corrected by subtracting the Visible frame from the subsequent sequence (VIS3), wherein the second NIR or IR image=(NIR3−VIS3)+(NIR4−VIS3). A second VIS image is formed by adding VIS2 and VIS3, whereafter the second NIR or IR image and the second VIS image are overlaid to form the second overlaid image. This example shows correction of the NIR or IR frames in a sequence by subtraction of the VIS frame from a subsequent sequence. It is understood that the sequences described herein are obtained in a continuum throughout the imaging process. It is also understood that the first frame in a sequence can be an NIR frame or a VIS frame, and that one or more VIS frames may be present in in any order within a sequence by applying these concepts. Other aspects are exemplified in FIGS. 30-37 and described herein.

### Example 4. Example of Creating an Overlay Image-Third Example of Forming First and Second Overlaid Images

In the example is provided per Table 3 below of a method of forming an overlaid image, wherein the primary quantity is 2, wherein N=2, and wherein V=2.

TABLE 3

| | Sequence | | | Sequence | | | Sequence | | |
|---|---|---|---|---|---|---|---|---|---|
| Frame Name | VIS1 | NIR1 | NIR2 | VIS2 | NIR3 | NIR4 | VIS3 | NIR5 | NIR6 |
| Frame # | 1 | 2 | 3 | 5 | 6 | 7 | 9 | 10 | 11 |

The NIR or IR frames (NIR1, NIR2, NIR3) are corrected by subtracting a nearest correcting VIS frame to yield the following corrected NIR or IR frames: NIR1−VIS1; NIR2-VIS2; and NIR3−VIS2. As N=2, the first NIR or IR image is generated by the sum of the first 3 corrected NIR or IR frames, the first NIR or IR image=(NIR1−VIS1)+(NIR2−VIS2)+(NIR3-VIS2). As V=2, the first VIS image is equal to VIS1+VIS2. Thereafter, the first NIR or IR image and the first VIS image are overlaid. This example shows correction of NIR or IR frames by subtraction of the VIS frame that is temporally closest or the nearest neighbor to the given NIR or IR frame. It is understood that the VIS frame subtracted that is temporally closest or the nearest neighbor to the NIR or IR frame, can be previous, current (i.e., same) or subsequent (i.e., future) sequence to the NIR or IR frame from which it is subtracted.

It is understood that the sequences described herein are obtained in a continuum throughout the imaging process. It is also understood that the first frame in a sequence can be an NIR frame or a VIS frame, and that one or more VIS frames may be present in in any order within a sequence by applying these concepts. Other aspects are exemplified in FIGS. 30-37 and described herein.

### Example 5. Use of System for Angiography in Repair of CNS Vascular Defects

This example describes using the imaging systems and methods herein for the imaging, detection, monitoring, diagnosis or treatment of vascular defects (e.g., arteriovenous malformation, cavernous malformation, intracranial aneurysm) in a subject, comprising any contrast or imaging agents including an indocyanine green (ICG) or fluorescein alone or in conjunction with a peptide or active agent. The agent is administered to a subject. The subject is a human or an animal and has a vascular defect. Administration is intravenous, subcutaneous, intranasal, oral, intraperitoneal, intramuscular, or intradermal. Upon administration, the agent is targeted to vascular tissues and cells thereof or is selectively retained within the blood. The agent is then visualized using the imaging system, in conjunction with a neurosurgical operating microscope, a neuroendoscope, a vascular endoscope, or as an open imaging system. The selection of the appropriate imaging system is made by the surgeon and is dependent on the size and location of the vascular defect as well as surgical approach. The surgeon exposes the defect, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 6. Use of System for Angiography in Blood or Lymph and Arteriography

This example describes use of the imaging system and/or method disclosed herein for coaxial illumination and visualization of blood or lymph in a subject. The imaging system of the present invention is used to image vascular or lymph vessels to image, monitor, diagnose, or guide treatment of disease. Surgery is performed to remove or bypass occlusions, repair vascular defects, provide for lymphatic drainage into the circulatory system to treat lymphedema, or to remove cancer or other abnormal tissue, such as endometriosis, from the subject. A contrast or imaging agent, including an indocyanine green (ICG) or fluorescein alone or in conjunction with a peptide or active agent, is administered to a subject. The subject is a human or an animal and has an occlusion that necessitates removal or bypass, or a tumor or other abnormal tissue that requires removal. Administration is intravenous, subcutaneous, intranasal, oral, intraperitoneal, intramuscular, intradermal, or by intratumoral injection. Upon administration, the agent is targeted to vascular tissues and cells thereof, lymphatic tissues and cells thereof, tumor or other abnormal vasculature, or is selectively retained within the blood or lymph. The agent is then visualized using the imaging system, in conjunction with a neurosurgical operating microscope, a neuroendoscope, a vascular endoscope, an endoscope, thoracoscope, telescope, robotic surgical system, other surgical microscope, or as an open imaging system. The selection of the appropriate imaging system is made by the surgeon and is dependent on the surgical approach. The surgeon exposes the occlusion, cancer, or other tissue, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 7. Use of System for Angiography in the Eye

This example describes using the imaging systems and methods herein for the imaging, detection, monitoring, diagnosis or treatment of disease, injury, or malformation of ocular structures (e.g., diabetic macular edema, diabetic macular ischemia, diabetic retinopathy, macular degeneration, retinal artery occlusion, retinal vein occlusion, Susac's syndrome, glaucoma, retinal detachment) in a subject, comprising any contrast or imaging agents including an indocyanine green (ICG) or fluorescein alone or in conjunction with a peptide or active agent. The agent is administered to a subject. The subject is a human or an animal and has a disease, injury, or malformation of ocular structures. Administration is intravenous, subcutaneous, intranasal, oral, intraperitoneal, intramuscular, intra-ocular, topical, or intradermal. Upon administration, the agent is targeted to vascular tissues and cells thereof or is selectively retained within the blood. The agent is then visualized using the imaging system, in conjunction with an ophthalmoscope, retinal or fundus camera system, optical coherence tomography (OCT) system, surgical microscope, or other ophthalmic imaging system. Ophthalmic angiogram of the choroid may similarly utilize the imaging system and/or methods disclosed herein. The imaging system enables the operator to view fluorescence and visible imaging together and simultaneously with the operating microscope or other imaging system. There is no need to reposition the operating microscope or other imaging system to view the fluorescence and visible images thus providing color imaging of the ocular structures together with the fluorescence imaging, which decreases disruption to the surgical or diagnostic workflow. Other contrast or imaging agents can be used as described herein.

### Example 8. Use of System for Perfusion Imaging in Surgery

This example describes use of the imaging system and/or method disclosed herein for coaxial illumination and visualization of tissue perfusion in a subject. The imaging system of the present invention is used to image blood flow in tissues during surgeries requiring adequate perfusion to promote healing of joined tissues (e.g., anastomosis, reconstructive surgery, or plastic surgery). A contrast or imaging agent, including an indocyanine green (ICG) or fluorescein alone or in conjunction with a peptide or active agent, is administered to a subject. The subject is a human or an animal and has a condition such as occlusion, cancer, or trauma. Administration is intravenous, subcutaneous, intranasal, oral, intraperitoneal, intramuscular, or intradermal. Upon administration, the agent is targeted to vascular tissues and cells thereof, or is selectively retained within the blood or lymph. The agent is then visualized using the imaging system, in conjunction with a neurosurgical operating microscope, a neuroendoscope, a vascular endoscope, an endoscope, thoracoscope, telescope, robotic surgical system, other surgical microscope, or as an open imaging system. The selection of the appropriate imaging system is made by the surgeon and is dependent on the surgical approach. The surgeon exposes the occlusion, cancer, or other tissue, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 9. Use of System for Detection of Plaque Instability and Restenosis in Atherosclerosis

This example describes use of the imaging system and/or method disclosed herein for coaxial illumination and visualization of atherosclerotic plaques and restenosis in a subject. The imaging system of the present invention is used to image atherosclerotic plaques within blood vessels in order to assess their stability, and to image blood flow through stented blood vessels for diagnosis of restenosis. A contrast or imaging agent, including an indocyanine green (ICG) or fluorescein alone or in conjunction with a peptide or active agent, is administered to a subject. The subject is a human or an animal and has atherosclerosis. Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with an endovascular endoscope, a vascular endoscope, an endoscope, thoracoscope, telescope, robotic surgical system, other surgical microscope, or as an open imaging system. The selection of the appropriate imaging system is made by the surgeon and is dependent on the surgical or diagnostic approach. The surgeon exposes the plaque, stent, or other tissue, and the imaging system is initialized and used continuously. The imaging system enables the surgeon to view fluorescence and visible imaging together and simultaneously with the operating microscope or other imaging system. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 10. Use of System for Imaging Vital Organs or Structures

This example describes use of the imaging system and/or method disclosed herein for imaging vital organs or structures in a subject during surgery. The imaging system of the present invention is used to image contrast between vital organs or structures (e.g., kidney, ureters, thyroid, liver or liver segments, nerves) and other surrounding tissues. A contrast or imaging agent, including an indocyanine green (ICG), methylene blue, or fluorescein, alone or in conjunction with a peptide or active agent, is administered to a subject. The subject is a human or an animal and has a disease or condition that requires surgical intervention near vital organs or structures. Administration is intravenous, subcutaneous, intranasal, oral, intraperitoneal, intramuscular, intradermal, or by intratumor injection. Upon administration, the agent is targeted to vascular tissues and cells thereof, to vital organ tissues and cells thereof (e.g., nerves), or is selectively retained within the blood. The agent is then visualized using the imaging system, in conjunction with a laparoscope, a vascular endoscope, an endoscope, thoracoscope, telescope, robotic surgical system, other surgical microscope, or as an open imaging system. The selection of the appropriate imaging system is made by the surgeon and is dependent on the surgical or diagnostic approach. The surgeon exposes the area of interest, and the imaging system is initialized and used continuously. Contrast results either from differential blood flow to the organ or tissue (e.g., kidney contrasting with adrenal gland, thyroid contrasting with parathyroid, or liver segment following selective injection to an artery supplying the segment), from elimination pathways (e.g. ureters or kidney following administration of a dye or conjugate with renal clearance), or from selective targeting to the organ or structure (e.g., using a peptide that targets proteins found on nerve sheaths). The imaging system enables the surgeon to view fluorescence and visible imaging together and simultaneously with the operating microscope or other imaging system. The contrast enables the surgeon to avoid injury to normal tissues and to selectively remove organs, organ segments, or other tissues as appropriate. There is no need to reposition the operating microscope or other imaging system to view the fluorescence and visible images thus providing imaging of the surgical area together with the fluorescence imaging system during the operation, which decreases disruption to the surgical workflow. Other contrast or imaging agents can be used as described herein.

### Example 11. Use of System for Diagnosis of Ischemia

This example describes use of the imaging system and/or method disclosed herein for imaging and diagnosis of tissue ischemia. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence of fluorescence signal from the tissue of interest indicates reduced or absent blood flow, and ischemia. Other contrast or imaging agents can be used as described herein.

### Example 12. Use of System During Venography

This example describes use of the imaging system and/or method disclosed herein for imaging of veins and diagnosis of deep vein thrombosis (DVT) or other vein abnormalities. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and DVT or other vein abnormalities. Other contrast or imaging agents can be used as described herein.

### Example 13. Use of System for to Monitor Cerebral Vascular Flow

This example describes use of the imaging system and/or method disclosed herein for imaging and diagnosis of vessel narrowing (stenosis), clot formation (thrombosis), blockage (embolism) or blood vessel rupture (hemorrhage) in the brain. Lack of sufficient blood flow (ischemia) affects brain tissue and may cause a stroke. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia. Other contrast or imaging agents can be used as described herein.

### Example 14. Use of System to Image and Monitor Vascular Flow to Tumors

This example describes use of the imaging system and/or method disclosed herein for imaging of tumor vasculature for monitoring, diagnosis and treatment of tumors. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Presence of enhanced and abnormal fluorescence signal from the tissue of interest indicates angiogenesis, or stimulation of blood vessel growth indicative of tumors. Other contrast or imaging agents can be used as described herein.

### Example 15. Use of System for Coronary Angiography, Angiogram and Cardiac Catheterization

During coronary angiography, a contrast or imaging dye is injected into a subject artery through a catheter or other. Using the system and methods herein blood flow is monitored through the subject's heart. This test is also known as a cardiac angiogram, catheter arteriography, or cardiac catheterization. This example describes use of the imaging system and/or method disclosed herein for imaging of heart vasculature. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has known or suspected coronary artery disease. Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia. Catheterization, angioplasty, plaque ablation, stent insertion or replacement, or other treatment can accompany the imaging. Other contrast or imaging agents can be used as described herein.

Similarly, during a coronary angiogram, a contrast or imaging dye is injected into the blood vessels of the heart. Using the system and methods herein blood flow is monitored through the subject's heart. The system is used to take a series of images (angiograms), to visualize the cardiovasculature and blood vessels feeding blood to the heart. Concurrent with imaging and monitoring of vessels using this method, clogged heart arteries can be opened (angioplasty) during the coronary angiogram. Coronary computed tomography angiography (CCTA) can also similarly employed.

### Example 16. Use of System for Imaging and Monitoring Stroke, Coronary Artery Disease or Congestive Heart Failure

This example describes use of the imaging system and/or method disclosed herein for imaging and diagnosis of stroke, coronary artery disease or congestive heart failure, or in cardiography. Lack of sufficient blood flow (ischemia) affects brain tissue and may cause a stroke. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia indicative of stroke, coronary artery disease or congestive heart failure. Other contrast or imaging agents can be used as described herein.

### Example 17. Use of System During Aneurysm, Arteriovenous Malformation, or Cavernous Malformation, Resection

This example describes use of the imaging system and/or methods disclosed herein for coaxial illumination and visualization of tozuleristide fluorescence during surgical resection of an arteriovenous malformation or cavernous malformation in a pediatric patient. The imaging system of the present invention was used to image brain tissue to detect abnormal vasculature using fluorescence imaging. Surgery was performed to remove the abnormality from the subject.

A pediatric subject with a history of anosmia was found on MRI to have a 3.5 cm T1-hypointense, T2/FLAIR-hyperintense mass in the right middle frontal gyrus with a central enhancing nodule, initially diagnosed pre-operatively to be a low-grade glioma. The subject did not have any prior history of neurosurgery. The patient received 22 mg tozuleristide via IV injection approximately 5-6 hours before surgery and image collection. The imaging system head was attached to the Zeiss Pentero surgical microscope along with two eyepieces prior the start of surgery.

After the lesion was exposed, the imaging system was initialized and used continuously. The imaging system enabled the surgeon to view fluorescence and visible imaging together and simultaneously with the operating microscope. A microsurgical resection was performed through a right frontal craniotomy. The abnormal tissue had a dark blue mulberry appearance that fluoresced avidly with tozuleristide. The surrounding tissue showed no fluorescence. The abnormal tissue was completely resected. The patient recovered without deficit. Pathology confirmed the absence of tumor. Pathology data suggested the abnormality was vascular in nature and demonstrated that tozuleristide fluorescence was detected in a cerebral non-tumoral lesion. While ICG alone transiently lights up blood vessels immediately after injection, tozuleristide fluorescence is dependent on pathological tissue binding and uptake and can highlight diseased tissue up to 30 hours after injection.

Video was captured for the duration of the resection, and still images were captured of the exposed lesion. Tozuleristide fluorescence was observed in situ in the exposed vascular lesion. FIGS. 29A-I show images taken from the vascular lesion with the near-infrared (NIR) fluorescence images of the vascular lesion using the imaging system. The vascular abnormality appeared to the surgeon as a bright green mass (arrows labeled “VL”) in the NIR or IR fluorescence image and in the overlay image (shown as a bright white mass in grey-scale), while the normal brain tissue (labeled “NB”) and vasculature (labeled “BV”) appeared darker than the vascular lesion in the NIR or IR fluorescence image indicating no discernable background fluorescence in non-lesion or normal brain tissue or in normal vasculature. In the overlay image, the normal brain tissue and normal blood vessels appeared pink or light tan to red, as it does under normal visible light or white light. The surgeon noted that only the abnormal vascular tissue appeared fluorescent. The fluorescent tissue samples were demonstrated and confirmed to be non-cancerous and vascular in nature by histopathology.

FIGS. 29A-I show representative images of in situ or intra-operative tissue during surgery on a vascular lesion in a patient, wherein 22 mg of tozuleristide was administered to the human subject. FIG. 29D shows a near-infrared (NIR) image of the in situ specimen. Fluorescence signal, corresponding to lighter and brighter areas in the NIR or IR images, is indicative of the presence of tozuleristide in the vascular lesion. Labeled arrows indicate non-fluorescent regions of normal blood vessels (“BV”) and normal brain tissue (“NB”). In contrast, fluorescence signal corresponding to lighter and brighter areas in the NIR or IR image, was indicative of the presence of tozuleristide on the abnormal vascular lesion (“VL”), and not in normal tissue. FIG. 29E shows the white light image corresponding to FIG. 29D that represents what the surgeon would normally see without fluorescence guidance. The arrows mark the same locations as shown in the NIR or IR image in FIG. 29E. The vascular lesion (“VL”) had a similar appearance to the normal blood vessels (“BV”) in this image. FIG. 29F shows the NIR or IR fluorescence and white light composite image of FIG. 29E and FIG. 29D, with arrows marking the same locations as shown in FIG. 29E and FIG. 29D. Fluorescence in the vascular lesion (“VL”) clearly differentiated it from the surrounding normal tissues, including normal blood vessels (“BV”). FIG. 29G shows a near-infrared (NIR) image of the vascular lesion during the surgery. Arrows indicate the vascular lesion (labeled “VL”) and adjacent normal brain (labeled “NB”), which is non-fluorescent. FIG. 29H shows the white light image corresponding to FIG. 29G. While the normal brain has a light tan to pink color (light gray in a gray scale image), it is perfused with normal blood vessels that can be differentiated from the vascular lesion by the absence of fluorescence. FIG. 29I shows the composite white light and NIR or IR image shown in FIG. 29G and FIG. 29H.

The fluorescent tissue samples were demonstrated and confirmed to be non-cancerous and vascular in nature by histopathology. The pathology did not indicate cancer or neoplastic abnormalities but rather confirmed a vascular abnormality that did not indicate cancer. Intraoperative pathology was performed on two specimens. One showed dilated vessels most compatible with vascular malformation, and the other was normal brain parenchyma with no evidence of neoplasm. Post-operative pathology was performed on 19 excised specimens with the numbered annotations for reference are shown below in TABLE 3.

TABLE 3
Pathology Specimens

| Specimen | | |
|---|---|---|
| Number | Description | Observation |
| 1 | Right brain specimen 1 | Blood vessels with hyalinization |
| | (Excision) | and chronic inflammation |
| 2 | Right brain specimen 2 | Gray and white matter and blood |
| | (Excision) | vessels with chronic inflammation, |
| | | and microcalcifications |
| 3 | Right brain specimen 3 | Blood vessels, interspersed neuropil, |
| | (Excision) | chronic inflammation, and |
| | | calcifications |
| 4 | Right brain specimen 4 | Blood vessels with chronic |
| | (Excision) | inflammation and interspersed gray |
| | | matter |
| 5 | Right brain specimen 5 | Blood vessels with interspersed gray |
| | (Excision) | and white matter and proteinaceous |
| | | aggregates |
| 6 | Right brain specimen 6 | Minute cluster of blood vessels |
| | (Excision) | |
| 7 | Deep lateral equivocal | Minute fragment of white matter |
| | tissue specimen 7 | |
| | (Excision) | |
| 8 | Posterior equivocal tissue | Gray matter with focus of blood |
| | specimen 8 (Excision) | vessels |
| 9 | Inferior equivocal tissue | Minute fragment of gray matter |
| | specimen 9 (Excision) | |
| 10 | Anterior equivocal tissue | Minute fragment of gray and white |
| | specimen 10 (Excision) | matter |
| 11 | Anterior lateral equivocal | Minute fragments of gray and white |
| | tissue specimen 11 | matter with focus of blood vessels |
| | (Excision) | |
| 12 | Anterior equivocal tissue | Minute fragment of gray matter |
| | specimen 12 (Excision) | |
| 13 | Posterior equivocal tissue | Minute fragments of gray and white |
| | specimen 13 (Excision) | matter |
| 14 | Deep lateral equivocal | Minute fragment of white matter |
| | tissue specimen 14 | |
| | (Excision) | |
| 15 | Anterior equivocal tissue | Minute fragments of gray and white |
| | specimen 15 (Excision) | matter |
| 16 | Inferior equivocal tissue | Minute fragments of gray matter |
| | specimen 16 (Excision) | with reactive changes |
| 17 | Anterior lateral equivocal | Small fragments of gray and white |
| | tissue specimen 17 | matter |
| | (Excision) | |
| 18 | Right deep specimen 18 | Small fragments of gray matter |
| | (Excision) | with reactive surgical changes |
| 19 | Right deep specimen 19 | Small fragment of gray and white |
| | (Excision) | matter |

Specimens with substantial vascular components were considered for examination. In specimen 8 the vessels were not separated by neuropil. In specimens 3, 4, and 5, neuropil intervened between the vessels, indicating an overall diagnosis of vascular malformation.

This case demonstrated that the imaging system could be used continuously in an intraoperative setting to capture images and video of white light and NIR or IR fluorescence, without disrupting the normal surgical flow. The data further demonstrated that the coaxial illumination and imaging system enabled the surgeon to visualize and precisely localize fluorescence in non-neoplastic pathologies during surgery and use this information to remove abnormal vascular tissue during resection. The systems and methods herein can be used to detect, image and treat cavernous malformation, cavernous angiomas, cavernous hemangiomas, or cerebral cavernous malformation (CCM), and arteriovenous malformation, including via surgery.

### Example 18. Use of System for Imaging and Monitoring Occlusion of Veins or Arteries Resulting in Organ Failure or Injury

This example describes use of the imaging system and/or methods disclosed herein for imaging and diagnosis of occlusion of arteries or veins or detection of hemorrhage or embolism in a variety of organ systems, including brain, heart, lung, kidney, liver, pancreas, or in the extremities (e.g., legs, neck, and arms). Lack of sufficient blood flow (ischemia) affects tissue and may cause organ damage or organ failure, hemorrhagic stroke, and the like. The imaging system of the present invention is used to image blood flow in a subject. A contrast or imaging agent, including an indocyanine green (ICG) or fluorescein alone or in conjunction with a peptide or active agent, is administered to a subject. The subject is a human or an animal and has a chronic wound or suspected ischemia (e.g. in extremities or limb). Administration is intravenous, subcutaneous, intranasal, oral, intraperitoneal, intramuscular, or intradermal. Upon administration, the agent is targeted to vascular tissues and cells thereof, or is selectively retained within the blood. The agent is then visualized using the imaging system, in conjunction with a surgical microscope, other imaging system, or as an open imaging system. Absence, blockage or hemorrhage of fluorescence signal from the tissue of interest indicates reduced or absent blood flow, and ischemia indicative of organ damage or organ failure, hemorrhagic stroke, and the like. Other contrast or imaging agents can be used as described herein.

### Example 19. NIR or IR and VIS Acquisition

As shown in FIG. 30, NIR or IR and VIS images acquired during one laser cycle are referred to as a “Sequence,” wherein each sequence comprises at least one VIS_DRK frame and one NIR or IR frame. In some embodiments, the raw NIR or IR frame comprises visible light reflection from the target, ambient NIR or IR from the environment, and fluorescence from the target fluorophore, wherein the VIS_DRK frame comprises the visible light reflection from the target and ambient NIR or IR from the environment and any NIR or IR fluorescence excited by the ambient light. In each sequence, a ratio between the number of NIR or IR frames and the number of VIS_DRK frames is defined as an NIR-ratio: VISratio. In the example shown in FIG. 30, the NIR-ratio: VISratio=2. In some examples, an exposure time for the VIS_DRK frame is equal to an exposure time of the NIR or IR frame. In some examples, an exposure time for the VIS_DRK frame (DRK-exp) is unequal to an exposure time of the NIR or IR frame (NIRexp). In some cases, the ratio of NIRexp to DRK-exp is an integer greater than 1. In other cases the ratio of NIRexp to DRK-exp is a fraction greater than 1.

### Example 20. NIR Correction

Subtracting the VIS_DRK frame from the raw NIR or IR frame yields an image containing only the fluorescence from the fluorophore. If the exposure ratio between the NIRexp and the DRKexp (EXP_RATIONIR-DRK) is not one, then a dark-corrected NIR frame (NIR*) is equal to the NIR or IR frame minus the VIS_DRK frame times the exposure ratio (EXP_RATIONIR-DRK). (EXP_RATIONIR-DRK) may be scalar or non-scalar. EXP_RATIONIR-DRK may be a mapping or any other function that maps the VIS_DRK frame exposure to the NIR or IR frame exposure. As further described herein, the following formula also describes this relationship: NIR*=NIR-(DRK*×EXP_RATIONIR-DRK). The Overlay image (OVL) is the sum of the VIS and NIR* images.

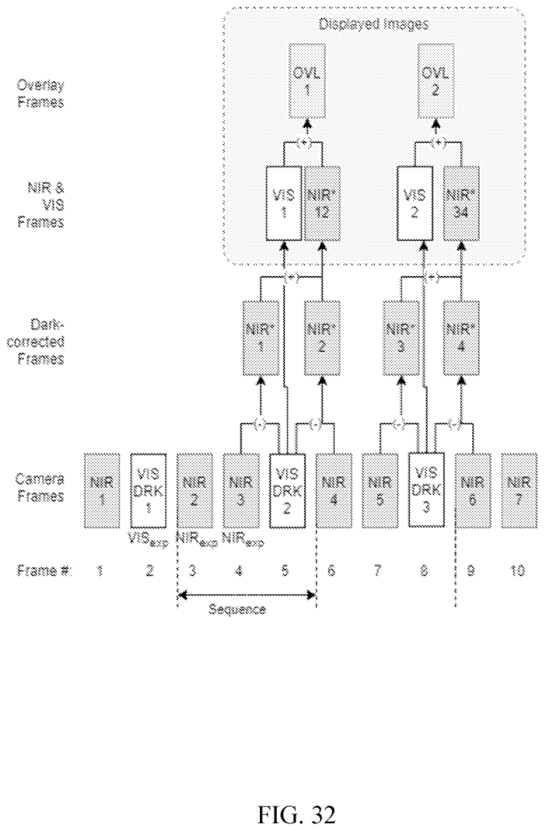

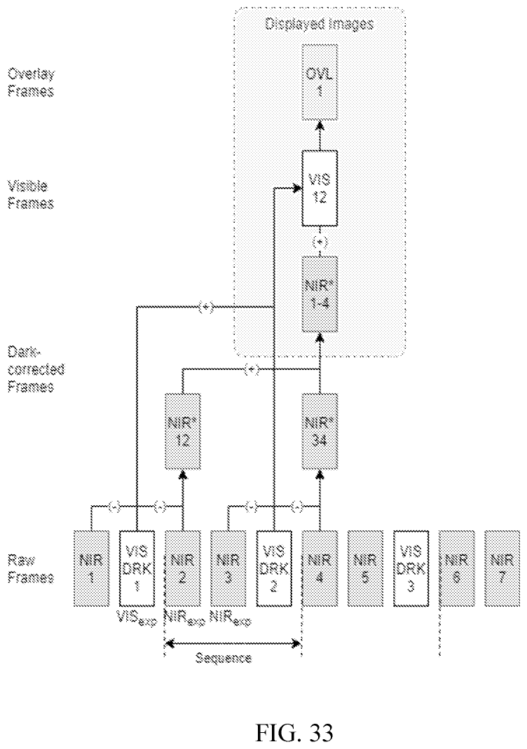

When the ratio of NIR or IR frames to VIS_DRK frames in a sequence is greater than 1 (RATIOnir-vis>1), there are several alternatives for the VIS_DRK frame to use for subtraction to generate the dark-corrected NIR frame (NIR*). As shown in FIG. 31, the VIS_DRK frame in the same sequence may be used. Alternatively, the VIS_DRK frame in the following sequence may be used. Alternatively, as shown in FIG. 32 and FIG. 33, the VIS_DRK frame that is temporally closest to the given NIR frame may be used, whether or not it is in the same, previous, or the subsequent sequence. Selecting the VIS_DRK frame that is temporally closest to the given NIR frame reduces the subtraction-related motion artifact, relative to approaches that subtract VIS_DRK frames that are temporally more distant from the given NIR frame. It is understood that subtraction of the VIS_DRK frame that is temporally closest to the given NIR frame, regardless of whether it is in the previous, current (i.e., same), or subsequent (i.e., future) sequence offers advantages in reducing or minimizing, or correcting the subtraction-related motion artifact.

### Example 21. Summing NIR or IR and VIS Frames

When the acquisition sequence has more than one VIS_DRK and/or NIR* frame in the sequence, multiple frames are summed to increase sensitivity. Subtracting the VIS_DRK frame (i.e., “DRK” frame for this purpose) from the raw NIR frame yields an image containing only the fluorescence from the fluorophore. This image is referred to as the dark-corrected NIR image (NIR*). Exemplary acquisition sequences utilize a NIR:VIS_DRK ratio that is greater than 1 to allow summing the NIR data to enhance sensitivity to the NIR fluorescence signal. In these cases, certain VIS_DRK frames are used for the NIR dark correction. In the simplest model, the VIS_DRK frame is from the same acquisition sequence. As shown in FIG. 32, using the nearest neighbor approach, the NIR-VISratio is 2, whereas two NIR or IR frames are summed with the original or primary NIR frame and 1 VIS_DRK frame is summed with the original or primary VIS_DRK frame for each sequence. As such, each dark-corrected NIR frame has an effective exposure equal to that of two raw NIR frames (e.g. NIR*12=NIR*1+NIR*2).

As shown in FIG. 32, the NIR: VIS_DRK ratio is 2, whereas 2 corrected NIR or IR frames are summed for every 1 VIS_DRK frames that is used to create the images. As such the effective exposure for the NIR* image is tow times the NIRexp and the effective VIS exposure is the VISexp.

As shown in FIG. 33, image calculations can be repeated for subsequent display images, whereas display update frequency will be half the sequence frequency, two sequences are required to accumulate all the NIR and VIS images.

In some embodiments, the number of NIR or IR frames or VIS_DRK frames required to form an image may be greater than the number available in a sequence. In these cases, frames may be acquired until the required count of frames is obtained. Then, an image for display may be generated using the techniques previously described herein. Because this approach requires more than one sequence to acquire all the frames to generate an image, an image is displayed less frequently than sequences are acquired. To prevent this reduction of the display frequency, as exemplified in FIG. 34, the image made from a number of frames that exceed the number in the sequence are summed, using a ‘boxcar’ accumulator. As shown, the display frame rate is the same as the sequence rate, whereas the four NIR images in the first displayed NIR image (NIR*1-4) are accumulated from NIR*1 through NIR*4. After this image, two more NIR frames (NIR*56) are added to the boxcar accumulator, pushing out the first two NIR frames (NIR*12). This allows a new NIR frame that is the sum of 4 raw frames to be displayed with every sequence, as opposed to every other sequence. The same approach is used to update the VIS image displayed, which is the sum of two raw VIS_DRK frames. Thus, in this embodiment, an image for display is generated every sequence, even though the number of frames required to generate the image exceeds the count available in a single sequence. If the boxcar accumulation approach described herein and shown in FIG. 34 were not used, an image for display would only be generated after the number of sequences required to acquire the required NIR or IR or VIS_DRK frames, leading to a slower display rate.

### Example 22. Additional Methods of Generating Corrected NIR Images

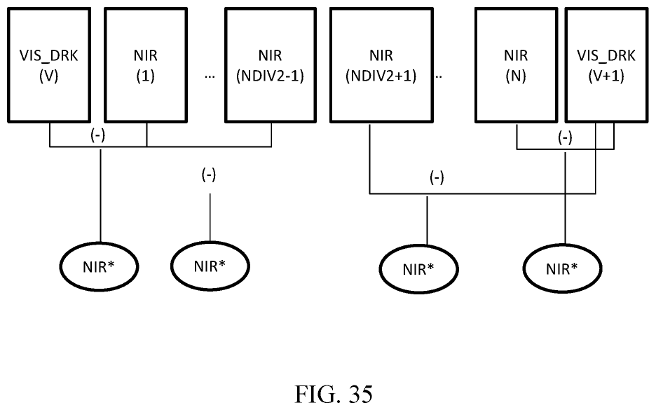

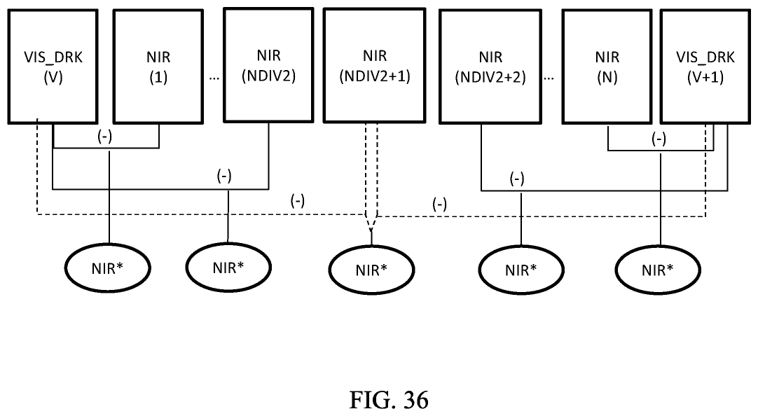

Another example of generating corrected NIR images is shown in FIGS. 35 and 36. In this example there may be several NIR frames within a sequence such that the ratio of NIR frames to VIS_DRK frames in each sequence is greater than or equal to 3. In this example: “N” refers to the number of NIR frames between VIS_DRK frames (i.e. the number of NIR frames in a laser on/off sequence); “V” is the number of VIS_DRK frames obtained; and “DIV” refers to an in integer division function used to identify an approximately equal distribution of whole NIR frames within the sequence.

As shown in FIG. 35, N is an even number such that an equal number of NIR frames may be classified or identified as being temporally closer to each VIS_DRK frame. In this example, an even number of corrected NIR images (NIR*) may be obtained. A first VIS_DRK frame “VIS_DRK (V)” is subtracted from each NIR frame that is temporally closer to VIS_DRK (V). As shown, these frames include NIR frames (1) through (N/2−1). Likewise, a second VIS_DRK frame “VIS_DRK (V+1)” is subtracted from each NIR frame that is temporally closer to VIS_DRK (V+1). As shown, this includes NIR frames (N/2+1) through (N). The resulting corrected images NIR* may optionally be further processed to further reduce or eliminate motion artifacts, if present.

Alternatively, as shown in FIG. 36, Nis an odd number such that an equal number of NIR frames may be classified or identified as being temporally closer to each VIS_DRK frame and an additional NIR frame may be corrected using either VIS_DRK frame. In this example, an odd number of corrected NIR images (NIR*) may be obtained. A first VIS_DRK frame “VIS_DRK (V)” is subtracted from each NIR frame that is temporally closer to VIS_DRK (V). As shown, these frames include NIR frames (1) through (N/2). Likewise, a second VIS_DRK frame “VIS_DRK (V+1)” is subtracted from each NIR frame that is temporally closer to VIS_DRK (V+1). These frames include As shown, this includes NIR frames (N/2+2) through (N). The remaining NIR frame NIR (N/2+1) may be processed using one or bother of VIS_DRK (V) or VIS_DRK (V+1). The resulting corrected images NIR* may optionally be further processed to further reduce or eliminate motion artifacts, if present.

### Example 23. Multispectral Cameras

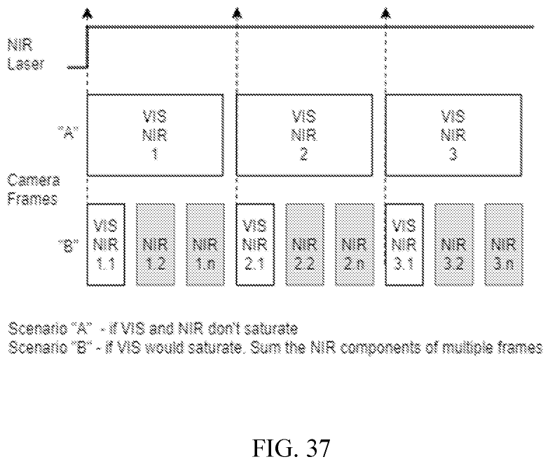

The pixels of some cameras simultaneously image visible and NIR or IR wavelengths. For example, such pixel sensors have arrays of four pixels, for example, where each pixel detects different parts of the VIS-NIR optical spectrum (e.g. red, green, blue, and NIR or IR wavelengths), whereas the visible image is created from the output of each of the four pixels. Thus, for each frame, the camera captures a VIS frame and a NIR or IR frame. In a scenario A neither of the VIS nor NIR frames are saturated at the frame exposure time, and whereas if both the VIS and NIR images are saturated, the exposure time is reduced until they are not saturated. However, if only one frame (VIS or NIR) is saturated, a scenario B is used, whereas the frame exposure time of the saturated frame is reduced so that neither frame is saturated. As shown in FIG. 37, the VIS frame is saturated but the NIR frame is not. As such, the frame exposure is reduced until the VIS frame was not saturated. Then, to maintain NIR sensitivity, the NIR data from several frames is summed to create the NIR image. As shown, the visible image is the VIS component of the first frame (VIS NIR 1.1) and the NIR image is the sum of the NIR components of several frames (e.g. 1.1, 1.2 . . . 1.n). The NIR sensitivity may be adjusted by summing fewer/more NIR frames. As the laser is on continuously for such a scenario, however, there is no way to subtract ambient NIR from the NIR image.

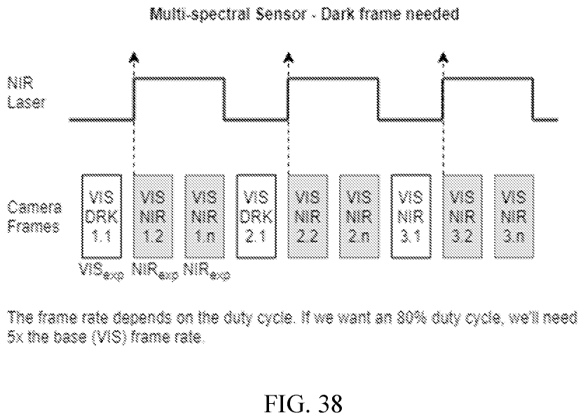

To eliminate ambient NIR, for example as shown in FIG. 38, the laser is modulated to create a VIS_DRK frame, wherein the laser is turned off for one (or more) frames. The NIR component of this frame(s) contains only the ambient NIR signal, and any fluorescence excited by the ambient light. Any dark correction scheme (e.g. sequence, nearest-neighbor) can be used to calculate the dark-corrected NIR frames (NIR*). Multiple NIR* frames can be summed to achieve the desired NIR sensitivity. Similarly, the VIS components of multiple frames can be summed to achieve the desired VIS image sensitivity.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure.

Although certain embodiments and examples are provided in the foregoing description, the inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures, systems, and/or devices described herein can be embodied as integrated components or as separate components.

1. An imaging system for imaging an emission light, the system comprising:
(a) an excitation channel to receive an excitation light;
(b) an excitation diffuser that diffuses the excitation light;
(c) a visible channel to receive and direct a visible light to a sample;
(d) an optical device directing the diffused excitation light to the sample and allowing the emission light and a reflected visible light to pass therethrough to an imaging assembly; and
(e) the imaging assembly comprising:
(i) a first notch filter;
(ii) a lens; and
(iii) an image sensor configured to detect both the emission light and the reflected visible light from the sample and configured to generate image frames based on the emission light and the reflected visible light.

2. The system of claim 1, further comprising a longpass filter or a second notch filter, or a longpass filter and a second notch filter, wherein the emission light and the reflected visible light are directed from the sample through a notch beam splitter, the first notch filter, the longpass filter, the lens, and/or the second notch filter, or combination of one or more of the foregoing in any order.

3. (canceled)

4. (canceled)

5. The system of claim 1, wherein the excitation light has a wavelength of about 650 nm to about 1000 nm, 700 nm to about 800 nm, about 800 nm to about 950 nm, about 775 nm to about 795 nm, or about 785 nm, or any combination of the foregoing and the visible light has a wavelength of about 400 nm to about 800 nm.

6. The system of claim 1, wherein the emission light is emitted by a fluorophore within the sample and wherein the sample comprises at least one of a tissue, a physiological structure, or an organ.

7. (canceled)

8. (canceled)

9. (canceled)

10. The system of claim 1, wherein the excitation diffuser is a circular excitation diffuser having a diffusion angle of about 4 degrees to about 25 degrees or a rectangular diffuser having a first diffusion angle of about 4 degrees to about 25 degrees and a second diffusion angle of about 4 degrees to about 25 degrees perpendicular to the first diffusion angle.

11. (canceled)

12. (canceled)

13. (canceled)

14. (canceled)

15. (canceled)

16. (canceled)

17. (canceled)

18. The system of claim 1, wherein the optical device is a hot mirror, a dichroic mirror, a shortpass filter, or any combination thereof and the optical device directs the diffused excitation light to the sample in a first direction and allows the emission light and a reflected visible light to pass therethrough in a second direction opposite the first direction.

19. (canceled)

20. (canceled)

21. The system of claim 2, wherein at least one of the first notch filter or the second notch filter has a width greater than a spectral width of a source of excitation light and block or attenuate or inhibit or reduce the excitation light from passing therethrough.

22. (canceled)

23. (canceled)

24. (canceled)

25. (canceled)

26. (canceled)

27. (canceled)

28. The system of claim 1, further comprising a white light that emits the visible light and a polarizer within the imaging assembly.

29. The system of claim 1, further comprising a shortpass dichroic mirror between the imaging assembly and the sample and between the excitation diffuser and the sample, the shortpass dichroic mirror configured to transmit wavelengths less than the excitation wavelength and reflect wavelengths at the excitation wavelength or greater.

30. (canceled)

31. (canceled)

32. The system of claim 29, further comprising a window between the shortpass dichroic mirror and the sample and/or a window between the notch filter and the sample.

33. (canceled)

34. (canceled)

35. (canceled)

36. The system of claim 2, wherein the longpass filter comprises a visible light attenuator configured to transmit near infrared or infrared wavelengths.

37. (canceled)

38. The system of claim 1, further comprising a laser monitor sensor comprising:
(a) an excitation light power gauge configured to measure a power of the excitation light (excitation power); and
(b) a diffused beam shape sensor measuring a diffused beam shape comprising at least one diffused beam shape gauge.

39. The system of claim 38, further comprising a first diffused beam shape gauge, a second diffused beam shape gauge, at least one or more additional beam shape gauges, and a reflector positioned between the excitation channel and the excitation diffuser, wherein the reflector is configured to redirect a portion of the excitation light to the excitation light power gauge.

40. (canceled)

41. (canceled)

42. The system of claim 38, wherein the optical device allows a portion of the diffused excitation light to pass therethrough in a direction parallel to the diffused excitation light, and wherein the diffused beam shape sensor receives the portion of the diffused excitation light.

43. The system of claim 39, wherein the first diffused beam shape gauge measures a power of the diffused beam at a center of the diffused beam shape, wherein the second diffused beam shape gauge measures the power of the diffused beam at an edge of the diffused beam shape, and wherein the first diffused beam shape gauge, the second diffused beam shape gauge, the additional beam shape gauges, or any combination thereof are arranged in a one-dimensional array or a two dimensional array.

44. (canceled)

45. (canceled)

46. (canceled)

47. (canceled)

48. (canceled)

49. (canceled)

50. An imaging platform for imaging an emission light emitted by a fluorophore, the platform comprising:
(a) the imaging system of claim 1; and
(b) an imaging station comprising:
(i) a non-transitory computer-readable storage media encoded with a computer program including instructions executable by a processor to receive image frames from the image sensor via an imaging capable, a wireless connection, or both; and
(ii) an input device.

51. (canceled)

52. (canceled)

53. (canceled)

54. (canceled)

55. (canceled)

56. (canceled)

57. The platform of claim 50, further comprising a laser monitor sensor, wherein the platform further comprises a laser monitor electronics receiving data from the laser monitor sensor, and wherein the laser monitor electronics turns off the laser if:
(a) a measured power of the excitation light (excitation power) deviates from a set excitation light power by a first predetermined value;
(b) a diffused beam shape deviates from a set beam shape by a second predetermined value; or
(c) both.

58. The platform of claim 57, wherein the first predetermined value comprises excitation power as measured by a predetermined range value or a predetermined maximum magnitude of a rate of change value or both and wherein the second predetermined value determines that the diffused beam shape has deviated from the set beam shape based on the power of the diffused beam at at least one point along the diffused beam shape as measured by a first diffused beam shape gauge as compared to the excitation light power of the diffused beam at at least one other point along the diffused beam shape as measured by at least a second diffused beam shape gauge.

59. (canceled)

60. (canceled)

61. (canceled)

62. (canceled)

63. (canceled)

64. A method for imaging an emission light emitted by a fluorophore, the method comprising:
(a) emitting an excitation light;
(b) diffusing the excitation light;
(c) receiving and directing a visible light to a sample;
(d) directing the diffused excitation light to the sample;
(e) directing the emission light and a reflected visible light to an imaging assembly;
(f) filtering the excitation light and the reflected visible light from the emission light;
(g) detecting both the emission light and the reflected visible light from the sample to generate image frames based on the emission light and the reflected visible light.

65. The method of claim 64, wherein the fluorophore is within the sample and wherein the sample comprises at least one of a tissue, a physiological structure, or an organ.

66. (canceled)

67. The method of claim 64, wherein filtering the excitation light and the reflected visible light from the emission light comprises directing the emission light and the reflected visible light from the sample through a notch beam splitter, a first notch filter, a longpass filter, a lens, and a second notch filter or any combination of the foregoing.

68. (canceled)

69. (canceled)

70. (canceled)

71. The method of claim 64, wherein the visible light has a wavelength of about 400 nm to about 800 nm and the excitation light has a wavelength of about 775 nm to about 950 nm.

72. (canceled)

73. The method of claim 64, wherein the excitation light is diffused by a circular excitation diffuser having a diffusion angle of about 4 degrees to about 25 degrees or a rectangular diffuser having a first diffusion angle of about 4 degrees to about 25 degrees and a second diffusion angle of about 4 degrees to about 25 degrees perpendicular to the first diffusion angle.

74. (canceled)

75. (canceled)

76. (canceled)

77. (canceled)

78. (canceled)

79. The method of claim 64, wherein the diffused excitation light is directed to the sample and/or the imaging assembly by a hot mirror, a dichroic mirror, a shortpass filter, or any combination thereof, wherein the diffused excitation light is directed to the sample in a first direction, and wherein the emission light and the reflected visible light are directed in a second direction opposite the first direction.

80. (canceled)

81. (canceled)

82. (canceled)

83. The method of claim 64, wherein filtering the emission light and the reflected visible light comprises blocking light having a wavelength of about 775 nm to about 795 nm from passing therethrough.

84. (canceled)

85. The method of claim 64, further comprising:
(a) polarizing the emission light and the reflected visible light; and
(b) filtering the diffused excitation light by filtering out wavelengths less than about 720 nm, 725 nm, 730 nm, 735 nm, 740 nm, 750 nm, 755 nm, 760 nm, 770 nm, 780 nm, 900 nm, or more including increments therein.

86. (canceled)

87. (canceled)

88. (canceled)

89. The method of claim 64, further comprising monitoring the excitation light by:
(a) measuring a power of the excitation light with an excitation light monitor by receiving a redirected portion of the excitation light;
(b) measuring a diffused beam shape of the diffused excitation light with a sensor system; or
(c) both.

90. (canceled)

91. The method of claim 89, wherein a first diffused beam shape gauge measures a power of the diffused beam at a center of the diffused beam shape and wherein a second diffused beam shape gauge measures the power of the diffused beam at an edge of the diffused beam shape.

92. (canceled)

93. The method of claim 91, further comprising measuring the power of the diffused beam by 2, 3, 4, 5, 6, 7, 8, 9, 10, or more additional beam shape gauges, wherein the first beam gauge, the second beam gauge, the additional beam gauges, or any combination thereof are arranged in a one-dimensional array or a two-dimensional array.

94. (canceled)

95. (canceled)

96. (canceled)

97. (canceled)

98. The method of claim 64, further comprising turning off the excitation light if:
(a) a measured power of the excitation light deviates from a set excitation light power by a first predetermined value;
(b) a diffused beam shape deviates from a set beam shape by a second predetermined value; or
(c) both.

99. (canceled)

100. (canceled)

101. (canceled)

102. (canceled)

103. A computer-implemented method of forming a first overlaid image from laser induced fluorophore excitations, the method comprising:
(a) receiving a plurality of image frame sequences, each image frame sequence comprising:
(i) a VIS_DRK frame captured when the laser is in an off mode or in an on mode; and
(ii) a primary quantity of NIR or IR frames captured when the laser is in the on mode;
(b) correcting each NIR or IR frame by subtracting a correcting VIS_DRK frame; and
(c) generating a first NIR or IR image by adding a first corrected NIR or IR frame and N quantity of subsequent corrected NIR or IR frames.

104. The computer-implemented method of claim 103, the method further comprising:
(a) generating a first VIS image by adding a first VIS_DRK frame and a V quantity of VIS_DRK frames subsequent to the first VIS_DRK frame;
(b) overlaying the corrected NIR or IR image and the VIS image to form the first overlaid image;
(c) generating a second corrected NIR or IR image by adding a (N+1)^{th} or (N+2)th corrected NIR or IR frame and N quantity of subsequent corrected NIR or IR frames;
(d) generating a second VIS image by adding a second VIS frame and V quantity of VIS frames subsequent to the second VIS frame;
(e) overlaying the second corrected NIR or IR image and the second VIS image to form a second overlaid image; and
(f) forming a display image from two or more overlaid images, two or more NIR or IR images, or two or more VIS images, or any combination of the foregoing.

105.-137. (canceled)

138. A computer-implemented system comprising: a digital processing device comprising: at least one processor, an operating system configured to perform executable instructions, a memory, and a computer program including instructions executable by the digital processing device to create an application for forming a first overlaid image from laser induced fluorophore excitations, the application comprising:
(a) a module receiving a plurality of image frame sequences, each image frame sequence comprising:
(i) a VIS_DRK frame captured when the laser is in an off mode or in an on mode; and
(ii) a primary quantity of NIR or IR frames captured when the laser is in the on mode;
(b) a module correcting each NIR or IR frame by subtracting one correcting VIS_DRK frame; and
(c) a module generating a first NIR or IR image by adding a first corrected NIR or IR frame and N quantity of subsequent corrected NIR or IR frames.

139. The computer-implemented system of claim 138 comprising: a digital processing device further comprising:
(a) a module generating a first VIS_DRK image by adding a first VIS_DRK frame and a V quantity of VIS_DRK frames subsequent to the first VIS_DRK frame; and
(b) a module overlaying the NIR or IR image and the VIS_DRK image to form the first overlaid image.

140.-165. (canceled)

166. The system of claim 139, wherein the application further comprises:
(a) a module generating a second NIR or IR image by adding a (N+1)^{th} or (N+2)^{th} corrected NIR or IR frame and N quantity of subsequent corrected NIR or IR frames;
(b) a module generating a second VIS image by adding a second VIS frame and V quantity of VIS frames subsequent to the second VIS frame;
(c) a module overlaying the second NIR or IR image and the second VIS image to form a second overlaid image; and
(d) a module forming a display image from two or more overlaid images, two or more NIR or IR images, or two or more VIS images, or any combination of the foregoing.

167. (canceled)

168. (canceled)

169. (canceled)

170. (canceled)

171. (canceled)

172. (canceled)

173. A non-transitory computer-readable storage media encoded with a computer program including instructions executable by a processor to create an application for forming a first overlaid image from laser induced fluorophore excitations, the application comprising:
(a) a module receiving a plurality of image frame sequences, each image frame sequence comprising:
(i) a VIS frame captured when the laser is in an off mode or in an on mode; and
(ii) a primary quantity of NIR or IR frames captured when the laser is in the on mode;
(b) a module correcting each NIR or IR frame by subtracting one correcting VIS frame; and
(c) a module generating a first NIR or IR image by adding a first corrected NIR or IR frame and N quantity of subsequent corrected NIR or IR frames.

174. The media of claim 173, wherein the application further comprises:
(a) a module generating a first VIS image by adding a first VIS frame and a V quantity of VIS frames subsequent to the first VIS frame;
(b) a module overlaying the NIR or IR image and the VIS image to form the first overlaid image;
(c) a module generating a second NIR or IR image by adding a (N+1)^{th} or (N+2)^{th} corrected NIR or IR frame and N quantity of subsequent corrected NIR or IR frames;
(d) a module generating a second VIS image by adding a second VIS frame and V quantity of VIS frames subsequent to the second VIS frame;
(e) a module overlaying the second NIR or IR image and the second VIS image to form a second overlaid image; and
(f) a module forming a display image from two or more overlaid images, two or more NIR or IR images, or two or more VIS images, or any combination of the foregoing.

175.-200. (canceled)

201. (canceled)

202. (canceled)

203. (canceled)

204. (canceled)

205. (canceled)

206. (canceled)

207. (canceled)

208. A method of imaging an abnormal tissue, cancer, tumor, vasculature or structure in a sample from a subject, the method comprising producing an image of the vasculature or structure by imaging fluorescence using an imaging system, the system comprising:
(a) the imaging system of claim 1
(b).

209. A method of imaging an abnormal tissue, cancer, tumor, vasculature or structure in a sample from a subject, the method comprising producing an image of the abnormal tissue, cancer, tumor, vasculature or structure by imaging fluorescence using an imaging system method, the system method comprising:
(a) the method for imaging in accordance with claim 64.

210.-252. (canceled)

253. A method of imaging an abnormal tissue, cancer, tumor, vasculature or structure in a sample from a subject, the method comprising producing an image of the vasculature or structure by imaging fluorescence using an imaging system, the system comprising:
(a) the imaging platform of claim 50.
